SEQUENCE LISTING (194 sequences)

SEQ ID NO: 1 - protein, 109 aa, Artificial Sequence
  4D5 light chain variable domain
DIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKRT

SEQ ID NO: 2 - protein, 120 aa, Artificial Sequence
  4D5 heavy chain variable domain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 3 - protein, 35 aa, Artificial Sequence
  GNC4 leucine zipper
GRMKQLEDKVEELLSKNYHLENEVARLKKLVGERG

SEQ ID NO: 4 - protein, 5 aa, Artificial Sequence
  C-terminal of CDRH3 of 4D5
YAMDY

SEQ ID NO: 5 - protein, 7 aa, Artificial Sequence
  heavy chain CDR3
SRNAWAF

SEQ ID NO: 6 - protein, 11 aa, Artificial Sequence
  heavy chain CDR3
SRNLSENSYAM

SEQ ID NO: 7 - protein, 11 aa, Artificial Sequence
  heavy chain CDR3
SRAGWAGWYAM

SEQ ID NO: 8 - protein, 11 aa, Artificial Sequence
  heavy chain CDR3
SRAAKAGWYAM

SEQ ID NO: 9 - protein, 12 aa, Artificial Sequence
  heavy chain CDR3
SRSDGRDSAYAM

SEQ ID NO: 10 - protein, 13 aa, Artificial Sequence
  F63
SRXXXXXXXAMDY

SEQ ID NO: 11 - protein, 14 aa, Artificial Sequence
  F65
SRXXXXXXXYAMDY

SEQ ID NO: 12 - protein, 13 aa, Artificial Sequence
  F64
SRXXXXXXYAMDY

SEQ ID NO: 13 - protein, 13 aa, Artificial Sequence
  F66
SRXXXXXXYAMDY

SEQ ID NO: 14 - DNA, 51 bp, Artificial Sequence
  oligonucleotide F151
gcagcttctggcttcaccattavtrrtwmykmtatacactgggtgcgtcag

SEQ ID NO: 15 - DNA, 51 bp, Artificial Sequence
  oligonucleotide F152
gcagcttctggcttcaccattavtrrtwmykggatacactgggtgcgtcag

SEQ ID NO: 16 - DNA, 72 bp, Artificial Sequence
  oligonucleotide F153
aagggcctggaatgggttgstdggattwmtcctdmtrrcggtdmtactdactatgccgatagcgtcaagggc

SEQ ID NO: 17 - DNA, 72 bp, Artificial Sequence
  oligonucleotide F154
aagggcctggaatgggttgstdhtattwmtcctdmtrrcggtdmtactdactatgccgatagcgtcaagggc

SEQ ID NO: 18 - DNA, 1441 bp, Artificial Sequence
  single chain Fv
gaaatgagctgttgacaattaatcatcggctcgtataatgtgtggaattgtgagcggataacaatttcacacaggaaacagccagtccgtttaggtgttttcacgagcacttcaccaacaaggaccatagattatgaaaataaaaacaggtgcacgcatcctcgcattatccgcattaacgacgatgatgttttccgcctcggcttatgcatccgatatccagatgacccagtccccgagctccctgtccgcctctgtgggcgatagggtcaccatcacctgccgtgccagtcaggatgtgaatactgctgtagcctggtatcaacagaaaccaggaaaagctccgaagcttctgatttactcggcatccttcctctactctggagtcccttctcgcttctctggtagccgttccgggacggatttcactctgaccatcagcagtctgcagccggaagacttcgcaacttattactgtcagcaacattatactactcctcccacgttcggacagggtaccaaggtggagatcaaatcggatatgccgatggctgatccgaaccgtttccgcggtaagaacctggtttttcattctgagatctccgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctacaaatgaacagcttaagagctgaggacactgccgtctattattgtagccgctggggaggggacggcttctatgctatggactactggggtcaaggaacactagtcaccgtctccagcagtggcggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaa

SEQ ID NO: 19 - DNA, 1588 bp, Artificial Sequence
  single chain Fv with zipper domain
gaaatgagctgttgacaattaatcatcggctcgtataatgtgtggaattgtgagcggataacaatttcacacaggaaacagccagtccgtttaggtgttttcacgagcacttcaccaacaaggaccatagattatgaaaataaaaacaggtgcacgcatcctcgcattatccgcattaacgacgatgatgttttccgcctcggcttatgcatccgatatccagatgacccagtccccgagctccctgtccgcctctgtgggcgatagggtcaccatcacctgccgtgccagtcaggatgtgaatactgctgtagcctggtatcaacagaaaccaggaaaagctccgaagcttctgatttactcggcatccttcctctactctggagtcccttctcgcttctctggtagccgttccgggacggatttcactctgaccatcagcagtctgcagccggaagacttcgcaacttattactgtcagcaacattatactactcctcccacgttcggacagggtaccaaggtggagatcaaatcggatatgccgatggctgatccgaaccgtttccgcggtaagaacctggtttttcattctgagatctccgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctacaaatgaacagcttaagagctgaggacactgccgtctattattgtagccgctggggaggggacggcttctatgctatggactactggggtcaaggaacactagtcaccgtctccagcacatgcccgccgtgcccagcaccagaactgctgggcggccgcatgaaacagctagaggacaaggtcgaagagctactctccaagaactaccacctagagaatgaagtggcaagactcaaaaaacttgtcggggagcgcggaaagcttagtggcggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaa

SEQ ID NO: 20 - DNA, 2239 bp, Artificial Sequence
  Fab fragment
gaaatgagctgttgacaattaatcatcggctcgtataatgtgtggaattgtgagcggataacaatttcacacaggaaacagccagtccgtttaggtgttttcacgagcacttcaccaacaaggaccatagattatgaaaataaaaacaggtgcacgcatcctcgcattatccgcattaacgacgatgatgttttccgcctcggcttatgcatccgatatccagatgacccagtccccgagctccctgtccgcctctgtgggcgatagggtcaccatcacctgccgtgccagtcaggatgtgaatactgctgtagcctggtatcaacagaaaccaggaaaagctccgaagcttctgatttactcggcatccttcctctactctggagtcccttctcgcttctctggtagccgttccgggacggatttcactctgaccatcagcagtctgcagccggaagacttcgcaacttattactgtcagcaacattatactactcctcccacgttcggacagggtaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggtgccagctccggtatggctgatccgaaccgtttccgcggtaaggacctggcataactcgaggctgatcctctacgccggacgcatcgtggccctagtacgcaagttcacgtaaaaagggtaactagaggttgaggtgattttatgaaaaagaatatcgcatttcttcttgcatctatgttcgttttttctattgctacaaacgcgtacgctgagatctccgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctacaaatgaacagcttaagagctgaggacactgccgtctattattgtagccgctggggaggggacggcttctatgctatggactactggggtcaaggaaccctggtcaccgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacctcagtggcggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaa

SEQ ID NO: 21 - DNA, 2383 bp, Artificial Sequence
  Fab fragment with zipper domain
gaaatgagctgttgacaattaatcatcggctcgtataatgtgtggaattgtgagcggataacaatttcacacaggaaacagccagtccgtttaggtgttttcacgagcacttcaccaacaaggaccatagattatgaaaataaaaacaggtgcacgcatcctcgcattatccgcattaacgacgatgatgttttccgcctcggcttatgcatccgatatccagatgacccagtccccgagctccctgtccgcctctgtgggcgatagggtcaccatcacctgccgtgccagtcaggatgtgaatactgctgtagcctggtatcaacagaaaccaggaaaagctccgaagcttctgatttactcggcatccttcctctactctggagtcccttctcgcttctctggtagccgttccgggacggatttcactctgaccatcagcagtctgcagccggaagacttcgcaacttattactgtcagcaacattatactactcctcccacgttcggacagggtaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggtgccagctccggtatggctgatccgaaccgtttccgcggtaaggacctggcataactcgaggctgatcctctacgccggacgcatcgtggccctagtacgcaagttcacgtaaaaagggtaactagaggttgaggtgattttatgaaaaagaatatcgcatttcttcttgcatctatgttcgttttttctattgctacaaacgcgtacgctgagatctccgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctacaaatgaacagcttaagagctgaggacactgccgtctattattgtagccgctggggaggggacggcttctatgctatggactactggggtcaaggaaccctggtcaccgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacatgcccgccgtgcccagcaccagaactgctgggcggccgcatgaaacagctagaggacaaggtcgaagagctactctccaagaactaccacctagagaatgaagtggcaagactcaaaaaacttgtcggggagcgcggaaagcttagtggcggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaa

SEQ ID NO: 22 - protein, 12 aa, Artificial Sequence
  hinge sequence
TCPPCPAPELLG

SEQ ID NO: 23 - DNA, 57 bp, Artificial Sequence
  oligonucleotide F61
gcaacttattactgtcagcaanrtnrtrvmnnkccttdkacgttcggacagggtacc

SEQ ID NO: 24 - protein, 13 aa, Artificial Sequence
  CDRH3
SRWGGDGFYAMDY

SEQ ID NO: 25 - protein, 10 aa, Artificial Sequence
  F78
SRXXXXXFDY

SEQ ID NO: 26 - protein, 13 aa, Artificial Sequence
  F165
AXXXXXXXYAMDY

SEQ ID NO: 27 - protein, 13 aa, Artificial Sequence
  F166
AXWXXXXXXAMDY

SEQ ID NO: 28 - protein, 13 aa, Artificial Sequence
  F134
AXXXXXXXYAMDY

SEQ ID NO: 29 - protein, 14 aa, Artificial Sequence
  F136
AXWXXXXXXYAMDY

SEQ ID NO: 30 - protein, 14 aa, Artificial Sequence
  F137
AXXWXXXXXYAMDY

SEQ ID NO: 31 - protein, 14 aa, Artificial Sequence
  F138
AXXXWXXXXYAMDY

SEQ ID NO: 32 - protein, 14 aa, Artificial Sequence
  F142
AXXXXXXXWYAMDY

SEQ ID NO: 33 - protein, 14 aa, Artificial Sequence
  F155
AXWXXXXXXXAMDY

SEQ ID NO: 34 - protein, 14 aa, Artificial Sequence
  F156
AXXWXXXXXXAMDY

SEQ ID NO: 35 - protein, 14 aa, Artificial Sequence
  F157
AXXXWXXXXXAMDY

SEQ ID NO: 36 - protein, 14 aa, Artificial Sequence
  F158
AXXXXWXXXXAMDY

SEQ ID NO: 37 - protein, 14 aa, Artificial Sequence
  F160
AXXXXXXWXXAMDY

SEQ ID NO: 38 - protein, 14 aa, Artificial Sequence
  F160g
AXXXXXXXWXAMDY

SEQ ID NO: 39 - protein, 13 aa, Artificial Sequence
  F163a
AXXXXXXXXAMDY

SEQ ID NO: 40 - protein, 14 aa, Artificial Sequence
  F164a
ARXXXXXXXWAMDY

SEQ ID NO: 41 - protein, 14 aa, Artificial Sequence
  F164b
ARXXXXXXXXAMDY

SEQ ID NO: 42 - protein, 15 aa, Artificial Sequence
  F165a
ARXXXXXXXXYAMDY

SEQ ID NO: 43 - protein, 15 aa, Artificial Sequence
  F165b
ARXXXXXXXXXAMDY

SEQ ID NO: 44 - protein, 13 aa, Artificial Sequence
  F167
AXWXXXXXXAMDY

SEQ ID NO: 45 - protein, 13 aa, Artificial Sequence
  F135
AXWXXXXXXAMDY

SEQ ID NO: 46 - protein, 15 aa, Artificial Sequence
  F103
SRXXXXXXXXYAMDY

SEQ ID NO: 47 - protein, 13 aa, Artificial Sequence
  F66a
ARXXXXXXYAMDY

SEQ ID NO: 48 - protein, 13 aa, Artificial Sequence
  F66b
ARXXXXXXXAMDY

SEQ ID NO: 49 - protein, 12 aa, Artificial Sequence
  F66c
ARXXXXXYXMDY

SEQ ID NO: 50 - protein, 12 aa, Artificial Sequence
  F66d
ARXXXXXXXMDY

SEQ ID NO: 51 - protein, 11 aa, Artificial Sequence
  F66e
ARXXXXYXMDY

SEQ ID NO: 52 - protein, 11 aa, Artificial Sequence
  F66f
ARXXXXXXMDY

SEQ ID NO: 53 - protein, 13 aa, Artificial Sequence
  F66a1
ARXXXXXXYXMDY

SEQ ID NO: 54 - protein, 13 aa, Artificial Sequence
  F66b1
ARXXXXXXXXMDY

SEQ ID NO: 55 - protein, 14 aa, Artificial Sequence
  F66g
ARXXXXXXXXXMDY

SEQ ID NO: 56 - protein, 14 aa, Artificial Sequence
  F66h
ARXXXXXXXYXMDY

SEQ ID NO: 57 - protein, 15 aa, Artificial Sequence
  F66i
ARXXXXXXXXYXMDY

SEQ ID NO: 58 - protein, 15 aa, Artificial Sequence
  F66j
ARXXXXXXXXXXMDY

SEQ ID NO: 59 - protein, 10 aa, Artificial Sequence
  F171c
AXXXXXXFDY

SEQ ID NO: 60 - protein, 11 aa, Artificial Sequence
  F171d
AXXXXXXXFDY

SEQ ID NO: 61 - protein, 12 aa, Artificial Sequence
  F171e
AXXXXXXXXFDY

SEQ ID NO: 62 - protein, 9 aa, Artificial Sequence
  F171
AXXXXXFDY

SEQ ID NO: 63 - protein, 12 aa, Artificial Sequence
  F186
AXXXXXXXXFDY

SEQ ID NO: 64 - protein, 13 aa, Artificial Sequence
  F187
AXXXXXXXXXFDY

SEQ ID NO: 65 - protein, 16 aa, Artificial Sequence
  F190
AXXXXXXXXXXYAMDY

SEQ ID NO: 66 - protein, 15 aa, Artificial Sequence
  F190a
AXXXXXXXXXYXMDY

SEQ ID NO: 67 - protein, 17 aa, Artificial Sequence
  F190d
AXXXXXXXXXXXYXMDY

SEQ ID NO: 68 - protein, 11 aa, Artificial Sequence
  CDRH3
SRWKYATRYAM

SEQ ID NO: 69 - protein, 11 aa, Artificial Sequence
  CDRH3
SRSRGWWTAAM

SEQ ID NO: 70 - protein, 11 aa, Artificial Sequence
  CDRH3
SRASRDWYGAM

SEQ ID NO: 71 - protein, 5 aa, Artificial Sequence
  mVEGF-201 CDRH1
TTSNG

SEQ ID NO: 72 - protein, 8 aa, Artificial Sequence
  mVEGF-201 CDRH2
AYSSNYYR

SEQ ID NO: 73 - protein, 9 aa, Artificial Sequence
  mVEGF-201 CDRH3
ARWSRASFY

SEQ ID NO: 74 - protein, 5 aa, Artificial Sequence
  mVEGF-202 CDRH1
TTGTD

SEQ ID NO: 75 - protein, 8 aa, Artificial Sequence
  mVEGF-202 CDRH2
AITYDSYR

SEQ ID NO: 76 - protein, 9 aa, Artificial Sequence
  mVEGF-202 CDRH3
AKAGDREGY

SEQ ID NO: 77 - protein, 5 aa, Artificial Sequence
  mVEGF-203 CDRH1
TTDSG

SEQ ID NO: 78 - protein, 8 aa, Artificial Sequence
  mVEGF-203 CDRH2
GRSYSSNR

SEQ ID NO: 79 - protein, 9 aa, Artificial Sequence
  mVEGF-203 CDRH3
AKWPWYNAW

SEQ ID NO: 80 - protein, 5 aa, Artificial Sequence
  hFc-10 CDRH1
TNNYW

SEQ ID NO: 81 - protein, 7 aa, Artificial Sequence
  hFc-10 CDRH2
GYSYGTR

SEQ ID NO: 82 - protein, 9 aa, Artificial Sequence
  hFc-10 CDRH3
AKAXKGSLY

SEQ ID NO: 83 - protein, 5 aa, Artificial Sequence
  hFc-11 CDRH1
TTGNA

SEQ ID NO: 84 - protein, 5 aa, Artificial Sequence
  hFc-12 CDRH1
TNDYY

SEQ ID NO: 85 - protein, 5 aa, Artificial Sequence
  hFc-13 CDRH1
TSNTG

SEQ ID NO: 86 - protein, 5 aa, Artificial Sequence
  hFc-14 CDRH1
TTSYG

SEQ ID NO: 87 - protein, 7 aa, Artificial Sequence
  hFc-14 CDRH2
ASSYSYR

SEQ ID NO: 88 - protein, 9 aa, Artificial Sequence
  hFc-14 CDRH3
AKYXAREGX

SEQ ID NO: 89 - protein, 5 aa, Artificial Sequence
  hFc-15 CDRH1
TNNNS

SEQ ID NO: 90 - protein, 7 aa, Artificial Sequence
  hFc-15 CDRH2
GYNSGSR

SEQ ID NO: 91 - protein, 9 aa, Artificial Sequence
  hFc-15 CDRH3
AKWRTSWKY

SEQ ID NO: 92 - protein, 5 aa, Artificial Sequence
  hFc-16 CDRH1
TSSSA

SEQ ID NO: 93 - protein, 7 aa, Artificial Sequence
  hFc-16 CDRH2
AWSNGSR

SEQ ID NO: 94 - protein, 9 aa, Artificial Sequence
  hFc-16 CDRH3
AXTAGGAKY

SEQ ID NO: 95 - protein, 5 aa, Artificial Sequence
  hFc-17 CDRH1
TTNTW

SEQ ID NO: 96 - protein, 7 aa, Artificial Sequence
  hFc-17 CDRH2
GDYDGYR

SEQ ID NO: 97 - protein, 9 aa, Artificial Sequence
  hFc-17 CDRH3
AXWRWWGRY

SEQ ID NO: 98 - protein, 5 aa, Artificial Sequence
  hFc-18 CDRH1
TNGNY

SEQ ID NO: 99 - protein, 7 aa, Artificial Sequence
  hFc-18 CDRH2
GWSNGYR

SEQ ID NO: 100 - protein, 9 aa, Artificial Sequence
  hFc-18 CDRH3
ARYSGGRRY

SEQ ID NO: 101 - protein, 5 aa, Artificial Sequence
  hFc-19 CDRH1
TSNNA

SEQ ID NO: 102 - protein, 7 aa, Artificial Sequence
  hFc-19 CDRH2
GRSYNYR

SEQ ID NO: 103 - protein, 9 aa, Artificial Sequence
  hFc-19 CDRH3
AXGXTSGGY

SEQ ID NO: 104 - protein, 5 aa, Artificial Sequence
  hFc-20 CDRH1
TTSND

SEQ ID NO: 105 - protein, 7 aa, Artificial Sequence
  hFc-20 CDRH2
AWSYNYR

SEQ ID NO: 106 - protein, 9 aa, Artificial Sequence
  hFc-20 CDRH3
ARRSRWSRA

SEQ ID NO: 107 - protein, 5 aa, Artificial Sequence
  mVEGF-109 CDRH1
TGNSW

SEQ ID NO: 108 - protein, 6 aa, Artificial Sequence
  mVEGF-109 CDRH2
VATYYN

SEQ ID NO: 109 - protein, 7 aa, Artificial Sequence
  mVEGF-109 CDRH3
WGAKGTW

SEQ ID NO: 110 - protein, 5 aa, Artificial Sequence
  mVEGF-126 CDRH1
NADSA

SEQ ID NO: 111 - protein, 6 aa, Artificial Sequence
  mVEGF-126 CDRH2
YAYDYY

SEQ ID NO: 112 - protein, 7 aa, Artificial Sequence
  mVEGF-126 CDRH3
WGWTTNG

SEQ ID NO: 113 - protein, 5 aa, Artificial Sequence
  mVEGF-127 CDRH1
NDNTA

SEQ ID NO: 114 - protein, 6 aa, Artificial Sequence
  mVEGF-127 CDRH2
VSHDTY

SEQ ID NO: 115 - protein, 7 aa, Artificial Sequence
  mVEGF-127 CDRH3
WGWETDG

SEQ ID NO: 116 - protein, 6 aa, Artificial Sequence
  mVEGF-130 CDRH2
LDSSYD

SEQ ID NO: 117 - protein, 7 aa, Artificial Sequence
  mVEGF-130 CDRH3
SRAGYTY

SEQ ID NO: 118 - protein, 5 aa, Artificial Sequence
  mVEGF-136 CDRH1
NGKSS

SEQ ID NO: 119 - protein, 6 aa, Artificial Sequence
  mVEGF-136 CDRH2
WSYEAA

SEQ ID NO: 120 - protein, 7 aa, Artificial Sequence
  mVEGF-136 CDRH3
TSWSKPY

SEQ ID NO: 121 - protein, 5 aa, Artificial Sequence
  mVEGF-169 CDRH1
NTAYG

SEQ ID NO: 122 - protein, 6 aa, Artificial Sequence
  mVEGF-169 CDRH2
VTYDDT

SEQ ID NO: 123 - protein, 7 aa, Artificial Sequence
  mVEGF-169 CDRH3
WGWEANW

SEQ ID NO: 124 - protein, 5 aa, Artificial Sequence
  mVEGF-173 CDRH1
TGGSW

SEQ ID NO: 125 - protein, 6 aa, Artificial Sequence
  mVEGF-173 CDRH2
VYTYYD

SEQ ID NO: 126 - protein, 7 aa, Artificial Sequence
  mVEGF-173 CDRH3
WGAGGTW

SEQ ID NO: 127 - protein, 6 aa, Artificial Sequence
  mVEGF-174 CDRH2
VSDYYD

SEQ ID NO: 128 - protein, 7 aa, Artificial Sequence
  mVEGF-174 CDRH3
WGSGYTW

SEQ ID NO: 129 - protein, 5 aa, Artificial Sequence
  mVEGF-176 CDRH1
SAGYD

SEQ ID NO: 130 - protein, 6 aa, Artificial Sequence
  mVEGF-176 CDRH2
LAYAYN

SEQ ID NO: 131 - protein, 7 aa, Artificial Sequence
  mVEGF-176 CDRH3
AAAWASY

SEQ ID NO: 132 - protein, 5 aa, Artificial Sequence
  mVEGF-179 CDRH1
TTESG

SEQ ID NO: 133 - protein, 6 aa, Artificial Sequence
  mVEGF-179 CDRH2
VYHDKY

SEQ ID NO: 134 - protein, 7 aa, Artificial Sequence
  mVEGF-179 CDRH3
WWYSWNW

SEQ ID NO: 135 - DNA, 389 bp, Artificial Sequence
  VHH anti-HCG monobody
gatgttcagttgcaggaatcaggcggtggcttgtacaggccggaggttcgttgcgtttgtcctgtgctgcctcgggtcgtactggttctacttatgatatgggctggtttcgtcaggctccgggtaaagaacgtgaatcggttgccgccattaactgggattcggctcgtacttactatgcttcgtccgtccgtggtcgttttactatttcacgtgataatgccaaaaaaactgtctatttgcagatgaattcattgaaaccagaagatactgccgtctatacttgtggtgctggtgaaggcggtacttgggattcttggggtcagggtacccaggtcactgtctcctctgccggtggtatggattataaagatgatgatgataaatga

SEQ ID NO: 136 - protein, 129 aa, Artificial Sequence
  VHH anti-HCG monobody
DVQLQESGGGLVQAGGSLRLSCAASGRTGSTYDMGWFRQAPGKERESVAAINWDSARTYYASSVRGRFTISRDNAKKTVYLQMNSLKPEDTAVYTCGAGEGGTWDSWGQGTQVTVSSAGGMDYKDDDDK

SEQ ID NO: 137 - protein, 23 aa, Artificial Sequence
  CDRH3 17 amino acid insert
CGAGXXXXXXXXXXXXXXXXXWG

SEQ ID NO: 138 - protein, 4 aa, Artificial Sequence
  VHH RIG N terminal sequence
RIXC

SEQ ID NO: 139 - protein, 5 aa, Artificial Sequence
  VHH RIG C terminal sequence
CWVTW

SEQ ID NO: 140 - protein, 21 aa, Artificial Sequence
  CDRH3
XXXXXXXXXXXXXXXXXXXXX

SEQ ID NO: 141 - protein, 23 aa, Artificial Sequence
  CDRH3
XXXXXXXXXXXXXXXXXXXXXXX

SEQ ID NO: 142 - protein, 25 aa, Artificial Sequence
  CDRH3
XXXXXXXXXXXXXXXXXXXXXXXXX

SEQ ID NO: 143 - protein, 24 aa, Artificial Sequence
  CDRH3
RXXRXXXXXXXXXXXXXXXXXXXX

SEQ ID NO: 144 - protein, 23 aa, Artificial Sequence
  CDRH3
RXXXXXXXXXXXXXXXXWXXXXX

SEQ ID NO: 145 - protein, 17 aa, Artificial Sequence
  CDRH3
RIXXXXXXXXXXXWVXX

SEQ ID NO: 146 - protein, 17 aa, Artificial Sequence
  CDRH3
VLXXXXXXXXXFXRVXX

SEQ ID NO: 147 - protein, 17 aa, Artificial Sequence
  CDRH3
RLXXXXXXXXXWXXXXX

SEQ ID NO: 148 - protein, 17 aa, Artificial Sequence
  CDRH3
LLXXXXXXXXXWXXLXX

SEQ ID NO: 149 - protein, 16 aa, Artificial Sequence
  CDRH3
RIXCXXXXXXXCWVXX

SEQ ID NO: 150 - protein, 4 aa, Artificial Sequence
  N terminal sequence
RXXR

SEQ ID NO: 151 - DNA, 60 bp, Artificial Sequence
  ala scan wild type CDRH3
gccgtctatacttgtggtgctggtgmagstgstrctksggmtkcctggggtcagggtacc

SEQ ID NO: 152 - DNA, 55 bp, Artificial Sequence
  framework scan of residue 37-47 of wild type
gatatgggctggnnscgtcaggctccgggtaaagaannsgaannsgttgccgcca

SEQ ID NO: 153 - DNA, 60 bp, Artificial Sequence
  framework scan of residue 91 of wild type
gatactgccgtctatnnstgtggtgctggtgaaggcggtacttgggattcttggggtcag

SEQ ID NO: 154 - DNA, 87 bp, Artificial Sequence
  NNS library
gccgtctatacttgtggtgctggtnnsnnsnnsnnsnnsnnsnnsnnsnnsnnsnnsnnsnnsnnsnnsnnsnnstggggtcagggt

SEQ ID NO: 155 - DNA, 87 bp, Artificial Sequence
  RIG ala scan
gccgtctatacttgtggtgctggtsstrytgstsstkccgytkytrmcsytsstsstgmakccksggytrctksgtggggtcagggt

SEQ ID NO: 156 - DNA, 87 bp, Artificial Sequence
  VLK ala scan
gccgtctatacttgtggtgctggtgytsytrmasstsstgstkcckccgytgstrytkytrctsstgytsmakcctggggtcagggt

SEQ ID NO: 157 - DNA, 87 bp, Artificial Sequence
  LLR ala scan
gccgtctatacttgtggtgctggtsytsytsstsstgstgytrmcgcgrctscarmcksgkytgstsytgytgsttggggtcagggt

SEQ ID NO: 158 - DNA, 86 bp, Artificial Sequence
  RLV ala scan
gccgtctatacttgtggtgctggtsstsytgytrmcgstsytkccgstsytgytkccksggmarygscasytgcgtggggtcaggg

SEQ ID NO: 159 - protein, 4 aa, Artificial Sequence
  N terminal sequence of CDRH3 scaffold
RIXC

SEQ ID NO: 160 - protein, 4 aa, Artificial Sequence
  C terminal sequence of CDRH3 scaffold
FXRV

SEQ ID NO: 161 - protein, 4 aa, Artificial Sequence
  C terminal sequence of CDRH3 scaffold
WXXL

SEQ ID NO: 162 - protein, 4 aa, Artificial Sequence
  C terminal sequence of CDRH3 scaffold
WXMP

SEQ ID NO: 163 - protein, 17 aa, Artificial Sequence
  CDRH3
RIGRSVFNLRRESWVTW

SEQ ID NO: 164 - protein, 17 aa, Artificial Sequence
  CDRH3
LLRRGVNATPNWFGLVG

SEQ ID NO: 165 - protein, 17 aa, Artificial Sequence
  CDRH3
VLKRRGSSVAIFTRVQS

SEQ ID NO: 166 - protein, 17 aa, Artificial Sequence
  CDRH3
RLVNGLSGLVSWEMPLA

SEQ ID NO: 167 - protein, 17 aa, Artificial Sequence
  CDRH3
FVAGPWWWRWRTPSGVA

SEQ ID NO: 168 - protein, 17 aa, Artificial Sequence
  CDRH3
VLELRSSGGNARWMSLY

SEQ ID NO: 169 - protein, 17 aa, Artificial Sequence
  CDRH3
LRISPYAFWLGTWAPSY

SEQ ID NO: 170 - protein, 17 aa, Artificial Sequence
  CDRH3
LWTRARSWRWWWRREQF

SEQ ID NO: 171 - protein, 17 aa, Artificial Sequence
  CDRH3
WRSWISSILGLRTWWYA

SEQ ID NO: 172 - protein, 17 aa, Artificial Sequence
  CDRH3
KSTRWRAGHGRTFHWLS

SEQ ID NO: 173 - protein, 14 aa, Artificial Sequence
  F139
AXXXXWXXXYAMDY

SEQ ID NO: 174 - protein, 13 aa, Artificial Sequence
  F140
AXXXXWXXYAMDY

SEQ ID NO: 175 - protein, 14 aa, Artificial Sequence
  F141
AXXXXXXWXYAMDY

SEQ ID NO: 176 - protein, 14 aa, Artificial Sequence
  F142
AXXXXXXXWYAMDY

SEQ ID NO: 177 - protein, 9 aa, Artificial Sequence
  F170
AXXXXXFDY

SEQ ID NO: 178 - protein, 12 aa, Artificial Sequence
  F171a
AXXXXXXXXFDY

SEQ ID NO: 179 - protein, 13 aa, Artificial Sequence
  F171b
AXXXXXXXXXFDY

SEQ ID NO: 180 - protein, 14 aa, Artificial Sequence
  F181
AXXXXXXXXXAMDY

SEQ ID NO: 181 - protein, 14 aa, Artificial Sequence
  F179
AXXXXXXXXYAMDY

SEQ ID NO: 182 - protein, 15 aa, Artificial Sequence
  F182
AXXXXXXXXXXAMDY

SEQ ID NO: 183 - protein, 11 aa, Artificial Sequence
  F183
AXXXXXYAMDY

SEQ ID NO: 184 - protein, 12 aa, Artificial Sequence
  F184
AXXXXXXYAMDY

SEQ ID NO: 185 - protein, 9 aa, Artificial Sequence
  F185
AXXXXXFXY

SEQ ID NO: 186 - protein, 15 aa, Artificial Sequence
  F190b
AXXXXXXXXXXXMDY

SEQ ID NO: 187 - protein, 16 aa, Artificial Sequence
  F190c
AXXXXXXXXXXXXMDY

SEQ ID NO: 188 - protein, 17 aa, Artificial Sequence
  F190e
AXXXXXXXXXXXXXMDY

SEQ ID NO: 189 - protein, 7 aa, Artificial Sequence
  4D5CDH3
WGGDGFY

SEQ ID NO: 190 - protein, 13 aa, Artificial Sequence
  F59
SRWGXXXXXAMDY

SEQ ID NO: 191 - protein, 5 aa, Artificial Sequence
  mVEGF 130
NADSA

SEQ ID NO: 192 - protein, 5 aa, Artificial Sequence
  mVEGF 174
TGGSW

SEQ ID NO: 193 - protein, 25 aa, Artificial Sequence
  CDRH3
XXXXXXXXXXXXXXXXXXXXXXXXX

SEQ ID NO: 194 - protein, 15 aa, Artificial Sequence
  CDRH3
RIXCXXXXXXCWVXX

(19) United States
(12) Patent Application Publication
(10) Pub. No.: US 2005/0079574 A1
(43) Pub. Date: Apr. 14, 2005

(54) Synthetic antibody phage libraries

(75) Inventor: Christopher J. Bond, San Francisco, CA (US)

Correspondence Address: MERCHANT & GOULD PC, P.O. BOX 2903, MINNEAPOLIS, MN, 55402-0903, US

(73) Assignee: Genentech, Inc., San Francisco, CA (US)

(21) Appl. No.: 10/759,731

(22) Filed: Jan. 16, 2004

Related U.S. Application Data
(60) Provisional application No. 60/441,059, filed on Jan. 16, 2003.
(60) Provisional application No. 60/488,610, filed on Jul. 18, 2003.
(60) Provisional application No. 60/510,314, filed on Oct. 8, 2003.

(51) Int. Cl. C07H 21/04; C07K 16/28; C07K 14/705; C12N 5/06

(52) U.S. Cl. 435/69.1; 435/320.1; 435/325; 435/328; 530/350; 530/388.22; 536/23.53

(57) ABSTRACT

The invention provides immunoglobulin polypeptides comprising variant amino acids in CDRs of antibody variable domains. In one embodiment, the polypeptide is a variable domain of a monobody and has a variant CDRH3 region. These polypeptides provide a source of great sequence diversity that can be used as a source for identifying novel antigen binding polypeptides. The invention also provides these polypeptides as fusion polypeptides to heterologous polypeptides such as at least a portion of phage or viral coat proteins, tags and linkers. Libraries comprising a plurality of these polypeptides are also provided. In addition, methods of and compositions for generating and using these polypeptides and libraries are provided.

### CROSSREFERENCE TO RELATED APPLICATIONS

This applications claims priority under 35 U.S.C. 119 (e) to U.S. Ser. No. 60/441,059 filed Jan. 16, 2003, U.S. Ser. No. 60/488,610, filed Jul. 18, 2003, and U.S. Ser. No. 60/510,314, filed Oct. 8, 2003 which are hereby incorporated by reference.

### FIELD OF THE INVENTION

The invention generally relates to libraries of antibodies or antibody variable domains. The libraries include a plurality of different antibody variable domains generated by creating diversity in the CDR regions. In particular, diversity in CDR regions is designed to maximize the diversity while minimizing the structural perturbations of the antibody variable domain. The invention also relates to fusion polypeptides of one or more antibody variable domain and a heterologous protein such as a coat protein of a virus. The invention also relates to replicable expression vectors which include a gene encoding the fusion polypeptide, host cells containing the expression vectors, a virus which displays the fusion polypeptide on the surface of the virus, libraries of the virus displaying a plurality of different fusion polypeptides on the surface of the virus and methods of using those compositions. The methods and compositions of the invention are useful for identifying novel antibodies and antibody variable domains that can be used therapeutically or as reagents.

### BACKGROUND

Phage display technology has provided a powerful tool for generating and selecting novel proteins which bind to a ligand, such as an antigen. Using the techniques of phage display allows the generation of large libraries of protein variants which can be rapidly sorted for those sequences that bind to a target molecule with high affinity. Nucleic acids encoding variant polypeptides are fused to a nucleic acid sequence encoding a viral coat protein, such as the gene III protein or the gene VIII protein. Monovalent phage display systems where the nucleic acid sequence encoding the protein or polypeptide is fused to a nucleic acid sequence encoding a portion of the gene III protein have been developed. (Bass, S., Proteins, 8:309 (1990); Lowman and Wells, Methods: A Companion to Methods in Enzymology, 3:205 (1991)). In a monovalent phage display system, the gene fusion is expressed at low levels and wild type gene III proteins are also expressed so that infectivity of the particles is retained. Methods of generating peptide libraries and screening those libraries have been disclosed in many patents (e.g. U.S. Pat. No. 5,723,286, U.S. Pat. No. 5,432, 018, U.S. Pat. No. 5,580,717, U.S. Pat. No. 5,427,908 and U.S. Pat. No. 5,498,530).

The demonstration of expression of peptides on the surface of filamentous phage and the expression of functional antibody fragments in the periplasm of E. coli was important in the development of antibody phage display libraries. (Smith et al., Science (1985), 228:1315; Skerra and Pluckthun, Science (1988), 240:1038)., Libraries of antibodies or antigen binding polypeptides have been prepared in a number of ways including by altering a single gene by inserting random DNA sequences or by cloning a family of related genes. Methods for displaying antibodies or antigen binding fragment or polypeptides using phage display have been described in U.S. Pat. Nos. 5,750,373, 5,733,743, 5,837,242, 5,969,108, 6,172,197, 5,580,717, and 5,658,727. The library is then screened for expression of antibodies or antigen binding proteins with the desired characteristics.

Phage display technology has several advantages over conventional hybridoma and recombinant methods for preparing antibodies with the desired characteristics. This technology allows the development of large libraries of antibodies with diverse sequences in less time and without the use of animals. Preparation of hybridomas or preparation of humanized antibodies can easily require several months of preparation. In addition, since no immunization is required, phage antibody libraries can be generated for antigens which are toxic or have low antigenicity (Hogenboom, Immunotechniques (1988), 4:1-20). Phage antibody libraries can also be used to generate and identify novel human antibodies.

Human antibodies have become very useful as therapeutic agents for a wide variety of conditions. For example, humanized antibodies to HER-2, a tumor antigen, are useful in the diagnosis and treatment of cancer. Other antibodies, such as anti-INF-γ antibody, are useful in treating inflammatory conditions such as Crohn's disease. Phage display libraries have been used to generate human antibodies from immunized, non-immunized humans, germ line sequences, or naïve B cell Ig repertories (Barbas & Burton, Trends Biotech (1996), 14:230; Griffiths et al., EMBO J. (1994), 13:3245; Vaughan et al., Nat. Biotech. (1996), 14:309; Winter EP 0368 684 B1). Naïve, or nonimmune, antigen binding libraries have been generated using a variety of lymphoidal tissues. Some of these libraries are commercially available, such as those developed by Cambridge Antibody Technology and Morphosys (Vaughan et al., Nature Biotech 14:309 (1996); Knappik et al., J. Mol. Biol. 296:57 (1999)). However, many of these libraries have limited diversity.

The ability to identify and isolate high affinity antibodies from a phage display library is important in isolating novel human antibodies for therapeutic use. Isolation of high affinity antibodies from a library is dependent on the size of the library, the efficiency of production in bacterial cells and the diversity of the library. See, for e.g., Knappik et al., J. Mol. Biol. (1999), 296:57. The size of the library is decreased by inefficiency of production due to improper folding of the antibody or antigen binding protein and the presence of stop codons. Expression in bacterial cells can be inhibited if the antibody or antigen binding domain is not properly folded. Expression can be improved by mutating residues in turns at the surface of the variable/constant interface, or at selected CDR residues. (Deng et al., J. Biol. Chem. (1994), 269:9533, Ulrich et al., PNAS (1995), 92:11907-11911; Forsberg et al., J. Biol. Chem. (1997), 272 :12430). The sequence of the framework region is important in providing for proper folding when antibody phage libraries are produced in bacterial cells.

Generating a diverse library of antibodies or antigen binding proteins is also important to isolation of high affinity antibodies. Libraries with diversification in limited CDRs have been generated using a variety of approaches. See, for e.g., Tomlinson, Nature Biotech. (2000), 18:989-994. CDR3 regions are of interest in part because they often are found to participate in antigen binding. CDR3 regions on the heavy chain vary greatly in size, sequence and structural conformation.

Others have also generated diversity by randomizing CDR regions of the variable heavy and light chains using all 20 amino acids at each position. It was thought that using all 20 amino acids would result in a large diversity of sequences of variant antibodies and increase the chance of identifying novel antibodies. (Barbas, PNAS 91:3809 (1994); Yelton, D E, J. Immunology, 155:1994 (1995); Jackson, J. R., J. Immunology, 154:3310 (1995) and Hawkins, R E, J. Mol. Biology, 226:889 (1992)).

There have also been attempts to create diversity by restricting the group of amino acid substitutions in some CDRs to reflect the amino acid distribution in naturally occurring antibodies. See, Garrard & Henner, Gene (1993), 128:103; Knappik et al., J. Mol. Biol. (1999), 296:57. However, these attempts have had varying success and have not been applied in a systematic and quantitative manner. Creating diversity in the CDR regions while minimizing the number of amino acid changes has been a challenge.

There is a need to isolate novel high affinity antibodies for clinical uses, for example therapeutic and diagnostic uses. To meet this need, there remains a need to generate a highly diverse library of antibody variable domains that can be expressed in high yield in cells. The invention described herein meets this need and provides other benefits.

### SUMMARY OF INVENTION

The present invention provides methods of systematically and efficiently generating polypeptides comprising diversified CDRs. Unlike conventional methods that propose that adequate diversity of target binders can be generated only if a particular CDR(s), or all CDRs should be diversified, and unlike conventional notions that adequate diversity is dependent upon the broadest range of amino acid substitutions (generally by substitution using all or most of the 20 amino acids), the invention provides methods capable of generating high quality target binders that are not necessarily dependent upon diversifying a particular CDR(s) or a particular number of CDRs of a reference polypeptide or source antibody. The invention is based, at least in part, on the surprising and unexpected findings that highly diverse libraries of high quality can be generated by systematic and selective substitutions of a minimal number of amino acid positions with a minimal number of amino acid residues. Methods of the invention are convenient, based on objective and systematic criteria, and rapid. Candidate binder polypeptides generated by the invention possess high-quality target binding characteristics. The invention also provides unique dimerization/multimerization techniques that further enhance library characteristics, and the binding characteristics of candidate fusion polypeptide binders therein.

In particular, fusion polypeptides comprising diversified CDR(s) and a heterologous polypeptide sequence (preferably that of at least a portion of a viral polypeptide) are generated, individually and as a plurality of unique individual polypeptides that are candidate binders to targets of interest. Compositions (such as libraries) comprising such polypeptides find use in a variety of applications, in particular as large and diverse pools of candidate immunoglobulin polypeptides (in particular, antibodies and antibody fragments) that bind to targets of interest. The invention encompasses various aspects, including polypeptides generated according to methods of the invention, and systems, kits and articles of manufacture for practicing methods of the invention, and/or using polypeptides and/or compositions of the invention.

Accordingly, in one aspect of the invention, a polypeptide comprising a variant CDRH3 ( CDR3 of the heavy chain) region is provided. A CDRH3 region is designed to provide for amino acid sequence diversity at certain positions while minimizing the structural perturbations. Diversity is limited at structural amino acid positions. The polypeptide comprises a variant CDRH3, wherein the variant CDRH3 comprises at least one structural amino acid position wherein said structural amino acid position has a variant amino acid, wherein the variant amino acid is an amino acid found at that position in a randomly generated CDRH3 population at a frequency of at least one standard deviation above the average frequency for any amino acid at that position, and at least one non-structural position, wherein the non-structural position has a variant amino acid.

A polypeptide or source antibody can include an antibody, antibody variable domain, antigen binding fragment thereof, a monobody, variable domain of a monobody (VHH), a monobody or antibody variable domain obtained from a naïve or synthetic library, camelid antibodies, naturally occurring antibody or monobody, synthetic antibody or monobody, recombinant antibody or monobody, humanized antibody or monobody, germline derived antibody or monobody, chimeric antibody or monobody, and affinity matured antibody or monobody.

Monobodies can bind to antigens in the absence of a light chain and may be utilized, inter alia, for modular antigen binding domains in bispecific antibodies, intracellular antibodies, proteomics, and/or novel therapeutic agents. In one embodiment, the polypeptide is an antibody variable domain that can bind to a molecule that specifically binds to folded polypeptide and does not bind to unfolded polypeptide, such as protein A. In another embodiment, the polypeptide is an antibody variable domain that is a member of the Vh3 subgroup and preferably, is a variable domain of a camelid monobody.

A structural amino acid position refers to an amino acid position in a CDRH3 region of a polypeptide that contributes to the stability of the structure of the polypeptide such that the polypeptide retains at least one biological function such as specifically binding to a molecule that binds to folded polypeptide and does not bind to unfolded polypeptide, such as Protein A and or binding to antigen. Structural amino acid positions of a CDRH3 region are identified as amino acid positions less tolerant to amino acid substitutions without affecting the structural stability of the polypeptide. Amino acid positions less tolerant to amino acid substitutions can be identified using a method such as alanine scanning mutagenesis or shotgun scanning as described in WO 01/44463 and analyzing the effect of loss of the wild type amino acid on structural stability at positions in the CDRH3.

In some embodiments, structural amino acid positions in a CDRH3 are located near the N and C terminus of the CDRH3 allowing for a central portion that can be varied. The variant CDRH3 regions can have a N terminal flanking region in which some or all of the amino acid positions have limited diversity, a central portion comprising at least one or more non-structural amino acid positions that can be varied in length and sequence, and C-terminal flanking sequence in which some or all amino acid positions have limited diversity. The length of the CDRH3 region is selected to reflect the length of CDRH3 regions found in naturally occurring antibody variable domains found in humans, camelids and/or mice, for example, as shown in FIG. 41. In some embodiments, the length of CDRH3 is from about 3 amino acids up to about 24 amino acids. The length of the N terminal flanking region, central portion, and C-terminal flanking region is determined by selecting the length of CDRH3, randomizing each position and identifying the structural amino acid positions at the N and C-terminal ends of the CDRH3. The length of the N and C terminal flanking sequences should be long enough to include at least one structural amino acid position in each flanking sequence. In some embodiments, the length of the N-terminal flanking region is at least about from 1 to 4 contiguous amino acids, the central portion of at least one non-structural position(s) can vary from about 1 to 20 contiguous amino acids, and the C-terminal portion is at least about from 1 to 6 contiguous amino acids.

For example, in a 17 amino acid CDRH3 region, structural amino acid positions are selected from the group consisting of the first N-terminal amino acid, the second N-terminal amino acid, at least one of the last 6 amino acids at the C-terminus of a heavy chain CDRH3 or mixtures thereof. The central portion has a length of 9 amino acids that can vary in sequence. In another embodiment, at least one structural amino acid position is one or both of the first two amino acid positions at the N-terminus of a heavy chain CDRH3. In another embodiment, said at least one structural amino acid position is a third, fourth and/or sixth amino acid position counting from the C-terminus.

Once at least one structural amino acid position in a heavy chain CDRH3 is identified, a limited set of amino acids is selected for substitution at this position. The diversity at least one structural amino acid position is limited to provide for maximal diversity while minimizing the structural perturbations. The number of amino acids that are substituted at a structural amino acid position is no more than about 1 to 7, about 1 to 4, or about 1 to 2 amino acids. In some embodiments, a variant amino acid at a structural amino acid position is encoded by one or more nonrandom codon sets. The nonrandom codon sets encode multiple amino acids for a particular positions, for example, about 1 to 7, about 1 to 4 amino acids or about 1 to 2 amino acids. The amino acids that are substituted at structural positions are those that are found at that position in a randomly generated CDRH3 population at a frequency at least one standard deviation above the average frequency for any amino acid at the position.

In one embodiment, the polypeptide is an antibody variable domain of a monobody. In some embodiments, the limited set of amino acids substituted at a structural amino acid position in a CDRH3 are those that provide for stabilization of the protein at the former light chain interface. The limited set of amino acids at a structural amino acid position are selected from the group consisting of a hydrophobic amino acid and/or arginine. The hydrophobic amino acids are preferably selected from the group consisting of leucine, isoleucine, valine, tryptophan, tyrosine, and phenylalanine. In a VHH variable domain, the structural amino acids positions in a CDRH3 are preferably substituted with hydrophobic amino acids to stabilize the VHH in the absence of the light chain at the former light chain interface.

In one embodiment, a polypeptide comprises a variant CDRH3 wherein the said at least one structural amino acid position is a first N-terminal amino acid position that has a variant amino acid selected from the group consisting of amino acids R, L, and V.

In another embodiment, a polypeptide comprises a variant CDRH3 comprising at least one structural amino acid position, wherein the structural amino acid position is the first and second amino acid positions at the N-terminus, wherein the first amino acid position has a variant amino acid selected from the group consisting of R, L, and V, and the second amino acid position at the N-terminus has a variant amino acid selected from the group consisting of I and L.

Another embodiment is a polypeptide comprising a variant CDRH3 comprising at least one structural amino acid position, wherein said at least one structural amino acid position is the third, fourth and/or sixth position from the C-terminus of the CDRH3, wherein the CDRH3 is at least 8 amino acids long and in one embodiment, is up to 24 amino acids long; wherein the fourth position from the C-terminus has a variant amino acid selected from the group consisting of M, R, G, and W, and the third amino acid position from the C-terminus has a variant amino acid selected from the group consisting of P, V, L, and W, and the sixth position from the C-terminus has a variant amino acid selected from the group consisting of E, W, and F. In an embodiment, at least one of the third, fourth, and/or sixth position from the C terminal has a tryptophan.

The variant CDRH3 is typically positioned between the third framework region and the fourth framework region in an antibody variable domain and may be inserted within a CDRH3 in a source variable domain. Typically, when the variant CDRH3 is inserted into a source or wild type CDRH3 the variant CDRH3 replaces all or a part of the source or wild type CDRH3. The location of insertion of the CDRH3 can be determined by comparing the location of CDRH3s in naturally occurring antibody variable domains. In one embodiment, a comparison of the naturally occurring antibody variable domains of monobodies indicated that the synthetic CDRH3 may be inserted after amino acid position 95 and before amino acid position 103 of wild type VHH CDRH3.

The amino acid numbering may vary depending on the exact location of insertion of the CDRH3 region. In one embodiment, a 17 amino acid CDRH3 region is inserted in the CDRH3 of a VHH of a monobody between amino acid residues 95 (amino acid glycine) and 103 (amino acid tryptophan) (numbering according to Kabat, Sequences of Proteins of immunological Interest, 1991, NIH publication No. 32919). The 17 residue CDRH3, CGAGXXXXXXXXXXXXXXXXXWG, is then numbered starting at amino acid position of the first X as position 96, 97, 98, 99, 100, 100a, 100b, 100c, 100d, 100e, 100f, 100g, 100h, 100i, 100j, 101 and 102 (SEQ ID NO:137) as shown in FIG. 37c. The two amino acid positions at the N-terminus in this embodiment are 96 and 97, respectively. The last 6 amino acids from the C-terminus in this embodiment are 100g, 100h, 100i, 100j, 101, and 102.

The variant CDRH3 region also comprises a non-structural amino acid position that has a variant amino acid. Non-structural amino acid positions can vary in sequence and in length. In some embodiments, one or more non-structural amino acid positions are located in between the N terminal and C terminal flanking regions. Said at least one non-structural position is or comprises a contiguous sequence of about 1 to 20 amino acids; more preferably 1 to 17 amino acids; more preferably 5 to 15 amino acids and most preferably about 9 to 12 amino acids. The non-structural amino acid positions can be substituted randomly with any of the naturally occurring amino acids or with selected amino acids. In some embodiments, said at least one non-structural position can have a variant amino acid encoded by a random codon set or a nonrandom codon. The nonrandom codon set preferably encodes amino acids that are commonly occurring at that position in naturally occurring known antibodies. Examples of nonrandom codon sets include DVK, XYZ, and NVT.

When the polypeptide is an antibody heavy chain variable domain, diversity at framework region residues may also be limited in order to preserve structural stability of the polypeptide. The diversity in framework regions is limited at those positions that form the light chain interface. Amino acids in positions at the light chain interface can be modified to provide for binding of the heavy chain to antigen in absence of the light chain. The amino acid positions that are found at the light chain interface in the VHH of camelid monobodies include amino acid position 37, amino acid position 45, amino acid position 47 and amino acid position 91. Heavy chain interface residues are those residues that are found on the heavy chain but have at least one side chain atom that is within 6 angstroms of the light chain. The amino acid positions in the heavy chain that are found at the light chain interface in human heavy chain variable domains include positions 37, 39, 44, 45, 47, 91, and 103.

In one embodiment, the polypeptide is a variable domain of a monobody and further comprises a framework 2 region of a heavy chain variable domain of a naturally occurring monobody, wherein amino acid position 37 of framework 2 has a phenylalanine, tyrosine, valine or tryptophan in that position. In another embodiment, the monobody variable domain further comprises a framework 2 region of a heavy chain, wherein the amino acid position 45 of the framework 2 region has an arginine, tryptophan, phenylalanine or leucine in that position. In another embodiment, the monobody variable domain further comprises a framework 2 region, wherein the amino acid position 47 has a phenylalanine, leucine, tryptophan or glycine residue in that position. In another embodiment, the monobody further comprises a framework 3 region of a heavy chain, wherein amino acid position 91 of the framework 3 region is a phenylalanine, threonine, or tyrosine.

In another aspect of the invention, CDRH1 and CDRH2 residues are those of naturally occurring antibody variable domains or can be those from known antibody variable domains that bind to a particular antigen whether naturally occurring or synthetic. In some embodiments, the CDRH1 And CDRH2 regions may be randomized at each position. It will be understood by those of skill in the art that antigen binding molecules isolated using the methods of the invention may require further optimization of antigen binding affinity using standard methods. In one embodiment, the CDRH1 and CDRH2 sequences are those that are from the closest human germline sequence for CDRH1 and CDRH2 of the naturally occurring camelid monobody sequences.

The invention also provides for 1) fusion polypeptides; 2) fusion polypeptides to viral coat proteins or portions thereof; 3) polynucleotides encoding any of the polypeptides; 4) replicable expression vectors comprising a polynucleotide encoding the polypeptides of the invention; 5) host cells comprising the vectors; 6) a library comprising a plurality of vectors of the invention and 7) a population of variant polypeptides or polynucleotides of the invention.

Another aspect of the invention concerns CDRH3 regions that are designed to generate libraries or populations of variant polypeptides that may provide for identification of novel peptides binding to target molecules, including antigens. In a CDRH3 designed in accord with the invention, amino acid positions that are primarily structural have limited diversity and other amino acids not as important for structural stability can be varied both in length and sequence diversity. CDRH3 regions can be designed so that the diversity is limited at structural amino acid positions and varied at non-structural amino acid positions that can vary in size, for example, from 1 to 20 amino acids, preferably 1 to 17 amino acids, preferably 5 to 15 amino acids and more preferably, 9-12 amino acids. In a preferred embodiment, a CDRH3 is selected that has structural amino acid positions at the N and C-terminal ends of the CDRH3 and has a central portion of the CDRH3 that can be varied more extensively, for example, using random or nonrandom codon sets as described herein.

Polypeptides comprising a CDRH3 having such a design include camelid monobody, VHH, camelized antibodies, antibody or monobody variable domain obtained from a naïve or synthetic library, naturally occurring antibody or monobody, recombinant antibody or monobody, humanized antibody or monobody, germline derived antibody or monobody, chimeric antibody or monobody, and affinity matured antibody or monobody.

A number of different combinations of structural amino acid positions and nonstructural amino acid positions can be designed in a CDRH3 region. For example, one CDRH3 comprises an amino acid sequence having the formula of A₁-A₂-(A₃)ₙ-A₄-A₅, wherein
A₁ is an amino acid selected from the group consisting of R, L, V, F, W and K;
A₂ is an amino acid selected from the group consisting of I, L, V, R, W and S;
A₃ is any naturally occurring amino acid and n can be 1-20;
A₄ is an amino acid selected from the group consisting of W, G, R, M, S, A and H; and
A₅ is an amino acid selected from the group consisting of V, L, P, G, S, E and W.

The amino acids to the left of the central portion of contiguous amino acids are referred to as the N terminal amino acids, and the amino acids to the right of the contiguous sequence are referred to as C terminal amino acids. Amino acids positions A₁ and A₂ are N terminal positions, A₃ represents the central portion that can be randomized, and A₄ and A₅ are C terminal positions. In some embodiments, the first two N-terminal amino acid positions have limited diversity; A₁ is an amino acid selected from the group consisting of R, L, V, F, W and K; and A₂ is an amino acid selected from the group consisting of I, L, V, R, W and S. Other amino positions that have limited diversity include A₄ and A₅. A₄ is the fourth amino acid from the C-terminus and is selected from the group consisting of W, G, R, M, S, A and H. A₅ is the third amino acid position from the C-terminus and is selected from the group consisting of V, L, P, G, S, E, and W. Amino acid(s) at A₃ can be any of the 20 naturally occurring amino acids, preferably L-amino acids.

A₃ is or comprises a contiguous amino acid sequence of about 1 to 17 amino acids, 5 to 15 amino acids, or 9 to 12 amino acids. The amino acids can each be any of one of the 20 naturally occurring amino acids (preferably L amino acids) or amino acids can be selected at one or more positions. In some embodiments, one or more positions can be encoded by a nonrandom codon set. The nonrandom codon set preferably encodes amino acids found at those positions in naturally occurring antibody or monobodies such as DVK or NVT.

A number of different combinations of structural amino acid positions and nonstructural amino acid positions can be designed in a CDRH3 region. For example, one CDRH3 comprises an amino acid sequence having the formula of A₁-A₂-(A₃)ₙ-A₄-A₅-A₆-A₇, wherein
A₁ is an amino acid selected from the group consisting of R, L, V, F, W and K;
A₂ is an amino acid selected from the group consisting of I, L, V, R, W and S;
A₃ is any naturally occurring amino acid and n can be 1-17;
A₄ is an amino acid selected from the group consisting of W, G, R, M, S, A and H; and
A₅ is an amino acid selected from the group consisting of V, L, P, G, S, E and W; and
A₆ and A₇ are any of the naturally occurring amino acids.

The amino acids to the left of the central portion of contiguous amino acids are referred to as the N terminal amino acids, and the amino acids to the right of the contiguous sequence are referred to as C terminal amino acids. Amino acids positions A₁ and A₂ are N terminal positions, A₃ represents the central portion that can be randomized, and A₄, A₅, A₇ and A₈ are C terminal positions. In this embodiment, the first two N-terminal amino acid positions have limited diversity; A₁ is an amino acid selected from the group consisting of R, L, V, F, W and K; and A₂ is an amino acid selected from the group consisting of I, L, V, R, W and S. Other amino positions that have limited diversity include A₄ and A₅. A₄ is the fourth amino acid from the C-terminus and is selected from the group consisting of W, G, R, M, S, A and H. A₅ is the third amino acid position from the C-terminus and is selected from the group consisting of V, L, P, G, S, E, and W. Amino acid positions at A₃, A₆ and A₇ can be any of the 20 naturally occurring amino acids, preferably L-amino acids. In some embodiments, amino acid positions A₆ and A₇ may be structural amino acid positions.

A₃ is or comprises a contiguous amino acid sequence of about 1 to 17 amino acids, 5 to 15 amino acids, or 9 to 12 amino acids. The amino acids can each be any of one of the 20 naturally occurring amino acids (preferably L amino acids) or amino acids can be selected at one or more positions. In some embodiments, one or more positions can be encoded by a nonrandom codon set. The nonrandom codon set preferably encodes amino acids found at those positions in naturally occurring antibody or monobodies such as DVK or NVT.

Another embodiment comprises a CDRH3 that comprises an amino acid sequence having the formula of A₁-A₂-(A₃)ₙ-A₄-A₅-A₆-A₇-A₈-A₉; wherein
A₁ is an amino acid selected from the group consisting of R, L, and V;
A₂ is an amino acid selected from the group consisting of I, L, and V;
A₃ is any naturally occurring amino acid and n=1-17;
A₄ is an amino acid selected from the group consisting of E, W, and F;
A₅ is any naturally occurring amino acid;
A₆ is an amino acid selected from group consisting of W, G, R, and M;
A₇ is an amino acid selected from the group consisting of V, L, and P; and
A₈ and A₉ is any naturally occurring amino acid.

The amino acids to the left of the central portion of contiguous amino acids, (A₃)ₙ, are referred to as the N terminal amino acids, and the amino acids to the right of the contiguous sequence are referred to as C terminal amino acids. Amino acids positions A₁ and A₂ are N terminal positions, A₃ represents the central portion that can be randomized, and A₄, A₅, A₇ and A₈ are C terminal positions. In some embodiments, amino acid positions A₈ and A₉ may be structural amino acid positions.

Another embodiment of a CDRH3 region comprises an amino acid sequence having the formula of R-L/I/M-A₃-R-(A₅)ₙ, wherein A₃ and A₅ are any naturally occurring amino acid and n is 1 to 20. A library of randomly generated 17 amino acid CDRH3 indicated that a consensus sequence R-L/I/M-A₃-R at the N-terminus may be preferred for some embodiments.

Another embodiment of a CDRH3 comprises an amino acid sequence having the formula of R-L/I/M-(A₃)ₙ-W-A₅-A₆-A₇-A₈-A₉, wherein A₆ is W, G, R or M; A₇ is V, L or P; A₃, A₅, A₈ and A₉ can be any naturally occurring amino acid and n is 1 to 15, about 5 to 15, or about 9 to 12. A library of randomly generated CDRH3 regions indicated that a consensus sequence may also include amino acids located near the C-terminal end of CDRH3, especially at the third, fourth, and sixth positions from the C-terminal end of CDRH₃.

In particular embodiments, one of 4 CDRH3 scaffolds may be especially useful in designing libraries of diverse CDRH3 regions while minimizing the structural perturbations of the polypeptide or antibody variable domain. A “CDRH3 scaffold” comprises a N-terminal portion in which some or all of the positions are structural and a C terminal portion in which some or all of the amino acid positions are structural and wherein the scaffold can accommodate the insertion of a central portion or loop of contiguous amino acids that may be randomized. In another embodiment, a CDRH3 scaffold comprises a N-terminal portion having a cysteine residue and a C terminal portion having a cysteine residue, wherein the cysteine residues in the N terminal and C-terminal portion of the CDRH3 form a disulfide bond that stabilizes the central portion insert that can vary in sequence and in length. In one embodiment, the scaffold has a N terminal sequence of R-L/I/M-A₃-R, wherein A₃ is any naturally occurring amino acid. In another embodiment, the N terminal sequence is R—I-A₃—C, wherein A₃ is any naturally occurring amino acid. In other embodiments, the N terminal sequence comprises R—I, L-L, V-L, or R-L. In some embodiments, the C terminus has a sequence of CWVTW. In other embodiments the C-terminal sequence comprises F—X—R—V, W—X—X-L, W—X-M-P, or W—V, wherein X can be any naturally occurring amino acid.

One CDRH3 comprises an amino acid sequence having the formula of A₁-A₂-(A₃)ₙ-A₄-A₅-A₆-A_{7,} wherein A₁ is R; A₂ is I; A₄ is W; A₅ is V; A₃, A₆ and A₇ can be any naturally occurring amino acid, and n=11. Another CDRH3 of interest comprises an amino acid sequence having the formula of A₁-A₂-(A₃)ₙ-A₄-A₅-A₆-A₇-A₈-A₉, wherein A₁ is V; A₂ is L; A₄ is F; A₆ is R; A₇ is V; A₃, A₅, A_{8,} and A₉ can be any naturally occurring amino acid, and n=9. Another CDRH3 of interest comprises an amino acid sequence having the formula of A₁-A₂-(A₃)ₙ-A₄-A₅-A₆-A₇-A₈-A₉, wherein A₁ is R; A₂ is L; A₄ is W; A₃, A₅, A₆, A₇, A₈ and A₉ can be any naturally occurring amino acid, and n=9. Another CDRH3 of interest comprises an amino acid sequence having the formula of A₁-A₂-(A₃)ₙ-A₄-A₅-A₆-A₇-A₈-A₉, wherein A₁ is L; A₂ is L; A₄ is W; A₇ is L; A₃, A₅, A₆, A, and A₉ can be any naturally occurring amino acid, and n=9.

Another embodiment of a CDRH3 comprises an amino acid sequence having the formula of
A₁-A₂-A₃-A₄-(A₅)ₙ-A₆-A₇-A₈-A₉-A₁₀
wherein A₁ is an amino acid selected from the group consisting of R, L and V;
A₂ is an amino acid selected from the group consisting of I, L and V;
A₃ is any naturally occurring amino acid;
A₄ is selected from the group consisting of C, R and N;
A₅ is any naturally occurring amino acid and n=1-16;
A₆ is an amino acid selected from the group consisting of C, S, F, T, E and D;
A₇ is an amino acid selected from the group consisting of W, G, R and M;
A₈ is an amino acid selected from the group consisting of V, L and P;
A₉ is an amino acid selected from the group consisting of T, V, L and Q; and
A₁₀ is an amino acid selected from the group consisting of W, G, S and A.

The amino acids to the left of the central portion of contiguous amino acids are referred to as the N terminal amino acids, and the amino acids to the right of the contiguous sequence are referred to as C terminal amino acids. Amino acids positions A₁, A₂, A₃, and A₄ are N terminal positions, A₅ represents the central portion that can be randomized, and A₆, A₇, A₈, A₉, and A₁₀ are C terminal positions. In some embodiments, amino acid positions A₈ and A₉ may be structural amino acid positions.

A particular embodiment of this CDRH3 region comprises the sequence A₁ is R; A₂ is I, A₄ is C; A₆ is C; A₇ is W; A₈ is V and n=7. In another embodiment, A₁ is R; A₂ is I, A₄ is C; A₆ is C; A₇ is W; A₈ is V and n=6.

Another aspect of the invention involves a method of designing a CDRH3 region that is well folded and stable for phage display. The method involves generating a library comprising polypeptides with variant CDRH3 regions, selecting the members of the library that bind to a target molecule that binds to folded polypeptide and does not bind to unfolded polypeptide, analyzing the members of the library to identify structural amino acid positions in the CDRH3 region, identifying at least one amino acid that can be substituted at the structural amino acid position, wherein the amino acid identified is one that occurs significantly more frequently than random (one standard deviation or greater than the frequency of any amino acid at that position) in polypeptides selected for stability, and designing a CDRH3 region that has at least one of the identified amino acid in the structural amino acid position. The method further comprises selecting a CDRH3 design with structural amino acid positions in one or more of the first two N-terminal amino acid positions or in one or more of the last six amino acid positions from the C-terminal end of the CDRH3 or both. The design preferably allows for a central portion that can be randomized and is not structurally constrained. In one embodiment, all of the structural amino acid positions have one of the identified amino acids at each of those positions. Libraries with variant CDRH3 regions can be generated and sorted for members of the library that bind to a target antigen such as a cytokine.

Another aspect of the invention provides methods for generating a polypeptide comprising a variant CDRH3 comprising identifying at least one structural amino acid position in a CDRH3, and replacing an amino acid at said structural amino acid position with a variant amino acid found at that position in a population of polypeptides with randomized CDRH3 at a frequency at least one standard deviation above the average frequency for any amino acid at that position. The CDRH3 also comprises at least one non-structural amino acid position that can vary in sequence or length. The polypeptide is preferably a monobody or VHH and the variant amino acid at least one structural position is preferably a hydrophobic amino acid or an arginine. The hydrophobic amino acid is selected from the group consisting of leucine, valine, isoleucine, tyrosine, tryptophan, and phenylalanine.

A structural amino acid position of a CDRH3 can be identified using a variety of methods. In one embodiment, structural amino acid positions for CDRH3 sequence can be identified using a method such as alanine scanning mutagenesis or shotgun scanning as described in WO 01/44463 and analyzing the effect of loss of the wild type amino acid on structural stability at positions in the CDRH3. An embodiment for identifying structural amino acids in a CDRH3 involves generating a library of antibody variable domains randomized at each amino acid position in the CDRH3. The library is sorted against a target molecule that specifically binds to folded polypeptide and does not bind to unfolded polypeptide and does not bind at an antigen binding site, such as Protein A. The sequence of the members of the library selected by interaction with the target molecule is determined. The most commonly occurring sequences in the CDRH3 region are identified. Structural amino acid positions in each of those commonly occurring sequences can be identified using a method such as shotgun scanning. A structural amino acid position is identified as an amino acid position in the CDRH3 that when substituted with the scanning amino acid has a decrease in the interaction with the target molecule, such as Protein A, as compared to a polypeptide having a source or wild type CDRH3 amino acid at that position. Preferably, a structural amino acid position is identified as a position in which the ratio of sequences with the wild type amino acid at a position to sequences with the scanning amino acid at that position is at least about 3 to 1, about 5 to 1, about 8 to 1, more preferably about 10 to 1 or greater.

A target molecule is a molecule that binds to folded polypeptide and does not bind to unfolded polypeptide and preferably, does not bind at an antigen binding site. For example, for Protein A, the Protein A binding site of Vh3 antibody variable domains is found on the opposite B sheet from the antigen binding site. Another example of a target molecule includes an antibody or antigen binding fragment or polypeptide that does not bind to the antigen binding site and binds to folded polypeptide and does not bind to unfolded polypeptide, such as an antibody to the Protein A binding site.

The invention also provides for 1) fusion polypeptides; 2) fusion polypeptides to viral coat proteins or portions thereof, 3) polynucleotides encoding any of the polypeptides; 4) replicable expression vectors comprising a polynucleotide encoding the polypeptides of the invention; 5) host cells comprising the vectors; 6) a library comprising a plurality of vectors of the invention and 7) a population of variant polypeptides or polynucleotides of the invention.

In another aspect, the invention provides a method of generating a polypeptide comprising at least one, two, three, four or five variant CDRs (i.e., selected from the group consisting of CDRs L1, L2, L3, H1 and H2) wherein said polypeptide is capable of binding a target molecule of interest, and wherein said CDR is not CDRH3, said method comprising: (a) identifying at least one (or any number up to all) solvent accessible and highly diverse amino acid position in a CDR; and (b) replacing the amino acid at the solvent accessible and high diverse position with a target amino acid (as defined herein) by generating variant copies of the CDR using a non-random codon set, wherein at least about 50%, 60%, 70%, 80%, 90% or all of the amino acids encoded by the non-random codon set are target amino acids (as defined herein) for that position in known antibodies or antigen binding fragment or polypeptides.

In another aspect, the invention provides a method of generating a polypeptide comprising at least one, two, three, four, five or all of variant CDRs selected from the group consisting of H1, H2, H3, L1, L2 and L3, wherein said polypeptide is capable of binding a target molecule of interest, said method comprising: (a) with respect to L1, L2, L3, H1 and H2, (i) identifying at least one (or any number up to all) solvent accessible and highly diverse amino acid position in a reference CDR corresponding to the variant CDR; and (ii) replacing the amino acid at the solvent accessible and high diverse position with a target amino acid by generating variant copies of the CDR using a non-random codon set, wherein at least about 50%, 60%, 70%, 80%, 90% or all of the amino acids encoded by the non-random codon set are target amino acids for that position in known antibodies or antigen binding fragment or polypeptides; and (b) with respect to H3, replacing at least one (or any number up to all) position with a variant amino acid.

In another aspect, the invention provides a method of generating a polypeptide comprising at least one, two, three, four, five or all of variant CDRs selected from the group consisting of L1, L2, L3, H1, H2 and H3, said method comprising: (a) substituting at least one (or any number up to all) solvent accessible and highly diverse amino acid position in L1, L2, L3, H1 and/or H2 with a variant amino acid which is encoded by a nonrandom codon set, wherein at least 50%, 60%, 70%, 80%, 90% or all of amino acids encoded by the nonrandom codon set are target amino acids for said amino acid position in known antibodies or antigen binding fragment or polypeptides; and (b) substituting at least one (or any number up to all) amino acid position in H3 with a variant amino acid.

Various aspects and embodiments of methods of the invention are useful for generating and/or using a pool comprising a plurality of polypeptides of the invention, in particular for selecting and identifying candidate binders to target molecules of interest. For example, the invention provides a method of generating a composition comprising a plurality of polypeptides, each polypeptide comprising at least one, two, three, four, five or all of variant CDRs selected from the group consisting of L1, L2, L3, H1, H2 and H3, said method comprising: (a) substituting at least one (or any number up to all) solvent accessible and highly diverse amino acid position in L1, L2, L3, H1 and/or H2 with a variant amino acid which is encoded by a nonrandom codon set, wherein at least 50%, 60%, 70%, 80%, 90% or all of amino acids encoded by the nonrandom codon set are target amino acids for said amino acid position in known antibodies or antigen binding fragment or polypeptides; and/or (b) substituting at least one (or any number up to all) amino acid position in H3 with a variant amino acid; wherein a plurality of polypeptides are generated by amplifying a template polynucleotide with a set of oligonucleotides comprising degeneracy in the sequence encoding a variant amino acid, wherein said degeneracy reflects the multiple codon sequences of the nonrandom codon set.

In another example, the invention provides a method comprising: constructing an expression vector comprising a polynucleotide sequence which encodes a light chain, a heavy chain, or both the light chain and the heavy chain variable domains of a source antibody comprising at least one, two, three, four, five or all CDRs selected from the group consisting of CDR L1, L2, L3, H1, H2 and H3; and mutating at least one, two, three, four, five or all CDRs of the source antibody at least one (or any number up to all) solvent accessible and highly diverse amino acid position using a nonrandom codon set, wherein at least about 50%, 60%, 70%, 80%, 90% or all of the amino acids encoded by the non-random codon set are target amino acids for that position in known antibodies or antigen binding fragment or polypeptides.

In another example, the invention provides a method comprising: constructing a library of phage or phagemid particles displaying a plurality of polypeptides of the invention; contacting the library of particles with a target molecule under conditions suitable for binding of the particles to the target molecule; and separating the particles that bind from those that do not bind to the target molecule.

In any of the methods of the invention described herein, a solvent accessible and/or highly diverse amino acid position can be any that meet the criteria as described herein, in particular any combination of the positions as described herein, for example any combination of the positions described for the polypeptides of the invention (as described in greater detail below). Suitable variant amino acids can be any that meet the criteria as described herein, for example variant amino acids in polypeptides of the invention as described in greater detail below.

In some embodiments of any of the methods described herein, the position in H3 is any of positions 95 to 100a. In some embodiments of any of the methods described herein, the variant H3 amino acid is encoded by codon set NNK, NNS, DVK or NVT. In some embodiments, the nucleotide ratios/proportions of these codon sets are modified to reflect amino acid preferences, for example in accordance with the natural diversity profile of a particular amino acid position.

In some embodiments of methods described herein, a nonrandom codon set does not encode cysteine. In some embodiments of methods of the invention, a nonrandom codon set does not include a stop codon.

Methods of the invention are capable of generating a large variety of polypeptides comprising a diverse set of CDR sequences. For example, in one embodiment, the invention provides a polypeptide comprising at least one, two, three, four, five or all of variant CDRs selected from the group consisting of CDRL1, CDRL2, CDRL3, CDRH1, CDRH2 and CDRH3; wherein (i) each of CDRs L1, L2, L3, H1 and H2 has a variant amino acid in at least one (or any number up to all) solvent accessible and highly diverse amino acid position, wherein the variant amino acid is encoded by a non-random codon set, and wherein at least about 50%, 60%, 70%, 80%, 90% or all of the amino acids encoded by the non-random codon set are target amino acids for that position in known antibodies or antigen binding fragment or polypeptides; and (ii) variant CDRH3 has a variant amino acid in at least one (or any number up to all) amino acid position.

In another embodiment, the invention provides a polypeptide comprising: (a) at least one, two, three, four or all of variant CDRs selected from the group consisting of CDRL1, CDRL2, CDRL3, CDRH1 and CDRH2; wherein each of CDRs L1, L2, L3, H1 and H2 has a variant amino acid in at least one (or any number up to all) solvent accessible and highly diverse amino acid position, wherein the variant amino acid is encoded by a non-random codon set, and wherein at least about 50%, 60%, 70%, 80%, 90% or all of the amino acids encoded by the non-random codon set are target amino acids for that position in known antibodies or antigen binding fragment or polypeptides; and (b) a variant CDRH3 comprising a variant amino acid in at least one (or any number up to all) amino acid position.

In one embodiment, the invention provides a polypeptide comprising at least one, two, three, four, five or all of CDRs selected from the group consisting of CDRL1, CDRL2, CDRL3, CDRH1, CDRH2 and CDRH3, wherein: (a) CDRL1 comprises a variant amino acid in at least one, two, three, four or all of amino acid positions 28, 29, 30, 31 and 32; (b) CDRL2 comprises a variant amino acid in at least one or both of amino acid positions 50 and 53; (c) CDRL3 comprises a variant amino acid in at least one, two, three, four or all of amino acid positions 91, 92, 93, 94 and 96; (d) CDRH1 comprises a variant amino acid in at least one, two, three, four or all of amino acid positions 28, 30, 31, 32 and 33; (e) CDRH2 comprises a variant amino acid in at least one, two, three, four, five or all of amino acid positions 50, 52, 53, 54, 56 and 58; and (f) CDRH3 comprises a variant amino acid in at least one, two, three, four, five, six or all of amino acid positions 95, 96, 97, 98, 99, 100 and 100a; wherein the amino acid positions correspond to the Kabat numbering system; and wherein each variant amino acid of (a) to (e) is encoded by a non-random codon set, and wherein at least about 50%, 60%, 70%, 80%, 90% or all of the amino acids encoded by the non-random codon set are target amino acids for that amino acid position in known antibodies or antibody fragments. In some embodiments of these polypeptides, the variant amino acid of (f) is encoded by codon set NNK, NNS, DVK or NVT.

In some embodiments, the invention provides a polypeptide comprising antibody light chain and heavy chain variable domains, wherein: (a) CDRL1 comprises a variant amino acid in at least one, two, three, four or all of amino acid positions 28, 29, 30, 31 and 32; (b) CDRL2 comprises a variant amino acid in at least one or both of amino acid positions 50 and 53; (c) CDRL3 comprises a variant amino acid in at least one, two, three, four or all of amino acid positions 91, 92, 93, 94 and 96; (d) CDRH1 comprises a variant amino acid in at least one, two, three, four or all of amino acid positions 28, 30, 31, 32 and 33; (e) CDRH2 comprises a variant amino acid in at least one, two, three, four, five or all of amino acid positions 50, 52, 53, 54, 56 and 58; and (f) CDRH3 comprises a variant amino acid in at least one, two, three, four, five, six or all of amino acid positions 95, 96, 97, 98, 99, 100 and 100a; wherein the amino acid positions correspond to the Kabat numbering system; and wherein each variant amino acid of (a) to (e) is encoded by a non-random codon set, and wherein at least about 50%, 60%, 70%, 80%, 90% or all of the amino acids encoded by the non-random codon set are target amino acids for that amino acid position in known antibodies or antibody fragments. In some embodiments, the variant amino acid of (f) is encoded by codon set NNK, NNS, DVK or NVT.

In some embodiments, the invention provides a polypeptide comprising at least one, two, three, four, five or all of CDRs selected from the group consisting of CDRL1, CDRL2, CDRL3, CDRH1, CDRH2 and CDRH3, wherein: (a) CDRL1 comprises a variant amino acid in amino acid positions 28, 29, 30, 31 and 32; (b) CDRL2 comprises a variant amino acid in amino acid positions 50 and 53; (c) CDRL3 comprises a variant amino acid in amino acid positions 91, 92, 93, 94 and 96; (d) CDRH1 comprises a variant amino acid in amino acid positions 28, 30, 31, 32 and 33; (e) CDRH2 comprises a variant amino acid in amino acid positions 50, 52, 53, 54, 56 and 58; and (f) CDRH3 comprises a variant amino acid in amino acid positions 95, 96, 97, 98, 99, 100 and 100a; wherein the amino acid positions correspond to the Kabat numbering system; and wherein each variant amino acid of (a) to (e) is encoded by a non-random codon set, and wherein at least about 50%, 60%, 70%, 80%, 90% or all of the amino acids encoded by the non-random codon set are target amino acids for that amino acid position in known antibodies or antibody fragments. In some embodiments, the variant amino acid of (e) is encoded by codon set NNK, NNS, DVK or NVT.

In another embodiment, the invention provides a polypeptide comprising at least one, two, three, four, five or all of CDRs selected from the group consisting of CDRL1, CDRL2, CDRL3, CDRH1, CDRH2 and CDRH3, wherein: (a) CDRL1 comprises a variant amino acid in amino acid positions 28, 29, 30, 31 and 32, wherein: (i) the variant amino acid at position 28 is encoded by codon set RDT; (ii) the variant amino acid at position 29 is encoded by codon set RKT or RTT; (iii) the variant amino acid at position 30 is encoded by codon set RVW; (iv) the variant amino acid at position 31 is encoded by codon set RVW or ANW; (v) the variant amino acid at position 32 is encoded by codon set DHT or THT; (b) CDRL2 comprises a variant amino acid in amino acid positions 50 and 53, wherein: (i) the variant amino acid at position 50 is encoded by codon set KBG; (ii) the variant amino acid at position 53 is encoded by codon set AVC; (c) CDRL3 comprises a variant amino acid in amino acid positions 91, 92, 93, 94 and 96, wherein: (i) the variant amino acid at position 91 is encoded by codon set KMT or TMT or the combination of codon sets TMT and SRT; (ii) the variant amino acid at position 92 is encoded by codon set DHT or DMC; (iii) the variant amino acid at position 93 is encoded by codon set RVT or DHT; (iv) the variant amino acid at position 94 is encoded by codon set NHT or WHT; (v) the variant amino acid at position 96 is encoded by codon set YHT or HWT or HTT or the combination of codon sets YKG and TWT; (d) CDRH1 comprises a variant amino acid in amino acid positions 28, 30, 31, 32 and 33, wherein: (i) the variant amino acid at position 28 is encoded by codon set AVT or WCC or is threonine; (ii) the variant amino acid at position 30 is encoded by codon set RVM or AVT; (iii) the variant amino acid at position 31 is encoded by codon set RVM, RVT or RRT; (iv) the variant amino acid at position 32 is encoded by codon set WMY; (v) the variant amino acid at position 33 is encoded by codon set KVK, RNT, DMT, KMT, KGG or the combination of codon sets KMT and KGG; (e) CDRH2 comprises a variant amino acid in amino acid positions 50, 52, 53, 54, 56 and 58, wherein: (i) the variant amino acid at position 50 is encoded by codon set KDK or DBG or the combination of codon sets DGG and DHT; (ii) the variant amino acid at position 52 is encoded by codon set DHT or DMT; (iii) the variant amino acid at position 53 is encoded by codon set NMT or DMT; (iv) the variant amino acid at position 54 is encoded by codon set DMK, DMT or RRC; (v) the variant amino acid at position 56 is encoded by codon set DMK or DMT; (vi) the variant amino acid at position 58 is encoded by codon set DMT or DAC; and (f) CDRH3 comprises a variant amino acid in amino acid positions 95, 96, 97, 98, 99, 100 and 100a, wherein: the variant amino acid at each of positions 95, 96, 97, 98, 99, 100 and 100a is encoded by codon set NNK, NNS, DVK or NVT; wherein the amino acid positions correspond to the Kabat numbering system, and wherein codon set symbols are according to the IUB code.

In another embodiment, the invention provides a polypeptide comprising a light chain and a heavy chain variable domain, wherein: (a) CDRL1 comprises a variant amino acid in positions 28, 29, 30, 31 and 32, wherein: (i) the variant amino acid at position 28 is encoded by codon set RDT; (ii) the variant amino acid at position 29 is encoded by codon set RKT or RTT; (iii) the variant amino acid at position 30 is encoded by codon set RVW; (iv) the variant amino acid at position 31 is encoded by codon set RVW or ANW; (v) the variant amino acid at position 32 is encoded by codon set DHT or THT; (b) CDRL2 comprises a variant amino acid in positions 50 and 53, wherein: (i) the variant amino acid at position 50 is encoded by codon set KBG; (ii) the variant amino acid at position 53 is encoded by codon set AVC; (c) CDRL3 comprises a variant amino acid in positions 91, 92, 93, 94 and 96, wherein: (i) the variant amino acid at position 91 is encoded by codon set KMT or TMT or the combination of codon sets TMT and SRT; (ii) the variant amino acid at position 92 is encoded by codon set DHT or DMC; (iii) the variant amino acid at position 93 is encoded by codon set RVT or DHT; (iv) the variant amino acid at position 94 is encoded by codon set NHT or WHT; (v) the variant amino acid at position 96 is encoded by codon set YHT, HWT, HTT, TDK or the combination of codon sets YKG and TWT; (d) CDRH1 comprises a variant amino acid in positions 28, 30, 31, 32 and 33, wherein: (i) the variant amino acid at position 28 is encoded by codon set AVT or WCC or is threonine; (ii) the variant amino acid at position 30 is encoded by codon set RVM or AVT; (iii) the variant amino acid at position 31 is encoded by codon set RVM, RVT or RRT; (iv) the variant amino acid at position 32 is encoded by codon set WMY; (v) the variant amino acid at position 33 is encoded by codon set KVK, RNT, DMT, KMT or the combination of codon sets KMT and KGG; (e) CDRH2 comprises a variant amino acid in positions 50, 52, 53, 54, 56 and 58, wherein: (i) the variant amino acid at position 50 is encoded by codon set KDK, DBG or the combination of codon sets DGG and DHT; (ii) the variant amino acid at position 52 is encoded by codon set DHT or DMT; (iii) the variant amino acid at position 53 is encoded by codon set NMT or DMT; (iv) the variant amino acid at position 54 is encoded by codon set DMK, DMT or RRC; (v) the variant amino acid at position 56 is encoded by codon set DMK or DMT; (vi) the variant amino acid at position 58 is encoded by codon set DMT or DAC; (f) CDRH3 comprises a variant amino acid in positions 95, 96, 97, 98, 99, 100 and 100a, wherein the variant amino acid at each of positions 95, 96, 97, 98, 99, 100 and 100a is encoded by codon set NNK, NNS, DVK or NVT; wherein the amino acid positions correspond to the Kabat numbering system, and wherein codon set symbols are according to the IUB code.

In various embodiments of polypeptides of the invention, amino acids encoded by a non-random codon set preferably include (generally are) amino acids found at the corresponding position in preferably at least about 50%, 60% or 70% of known antibodies or antigen binding fragment or polypeptides. In some embodiments, said known antibodies or antigen binding fragment or polypeptides are as in “Sequences of Proteins of Immunological Interest” (5th edition) published by the U.S. National Institutes of Health. In some embodiments, said known antibodies or antigen binding fragment or polypeptides are as in the database of Kabat at http://immuno.bme.nwu.edu.

In some embodiments of polypeptides of the invention comprising a variant CDRH3, the variant CDRH3 has a variant amino acid in at least one, two, three, four, five, six or all of amino acid positions 95 to 100a, wherein amino acid positions correspond to the Kabat numbering system. In some embodiments, the variant amino acid of variant CDRH3 is an amino acid encoded by codon set NNK, NNS, DVK or NVT.

In some embodiments of polypeptides of the invention, a nonrandom codon set does not encode cysteine. In some embodiments, a non-random codon set does not include a stop codon. In some embodiments of polypeptides of the invention, the variant amino acid at position 100a of CDRH3 is encoded by codon set DSG, KSG or is tyrosine.

As described herein, a variant CDR refers to a CDR with a sequence variance as compared to the corresponding CDR of a single reference polypeptide/source antibody. Accordingly, the CDRs of a single polypeptide of the invention preferably correspond to the set of CDRs of a single reference polypeptide or source antibody.

Polypeptides of the invention can be in a variety of forms as long as the target binding function of the polypeptides are retained. In some embodiments, a polypeptide of the invention is a fusion polypeptide (i.e., a fusion of two or more sequences from heterologous polypeptides). In some embodiments, the fusion polypeptide is fused to at least a portion of a viral coat protein, such as a viral coat protein selected from the group consisting of pill, pVIII, Soc, Hoc, gpD, pVI, and variants thereof.

In some embodiments, a polypeptide of the invention comprises a light chain and a heavy chain antibody variable domain, wherein the light chain variable domain comprises at least 1, 2 or 3 variant CDRs selected from the group consisting of CDRL1, L2 and L3, and the heavy chain variable domain comprises at least 1, 2 or 3 variant CDRs selected from the group consisting of CDRH1, H2 and H3.

In some embodiments, a polypeptide of the invention is an ScFv. In some embodiments, it is a Fab fragment. In some embodiments, it is a F(ab)′₂. In some embodiments, heavy chains of the F(ab)′₂ dimerize at a dimerization domain. The dimerization domain may comprise a leucine zipper sequence (for example, a GCN4 sequence as depicted in SEQ ID NO.: 3). Accordingly, in some embodiments, a polypeptide of the invention further comprises a dimerization domain. In some embodiments, the dimerization domain is located between an antibody heavy chain or light chain variable domain and at least a portion of a viral coat protein. In some embodiments, the dimerization domain comprises a leucine zipper sequence (for example, the GCN4 sequence as depicted in SEQ ID NO.: 3).

In some embodiments, a polypeptide of the invention further comprises a light chain constant domain fused to a light chain variable domain, which in some embodiments comprises at least one, two or three variant CDRs.

In some embodiments of polypeptides of the invention, the polypeptide comprises a heavy chain constant domain fused to a heavy chain variable domain, which in some embodiment comprises at least one, two or three variant CDRs.

A polypeptide of the invention may comprise a dimerization domain between the heavy chain constant domain and at least a portion of a viral protein. The dimerization domain may comprise a leucine zipper sequence (for example, the GCN4 sequence as depicted in SEQ ID NO.: 3).

In some embodiments, a polypeptide of the invention comprises an antibody light chain variable domain, wherein the variant CDR is CDRL1 and the amino acid positions that are diversified are those positions that correspond to amino acid positions 28, 29, 30, 31 and 32. In some embodiments, the variant amino acid at position 28 is encoded by codon set RDT, the variant amino acid at position 29 is encoded by codon set RKT or RTT, the variant amino acid at position 30 is encoded by codon set RVW, the variant amino acid at position 31 is encoded by codon set RVW or ANW, and the variant amino acid at position 32 is encoded by codon set DHT or THT.

In another embodiment, a polypeptide of the invention comprises an antibody light chain variable domain, wherein the variant CDR is CDRL2, and the amino acid positions that are diversified are those that correspond to amino acid positions 50 and 53. In some embodiments, the variant amino acid at position 50 is encoded by KBG codon set, and the variant amino acid at position 53 is encoded by codon set AVC.

In another embodiment, a polypeptide of the invention comprises an antibody light chain variable domain, wherein the variant CDR is CDRL3, and the amino acid positions that are diversified are selected from those that correspond to amino acid positions 91, 92, 93, 94, or 96. In some embodiments, the variant amino acid at position 91 is encoded by codon set KMT, TMT or the combination of codon sets TMT and SRT, the variant amino acid at position 92 is encoded by codon set DHT or DMC, the variant amino acid at position 93 is encoded by codon set RVT or DHT, the variant amino acid at position 94 is encoded by codon set NHT or WHT, and the variant amino acid at position 96 is encoded by codon set YHT, HWT, HTT or the combination of codon sets YKG and TWT.

In another embodiment, a polypeptide of the invention comprises a heavy chain variable domain, the variant CDR is CDRH1, and the amino acid positions that are diversified are those selected from amino acids positions corresponding to amino acids 28, 30, 31, 32 or 33. In some embodiments, the variant amino acid at position 28 is encoded by codon set AVT, WCC or is threonine, the variant amino acid at position 30 is encoded by codon set RVM or AVT, the variant amino acid at position 31 is encoded by codon set RVM, RVT or RRT, the variant amino acid at position 32 is encoded by codon set WMY, and the variant amino acid at position 33 is encoded by codon set KVK, RNT, DMT, KMT or the combination of codon sets KMT and KGG.

In another embodiment, a polypeptide of the invention comprises a heavy chain variable domain, the variant CDR is CDRH2, and the amino acid positions that are diversified are those selected from amino acid positions corresponding to amino acids 50, 52, 53, 54, 56 or 58. In some embodiments, the variant amino acid at position 50 is encoded by codon set KDK, DBG or the combination of codon sets DGG and DHT, the variant amino acid at position 52 is encoded by codon set DHT or DMT, the variant amino acid at position 53 is encoded by codon set NMT or DMT, the variant amino acid at position 54 is encoded by codon set DMK, DMT or RRC, the variant amino acid at position 56 is encoded by codon set DMK or DMT, and the variant amino acid at position 58 is encoded by codon set DMT or DAC.

In another embodiment, a polypeptide of the invention comprises a heavy chain variable domain, and the variant CDR is CDRH3 comprising a variant amino acid in at least one, two, three, four, five, six or all of amino acid positions 95 to 100a, wherein the variant amino acids is encoded by codon set NNK, NNS, DVK or NVT.

In some instances, it may be preferable to mutate a framework residue such that it is variant with respect to a reference polypeptide or source antibody. For example, framework residue 71 of the heavy chain may be R, V or A. In another example, framework residue 93 of the heavy chain may be S or A. In yet another example, framework residue 94 of the heavy chain may be R, K or T.

Methods of the invention can be used to diversify any reference polypeptide or source antibody, and polypeptides of the invention can comprise a sequence derivative of any reference polypeptide or source antibody. For example, a source antibody may comprise the amino acid sequence of the variable domains of humanized antibody 4D5 (SEQ ID NO: 1, SEQ ID NO: 2).

As described herein, polypeptides of the invention may comprise a heterologous polypeptide sequence, such as the sequence of at least a portion of a viral polypeptide or a tag sequence (such as polyhistidine).

Polypeptides of the invention may comprise any one or combinations of variant CDRs. For example, a polypeptide of the invention may comprise a variant CDRH1 and CDRH2. A polypeptide of the invention may comprise a variant CDRH1, variant CDRH2 and a variant CDRH3. In another example, a polypeptide of the invention may comprise a variant CDRH1, H2, H3 and L3. In another example, a polypeptide of the invention comprises a variant CDRL1, L2 and L3. Any polypeptide of the invention may further comprise a variant CDRL3. Any polypeptide of the invention may further comprise a variant CDRH3.

Polypeptides of the invention may be in a complex with one another. For example, the invention provides a polypeptide complex comprising two polypeptides, wherein each polypeptide is a polypeptide of the invention, and wherein one of said polypeptides comprises at least one, two or all of variant CDRs H1, H2 and H3, and the other polypeptide comprises a variant CDRL3. A polypeptide complex may comprise a first and a second polypeptide (wherein the first and second polypeptides are polypeptides of the invention), wherein the first polypeptide comprises at least one, two or three variant light chain CDRs, and the second polypeptide comprises at least one, two or three variant heavy chain CDRs. The invention also provides complexes of polypeptides that comprise the same variant CDR sequences. Complexing can be mediated by any suitable technique, including by dimerization/multimerization at a dimerization/multimerization domain such as those described herein or covalent interactions (such as through a disulfide linkage).

In another aspect, the invention provides compositions comprising polypeptides and/or polynucleotides of the invention. For example, the invention provides a composition comprising a plurality of any of the polypeptides of the invention described herein. Said plurality may comprise polypeptides encoded by a plurality of polynucleotides generated using a set of oligonucleotides comprising degeneracy in the sequence encoding a variant amino acid, wherein said degeneracy is that of the multiple codon sequences of the nonrandom or random codon set encoding the variant amino acid.

In one aspect, the invention provides a polynucleotide encoding a polypeptide of the invention as described herein. A polynucleotide of the invention may be a replicable expression vector comprising a sequence encoding a polypeptide of the invention.

In another aspect, the invention provides a library comprising a plurality of vectors of the invention, wherein the plurality of vectors encode a plurality of polypeptides.

The invention also provides a host cell comprising any of the polynucleotides and/or vectors of the invention described herein.

In another aspect, the invention provides a virus or virus particle displaying a polypeptide of the invention on its surface. The invention also provides a library comprising a plurality of the viruses or virus particles of the invention, each virus or virus particle displaying a polypeptide of the invention. A library of the invention may comprise any number of distinct polypeptides (sequences), preferably at least about 1×10⁸, preferably at least about 1×10⁹, preferably at least about 1×10¹⁰ distinct sequences.

The invention also provides libraries containing a plurality of polypeptides, wherein each type of polypeptide is a polypeptide of the invention as described herein.

### BRIEF DESCRIPTION OF THE FIGURES

FIG. 1 shows the frequency of amino acids (identified by single letter code) in human antibody light chain CDR sequences from the Kabat database. The frequency of each amino acid at a particular amino acid position is shown starting with the most frequent amino acid at that position at the left and continuing on to the right to the least frequent amino acid. The number below the amino acid represents the number of naturally occurring sequences in the Kabat database that have that amino acid in that position.

FIG. 2 shows the frequency of amino acids (identified by single letter code) in human antibody heavy chain CDR sequences from the Kabat database. The frequency of each amino acid at a particular amino acid position is shown starting with the most frequent amino acid at that position at the left and continuing on to the right to the least frequent amino acid. The number below the amino acid represents the number of naturally occurring sequences in the Kabat database that have that amino acid in that position. Framework amino acid positions 71, 93 and 94 are also shown.

FIG. 3 shows illustrative embodiments of suitable codon set design for amino acid positions in CDRL1, CDRL2, CDRL3, CDRH1 and CDRH2. The codon sets are identified by three capital letters in italics and are bracketed by < and >, e.g. <RDT>. The amino acids encoded by that codon set are indicated by single letter code under the column labeled Diversity<DNA codon>. The column labeled Natural Diversity shows the most commonly occurring amino acids at those positions in the naturally occurring antibody variable domains in the Kabat database. The % good is the % of amino acids that are encoded by the codon set that are target amino acids for that position. The % covering is the % of natural occurring antibodies in the Kabat database that have the amino acids encoded by the codon set at that position.

FIGS. 4A & B & C show illustrative embodiments of designed diversity in CDRH3 from antibody 4D5. The different oligonucleotides encode for diversity at amino acid positions in CDRH3 as well as diversity in sequence length. The oligonucleotides are identified as F59, F63, and F64 etc in the left hand column. The amino acid sequence at each amino acid position for CDRH3 for each oligonucleotide is shown. The CDRH3 sequence in the source antibody 4D5 is shown across the top: S₉₃, R₉₄, W₉₅, G₉₆, G₉₇, D₉₈, G₉₉, F₁₀₀, Y₁₀₀ₐ, A_{100b}, M_{100c}, D₁₀₁, and Y₁₀₂. Amino acid positions 93 and 94 are considered framework positions. In some embodiments, certain positions may have a fixed amino acid shown in single letter code, e.g. position 93 is S(serine); amino acid position 94 may be R/K/T (arginine/lysine/threonine); amino acid position 100a may be G/S/A/W (glycine/serine/alanine/tryptophan). Other amino acid positions are diversified using codon sets identified by three capital letters in italics, e.g. DVK, NVT, DSG, KSG. The length of the CDRH3 is indicated at the right column. The lengths of the CDRH3 regions varied from 7 to 15. The diversity of the library generated with the strategy shown for each oligonucleotide is also shown on the right. A single oligonucleotide may be used or oligonucleotides may be pooled to generate a library.

FIG. 5 shows an illustrative embodiment of designed diversity for CDR's L1 and L2 and L3. The codon sets for each position is shown. The amino acids (in single letter code) encoded by the codon set at each position are shown below in a column. The diversity generated with this design results in a library with 2.9×10⁹ sequences.

FIG. 6 shows an illustrative embodiment of designed diversity using nonrandom codon sets for amino acid positions in CDRL1, L2 and L3. The amino acids (in single letter code) encoded by the codon set at each position are shown below in a column. The diversity generated with this design results in 6.1×108 sequences.

FIG. 7 shows an illustrative embodiment of designed diversity using nonrandom codon sets at amino acid positions in CDRL3. The amino acids (in single letter code) encoded by the codon set at each position are shown below in a column.

FIG. 8 shows an illustrative embodiment of designed diversity using nonrandom codon sets for CDRs L1, L2 and L3. At some positions, the codon set may encode an increased proportion of one or more amino acids. For example, at position 93 in CDRL3, codon set RVM encodes an increased proportion of alanine (A2), glycine (G2) and threonine (T2). The amino acids (in single letter code) encoded by the codon set at each position are shown below in a column.

FIG. 9 shows an illustrative embodiment of designed diversity using nonrandom codon sets at amino acid positions in CDRH1, H2 and H3 in antibody 4D5. The amino acids (in single letter code) encoded by the codon set at each position are shown below in a column.

FIG. 10 shows an illustrative embodiment of designed diversity using nonrandom codon set at amino acid positions in CDRs H1, H2 and H3 in antibody 4D5. The amino acids (in single letter code) encoded by the codon set at each position are shown below in a column.

FIG. 11 shows an illustrative embodiment of designed diversity using nonrandom codon sets at amino acid positions in CDRs in H1, H2, H3 and L3 of antibody 4D5. The amino acids (in single letter code) encoded by the codon set at each position are shown below in a column.

FIG. 12 shows an illustrative embodiment of designed diversity using nonrandom codon sets at amino acid positions in CDRs in H1, H2, H3 and L3 of antibody 4D5. The amino acids(in single letter code) encoded by the codon set at each position are shown below in a column.

FIG. 13 shows an illustrative embodiment of designed diversity using nonrandom codon sets at amino acid positions in CDRs H1, H2, H3 and L3 in antibody 4D5. The amino acids (in single letter code) encoded by the codon set at each position are shown below in a column.

FIGS. 14A & B shows nucleotide sequence of Ptac promoter driver cassette for display of ScFv (SEQ ID NO: 23). Sequences encoding malE secretion signal, humanized antibody 4D5 light chain variable domain, linker, gD tag, humanized 4D5 heavy chain variable domain, and C-terminal domain of p3 (cP3) are indicated.

FIGS. 15A & B shows the DNA sequence of the Ptac promoter driver cassette for display ScFv-zip (SEQ ID NO: 24). Sequences encoding malE secretion signal, humanized 4D5 light chain variable domain, linker, gD tag, humanized 4D5 heavy chain variable domain, zipper sequence, and C-terminal domain p3 (cP3) are indicated.

FIGS. 16A & B shows DNA sequence of the Ptac promoter driven cassette for display of Fab (SEQ ID NO: 25). Two open reading frames are indicated. The first open reading frame encodes a malE secretion signal, humanized 4D5 light chain variable and constant domain. The second open reading frame encodes stII secretion signal, humanized heavy chain variable domain, humanized 4D5 heavy chain first constant region (CH1) and C-terminal domain of p3.

FIGS. 17A & B shows the DNA sequence of Ptac promoter driven cassette for display of Fab-zip (SEQ ID NO: 26). Two open reading frames are indicated. The first open reading frame encodes a malE secretion signal, humanized 4D5 light chain variable and constant domain. The second open reading frame encodes a stII secretion signal, humanized 4D5 heavy chain variable domain, humanized 4D5 heavy chain first constant domain (CH1), zipper sequence, and C-terminal of p3 (cP3).

FIG. 18 shows a schematic representation of display of different constructs including F(ab) and F(ab′)₂. (A) shows a Fab with a light chain, and a heavy chain variable and CH1 domain fused to at least a portion of a viral coat protein; (B) shows a F(ab′)₂ with two light chains, and one heavy chain with a dimerization domain (zip) fused to at least a portion of the viral coat protein; an amber stop codon is present after the dimerization domain and (C) shows a F(ab′)2 with two light chains, and both heavy chain variable and CH1 domains, each with a dimerization domain, and each fused to at least a portion of the viral coat protein

FIG. 19 shows a graph of the % bound of Fab phage constructs in the presence of increasing amounts of HER-2ecd (target antigen). The constructs are Fab phage (-∘-) or zipped F(ab′)₂ phage (-●-). The F(ab) or zipped F(ab′)₂ phage, each was incubated with increasing concentrations of Her-2ECD (0.001 to 1000 nM) in solution for 5 hours at 37° C. The unbound phage was captured with plates coated with Her-2ECD and measured with HRP-anti-M13 conjugate.

FIG. 20 shows the differences in off rate between Fab (-∘-) or zipped F(ab′)₂ (-●-)phage. Serial dilutions of Her-2ECD (0.01 nm to 1000 nM) were added to Fab or zipped F(ab′)₂ phage bound to Her-2ECD coated wells. The phage remaining bound to the plate was quantified using HRP-anti-M13 conjugate. The relative proportion of remaining phage bound as a percentage was calculated by dividing OD at a particular Her-2ECD concentration with OD in absence of Her-2ECD.

FIG. 21 shows the differences in the amount of phage F(ab) phage (-∘-) or zipped F(ab′)₂ (-●-) that is required to give detectable binding on a ligand coated support by standard phage ELISA. Differing concentrations of phage were diluted and the binding signal on Her-2ECD coated plates was detected with HRP anti-M13 measured at an O.D. of 450 nm.

FIG. 22 shows the ability to detect a low affinity binder using divalent display. A humanized 4D5 mutant was prepared with arginine 50 changed to alanine (R50A) in both F(ab) phage (-∘-) or zipped F(ab′)₂ (-●-) format. The phage was diluted and the binding on Her-2ECD coated plates was detected with HRP anti-M13.

FIG. 23 shows the comparison of the frequency of amino acid types in CDRH3 regions in naturally occurring human antibodies (solid bar) and in antibody variable domains with diversity generated with NNK codon sets (stippled bar). Amino acids are grouped as follows: phenyalanine (F), trytophan (W) and tyrosine (Y) are aromatic amino acids; isoleucine (I), leucine (L), valine (V), alanine (A), and methionine (M) are aliphatic, lysine (K), arginine (R), and histidine (H) are basic; aspartic acid (D) and glutamic acid (E) are acidic; serine (S), threonine (T), asparagine (N), and glutanine (Q) are polar; and proline (P), glycine (G), and cysteine (C) are conformational.

FIG. 24 shows an illustrative embodiment of designed diversity using nonrandom codon sets for amino acid positions in CDRs H1, H2 and H3 of antibody 4D5. The amino acids(in single letter code) encoded by the codon set at that position are shown below in a column.

FIG. 25 shows the results from sorting ScFv libraries for binding to target antigens Her-2, IGF-1 and mVEGF. The ScFv-1 library was generated with a vector having a zipper sequence, 4D5 heavy and light chain sequences, and with diversity in CDRH1, H2 and H3. The ScFv-2 was generated with a vector having a zipper sequence and with diversity in H1, H2, H3 and L3. The ScFv-3 was generated with a vector with a zipper sequence and with diversity in H3 and L3. The ScFv-4 has no zipper region and the CDR diversity was generated in CDR H1, H2 and H3. The ScFv-5 was generated with no zipper sequence and with CDR diversity in CDR's H1, H2, H3 and L3. The results for each library after three rounds of sorting are shown as a % of clones binding to a target antigen.

FIG. 26 shows the results of specific binders isolated from ScFv-1, ScFv-2 and ScFv-3 libraries. Phage clones from 3 rounds of selection against IGF-1 or mVEGF were analyzed for specific binding by ELISA assays using both IGF1 and mVEGF. Clones that bound to the target for which they were selected and not to the other antigen were identified as specific. The percentage of clones from each selection that bound targets (Total) and the percentage of clones that bound only the target against which they were selected (specific) are shown.

FIG. 27 shows the total number of sequences and of those sequences the number of unique sequences of anti-VEGF or anti-IGF antibody variable domains identified from each library of scFv-1 and scFv-4 using 4D5 template after two or three rounds of sorting.

FIG. 28 shows an example of CDRH3 codon/amino acid usage distribution in one set of binders. “X” denotes codon set usage as shown for each oligonucleotides in FIG. 4. The percentage of the CDR-H3 design of each oligonucleotides in the sequences of binders isolated from the library are shown.

FIG. 29 shows H3 sequences and affinities of some anti-IGF1 and anti-VEGF binders from a F(ab′)₂ L3/H3 library generated using 4D5 anitbody template. Underlined residues represent residues that were fixed in the source library of the clones.

FIG. 30 shows the identity of the epitope bound by some of the clones. Murine VEGF was coated on plate and phage clones competitively inhibited in the present of KDR-7igg were identified. Clones V1 (-●-), V2(-∘-), V4 (-♦-), V5 (-▴-), V6 (-+-), V7 (-Δ-), V8 (- -), V9 (-▪-), V10 (-▾-) were tested.

FIG. 31 shows the identity of the epitope bound by some of the clones. Murine VEGF was coated on a plate and phage clones competitively inhibited in the present of Flt1-D2 were identified. Clones V1 (-●-), V2(-∘-), V4 (-♦-), V5 (-▴-), V6 (-+-), V7 (-Δ-), V8 (- -), V9 (-▪-), V10 (-▾-) were tested.

FIG. 32 shows the Fab polypeptide phage CDRH3 amino acid sequences from libraries generated form the 4D5 template, affinities, epitope specificity and production of Fab in cell culture for clones V1, V2, V3, and V8.

FIG. 33 shows the heavy chain variable domain CDR amino acid sequences and affinities of binders to mVEGF and human Fc receptor from a F(ab) or F(ab′)₂ library of the 4D5 template. The amino acid sequence of heavy chain framework positions 49, 71, 93 and 94 are also shown.

FIGS. 34A-D are a schematic illustration of phagemid constructs. FIG. 34A shows a bicistronic vector allowing expression of separate transcripts for display of Fab. A suitable promoter, such as Ptac or PhoA promoter drives expression of the bicistronic message. The first cistron encodes a malE or heat stable enterotoxin II (stII) secretion signal connected to a sequence encoding a light chain variable and constant domain and a gD tag. The second cistron encodes a secretion signal sequence, a heavy chain variable domain and constant domain 1 (CH1) and at least a portion of a viral coat protein. FIG. 34B shows a bicistronic message for display of F(ab′)₂. A suitable promoter drives expression of the first and second cistron. The first cistron encodes a secretion signal sequence (malE or stII), a light chain variable and constant domain and a gD tag. The second cistron encodes a secretion signal, a sequence encoding heavy chain variable domain and constant domain 1 (CH1) and dimerization domain and at least a portion of the viral coat protein. FIG. 34C is a monocistronic vector for display of ScFv. A suitable promotor drives expression of V_{L} and V_{H} domains linked by a peptide linker. The cistronic sequence is connected at the 5′ end to a secretion signal sequence and at 3′ end to at least a portion of a viral coat protein (pIII). FIG. 34D shows a vector for diplay of ScFv₂. The vector is similar to FIG. 34C, but comprises a dimerization domain between V_{H} and the coat protein.

FIG. 35 shows the amino acid sequences for heavy chain variable CDR sequences and affinities of anti-VEGF binders from a ScFv and ScFv₂ library prepared from 4D5 template.

FIG. 36 shows a 3-D modeled structure of humanized 4D5 showing CDR residues that form contiguous patches. Contiguous patches are formed by amino acid residues 28, 29,30,31 and 32 in CDRL1; amino acids residues 50 and 53 of CDRL2; amino acid residues 91,92, 93, 94 and 96 of CDRL3; amino acid residues 28, 30, 31, 32,33 in CDRH1; and amino acid residues 50,52,53,54,56, and 58 in CDRH2.

FIG. 37 shows the nucleotide (SEQ ID NO: 135) (a) and amino acid sequence (SEQ ID NO: 136 (b) of the llama anti-HCG monobody variable heavy chain. The numbering for the 17 residue CDRH3 region is shown in (SEQ ID NO:137) (c).

FIG. 38 shows an alanine scan of wild type CDRH3 from the variable heavy chain (VHH) of anti-HCG monobody. The graph shows the ratio of sequences with wild type amino acid at the selected amino acid positions (96, 97, 98, 99, 100, 101, and 102) to sequences with alanine at each of those positions.

FIG. 39 shows the crystal structure of camelid monobody anti-HCG and camelid monobody anti-RNAse A showing interface packing by CDRH3 at the former light chain interface.

FIG. 40 shows the analysis of the amino acid distribution at the framework positions in the VHH of anti-HCG monobody. Positions 37, 45, 47 and 91 were each substituted with all 20 amino acids using NNS degenerate codons. The variants were sorted for binding to protein A and sequenced. The tabulated totals were corrected for codon bias and normalized totals were used to calculate the percent occurrence of each amino acid type at each position. The results show that positions 37 and 45 have a bias for certain amino acids; in amino acid position 37 phenylalanine is preferred and at position 45 leucine or arginine is preferred.

FIG. 41 shows the distribution of CDRH3 lengths in camel monobodies as compared to human and murine antibodies.

FIG. 42 shows the amino acid bias in CDRH3 using human anti-HCG as monobody and a 17 amino acid insert of CDRH3. The 17 amino acid insert was randomized at each position and the library was sorted for binding to protein A. The frequency of each amino acid at each position in the CDRH3 is shown.

FIG. 43 shows the aggregate analysis of the amino acid distribution in CDRH3 of library NNS17 of a VHH following 3 rounds of selection for binding to protein A. The tabulated totals were corrected for codon bias to obtain (a) the normalized totals for each amino acid at each position. The total, and frequency of occurrence were determined across each row and down each column. The positional dependence for each amino acid was measured by calculating the Pearson residuals for the entire data set (b). Highlighted values are large for those residues where there is a strong selection bias. Highlighted residues show residue positions for which the distribution is significantly different from a random distribution (p<0.05). The numbering follows the Kabat nomenclature.

FIG. 44 shows amino acid bias by position type. A library of CDRH3 variant was prepared as described previously with each position of the 17 amino acid insert randomized. The library was sorted using protein A and the binders were sequenced. The data was analyzed for bias for particular amino acids at certain positions. Those amino acids found at a position at a frequency one standard deviation greater than would be expected randomly for that amino acid are shown.

FIG. 45 shows the 10 most abundant CDRH3 sequences in library NNS17 of VHH following 4 rounds of selection for binding to protein A. The top 10 sequences (a) are shown in rank order along with the percent of the total population (percent abundance) that each represented following 3 or 4 rounds of selection. Sequences that match the aggregate consensus are in underlined bold text. The results of the shotgun alanine-scanning analysis (b) are shown for the top 4 scaffolds. The wt/Ala ratios for each residue in CDR3 are shown for the scaffolds RIG (white bars), LLR (cross-hatched bars), VLK (grey bars), and RLV (black bars). The name of each scaffold is derived from the sequence at positions 96, 97 and 98. The numbering follows the Kabat nomenclature.

FIG. 46 shows distribution of randomized/nonstructural lengths of contiguous amino acid sequence in CDRH3 that can be accommodated by a VHH RIG scaffold without affecting structural stability.

FIG. 47 shows an alanine scan of CDR3 of an RIG VHH scaffold. A library was generated using the RIG scaffold with positions 96, 97, 100i, and 100j with fixed amino acids:amino acid position 96 was arginine, amino acid position 97 was isoleucine, amino acid position 100i was tryptophan and amino acid position 100j was valine. An insert of 11 amino acids was inserted between residue number 97 and 100i. This insert was randomized. The resulting library was sorted against VEGF.

FIG. 48 shows amino acid bias in naïve anti-VEGF library generated using the RIG VHH scaffold. The VEGF binders were isolated and sequenced. The sequences of the binders were analyzed for amino acid bias at certain positions using the Pearson analysis as described previously. Highlighted numbers indicates a bias for that amino acid at that position.

FIG. 49 shows a two-point competition ELISA to measure binding of clones from the VHH RIG library prepared with an N terminal sequence R—I—X—C (SEQ ID NO:138) and with a C terminal sequence C—W—V-T-W (SEQ ID NO:139) with a randomized central portion of 6 amino acids in between. VEGF binders were analyzed for binding to VEGF using two concentrations of VEGF (2 μm and 20 μm). The clones identified with asterisks were characterized further.

FIG. 50 shows representative phage ELISAs from VEGF positive clones after four rounds of sorting from the VHH RIG generated by fixing cysteines at the N terminal and C terminal ends of the randomized central portion or insert.

FIG. 51 shows a ribbon diagram of the x-ray crystal structure of the VHH RIG. Protein crystals were grown in 30% PEG 4K, 0.3 ammonium sulfate, pH 7.0 at 20 C. A molecular replacement solution was found using the published anti-HCG VHH domain structure minus 96-102, as search model. Structures were rendered in Pymol (Delano Scientific, San Carlos, Calif.)

FIG. 52 shows the results of randomizing framework positions 37, 45, 37 and 91 in the VHH RIG. The positions were randomized using all 20 amino acids and sorted for binding to Protein A. The binders were sequenced and the sequences were analyzed for amino acid bias using the Pearson analysis as described previously. Highlighted amino acids are those that show a bias at that amino acid position.

FIG. 53 shows the framework and CDRH3 residues involved in VHH domain stabilization. Ribbon views are shown for the a) the anti-HCG domain and b) the RIG domain.

TABLE OF SEQUENCES

| | | | |
|---|---|---|---|
| SEQ ID NO: | Name | Sequence | Page |

| | | | |
|---|---|---|---|
| 1 | 4D5 light chain | Asp Ile Gln Met Thr Gln Ser Pro Ser | 29 |
| | variable domain | Ser Leu Ser Ala Ser Val Gly Asp Arg | |
| | | Val Thr Ile Thr Cys Arg Ala Ser Gln | |
| | | Asp Val Asn Thr Ala Val Ala Trp Tyr | |
| | | Gln Gln Lys Pro Gly Lys Ala Pro Lys | |
| | | Leu Leu Ile Tyr Ser Ala Ser Phe Leu | |
| | | Glu Ser Gly Val Pro Ser Arg Phe Ser | |
| | | Gly Ser Arg Ser Gly Thr Asp - #Phe | |
| | | Thr Leu Thr Ile Ser Ser Leu Gln Pro | |
| | | Glu Asp Phe Ala Thr Tyr Tyr Cys Gln | |
| | | Gln His Tyr Thr Thr Pro Pro Thr Phe | |
| | | Gly Gln Gly Thr Lys Val Glu Ile Lys | |
| | | Arg Thr | |
| 2 | 4D5 heavy chain | Glu Val Gln Leu Val Glu Ser Gly Gly | 29 |
| | variable domain | Gly Leu Val Gln Pro Gly Gly Ser Leu | |
| | | Arg Leu Ser Cys Ala Ala Ser Gly Phe | |
| | | Asn Ile Lys Asp Thr Tyr Ile His Trp | |
| | | Val Arg Gln Ala Pro Gly Lys Gly Leu | |
| | | Glu Trp Val Ala Arg Ile Tyr Pro Thr | |
| | | Asn Gly Tyr Thr Arg Tyr Ala Asp Ser | |
| | | Val Lys Gly Arg Phe Thr Ile Ser Ala | |
| | | Asp Thr Ser Lys Asn Thr Ala Tyr Leu | |
| | | Gln Met Asn Ser Leu Arg Ala Glu Asp | |
| | | Thr Ala Val Tyr Tyr Cys Ser Arg Trp | |
| | | Gly Gly Asp Gly Phe Tyr Ala Met Asp | |
| | | Val Trp Gly Gln Gly Thr Leu Val Thr | |
| | | Val Ser Ser | |
| 3 | GNC4 leucine | GRMKQLEDKVEELLSKNYHLENE | 26 |
| | zipper | VARLKKLVGERG | |
| 4 | C-terminal of | YAMDY | 99 |
| | CDRH3 of 4D5 | | |
| 5 | heavy chain CDR3 | SRNAWAF | 118 |
| 6 | heavy chain CDR3 | SRNLSENSYAM | 118 |
| 7 | heavy chain CDR3 | SRAGWAGWYAM | 118 |
| 8 | heavy chain CDR3 | SRAAKAGWYAM | 118 |
| 9 | heavy chain CDR3 | SRSDGRDSAYAM | 118 |
| 10 | F63 | SRXXXXXXXAMDY | |
| 11 | F65 | SRXXXXXXXYAMDY | |
| 12 | F64 | SRXXXXXXYAMDY | |
| 13 | F66 | SRXXXXXXYAMDY | |
| 14 | oligonucleotide | gca gct tct ggc ttc acc att ant nnt nnn | 137 |
| | F151 | nnt ata cac tgg gtg cgt cag | |
| 15 | oligonucleotide | gca gct tct ggc ttc acc att ant nnt nnn | 137 |
| | F152 | ngg ata cac tgg gtg cgt cag | |
| 16 | oligonucleotide | aag ggc ctg gaa tgg gtt gst dgg att wmt | 137-138 |
| | F153 | cct dmt rrc ggt dmt act dac tat gcc gat | |
| | | agc gtc aag ggc | |
| 17 | oligonucleotide | aag ggc ctg gaa tgg gtt gst dht att wmt | 138 |
| | F154 | cct dmt rrc ggt dmt act dac tat gcc gat | |
| | | agc gtc aag ggc | |
| 18 | single chain Fv | | |
| 19 | single chain Fv | | |
| | with zipper domain | | |
| 20 | Fab fragment | | |
| 21 | Fab fragment with | | |
| | zipper domain | | |
| 22 | hinge sequence | TCPPCPAPELLG | 124 |
| 23 | oligonucleotide F61 | gca act tat tac tgt cag caa nrt nrt rvm | 136 |
| | | nnk cct tdk acg ttc gga cag ggt acc | |
| 24 | F59 | SRWGGDGFYAMDY | |
| 25 | F78 | SRXXXXXFDY | |
| 26 | F165 | AXXXXXXXYAMDY | |
| 27 | F166 | AXWXXXXXXAMDY | |
| 28 | F134 | AXXXXXXXYAMDY | |
| 29 | F136 | AXWXXXXXXYAMDY | |
| 30 | F137 | AXXWXXXXXYAMDY | |
| 31 | F138 | AXXXWXXXXYAMDY | |
| 32 | F142 | AXXXXXXXWYAMDY | |
| 33 | F155 | AXWXXXXXXXAMY | |
| 34 | F156 | AXXWXXXXXXAMDY | |
| 35 | F157 | AXXXWXXXXXAMDY | |
| 36 | F158 | AXXXXWXXXXAMDY | |
| 37 | F160 | AXXXXXXWXXAMDY | |
| 38 | F160g | AXXXXXXXWXAMDY | |
| 39 | F163a | AXXXXXXXXAMDY | |
| 40 | F164a | ARXXXXXXXYAMDY | |
| 41 | F164b | ARXXXXXXXXAMDY | |
| 42 | F165a | ARXXXXXXXXYAMDY | |
| 43 | F165b | ARXXXXXXXXXAMDY | |
| 44 | F167 | AXWXXXXXXAMDY | |
| 45 | F135 | AXWXXXXXXAMDY | |
| 46 | F103 | SRXXXXXXXXYAMDY | |
| 47 | F66a | ARXXXXXXYAMDY | |
| 48 | F66b | ARXXXXXXXAMDY | |
| 49 | F66c | ARXXXXXYXMDY | |
| 50 | F66d | ARXXXXXXXMDY | |
| 51 | F66e | ARXXXXYXMDY | |
| 52 | F66f | ARXXXXXXMDY | |
| 53 | F66a1 | ARXXXXXXYXMDY | |
| 54 | F66b1 | ARXXXXXXXXMDY | |
| 55 | F66g | ARXXXXXXXXXMDY | |
| 56 | F66h | ARXXXXXXXYXMDY | |
| 57 | F66i | ARXXXXXXXXYXMDY | |
| 58 | F66j | ARXXXXXXXXXXMDY | |
| 59 | F171c | AXXXXXXFDY | |
| 60 | F171d | AXXXXXXXFDY | |
| 61 | F171e | AXXXXXXXXFDY | |
| 62 | F171 | AXXXXXFDY | |
| 63 | F186 | AXXXXXXXXFDY | |
| 64 | F187 | AXXXXXXXXXFDY | |
| 65 | F190 | AXXXXXXXXXXYAMDY | |
| 66 | F190a | AXXXXXXXXXYXMDY | |
| 67 | F190d | AXXXXXXXXXXXYXMDY | |
| 68 | CDRH3 | SRWKYATRYAM | 118, FIG. |
| | | | 29 |
| 69 | CDRH3 | SRSRGWWTAAM | 1118, FIG. |
| | | | 29 |
| 70 | CDRH3 | SRASRDWYGAM | 118, FIG. |
| | | | 29 |
| 71 | mVEGF-201 | TTSNG | |
| | CDRH1 | | |
| 72 | mVEGF-201 | AYSSNYYR | |
| | CDRH2 | | |
| 73 | mVEGF-201 | ARWSRASFY | |
| | CDRH3 | | |
| 74 | mVEGF-202 | TTGTD | |
| | CDRH1 | | |
| 75 | mVEGF-202 | AITYDSYR | |
| | CDRH2 | | |
| 76 | mVEGF-202 | AKAGDREGY | |
| | CDRH3 | | |
| 77 | mVEGF-203 | TTDSG | |
| | CDRH1 | | |
| 78 | mVEGF-203 | GRSYSSNR | |
| | CDRH2 | | |
| 79 | mVEGF-203 | AKWPWYNAW | |
| | CDRH3 | | |
| 80 | hFc-10 CDRH1 | TNNYW | |
| 81 | hFc-10 CDRH2 | GYSYGTR | |
| 82 | hFc-10 CDRH3 | AKAXKGSLY | |
| 83 | hFc-11 CDRH1 | TTGNA | |
| 84 | hFc-12 CDRH1 | TNDYY | |
| 85 | hFc-13 CDRH1 | TSNTG | |
| 86 | hFc-14 CDRH1 | TTSYG | |
| 87 | hFc-14 CDRH2 | ASSYSYR | |
| 88 | hFc-14 CDRH3 | AKYXAREGX | |
| 89 | hFc-15 CDRH1 | TNNNS | |
| 90 | hFc-15 CDRH2 | GYNSGSR | |
| 91 | hFc-15 CDRH3 | AKWRTSWKY | |
| 92 | hFc-16 CDRH1 | TSSSA | |
| 93 | hFc-16 CDRH2 | AWSNGSR | |
| 94 | hFc-16 CDRH3 | AXTAGGAKY | |
| 95 | hFc-17 CDRH1 | TTNTW | |
| 96 | hFc-17 CDRH2 | GDYDGYR | |
| 97 | hFc-17 CDRH3 | AXWRWWGRY | |
| 98 | hFc-18 CDRH1 | TNGNY | |
| 99 | hFc-18 CDRH2 | GWSNGYR | |
| 100 | hFc-18 CDRH3 | ARYSGGRRY | |
| 101 | hFc-19 CDRH1 | TSNNA | |
| 102 | hFc-19 CDRH2 | GRSYNYR | |
| 103 | hFc-19 CDRH3 | AXGXTSGGY | |
| 104 | hFc-20 CDRH1 | TTSND | |
| 105 | hFc-20 CDRH2 | AWSYNYR | |
| 106 | hFc-20 CDRH3 | ARRSRWSRA | |
| 107 | mVEGF-109 | TGNSW | |
| | CDRH1 | | |
| 108 | mVEGF-109 | VATYYN | |
| | CDRH2 | | |
| 109 | mVEGF-109 | WGAKGTW | |
| | CDRH3 | | |
| 110 | mVEGF-126 | NADSA | |
| | CDRH1 | | |
| 111 | mVEGF-126 | YAYDYY | |
| | CDRH2 | | |
| 112 | mVEGF-126 | WGWTTNG | |
| | CDRH3 | | |
| 113 | mVEGF-127 | NDNTA | |
| | CDRH1 | | |
| 114 | mVEGF-127 | VSHDTY | |
| | CDRH2 | | |
| 115 | mVEGF-127 | WGWETDG | |
| | CDRH3 | | |
| 116 | mVEGF-130 | LDSSYD | |
| | CDRH2 | | |
| 117 | mVEGF-130 | SRAGYTY | |
| | CDRH3 | | |
| 118 | mVEGF-136 | NGKSS | |
| | CDRH1 | | |
| 119 | mVEGF-136 | WSYEAA | |
| | CDRH2 | | |
| 120 | mVEGF-136 | TSWSKPY | |
| | CDRH3 | | |
| 121 | mVEGF-169 | NTAYG | |
| | CDRH1 | | |
| 122 | mVEGF-169 | VTYDDT | |
| | CDRH2 | | |
| 123 | mVEGF-169 | WGWEANW | |
| | CDRH3 | | |
| 124 | mVEGF-173 | TGGSW | |
| | CDRH1 | | |
| 125 | mVEGF-173 | VYTYYD | |
| | CDRH2 | | |
| 126 | mVEGF-173 | WGAGGTW | |
| | CDRH3 | | |
| 127 | mVEGF-174 | VSDYYD | |
| | CDRH2 | | |
| 128 | mVEGF-174 | WGSGYTW | |
| | CDRH3 | | |
| 129 | mVEGF-176 | SAGYD | |
| | CDRH1 | | |
| 130 | mVEGF-176 | LAYAYN | |
| | CDRH2 | | |
| 131 | mVEGF-176 | AAAWASY | |
| | CDRH3 | | |
| 132 | mVEGF-179 | TTESG | |
| | CDRH1 | | |
| 133 | mVEGF-179 | VYHDKY | |
| | CDRH2 | | |
| 134 | mVEGF-179 | WWYSWNW | |
| | CDRH3 | | |
| 135 | nucleotide sequence | GAT GTT CAG TTG CAG GAA TCA | |
| | of VHH anti-HCG | GGC GGT GGC TT GTA CAG GCC | |
| | monobody | GGA GGT TCG TTG CGT TTG TCC | |
| | | TGT GCT GCC TCG GGT CGT ACT | |
| | | GGT TCT ACT TAT GAT ATG GGC | |
| | | TGG TTT CGT CAG GCT CCG GGT | |
| | | AAA GAA CGT GAA TCG GTT | |
| | | GCC GCC ATT AAC TGG GAT TCG | |
| | | GCT CGT ACT TAC TAT GCT TCG | |
| | | TCC GTC CGT GGT CGT TTT ACT | |
| | | ATT TCA CGT GAT AAT GCC AAA | |
| | | AAA ACT GTC TAT TTG CAG ATG | |
| | | AAT TCA TTG AAA CCA GAA GAT | |
| | | ACT GCC GTC TAT ACT TGT GGT | |
| | | GCT GGT GAA GGC GGT ACT TGG | |
| | | GAT TCT TGG GGT CAG GGT ACC | |
| | | CAG GTC ACT GTC TCC TCT GCC | |
| | | GGT GGT ATG GAT TAT AAA GAT | |
| | | GAT GAT GAT AAA TGA | |
| 136 | amino acid | DVQLQESGGGLVQAGGSLRLSCA | |
| | sequence of VHH | ASGRTGSTYDMGWFRQAPGKERE | |
| | anti-HCG | SVAAINWDSARTYYASSVRGRFTI | |
| | monobody | SRDNAKKTVYLQMNSLKPEDTAV | |
| | | YTCGAGEGGTWDSWGQGTQVTV | |
| | | SSAGGMDYKDDDDK | |
| 137 | amino acid | CGAGXXXXXXXXXXXXXXXXX | |
| | sequence of | WG | |
| | CDRH3 17 amino | | |
| | acid insert | | |
| 138 | | RIXC | 41 |
| 139 | | CWVTW | 41 |
| 140 | | A1-A2-(A3)n-A4-A5; | 11 |
| | | A1 is R, L, V, F, W, or K; | |
| | | A2 is I, L, V, R, W, or S; | |
| | | A3 is any naturally occurring amino | |
| | | acid, n is 1 to 17; | |
| | | A4 is W, G, R, M, S, or A; | |
| | | A5 is V, L, P, G, S, E or W. | |
| 141 | | A1-A2-(A3)n-A4-A5-A6-A7; | 12 |
| | | A1 is R, L, V, F, W, or K; | |
| | | A2 is I, L, V, R, W, or S; | |
| | | A3 is any naturally occurring amino | |
| | | acid, n is 1 to 17; | |
| | | A4 is W, G, R, M, S, or A; | |
| | | A5 is V, L, P, G, S, E or W; | |
| | | A6 is any naturally occurring amino | |
| | | acid; | |
| | | A7 is any naturally occurring amino | |
| | | acids | |
| 142 | | A1-A2-(A3)n-A4-A5-A6-A7-A8-A9 | 13 |
| | | A1 is R, L, or V | |
| | | A2 is I, L, or V; | |
| | | A3 is any naturally occurring amino | |
| | | acid, n is 1 to 17; | |
| | | A4 is E, W, or F; | |
| | | A5 is any naturally occurring amino | |
| | | acid; | |
| | | A6 is W, G, R, or M; | |
| | | A7 is V, L, or P; | |
| | | A8 is any naturally occurring amino | |
| | | acid; | |
| | | A9 is any naturally occurring amino | |
| | | acid | |
| 143 | | R-A2-A3-R-(A5)n; | 14 |
| | | A2 is L, I, or M | |
| | | A3 is any naturally occurring amino | |
| | | acid; | |
| | | A5 is any naturally occurring amino | |
| | | acid, n is 1 to 20 | |
| 144 | | R-A2-(A3)n-W-A5-A6-A7-A8-A9; | 14 |
| | | A2 is L, I, or M; | |
| | | A3 is any naturally occurring amino | |
| | | acid, n is 1 to 15; | |
| | | A5 is any naturally occurring amino | |
| | | acid; | |
| | | A6 is W, G, R, or M; | |
| | | A7 is V, L, or P; | |
| | | A8 is any naturally occurring amino | |
| | | acid; | |
| | | A9 is any naturally occurring amino | |
| | | acid. | |
| 145 | | R-I-X-X-X-X-X-X-X-X-X-X-X-W-V- | 15 |
| | | A6-A7; | |
| | | X is any naturally occurring amino | |
| | | acid; | |
| | | A6 is any naturally occurring amino | |
| | | acid; | |
| | | A7 is any naturally occurring amino | |
| | | acid | |
| 146 | | V-L-X-X-X-X-X-X-X-X-X-F-A5-R-V | 15 |
| | | X is any naturally occurring amino | |
| | | acid; | |
| | | A5 is any naturally occurring amino | |
| | | acid | |
| 147 | | R-L-X-X-X-X-X-X-X-X-X-W-A5-A6- | 15 |
| | | A7-A8-A9 | |
| | | X is any naturally occurring amino | |
| | | acid; | |
| | | A5 is any naturally occurring amino | |
| | | acid; | |
| | | A6 is any naturally occurring amino | |
| | | acid; | |
| | | A7 is any naturally occurring amino | |
| | | acid; | |
| | | A8 is any naturally occurring amino | |
| | | acid; | |
| | | A9 is any naturally occurring amino | |
| | | acid | |
| 148 | | L-L-X-X-X-X-X-X-X-X-X-W-A5-A6- | 15 |
| | | A7-A8-A9 | |
| | | X is any naturally occurring amino | |
| | | acid; | |
| | | A5 is any naturally occurring amino | |
| | | acid; | |
| | | A6 is any naturally occurring amino | |
| | | acid; | |
| | | A7 is any naturally occurring amino | |
| | | acid; | |
| | | A8 is any naturally occurring amino | |
| | | acid; | |
| | | A9 is any naturally occurring amino | |
| | | acid | |
| 149 | | R-I-A3-C-X-X-X-X-X-X-X-C-W-V- | 15 |
| | | A8-A9-A10 | |
| | | X is any naturally occurring amino | |
| | | acid; | |
| | | A3 is any naturally occurring amino | |
| | | acid; | |
| | | A8 is any naturally occurring amino | |
| | | acid; | |
| | | A9 is any naturally occurring amino | |
| | | acid; | |
| | | A10 is any naturally occurring amino | |
| | | acids | |
| 150 | N terminal | R-L/I/M-A₃-R, | 15 |
| | sequence | A₃ is any naturally occurring amino | |
| | | acid | |
| 151 | ala scan wild type | 5′- | 141 |
| | CDRH3 | GCCGTCTATACTTGTGGTGCTGG | |
| | | TGMAGSTGSTRCTKSGGMTKCCT | |
| | | GGGGTCAGGGTACC-3′ | |
| 152 | framework scan of | 5′ | 141 |
| | residue 37-47 of | GATATGGGCTGGNNSCGTCAGGC | |
| | wild type | TCCGGGTAAAGAANNSGAANNSG | |
| | | TTGCCGCCA-3′ | |
| 153 | framework scan of | 5′- | 142 |
| | residue 91 of wild | GATACTGCCGTCTATNNSTGTGG | |
| | type | TGCTGGTGAAGGCGGTACTTGGG | |
| | | ATTCTTGGGGTCAG-3′ | |
| 154 | NNS library | 5′- | 146 |
| | | GCCGTCTATACTTGTGGTGCTGG | |
| | | TNNSNNSNNSNNSNNSNNSNNSN | |
| | | NSNNSNNSNNSNNSNNSNNSNNS | |
| | | NNSNNSTGGGGTCAGGGT-3′, | |
| 155 | RIG ala scan | 5′- | 151 |
| | | GCCGTCTATACTTGTGGTGCTGG | |
| | | TSSTRYTGSTSSTKCCGYTKYTRM | |
| | | CSYTSSTSSTGMAKCCKSGGYTRC | |
| | | TKSGTGGGGTCAGGGT-3′, | |
| 156 | VLK ala scan | 5′- | 151 |
| | | GCCGTCTATACTTGTGGTGCTGG | |
| | | TGYTSYTRMASSTSSTGSTKCCKC | |
| | | CGYTGSTRYTKYTRCTSSTGYTSM | |
| | | AKCCTGGGGTCAGGGT | |
| 157 | LLR ala scan | 5′- | 151 |
| | | GCCGTCTATACTTGTGGTGCTGG | |
| | | TSYTSYTSSTSSTGSTGYTRMCGC | |
| | | GRCTSCARMCKSGKYTGSTSYTG | |
| | | YTGSTTGGGGTCAGGGT-3′, | |
| 158 | RLV ala scan | 5′- | 152 |
| | | GCCGTCTATACTTGTGGTGCTGG | |
| | | TSSTSYTGYTRMCGSTSYTKCCGS | |
| | | TSYTGYTKCCKSGGMARYGSCAS | |
| | | YTGCGTGGGGTCAGGG-3′ | |
| 159 | N terminal | R-I-A₃-C | 15 |
| | sequence of | A3 can be any naturally occurring | |
| | CDRH3 scaffold | amino acid | |
| 160 | C terminal | F-X-R-V | 15 |
| | sequence of | X can be any naturally occurring amino | |
| | CDRH3 scaffold | acid | |
| 161 | C terminal | W-X-X-L | 15 |
| | sequence of | X can be any naturally occurring amino | |
| | CDRH3 scaffold | acid | |
| 162 | C terminal | W-X-M-P | 15 |
| | sequence of | X can be any naturally occurring amino | |
| | CDRH3 scaffold | acid | |
| 163 | | RIGRSVFNLRRESWVTW | |
| 164 | | LLRRGVNATPNWFGLVG | |
| 165 | | VLKRRGSSVAIFTRVQS | |
| 166 | | RLVNGLSGLVSWEMPLA | |
| 167 | | FVAGPWWWRWRTPSGVA | |
| 168 | | VLELRSSGGNARWMSLY | |
| 169 | | LRISPYAFWLGTWAPSY | |
| 170 | | LWTRARSWRWWWRREQF | |
| 171 | | WRSWISSILGLRTWWYA | |
| 172 | | KSTRWRAGHGRTFHWLS | |
| 173 | F139 | AXXXXWXXXYAMDY | |
| | | X is NVT | |
| 174 | F140 | AXXXXWXXYAMDY | |
| | | X is NVT | |
| 175 | F141 | AXXXXXXWXYAMDY | |
| | | X is NVT | |
| 176 | F142 | AXXXXXXWYAMDY | |
| | | X is NVT | |
| 177 | F170 | AXXXXXFDY | |
| | | X is NVT | |
| 178 | F171a | AXXXXXXXXFDY | |
| | | X is NVT | |
| 179 | F171b | AXXXXXXXXXFDY | |
| | | X is NVT | |
| 180 | F181 | AXXXXXXXXXAMDY | |
| | | X is XYZ or KSG | |
| 181 | F179 | AXXXXXXXXYAMDY | |
| | | X is XYZ | |
| 182 | F182 | AXXXXXXXXXXAMDY | |
| | | X is XYZ or KSG | |
| 183 | F183 | AXXXXXYAMDY | |
| | | X is XYZ | |
| 184 | F184 | AXXXXXXYAMDY | |
| | | X is XYZ of KSG | |

### DETAILED DESCRIPTION OF THE INVENTION

The invention provides novel and systematic methods for diversifying antibody variable domain sequences, and libraries comprising a multiplicity, generally a great multiplicity of diversified antibody variable domain sequences. Such libraries provide combinatorial libraries useful for, for example, screening for synthetic antibody or antigen binding polypeptides with desirable activities such as binding affinities and avidities and structural stability. These libraries provide a tremendously useful resource for identifying immunoglobulin polypeptide sequences that are capable of interacting with any of a wide variety of target molecules. For example, libraries comprising diversified immunoglobulin polypeptides of the invention expressed as phage displays are particularly useful for, and provide a high throughput, efficient and automatable systems of, screening for antigen binding molecules of interest. In some embodiments, the diversified antibody variable domains are provided in a monobody that binds to antigen in the absence of light chains. Also provided are methods for designing CDRH3 regions that can be used to generate a plurality of CDRH3 regions. The population of variant CDRH3 can then be utilized in libraries to identify novel antigen binding molecules.

Definitions

The term “affinity purification” means the purification of a molecule based on a specific attraction or binding of the molecule to a chemical or binding partner to form a combination or complex which allows the molecule to be separated from impurities while remaining bound or attracted to the partner moiety.

The term “antibody” is used in the broadest sense and specifically covers single monoclonal antibodies (including agonist and antagonist antibodies), antibody compositions with polyepitopic specificity, affinity matured antibodies, humanized antibodies, chimeric antibodies, single chain antigen binding molecules such as monobodies, as well as antigen binding fragments or polypeptides (e.g., Fab, F(ab′)₂, scFv and Fv), so long as they exhibit the desired biological activity.

As used herein, “antibody variable domain” refers to the portions of the light and heavy chains of antibody molecules that include amino acid sequences of Complementary Determining Regions (CDRs; ie., CDR1, CDR2, and CDR3), and Framework Regions (FRs). V_{H} refers to the variable domain of the heavy chain. V_{L} refers to the variable domain of the light chain. VHH refers to the heavy chain variable domain of a monobody. According to the methods used in this invention, the amino acid positions assigned to CDRs and FRs are defined according to Kabat (Sequences of Proteins of Immunological Interest (National Institutes of Health, Bethesda, Md., 1987 and 1991)). Amino acid numbering of antibodies or antigen binding fragment or polypeptides is also according to that of Kabat et al. cited supra.

As used herein, “codon set” refers to a set of different nucleotide triplet sequences used to encode desired variant amino acids. A set of oligonucleotides can be synthesized, for example, by solid phase synthesis, containing sequences that represent all possible combinations of nucleotide triplets provided by the codon set and that will encode the desired group of amino acids. A standard form of codon designation is that of the IUB code, which is known in the art and described herein. A “non-random codon set”, as used herein, thus refers to a codon set that encodes select amino acids that fulfill partially, preferably completely, the criteria for amino acid selection as described herein. Synthesis of oligonucleotides with selected nucleotide “degeneracy” at certain positions is well known in that art, for example the TRIM approach (Knappek et al.; J. Mol. Biol. (1999), 296:57-86); Garrard & Henner, Gene (1993), 128:103). Such sets of nucleotides having certain codon sets can be synthesized using commercial nucleic acid synthesizers (available from, for example, Applied Biosystems, Foster City, Calif.), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used according to the invention, have sequences that allow for hybridization to a variable domain. nucleic acid template and also can, but does not necessarily, include restriction enzyme sites useful for, for example, cloning purposes.

An “Fv” fragment is an antibody fragment which contains a complete antigen recognition and binding site. This antibody fragment comprises a dimer of one heavy and one light chain variable domain in tight association, which can be covalent in nature, for example in scFv. It is in this configuration that the three CDRs of each variable domain interact to define an antigen binding site on the surface of the V_{H}—V_{L} dimer. Collectively, the six CDRs or a subset thereof confer antigen binding specificity to the antibody. However, even a single variable domain (comprising only three CDRs specific for an antigen) has the ability to recognize and bind antigen.

The “Fab” fragment contains a variable and constant domain of the light chain and a variable domain and the first constant domain (CH1) of the heavy chain. F(ab)′₂ antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the VH and VL domains of antibody, wherein these domains are present in a single polypeptide chain. Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol. 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10):1057-1062 (1995). Briefly, these antibodies comprise a pair of tandem Fd segments (V_{H}—C_{H}1-V_{H}—C_{H}1) which, together with complementary light chain polypeptides, form a pair of antigen binding regions. Linear antibodies can be bispecific or monospecific.

The term “monobody” as used herein, refers to an antigen binding molecule with a heavy chain variable domain and no light chain variable domain. A monobody can bind to an antigen in the absence of light chains and typically has three CDR regions designated CDRH1, CDRH2 and CDRH3. A heavy chain IgG monobody has two heavy chain antigen binding molecules connected by a disulfide bond. The heavy chain variable domain comprises one or more CDR regions, preferably a CDRH3 region. A “VₕH” or “VHH” refers to a variable domain of a heavy chain antibody such as a monobody. A “camelid monobody” or “camelid VHH” refers to a monobody or antigen binding portion thereof obtained from a source animal of the camelid family, including animals with feet with two toes and leathery soles. Animals in the camelid family include camels, llamas, and alpacas.

“Cell”, “cell line”, and “cell culture” are used interchangeably herein and such designations include all progeny of a cell or cell line. Thus, for example, terms like “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

“Control sequences” when referring to expression means DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

The term “coat protein” means a protein, at least a portion of which is present on the surface of the virus particle. From a functional perspective, a coat protein is any protein which associates with a virus particle during the viral assembly process in a host cell, and remains associated with the assembled virus until it infects another host cell. The coat protein may be the major coat protein or may be a minor coat protein. A “major” coat protein is generally a coat protein which is present in the viral coat at preferably at least about 5, more preferably at least about 7, even more preferably at least about 10 copies of the protein or more. A major coat protein may be present in tens, hundreds or even thousands of copies per virion. An example of a major coat protein is the p8 protein of filamentous phage.

The “detection limit” for a chemical entity in a particular assay is the minimum concentration of that entity which can be detected above the background level for that assay. For example, in the phage ELISA of Example 4, the “detection limit” for a particular phage displaying a particular antigen binding fragment or polypeptide is the phage concentration at which the particular phage produces an ELISA signal above that produced by a control phage not displaying the antigen binding fragment or polypeptide.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target molecule, catalysis of a reaction, etc. The two portions may be linked directly by a single peptide bond or through a peptide linker containing one or more amino acid residues. Generally, the two portions and the linker will be in reading frame with each other.

“Heterologous DNA” is any DNA that is introduced into a host cell. The DNA may be derived from a variety of sources including genomic DNA, cDNA, synthetic DNA and fusions or combinations of these. The DNA may include DNA from the same cell or cell type as the host or recipient cell or DNA from a different cell type, for example, from a mammal or plant. The DNA may, optionally, include marker or selection genes, for example, antibiotic resistance genes, temperature resistance genes, etc.

As used herein, “highly diverse position” refers to a position of an amino acid located in the variable regions of the light and heavy chains that have a number of different amino acid represented at the position when the amino acid sequences of known and/or naturally occurring antibodies or antigen binding fragment or polypeptides are compared. The highly diverse positions are typically in the CDR regions. In one aspect, the ability to determine highly diverse positions in known and/or naturally occurring antibodies is facilitated by the data provided by Kabat, Sequences of Proteins of Immunological Interest (National Institutes of Health, Bethesda, Md., 1987 and 1991). An internet-based database located at http:/immuno.bme.nwu.edu provides an extensive collection and alignment of human light and heavy chain sequences and facilitates determination of highly diverse positions in these sequences. According to the invention, an amino acid position is highly diverse if it has preferably from about 2 to about 11, preferably from about 4 to about 9, and preferably from about 5 to about 7 different possible amino acid residue variations at that position. In some embodiments, an amino acid position is highly diverse if it has preferably at least about 2, preferably at least about 4, preferably at least about 6, and preferably at least about 8 different possible amino acid residue variations at that position.

As used herein, “library” refers to a plurality of antibody or antibody fragment sequences (for example, polypeptides of the invention), or the nucleic acids that encode these sequences, the sequences being different in the combination of variant amino acids that are introduced into these sequences according to the methods of the invention.

“Ligation” is the process of forming phosphodiester bonds between two nucleic acid fragments. For ligation of the two fragments, the ends of the fragments must be compatible with each other. In some cases, the ends will be directly compatible after endonuclease digestion. However, it may be necessary first to convert the staggered ends commonly produced after endonuclease digestion to blunt ends to make them compatible for ligation. For blunting the ends, the DNA is treated in a suitable buffer for at least 15 minutes at 15° C. with about 10 units of the Klenow fragment of DNA polymerase I or T4 DNA polymerase in the presence of the four deoxyribonucleotide triphosphates. The DNA is then purified by phenol-chloroform extraction and ethanol precipitation or by silica purification. The DNA fragments that are to be ligated together are put in solution in about equimolar amounts. The solution will also contain ATP, ligase buffer, and a ligase such as T4 DNA ligase at about 10 units per 0.5 μg of DNA. If the DNA is to be ligated into a vector, the vector is first linearized by digestion with the appropriate restriction endonuclease(s). The linearized fragment is then treated with bacterial alkaline phosphatase or calf intestinal phosphatase to prevent self-ligation during the ligation step.

A “mutation” is a deletion, insertion, or substitution of a nucleotide(s) relative to a reference nucleotide sequence, such as a wild type sequence.

As used herein, “natural” or “naturally occurring” antibodies, refers to antibodies identified from a nonsynthetic source, for example, from a differentiated antigen-specific B cell obtained ex vivo, or its corresponding hybridoma cell line, or from the serum of an animal. These antibodies can include antibodies generated in any type of immune response, either natural or otherwise induced. Natural antibodies include the amino acid sequences, and the nucleotide sequences that constitute or encode these antibodies, for example, as identified in the Kabat database. As used herein, natural antibodies are different than “synthetic antibodies”, synthetic antibodies referring to antibody sequences that have been changed, for example, by the replacement, deletion, or addition, of an amino acid, or more than one amino acid, at a certain position with a different amino acid, the different amino acid providing an antibody sequence different from the source antibody sequence.

“Operably linked” when referring to nucleic acids means that the nucleic acids are placed in a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contingent and in reading frame. However, enhancers do not have to be contiguous. Linking is accomplished by ligation at convenient restriction sites. If such sites do not exist, the synthetic oligonucleotide adapters or linkers are used in accord with conventional practice.

“Phage display” is a technique by which variant polypeptides are displayed as fusion proteins to a coat protein on the surface of phage, e.g., filamentous phage, particles. A utility of phage display lies in the fact that large libraries of randomized protein variants can be rapidly and efficiently sorted for those sequences that bind to a target molecule with high affinity. Display of peptide and protein libraries on phage has been used for screening millions of polypeptides for ones with specific binding properties. Polyvalent phage display methods have been used for displaying small random peptides and small proteins through fusions to either gene III or gene VIII of filamentous phage. Wells and Lowman, Curr. Opin. Struct. Biol., 3:355-362 (1992), and references cited therein. In monovalent phage display, a protein or peptide library is fused to a gene III or a portion thereof, and expressed at low levels in the presence of wild type gene III protein so that phage particles display one copy or none of the fusion proteins. Avidity effects are reduced relative to polyvalent phage so that sorting is on the basis of intrinsic ligand affinity, and phagemid vectors are used, which simplify DNA manipulations. Lowman and Wells, Methods: A companion to Methods in Enzymology, 3:205-0216 (1991).

A “phagemid” is a plasmid vector having a bacterial origin of replication, e.g., Co1E1, and a copy of an intergenic region of a bacteriophage. The phagemid may be used on any known bacteriophage, including filamentous bacteriophage and lambdoid bacteriophage. The plasmid will also generally contain a selectable marker for antibiotic resistance. Segments of DNA cloned into these vectors can be propagated as plasmids. When cells harboring these vectors are provided with all genes necessary for the production of phage particles, the mode of replication of the plasmid changes to rolling circle replication to generate copies of one strand of the plasmid DNA and package phage particles. The phagemid may form infectious or non-infectious phage particles. This term includes phagemids which contain a phage coat protein gene or fragment thereof linked to a heterologous polypeptide gene as a gene fusion such that the heterologous polypeptide is displayed on the surface of the phage particle.

The term “phage vector” means a double stranded replicative form of a bacteriophage containing a heterologous gene and capable of replication. The phage vector has a phage origin of replication allowing phage replication and phage particle formation. The phage is preferably a filamentous bacteriophage, such as an M13, fl, fd, Pf3 phage or a derivative thereof, or a lambdoid phage, such as lambda, 21, phi80, phi81, 82, 424, 434, etc., or a derivative thereof.

“Oligonucleotides” are short-length, single- or double-stranded polydeoxynucleotides that are chemically synthesized by known methods (such as phosphotriester, phosphite, or phosphoramidite chemistry, using solid-phase techniques such as described in EP 266,032 published 4 May 1988, or via deoxynucloside H-phosphonate intermediates as described by Froeshler et al., Nucl. Acids, Res., 14:5399-5407 (1986)). Further methods include the polymerase chain reaction defined below and other autoprimer methods and oligonucleotide syntheses on solid supports. All of these methods are described in Engels et al., Agnew. Chem. Int. Ed. Engl., 28:716-734 (1989). These methods are used if the entire nucleic acid sequence of the gene is known, or the sequence of the nucleic acid complementary to the coding strand is available. Alternatively, if the target amino acid sequence is known, one may infer potential nucleic acid sequences using known and preferred coding residues for each amino acid residue. The oligonucleotides can be purified on polyacrylamide gels or molecular sizing columns or by precipitation.

DNA is “purified” when the DNA is separated from non-nucleic acid impurities. The impurities may be polar, non-polar, ionic, etc.

A “scaffold”, as used herein, refers to a polypeptide or portion thereof that maintains a stable structure or structural element when a heterologous polypeptide is inserted into the polypeptide. The scaffold provides for maintenance of a structural and/or functional feature of the polypeptide after the heterologous polypeptide has been inserted. A “CDRH3 scaffold” comprises a N-terminal portion in which some or all of the positions are structural and a C terminal portion in which some or all of the amino acid positions are structural and wherein the scaffold can accommodate the insertion of a central portion or loop of contiguous amino acids that may be randomized. In another embodiment, a CDRH3 scaffold comprises a N-terminal portion having a cysteine residue and a C terminal portion having a cysteine residue, wherein the cysteine residues in the N terminal and C-terminal portion of the CDRH3 form a disulfide bond that stabilizes the central portion insert that can vary in sequence and in length. A “monobody scaffold” comprises a CDRH3 scaffold that interacts with framework residues in an antibody variable domain at the former light chain interface to form a stable variable domain and provide for a central portion of the CDRH3 that can vary in sequence and in length.

A “source antibody”, as used herein, refers to an antibody or antigen binding polypeptide whose antigen binding determinant sequence serves as the template sequence upon which diversification according to the criteria described herein is performed. An antigen binding determinant sequence generally includes an antibody variable region, preferably at least one CDR, preferably including framework regions.

As used herein, “solvent accessible position” refers to a position of an amino acid residue in the variable regions of the heavy and light chains of a source antibody or antigen binding polypeptide that is determined, based on structure, ensemble of structures and/or modeled structure of the antibody or antigen binding polypeptide, as potentially available for solvent access and/or contact with a molecule, such as an antibody-specific antigen. These positions are typically found in the CDRs and on the exterior of the protein. The solvent accessible positions of an antibody or antigen binding polypeptide, as defined herein, can be determined using any of a number of algorithms known in the art. Preferably, solvent accessible positions are determined using coordinates from a 3-dimensional model of an antibody or antigen binding polypeptide, preferably using a computer program such as the InsightII program (Accelrys, San Diego, Calif.). Solvent accessible positions can also be determined using algorithms known in the art (e.g., Lee and Richards, J. Mol. Biol. 55, 379 (1971) and Connolly, J. Appl. Cryst. 16, 548 (1983)). Determination of solvent accessible positions can be performed using software suitable for protein modeling and 3-dimensional structural information obtained from an antibody. Software that can be utilized for these purposes includes SYBYL Biopolymer Module software (Tripos Associates). Generally and preferably, where an algorithm (program) requires a user input size parameter, the “size” of a probe which is used in the calculation is set at about 1.4 Angstrom or smaller in radius. In addition, determination of solvent accessible regions and area methods using software for personal computers has been described by Pacios ((1994) “ARVOMOL/CONTOUR: molecular surface areas and volumes on Personal Computers.” Comput. Chem. 18(4): 377-386; and (1995). “Variations of Surface Areas and Volumes in Distinct Molecular Surfaces of Biomolecules.” J. Mol. Model. 1:46-53.)

The phrase “structural amino acid position” as used herein refers to an amino acid position in a CDRH3 region of a polypeptide that contributes to the stability of the structure of the polypeptide such that the polypeptide retains at least one biological function such as specifically binding to a molecule such as an antigen and/or binds to a target molecule that binds to folded polypeptide and does not bind to unfolded polypeptide such as Protein A. Structural amino acid positions of a CDRH3 region are identified as amino acid positions less tolerant to amino acid substitutions without affecting the structural stability of the polypeptide. Amino acid positions less tolerant to amino acid substitutions can be identified using a method such as alanine scanning mutagenesis or shotgun scanning as described in WO 01/44463 and analyzing the effect of loss of the wild type amino acid on structural stability at positions in the CDRH3. If a wild type amino acid is replaced with a scanning amino acid in a position in a CDRH3 region, and the resulting variant exhibits poor binding to a target molecule that binds to folded polypeptide, then that position is important to maintaining the structure of the polypeptide. A structural amino acid position is a position in which, preferably, the ratio of polypeptides with wild type amino acid at a position to a variant substituted with a scanning amino acid at that position is at least about 3 to 1, about 5 to l, about 8 to 1, about 10 to 1 or greater.

The term “stability” as used herein refers to the ability of a molecule to maintain a folded state under physiological conditions such that it retains at least one of its normal functional activities, for example, binding to an antigen or to a molecule like Protein A. The stability of the molecule can be determined using standard methods. For example, the stability of a molecule can be determined by measuring the thermal melt (“TM”) temperature. The TM is the temperature in ° Celsius at which ½ of the molecules become unfolded. Typically, the higher the TM, the more stable the molecule.

The phrase “randomly generated population” as used herein refers to a population of polypeptides wherein one or more amino acid positions in a CDR has a variant amino acid encoded by a random codon set which allows for substitution of all 20 naturally occurring amino acids at that position. For example, in one embodiment, a randomly generated population of polypeptides having randomized CDRH3 or portions thereof regions include a variant amino acid at each position in CDRH3 that is encoded by a random codon set. A random codon set includes codon sets designated NNS and NNK.

As used herein, “target amino acid” refers to an amino acid that belongs to the group of amino acids that are collectively the most commonly occurring amino acids found at a particular position of known and/or natural antibodies or antigen binding fragment or polypeptide. In some embodiments, the most commonly occurring amino acids” are those amino acids that are found in a particular position in preferably at least about 50%, preferably at least about 70%, preferably at least about 80%, preferably at least about 90%, preferably all of sequences of known and/or natural antibodies or antigen binding fragment or polypeptides. In some embodiments, the most commonly occurring amino acids” are those amino acids that are found in a particular position in preferably from about 50% to about 100%, preferably from about 60% to about 90%, preferably from about 70% to about 85%, preferably from about 80% to about 85% of the sequences of known and/or natural antibodies or antigen binding fragment or polypeptides. Known antibodies or antigen binding fragments are those whose sequences are available in the art, such as those available in publicly-accessible databases, such as the database of Kabat (“Sequence of Proteins of Immunological Interest, National Institutes of Health, Bethesda, Md., 1987 and 1991) and/or as located at http://immuno.bme.nwu.edu. The amino acid position is preferably a position in the CDR region. A target group of amino acids refers to a group of target amino acids for a particular position. Preferably, a target amino acid is not a cysteine residue. For positions in the light chain CDR1, CDR2, CDR3, and for heavy chain CDR1 and CDR2, typically, a target group of amino acids can include from preferably about two to about eleven, preferably from about 4 to about 9, preferably from about 5 to about 7, preferably about 6 amino acids at a particular highly diverse and solvent-accessible position of the source sequence.

A “transcription regulatory element” will contain one or more of the following components: an enhancer element, a promoter, an operator sequence, a repressor gene, and a transcription termination sequence. These components are well known in the art. U.S. Pat. No. 5,667,780.

A “transformant” is a cell which has taken up and maintained DNA as evidenced by the expression of a phenotype associated with the DNA (e.g., antibiotic resistance conferred by a protein encoded by the DNA).

“Transformation” means a process whereby a cell takes up DNA and becomes a “transformant”. The DNA uptake may be permanent or transient.

A “variant” or “mutant” of a starting or reference polypeptide (for e.g., a source antibody or its variable domain(s)/CDR(s)), such as a fusion protein (polypeptide) or a heterologous polypeptide (heterologous to a phage), is a polypeptide that 1) has an amino acid sequence different from that of the starting or reference polypeptide and 2) was derived from the starting or reference polypeptide through either natural or artificial (manmade) mutagenesis. Such variants include, for example, deletions from, and/or insertions into and/or substitutions of, residues within the amino acid sequence of the polypeptide of interest. For example, a fusion polypeptide of the invention generated using an oligonucleotide comprising a nonrandom codon set that encodes a sequence with a variant amino acid (with respect to the amino acid found at the corresponding position in a source antibody/antigen binding fragment or polypeptide) would be a variant polypeptide with respect to a source antibody or antigen binding fragment or polypeptide. Thus, a variant CDR refers to a CDR comprising a variant sequence with respect to a starting or reference polypeptide sequence (such as that of a source antibody or antigen binding fragment or polypeptide). A variant amino acid, in this context, refers to an amino acid different from the amino acid at the corresponding position in a starting or reference polypeptide sequence (such as that of a source antibody or antigen binding fragment or polypeptide). Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites. Methods for generating amino acid sequence variants of polypeptides are described in U.S. Pat. No. 5,534,615, expressly incorporated herein by reference.

A “wild type” or “reference” sequence or the sequence of a “wild type” or “reference” protein/polypeptide, such as a coat protein, or a CDR or variable domain of a source antibody, is the reference sequence from which variant polypeptides are derived through the introduction of mutations. In general, the “wild type” sequence for a given protein is the sequence that is most common in nature. Similarly, a “wild type” gene sequence is the sequence for that gene which is most commonly found in nature. Mutations may be introduced into a “wild type” gene (and thus the protein it encodes) either through natural processes or through man induced means. The products of such processes are “variant” or “mutant” forms of the original “wild type” protein or gene.

As used herein “Vh3” refers to a subgroup of antibody variable domains. The sequences of known antibody variable domains have been analyzed for sequence identity and divided into groups. Antibody heavy chain variable domains in subgroup III are known to have a Protein A binding site.

A “plurality” or “population” of a substance, such as a polypeptide or polynucleotide of the invention, as used herein, generally refers to a collection of two or more types or kinds of the substance. There are two or more types or kinds of a substance if two or more of the substances differ from each other with respect to a particular characteristic, such as the variant amino acid found at a particular amino acid position. For example, there is a plurality or population of polypeptides of the invention if there are two or more polypeptides of the invention that are substantially the same, preferably identical, in sequence except for the sequence of a variant CDR or except for the variant amino acid at a particular solvent accessible and highly diverse amino acid position or structural amino acid position. In another example, there is a plurality or population of polynucleotides of the invention if there are two or more polynucleotides of the invention that are substantially the same, preferably identical, in sequence except for the sequence that encodes a variant CDR or except for the sequence that encodes a variant amino acid for a particular solvent accessible and highly diverse amino acid position or structural amino acid position.

Modes of the Invention

The invention provides methods for generating and isolating novel antibodies or antigen binding fragments or polypeptides that preferably have a high affinity for a selected antigen. A plurality of different antibodies or antibody variable domains are prepared by mutating (diversifying) one or more selected amino acid positions in a source light chain variable domain and/or heavy chain variable domain to generate a diverse library of antigen binding variable domains with variant amino acids at those positions. The diversity in the variable domains is designed so that highly diverse libraries are obtained with minimal structural perturbation. In one aspect, the amino acid positions are those that are solvent accessible, for example as determined by analyzing the structure of a source antibody, and/or that are highly diverse among known and/or natural immunoglobulin polypeptides. In another aspect, the amino acid positions are those positions in a CDRH3 region that are structural, and for which diversity is limited while the remaining positions can be randomized to generate a library that is highly diverse and well folded.

In one aspect of the invention, structural amino acid positions in a CDRH3 region are identified. An amino acid position is a structural position if it contributes to the stability of the polypeptide, such as a variable domain. Once the structural amino acid positions are identified, diversity is limited at these positions in order to provide a library with a diverse CDRH3 region while minimizing the structural perturbations.

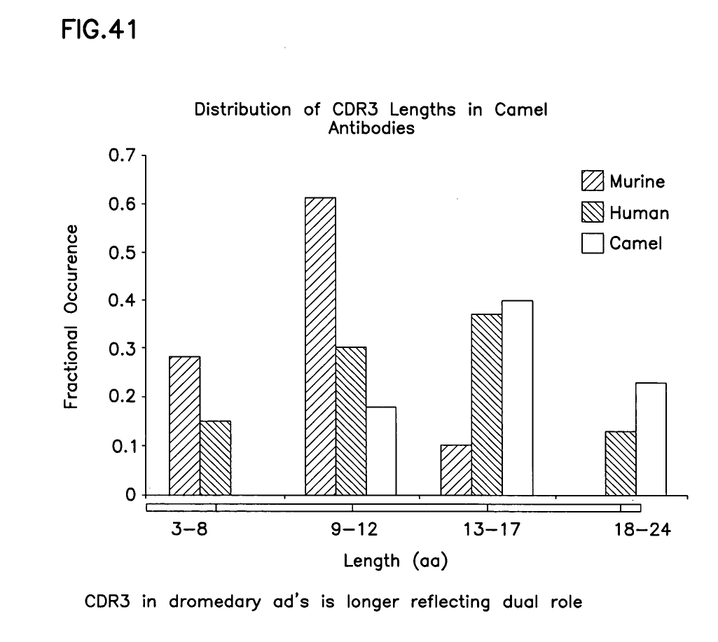

In some embodiments, structural amino acid positions in a CDRH3 are located near the N and C terminus of the CDRH3 allowing for a central portion that can be varied. The variant CDRH3 regions can have a N terminal flanking region in which some or all of the amino acid positions have limited diversity, a central portion comprising at least one or more non-structural amino acid position that can be varied in length and sequence, and C-terminal flanking sequence in which some or all amino acid positions have limited diversity. The length of the CDRH3 region is selected to reflect the length of CDRH3 regions found in naturally occurring antibody variable domains found in humans, camelids and/or mice, for example, as shown in FIG. 41. In some embodiments, the length of CDRH3 is from about 3 amino acids up to about 24 amino acids. The length of the N terminal flanking region, central portion, and C-terminal flanking region is determined by selecting the length of CDRH3, randomizing each position and identifying the structural amino acid positions at the N and C-terminal ends of the CDRH3. The length of the N and C terminal flanking sequences should be long enough to include at least one structural amino acid position in each flanking sequence. In some embodiments, the length of the N-terminal flanking region is at least about from 1 to 4 contiguous amino acids, the central portion of one or more non-structural positions can vary from about 1 to 20 contiguous amino acids, and the C-terminal portion is at least about from 1 to 6 contiguous amino acids.

Once at least one structural amino acid position in a heavy chain CDRH3 is identified, a limited set of amino acids is selected for substitution at this position. The diversity at least one structural amino acid position is limited to provide for maximal diversity while minimizing the structural perturbations. The number of amino acids that are substituted at a structural amino acid position is no more than about 1 to 7, about 1 to 4 or about 1 to 2 amino acids. In some embodiments, a variant amino acid at a structural amino acid position is encoded by one or more nonrandom codon sets. The nonrandom codon sets encode multiple amino acids for a particular positions, for example, about 1 to 7, about 1 to 4 amino acids or about 1 to 2 amino acids. The amino acids that are substituted at structural positions are those that are found at that position in a randomly generated CDRH3 population at a frequency at least one standard deviation above the average frequency for any amino acid at the position.

The variant CDRH3 region also comprises a non-structural amino acid position that has a variant amino acid. Non-structural amino acid positions can vary in sequence and in length. In some embodiments, one or more non-structural amino acid positions are located in between the N terminal and C terminal flanking regions. Said at least one non-structural position is or comprises a contiguous sequence of about 1 to 20 amino acids; more preferably 1 to 17 amino acids; more preferably 5 to 15 amino acids and most preferably about 9 to 12 amino acids. The non-structural amino acid positions can be substituted randomly with any of the naturally occurring amino acids or with selected amino acids. In some embodiments, said at least one non-structural position can have a variant amino acid encoded by a random codon set or a nonrandom codon. The nonrandom codon set preferably encodes amino acids that are commonly occurring at that position in naturally occurring known antibodies. Examples of nonrandom codon sets include DVK, XYZ, and NVT.

When the polypeptide is an antibody heavy chain variable domain, diversity at framework region residues may also be limited in order to preserve structural stability of the polypeptide. The diversity in framework regions is limited at those positions that form the light chain interface. Amino acids in positions at the light chain interface can be modified to provide for binding of the heavy chain to antigen in absence of the light chain. The amino acid positions that are found at the light chain interface in the VHH of camelid monobodies include amino acid position 37, amino acid position 45, amino acid position 47 and amino acid position 91. Heavy chain interface residues are those residues that are found on the heavy chain but have at least one side chain atom that is within 6 angstroms of the light chain. The amino acid positions in the heavy chain that are found at the light chain interface in human heavy chain variable domains include positions 37, 39, 44, 45, 47, 91, and 103.

In another aspect of the invention, CDRH1 and CDRH2 residues are those of naturally occurring antibody variable domains or can be those from known antibody variable domains that bind to a particular antigen whether naturally occurring or synthetic. In some embodiments, the CDRH1 And CDRH2 regions may be randomized at each position. It will be understood by those of skill in the art that antigen binding molecules isolated using the methods of the invention may require further optimization of antigen binding affinity using standard methods. In one embodiment, the CDRH1 and CDRH2 sequences are those that are from the closest human germline sequence for CDRH1 and CDRH2 of the naturally occurring camelid monobody sequences.

In one aspect, libraries or populations with diverse variable domains are generated using the heavy chain variable domain (VHH) of a monobody. The small size and simplicity make monobodies attractive scaffolds for peptidomimetic and small molecule design, as reagents for high throughput protein analysis, or as potential therapeutic agents. The diversified VHH domains are useful, inter alia, in the design of enzyme inhibitors, novel antigen binding molecules, modular binding units in bispecific or intracellular antibodies, as binding reagents in protein arrays, and as scaffolds for presenting constrained peptide libraries.

In another aspect, amino acid positions that are solvent accessible and highly diverse are preferably those in the CDR regions of the antibody variable domains selected from the group consisting of CDRL1, CDRL2, CDRL3, CDRH1, CDRH2, CDRH3, and mixtures thereof. Amino acid positions are each mutated using a non-random codon set encoding the commonly occurring amino acids at each position. In some embodiments, when a solvent accessible and highly diverse position in a CDR region is to be mutated, a codon set is selected that encodes preferably at least about 50%, preferably at least about 60%, preferably at least about 70%, preferably at least about 80%, preferably at least about 90%, preferably all the target amino acids (as defined above) for that position. In some embodiments, when a solvent accessible and highly diverse position in a CDR region is to be mutated, a codon set is selected that encodes preferably from about 50% to about 100%, preferably from about 60% to about 95%, preferably from at least about 70% to about 90%, preferably from about 75% to about 90% of all the target amino acids (as defined above) for that position.

The diversity of the library or population of the antibody variable domains is designed to maximize diversity while minimizing structural perturbations of the antibody variable domain to provide for increased ability to isolate high affinity antibodies. The number of positions mutated in the antibody variable domain is minimized or specifically targeted. In some cases, the variant amino acids at each position are designed to include the commonly occurring amino acids at each position, while preferably (where possible) excluding uncommonly occurring amino acids. In other cases, structural amino acid positions are identified and diversity is minimized at those positions to ensure a well folded polypeptide. Preferably, a single antibody or antigen binding polypeptide including at least one CDR, is used as the source polypeptide. It is surprising that a library of antibody variable domains with high affinity antigen binders having diversity in sequences and size can be generated using a single source polypeptide as a template and targeting diversity to particular positions using particular amino acid substitutions.

Design of Diversity of Antibody Variable Domains

In one aspect of the invention, high quality libraries of antibody variable domains are generated. The libraries have diversity in number of members of the library as well as in the diversity of different sequences of the antibody variable domains. The libraries include a plurality or population of high affinity binding antibody variable domains for one or more antigens, including, for example, insulin like growth factor-1 (IGF-1), vascular endothelial growth factor (VEGF), Human Chronic Gonadotropin (HCG), and Her-2.

In one aspect of the invention, a polypeptide comprising a variant CDRH3 region is provided. A CDRH3 region is designed to provide for amino acid sequence diversity at certain positions while minimizing the structural perturbations. Diversity is limited at structural amino acid positions. The polypeptide comprises a variant CDRH3, wherein the variant CDRH3 comprises at least one structural amino acid position. Structural amino acid positions in a CDRH3 region of a polypeptide, preferably a variable domain of a camelid monobody, are identified. An amino acid position is a structural position if it contributes to the stability of the polypeptide, such that the polypeptide retains at least one biological function such as binding to an antigen and/or Protein A.

Once the structural amino acid positions are identified, diversity is minimized or limited at these positions in order to provide a library with a diverse CDRH3 region while minimizing the structural perturbations. The number of amino acids that are substituted at a structural amino acid position is no more than about 1 to 7, about 1 to 4 or about 1 to 2 amino acids. In some embodiments, a variant amino acid at a structural amino acid position is encoded by one or more nonrandom codon sets. The nonrandom codon sets encode multiple amino acids for a particular positions, for example, about 1 to 7, about 1 to 4 amino acids or about 1 to 2 amino acids. The amino acids that are substituted at structural positions are those that are found at that position in a randomly generated CDRH3 population at a frequency at least one standard deviation above the average frequency for any amino acid at the position. Preferably, the frequency is at least 60% or greater than the average frequency for any amino acid at that position, more preferably the frequency is at least one standard deviation (as determined using standard statistical methods) greater than the average frequency for any amino acid at that position.

A polypeptide or source antibody variable domain can include an antibody, antibody variable domain, antigen binding fragment or polypeptide thereof, a monobody, VHH, a monobody or antibody variable domain obtained from a naïve or synthetic library, camelid antibodies, naturally occurring antibody or monobody, synthetic antibody or monobody, recombinant antibody or monobody, humanized antibody or monobody, germline derived antibody or monobody, chimeric antibody or monobody, and affinity matured antibody or monobody. In one embodiment, the polypeptide is an antibody variable domain that is a member of the Vh3 subgroup and preferably, is a camelid monobody

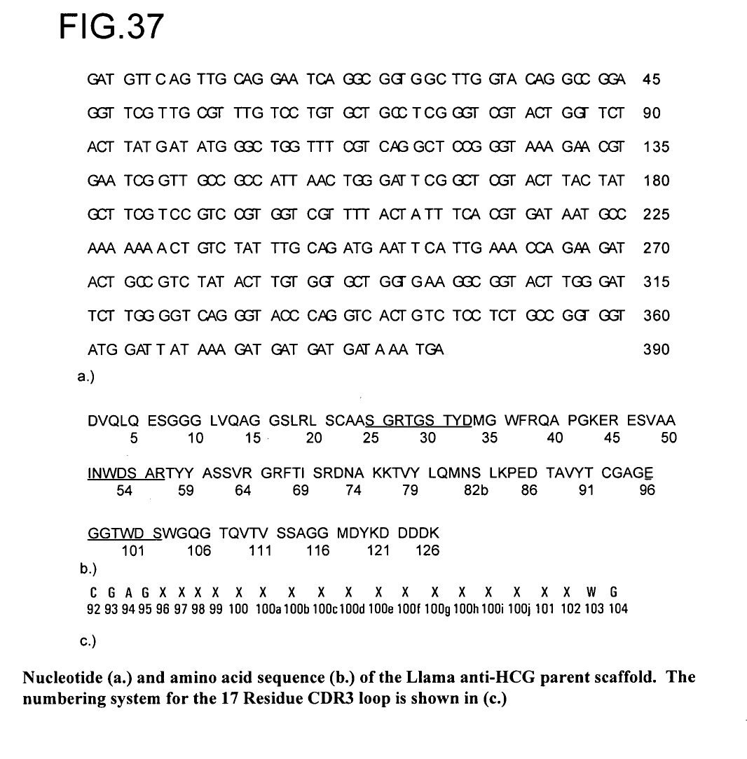

Monobodies can bind to antigens in the absence of a light chain and may be utilized, inter alia, for modular antigen binding domains in bispecific antibodies, intracellular antibodies, proteomics, and/or novel therapeutic agents. In one embodiment, the source antibody or antigen binding molecule is a VHH of a camelid monobody of the Vh3 family. A source antibody is a llama anti-HCG monobody. The nucleotide and amino acid sequence of the VHH of llama anti-HCG monobody is shown in FIG. 37. The crystal structure of the monobody has been described in Spinelli et al., Nature Structural Biology, 3:752-757 (1996).

A structural amino acid position refers to an amino acid position in a CDRH3 region of a polypeptide that contributes to the stability of the structure of the polypeptide such that the polypeptide retains at least one biological function such as specifically binding to a molecule such as an antigen or preferably, specifically binds to a target molecule that binds to folded polypeptide and does not bind to unfolded polypeptide such as Protein A. Structural amino acid positions of a CDRH3 region are identified as amino acid positions less tolerant to amino acid substitutions without affecting the structural stability of the polypeptide. Amino acid positions less tolerant to amino acid substitutions can be identified using a method such as alanine scanning mutagenesis or shotgun scanning as described in WO 01/44463 and analyzing the effect of loss of the wild type amino acid on structural stability at positions in the CDRH3.

In some embodiments, structural amino acid positions in a CDRH3 are located near the N and C terminus of the CDRH3 allowing for a central portion that can be varied. The variant CDRH3 regions can have a N terminal flanking region in which some or all of the amino acid positions have limited diversity, a central portion comprising at least one or more non-structural amino acid position that can be varied in length and sequence, and C-terminal flanking sequence in which some or all amino acid positions have limited diversity. The length of the CDRH3 region is selected to reflect the length of CDRH3 regions found in naturally occurring antibody variable domains found in humans, camelids and/or mice, for example, as shown in FIG. 41. In some embodiments, the length of CDRH3 is from about 3 amino acids up to about 24 amino acids. The length of the N terminal flanking region, central portion, and C-terminal flanking region is determined by selecting the length of CDRH3, randomizing each position and identifying the structural amino acid positions at the N and C-terminal ends of the CDRH3. The length of the N and C terminal flanking sequences should be long enough to include at least one structural amino acid position in each flanking sequence. In some embodiments, the length of the N-terminal flanking region is at least about from 1 to 4 contiguous amino acids, the central portion of one or more non-structural positions can vary from about 1 to 20 contiguous amino acids, and the C-terminal portion is at least about from 1 to 6 contiguous amino acids.

The variant CDRH3 is typically positioned between the third framework region and the fourth framework region in an antibody variable domain and may be inserted within a CDRH3 in a source variable domain. Typically, when the variant CDRH3 is inserted into a source or wild type CDRH3 the variant CDRH3 replaces all or a part of the source or wild type CDRH3. The location of insertion of the CDRH3 can be determined by comparing the location of CDRH3s in naturally occurring antibody variable domains. In one embodiment, a comparison of the naturally occurring antibody variable domains of monobodies indicated that the synthetic CDRH3 may be inserted after amino acid position 95 and before amino acid position 103 of wild type VHH CDRH3.

The amino acid numbering may vary depending on the exact location of insertion of the CDRH3 region. In one embodiment, a 17 amino acid CDRH3 region is inserted in the CDRH3 of a VHH of a monobody between amino acid residues 95 (amino acid glycine) and 103 (amino acid tryptophan) (numbering according to Kabat, Sequences of Proteins of immunological Interest, 1991, NIH publication No. 32919). The 17 residue CDRH3, CGAGXXXXXXXXXXXXXXXXXWG, is then numbered starting at amino acid position of the first X as position 96, 97, 98, 99, 100, 100a, 100b, 100c, 100d, 100e, 100f, 100g, 100h, 100i, 100j, 101 and 102 (SEQ ID NO:137) as shown in FIG. 37c. The two amino acid positions at the N-terminus in this embodiment are 96 and 97, respectively. The last 6 amino acids from the C-terminus in this embodiment are 100g, 100h, 100i, 100j, 101, and 102.

The variant CDRH3 region also comprises a non-structural amino acid position that has a variant amino acid. Non-structural amino acid positions can vary in sequence and in length. In some embodiments, one or more non-structural amino acid positions are located in between the N terminal and C terminal flanking regions. Said at least one non-structural position is or comprises a contiguous sequence of about 1 to 20 amino acids; more preferably 1 to 17 amino acids; more preferably 5 to 15 amino acids and most preferably about 9 to 12 amino acids. The non-structural amino acid positions can be substituted randomly with any of the naturally occurring amino acids or with selected amino acids. In some embodiments, said at least one non-structural position can have a variant amino acid encoded by a random codon set or a nonrandom codon. The nonrandom codon set preferably encodes amino acids that are commonly occurring at that position in naturally occurring known antibodies. Examples of nonrandom codon sets include DVK, XYZ, and NVT.

When the polypeptide is an antibody heavy chain variable domain, diversity at framework region residues may also be limited in order to preserve structural stability of the polypeptide. The diversity in framework regions is limited at those positions that form the light chain interface. Amino acids in positions at the light chain interface can be modified to provide for binding of the heavy chain to antigen in absence of the light chain. The amino acid positions that are found at the light chain interface in the VHH of camelid monobodies include amino acid position 37, amino acid position 45, amino acid position 47 and amino acid position 91. Heavy chain interface residues are those residues that are found on the heavy chain but have at least one side chain atom that is within 6 angstroms of the light chain. The amino acid positions in the heavy chain that are found at the light chain interface in human heavy chain variable domains include positions 37, 39, 44, 45, 47, 91, and 103 .

In one embodiment, the polypeptide is a variable domain of a monobody and further comprises a framework 2 region of a heavy chain variable domain of a naturally occurring monobody, wherein amino acid position 37 of framework 2 has a phenylalanine, tyrosine, valine or tryptophan in that position. In another embodiment, the monobody variable domain further comprises a framework 2 region of a heavy chain, wherein the amino acid position 45 of the framework 2 region has an arginine, tryptophan, phenylalanine or leucine in that position. In another embodiment, the monobody variable domain further comprises a framework 2 region, wherein the amino acid position 47 has a phenylalanine, leucine, tryptophan or glycine residue in that position. In another embodiment, the monobody further comprises a framework 3 region of a heavy chain, wherein amino acid position 91 of the framework 3 region is a phenylalanine, threonine, or tyrosine.

In another aspect of the invention, CDRH1 and CDRH2 residues are those of naturally occurring antibody variable domains or monobody domains or can be those from known antibody variable domains or monobodies that bind to a particular antigen whether naturally occurring or synthetic. In some embodiments, the CDRH1 And CDRH2 regions may be randomized at each position. It will be understood by those of skill in the art that antigen binding molecules isolated using the methods of the invention may require further optimization of antigen binding affinity using standard methods. In one embodiment, the CDRH1 and CDRH2 sequences are those that are from the closest human germline sequence for CDRH1 and CDRH2 of the naturally occurring camelid monobody sequences.

In another aspect, the diversity in the library is designed by selecting amino acid positions that are solvent accessible and highly diverse in a single source antibody and mutating those positions in at least one CDR using nonrandom codon sets. The nonrandom codon set preferably encodes at least a subset of the commonly occurring amino acids at those positions while minimizing nontarget sequences such as cysteine and stop codons.

One source antibody is humanized antibody 4D5, but the methods for diversity design can be applied to other source antibodies whose sequence is known. A source antibody can be a naturally occurring antibody, synthetic antibody, recombinant antibody, humanized antibody, germ line derived antibody, chimeric antibody, affinity matured antibody, monobody, or antigen binding fragment or polypeptide thereof. The antibodies can be obtained from a variety of mammalian species including humans, mice and rats, as well as animals such as camelids. In some embodiments, a source antibody is an antibody that is obtained after one or more initial affinity screening rounds, but prior to an affinity maturation step(s).

One source antibody is the humanized antibody 4D5. It is a humanized antibody specific for a cancer-associated antigen known as Her-2 (erbB2). The antibody includes variable domains having consensus framework regions; a few positions were reverted to mouse sequence during the process of increasing affinity of the humanized antibody. The sequence and crystal structure of humanized antibody 4D5 have been described in U.S. Pat. No. 6,054,297, Carter et al, PNAS 89:4285 (1992), the crystal structure is shown in J Mol. Biol. 229:969 (1993) and online at www.ncbi.nih.gov/structure/ mmdb (MMDB#s-990-992).

A criterion for generating diversity in antibody variable domains is to mutate residues at positions that are solvent accessible (as defined above). These positions are typically found in the CDRs, and are typically on the exterior of the protein. Preferably, solvent accessible positions are determined using coordinates from a 3-dimensional model of an antibody, using a computer program such as the InsightII program (Accelrys, San Diego, Calif.). Solvent accessible positions can also be determined using algorithms known in the art (e.g., Lee and Richards, J. Mol. Biol. 55, 379 (1971) and Connolly, J. Appl. Cryst. 16, 548 (1983)). Determination of solvent accessible positions can be performed using software suitable for protein modeling and 3-dimensional structural information obtained from an antibody. Software that can be utilized for these purposes includes SYBYL Biopolymer Module software (Tripos Associates). Generally and preferably, where an algorithm (program) requires a user input size parameter, the “size” of a probe which is used in the calculation is set at about 1.4 Angstrom or smaller in radius. In addition, determination of solvent accessible regions and area methods using software for personal computers has been described by Pacios ((1994) “ARVOMOL/CONTOUR: molecular surface areas and volumes on Personal Computers”, Comput. Chem. 18(4): 377-386; and “Variations of Surface Areas and Volumes in Distinct Molecular Surfaces of Biomolecules.” J. Mol. Model. (1995), 1:46-53).

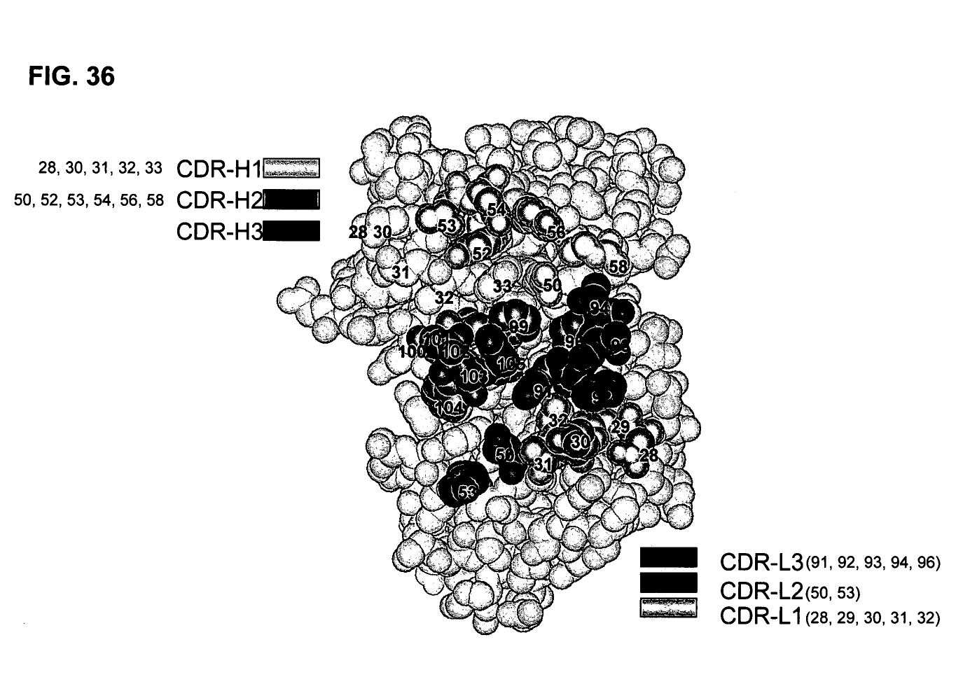

In some instances, selection of solvent accessible residues is further refined by choosing solvent accessible residues that collectively form a minimum contiguous patch, for example when the reference polypeptide or source antibody is in its 3-D folded structure. For example, as shown in FIG. 36, a compact (minimum) contiguous patch is formed by residues selected for CDRH1/H2/H3/L1/L2/L3 of humanized 4D5. A compact (minimum) contiguous may comprise only a subset (for example, 2-5 CDRs) of the full range of CDRs, for example, CDRH1/H2/H3/L3. Solvent accessible residues that do not contribute to formation of such a patch may optionally be excluded from diversification. Refinement of selection by this criterion permits the practitioner to minimize, as desired, the number of residues to be diversified. For example, residue 28 in H1 can optionally be excluded in diversification since it is on the edge of the patch. However, this selection criterion can also be used, where desired, to choose residues to be diversified that may not necessarily be deemed solvent accessible. For example, a residue that is not deemed solvent accessible, but forms a contiguous patch in the 3-D folded structure with other residues that are deemed solvent accessible may be selected for diversification. An example of this is CDRL-29. Selection of such residues would be evident to one skilled in the art, and its appropriateness can also be determined empirically and according to the needs and desires of the skilled practitioner.

The solvent accessible positions identified from the crystal structure of humanized antibody 4D5 for each CDR are as follows (residue position according to Kabat):
CDRL1: 28, 30, 31, 32
CDRL2: 50, 53
CDRL3: 91, 92, 93, 94, 96
CDRH1: 28, 30, 31, 32, 33
CDRH2: 50, 52, 52A, 53, 54, 55, 56, 57, 58.

In addition, residue 29 of CDRL1 was also selected based on its inclusion in a contiguous patch comprising other solvent accessible residues.

Another criterion for selecting positions to be mutated are those positions which show variability in amino acid sequence when the sequences of known and/or natural antibodies are compared. A highly diverse position refers to a position of an amino acid located in the variable regions of the light or heavy chains that have a number of different amino acids represented at the position when the amino acid sequences of known and/or natural antibodies/antigen binding fragment or polypeptides are compared. The highly diverse positions are preferably in the CDR regions. The positions of CDRH3 are all considered highly diverse. According to the invention, amino acid residues are highly diverse if they have preferably from about 2 to about 11 (although the numbers can range as described herein) different possible amino acid residue variations at that position.

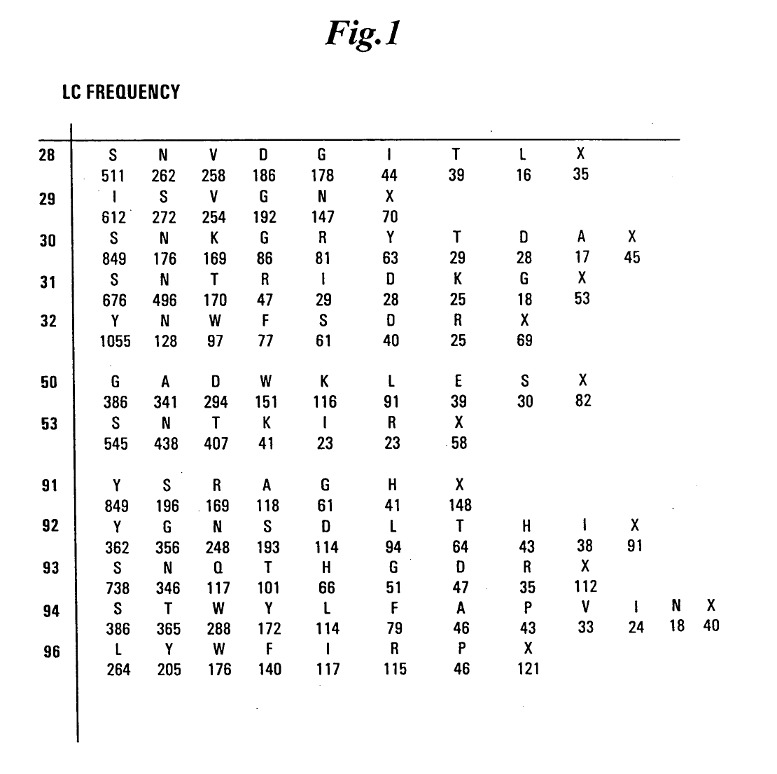

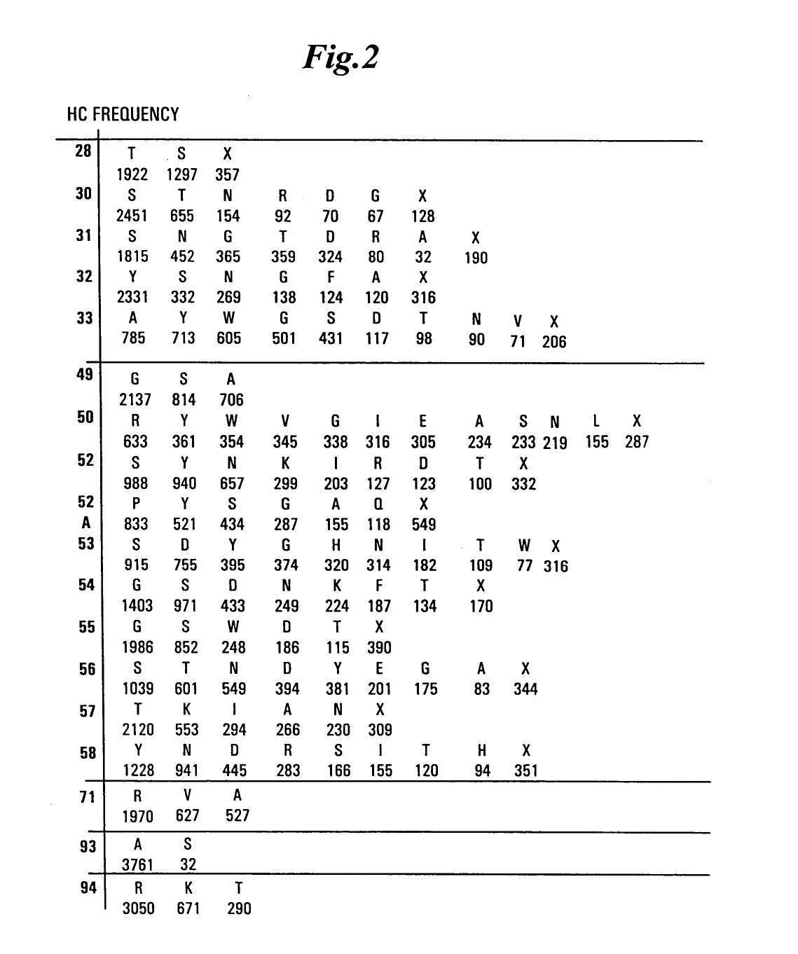

In one aspect, identification of highly diverse positions in known and/or naturally occurring antibodies is facilitated by the data provided by Kabat, Sequences of Proteins of Immunological Interest (National Institutes of Health, Bethesda, Md., 1987 and 1991). An internet-based database located at http://immuno.bme.nwu.edu provides an extensive collection and alignment of human light and heavy chain sequences and facilitates determination of highly diverse positions in these sequences. The diversity at the solvent accessible positions of humanized antibody 4D5 in known and/or naturally occurring light and heavy chains is shown in FIGS. 1 and 2.

In one aspect of the invention, the highly diverse and solvent accessible residues in at least one CDR selected from the group consisting of CDRL1, CDRL2, CDRL3, CDRH1, CDRH2 and mixtures thereof are mutated (i.e., randomized using codon sets as described herein). In some embodiments, the group also includes CDRH3. For example, the solvent accessible and/or highly diverse residues in CDRL3 and CDRH3 are mutated. Accordingly, the invention provides for a large number of novel antibody sequences formed by replacing the solvent accessible and highly diverse positions of at least one CDR of the source antibody variable domain with variant amino acids.

A target group of amino acids is the group of amino acids found at each solvent accessible and highly diverse position in a CDR in preferably at least about 50%, preferably at least about 70%, preferably at least about 80%, preferably at least about 90% of antibodies when the sequences of known and/or natural antibodies/antigen binding fragment or polypeptides are compared. The variant amino acids are a group of amino acids that include some or all of the target amino acids and are encoded by a nonrandom codon set. Of the amino acids encoded by the nonrandom codon set, preferably at least about 70% of the amino acids are target amino acids and more preferably at least about 80% of the amino acids are target amino acids. The nonrandom codon set for each position preferably encodes at least two amino acids and does not encode cysteine. Nontarget amino acids at each position are minimized and cysteines and stop codons are generally and preferably excluded because they can adversely affect the structure of the antibody variable domain for, in particular, L1, L2, L3, H1 and H2. For positions in the light chain CDR1, CDR2, CDR3, and for heavy chain CDR1 and CDR2, typically, a set of target amino acids can include from about two to eleven amino acids (described in detail above) at a particular highly diverse and solvent-accessible position of the source sequence.

Another criterion concerns diversifying residues in a CDRH3 region. CDRH3 regions vary greatly in length and in diversity at each amino acid position. In some antigen binding molecules, such as monobodies, some of the amino acid positions contribute to the stability of the variable domain. The amino acids substituted at these positions is limited or minimized so as to maintain the stability of the structure of the library of variant variable domains. Variant CDRH3 regions are formed by mutating at least one structural amino acid position using one or more nonrandom codon sets. One or more nonrandom codon sets encode an amino acid that are found at that position at a frequency greater than the average amino acid frequency at that position in a randomly substituted population of CDRH3 regions. Preferably, the amino acid is an amino acid that occurs most commonly in a randomized population. The frequency is preferably at least 60% or greater than average frequency for an amino acid at that position. The frequency is preferably at least about one standard deviation (determined using standard methods) greater than the average frequency for an amino acid at that position and more preferably at least two standard deviations above the average frequency for an amino acid at that position.

As discussed above, the variant amino acids are encoded by nonrandom codon sets. A codon set is a set of different nucleotide triplet sequences which can be used to form a set of oligonucleotides used to encode the desired group of amino acids. A set of oligonucleotides can be synthesized, for example, by solid phase synthesis, containing sequences that represent all possible combinations of nucleotide triplets provided by the codon set and that will encode the desired group of amino acids. Synthesis of oligonucleotides with selected nucleotide “degeneracy” at certain positions is well known in that art. Such sets of nucleotides having certain codon sets can be synthesized using commercial nucleic acid synthesizers (available from, for example, Applied Biosystems, Foster City, Calif.), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used according to the invention, have sequences that allow for hybridization to a variable domain nucleic acid template and also can include restriction enzyme sites for cloning purposes.

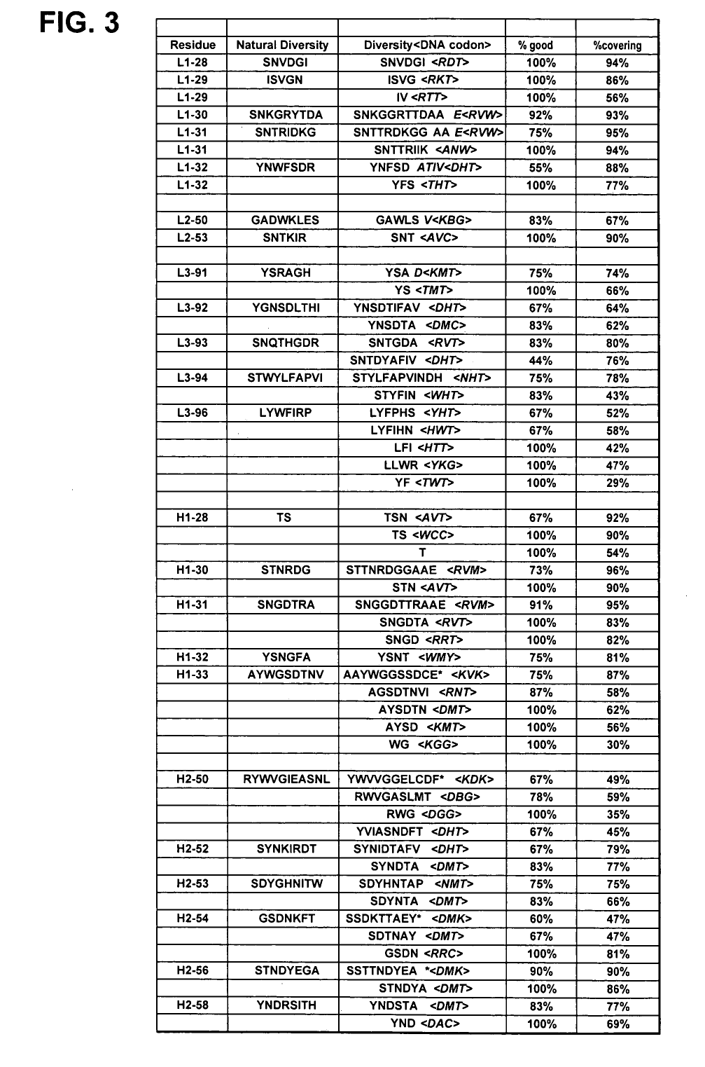

In one aspect, the target amino acids were identified for each solvent accessible and highly diverse position in CDRs of humanized antibody 4D5. The target amino acids were identified by identifying different amino acids at each of the solvent accessible and highly diverse positions in CDRL1, CDRL2, CDRL3, CDRH1 and CDRH2 using the sequences of known and/or naturally occurring antibodies in the Kabat database. Light chain diversity and heavy chain diversity from the Kabat database are shown in FIGS. 1 and 2, respectively. Based on the diversity as shown in FIGS. 1 and 2, the target amino acids identified at each position are shown in FIG. 3.

Illustrative nonrandom codon sets encoding a group of amino acids comprising preferably at least about 50%, preferably at least about 60%, preferably at least about 70%, preferably at least about 80%, preferably at least about 90%, preferably all of the target amino acids for each position are also shown in FIG. 3. The “% good” in FIG. 3 represents the percentage of amino acids encoded by the nonrandom codon set that are target amino acids for that position. Most preferably, the variant amino acids encoded by the codon set include the amino acids occurring with the highest frequency in known and/or naturally occurring antibodies. The high percentage means very low nontarget amino acids and this is more important than having more of the target amino acids in the design of the nonrandom codon set. The redundancy is included in all calculations.

The “% covering” in FIG. 3, represents the percentage of known and/or natural occurring antibody sequences that are encoded by the designed codons at each position. For example, for L3-91, the amino acids YSA (tyrosine, serine and alanine) are in the group of target amino acids which occur at position 91 in known and/or naturally occurring antibodies. The codon set is designed to encode YSAD (tyrosine, serine, alanine and aspartic acid), which encodes 75% of the target amino acids. These three amino acids are also found in 1190 out of 1580 natural antibody sequences at that site, which is 75% of the known and/or natural antibodies. It is preferable that codon sets are designed for each position in a CDR region to include amino acids found in those positions in at least about 50% of the known and/or naturally occurring antibodies and more preferably in at least about 60% of the known and/or naturally occurring antibodies and most preferably in at least about 70% of the known and/or naturally occurring antibodies.

Design of Diversity in Heavy Chain CDRH3 Regions

Heavy chain CDR3s (CDRH3s) in known antibodies or antigen binding polypeptides have diverse sequences, structural conformations, and lengths. CDRH3s are often found in the middle of the antigen binding pocket and often participate in antigen contact. The design of CDRH3 is thus preferably developed separately from that of the other CDRs because it can be difficult to predict the structural conformation of CDRH3 and the amino acid diversity in this region is especially diverse in known antibodies. In accordance with the present invention, in one embodiment, CDRH3 is designed to generate diversity at specific positions within CDRH3.

In one aspect of the invention, a polypeptide comprising a variant CDRH3 region is provided. A CDRH3 region is designed to provide for amino acid sequence diversity at certain positions while minimizing the structural perturbations. Diversity is limited at structural amino acid positions. Once at least one structural amino acid position in a heavy chain CDRH3 is identified, a limited set of amino acids is selected for substitution at this position. The diversity at at least one structural amino acid position is limited to provide for maximal diversity while minimizing the structural perturbations. The amino acids that are substituted at structural positions are those that are found at that position in a randomly generated CDRH3 population at a frequency at least one standard deviation above the average frequency for any amino acid at the position. The variant CDRH3 region also comprises a non-structural amino acid position that has a variant amino acid. Non-structural amino acid positions can vary in sequence and in length. Said at least one non-structural position is or comprises a contiguous amino acid sequence of about 1 to 20 amino acids; about 1 to 17 amino acids; more preferably 5 to 15 amino acids and most preferably about 9 to 12 amino acids. The non-structural amino acid positions can be substituted randomly or with selected amino acids. Methods for identifying structural amino acid positions and preparing variant CDRH3 regions are also provided.

Another embodiment involves generating diversity in CDRH3 at amino acid positions that were identified by comparing a randomly generated synthetic library to the frequency of amino acids at position in CDRH3 in known antibodies. Some amino acid positions may have a variant amino acid encoded by a nonrandom codon set that encodes the commonly occurring amino acids at that position. Other amino acid positions in the CDRH3 may be mutated using random codon sets.

Generating Diversity in a CDRH3 by Identifying Structural Amino Acid Positions and Limiting Diversity at those Positions

Monobodies are antigen binding molecules that lack light chains. Although their antigen combining site is found only in a heavy chain variable domain, the affinities for antigens have been found to be similar to those of classical antibodies (Ferrat et al., Biochem J., 366:415 (2002)). Libraries generated using variable domains of monobodies, such as camelid monobodies, have several advantages over libraries generated using other antibodies or antigen binding fragments or polypeptides. These molecules bind their targets with high affinity and specificity, and as such can be used as modules in the design of traditional antibodies. In certain cases, one may want to construct an antibody by first designing a high affinity heavy chain antibody or monobody which could then be converted to a Fab or IgG by pairing the monobody with an appropriately paired light chain. Secondly, these monobodies can be utilized to form novel antigen binding molecules, mini-antibodies, without the need for any light chain. These novel mini-antibodies or antigen binding molecules are similar to other single chain type antibodies, but the antigen binding domain is a heavy chain variable domain. Thirdly, these molecules are ideal for the design of bi-specific antibodies or intracellular antibodies. Fourthly, due to extensive use of CDRH3 and reduced binding surface due to absence of the light chain, monobody libraries may more successfully target enzyme active sites. Finally, monobody libraries may be useful as scaffolds for the presentation of peptide libraries, facilitating the design of smaller mimics of the antibody-antigen interface or isolating novel peptides that bind to a target antigens or enzymes and the like.

One aspect of the present invention concerns generating diversity in a CDRH3 region, preferably, in a CDRH3 region of a monobody. This aspect of the invention is based on the discovery that some amino acid positions in the CDRH3 contribute to the stability of the structure of the monobody and that the diversity at these amino acid positions should be minimized in order to generate a highly diverse library with minimal structural perturbations.

In some embodiments, the variant amino acid at at least one structural position is encoded by one or more nonrandom codon sets. The nonrandom codon set encodes amino acids found at that position in a randomly generated population at a frequency at least one standard deviation above the average frequency for any amino acid at that position. Preferably, the nonrandom codon set encodes 1 to 7 amino acids and more preferably 1 to 4 amino acids, and most preferably, as 1 to 2 amino acids. The polypeptides generated with variant CDRH3 regions in accord with the invention are useful in libraries to identify new antigen binding molecules.

The polypeptide or source antibody can include an antibody, antibody variable domain, antigen binding fragment or polypeptide thereof, a monobody, VHH, a monobody or antibody variable domain obtained from a naïve or synthetic library, camelid antibodies, naturally occurring antibody or monobody, synthetic antibody or monobody, recombinant antibody or monobody, humanized antibody or monobody, germline derived antibody or monobody, chimeric antibody or monobody, affinity matured antibody or monobody. In one embodiment, the polypeptide is an antibody variable domain that is a member of the Vh3 subgroup and preferably, is a camelid monobody.

Identifying Structural Amino Acid Positions in a CDRH3 Region of a Monobody and Preparing Variant CDRH3 Regions

Structural amino acid positions in a CDRH3 of a variable domain of a monobody can be identified using a variety of methods. Structural amino acid positions are identified as amino acid positions less tolerant to amino acid substitutions without affecting the structural stability of the polypeptide. Such positions can be identified using a method such as alanine scanning mutagenesis or shotgun scanning as described in WO 01/44463 and analyzing the effect of loss of the wild type amino acid on structural stability at positions in the CDRH3. If a wild type amino acid is replaced with a scanning amino acid in a position in a CDRH3 region, and the resulting variant exhibits poor binding to a target molecule that binds to folded polypeptide, then that position is important to maintaining the structure of the polypeptide.

An embodiment for identifying structural amino acids in a CDRH3 involves generating a library of antibody variable domains randomized at each amino acid position in the CDRH3. The library is sorted against a target molecule that specifically binds to folded polypeptide and does not bind to unfolded polypeptide and does not bind at an antigen binding site, such as Protein A. The sequence of the members of the library selected by interaction with the target molecule is determined. The most commonly occurring sequences in the CDRH3 region are identified and those positions that have fewer amino acid substitutions as compared to other positions can be selected as structural amino acid positions. Structural amino acid positions in each of those commonly occurring sequences can also be identified using a method such as shotgun scanning. A structural amino acid position is identified as an amino acid position in the CDRH3 that when substituted with the scanning amino acid has a decrease in the interaction with the target molecule, such as Protein A, as compared to a polypeptide having a source or wild type CDRH3 amino acid at that position. A structural amino acid position is, preferably, a position in which the ratio of sequences with the wild type amino acid at a position to sequences with the scanning amino acid at that position is at least about 3 to 1,5 to 1, 8 to 1, or about 10 to 1 or greater.

Methods for conducting alanine scanning mutagenesis are known to those of skill in the art and are described in WO 01/44463 and Morrison and Weiss, Cur. Opin. Chem. Bio., 5:302-307 (2001). Alanine scanning mutagenesis is a site directed mutagenesis method of replacing amino acid residues in a polypeptide with alanine to scan the polypeptide for residues involved in an interaction of interest. Standard site-directed mutagenesis techniques are utilized to systematically substitute individual positions in a protein with an alanine residue. Combinatorial alanine scanning allows multiple alanine substitutions to be assessed in a protein. Amino acid residues are allowed to vary only as the wild type or as an alanine. Utilizing oligonucleotide-mediated mutagenesis or cassette mutagenesis, binomial substitutions of alanine or seven wild type amino acids may be generated. For these seven amino acids, namely aspartic acid, glutamic acid, glycine, proline, serine, threonine, and valine, altering a single nucleotide can result in a codon for alanine. Libraries with alanine substitutions in multiple positions are generated by cassette mutagenesis or degenerate oligonucleotides with mutations in multiple positions. Shotgun scanning utilizes successive rounds of binding selection to enrich residues contributing binding energy to the receptor-ligand interaction.

Libraries of alanine-substituted proteins are constructed using standard oligonucleotide-mediated mutagenesis or cassette mutagenesis techniques. The pooled libraries are displayed on the surface of phage particles. Successive rounds of in vitro binding selection and amplification enrich residues with favorable contacts with the target ligand. A target molecule is a molecule that specifically binds to folded polypeptide and does not bind to unfolded polypeptide and preferably, does not bind at an antigen binding site. For example, for Protein A, the Protein A binding site of Vh3 antibody variable domains is found on the opposite B sheet from the antigen binding site. Another example of a target molecule, includes an antibody or antigen binding fragment or polypeptide that does not bind to the antigen binding site and binds to folded polypeptide and does not bind to unfolded polypeptide, such as an antibody to the Protein A binding site.

In some embodiments, structural amino acid positions in a CDRH3 are located near the N and C terminus of the CDRH3 allowing for a central portion that can be varied. The variant CDRH3 regions can have a N terminal flanking region in which some or all of the amino acid positions have limited diversity, a central portion comprising at least one or more non-structural amino acid position that can be varied in length and sequence, and C-terminal flanking sequence in which some or all amino acid positions have limited diversity. The length of the CDRH3 region is selected to reflect the length of CDRH3 regions found in naturally occurring antibody variable domains found in humans, camelids and/or mice, for example, as shown in FIG. 41. In some embodiments, the length of CDRH3 is from about 3 amino acids up to about 24 amino acids. The length of the N terminal flanking region, central portion, and C-terminal flanking region is determined by selecting the length of CDRH3, randomizing each position and identifying the structural amino acid positions at the N and C-terminal ends of the CDRH3. The length of the N and C terminal flanking sequences should be long enough to include at least one structural amino acid position in each flanking sequence. In some embodiments, the length of the N-terminal flanking region is at least about from 1 to 4 contiguous amino acids, the central portion of one or more non-structural positions can vary from about 1 to 20 contiguous amino acids, and the C-terminal portion is at least about from 1 to 6 contiguous amino acids.

In one embodiment, a 17 amino acid peptide is inserted into a CDRH3 region of a monobody. A library is generated in which each position in the 17 amino acid CDRH3 peptide is randomized. The randomized library is sorted or selected for binding to a target molecule that binds to folded polypeptide and does not bind to unfolded polypeptide. Optionally, multiple rounds of sorting and amplification may occur. The CDRH3 sequences of the most commonly occurring clones are determined. Each of the commonly occurring sequences is analyzed for structural residues by analyzing the polypeptide with that sequence using shotgun or alanine scanning mutagenesis.

In one embodiment, structural amino acid positions have been identified in a 17 amino acid CDRH3 region in a variable domain of a camelid monobody. At least one structural amino acid position includes one or both of the first two amino acid positions found at the N-terminus of the CDRH3. For example, in a CDRH3 comprising a formula of amino acid sequence of A₁-A₂-A₃)ₙ-A₄-A₅-A6-A₇-A₈-—A₉, the N-terminal amino acid positions correspond to amino acid positions A₁ and A₂, respectively. At least one structural amino acid position also includes one or more of the last 6 amino acids at the C-terminal end of the CDRH3. In the formula above, these amino acid positions correspond to A₄, A₅, A₆, A₇, A₈, and A₉. The amino acid position located sixth from the C-terminal end corresponds to A₄, the amino acid at the 5th position from the C-terminal end corresponds to A₅, the amino acid at the fourth position from the C-terminal end corresponds to A₆, the third position from the C-terminal end corresponds to A₇, the second position from the C-terminal end corresponds to A₈, and the first position at the C-terminal end corresponds to A₉. The central portion corresponds to A₃, which comprises or is a contiguous amino acid sequence of about 1 to 20 amino acids which may be randomized.

The variant CDRH3 is typically positioned between the third framework region and the fourth framework region in an antibody variable domain and may be inserted within a CDRH3 of a source variable domain. Typically, when the variant CDRH3 is inserted into a source or wild type CDRH3 the variant CDRH3 replaces all or a part of the source or wild type CDRH3. The location of insertion of the CDRH3 can be determined by comparing the location of CDRH3 in naturally occurring antibody variable domains. The amino acid numbering may vary depending on the exact location of insertion of the CDRH3 region. In one embodiment, a 17 amino acid CDRH3 region is inserted in the CDRH3 of a camelid monobody between amino acid residues 95 and 103 (numbering according to Kabat). The 17 residue CDRH3 is then numbered starting at amino acid position 96, 97, 98, 99, 100, 100a, 100b, 100c, 100d, 100e, 100f, 100g, 100h, 100i, 100j, 101 and 102 of SEQ ID NO:137. The two amino acid positions at the N-terminus in this embodiment are 96 and 97, respectively. The last 6 amino acids from the C-terminus in this embodiment are 100g, 100h, 100i, 100j, 101, and 102.

Once at least one structural amino acid position in a heavy chain CDRH3 is identified, a limited set of amino acids is selected for substitution at this position. The diversity at at least one structural amino acid position is limited to provide for maximal diversity while minimizing the structural perturbations. The number of amino acids that are substituted at a structural amino acid position is about 1 to 7, about 1 to 4 or about 1 to 2 amino acids. In some embodiments, a variant amino acid at a structural amino acid position is encoded by one or more nonrandom codon sets. The nonrandom codon sets encode multiple amino acids for a particular positions, for example, about 1 to 7, about 1 to 4 amino acids or about 1 to 2 amino acids. The amino acids that are substituted at structural positions are those that are found at that position in a randomly generated CDRH3 population at a frequency at least one standard deviation above the average frequency for any amino acid at the position.

In one embodiment, the polypeptide is an antibody variable domain of a monobody. The limited set of amino acids substituted at a structural amino acid position in a CDRH3 are those that provide for stabilization of the protein at the former light chain interface. The limited set of amino acids at a structural amino acid position are selected from the group consisting of a hydrophobic amino acid and/or arginine. The hydrophobic amino acids are preferably selected from the group consisting of leucine, isoleucine, valine, tryptophan, tyrosine, and phenylalanine. In a VHH variable domain, the structural amino acids positions in a CDRH3 are preferably substituted with hydrophobic amino acids to stabilize the VHH in the absence of the light chain at the former light chain interface.

In another embodiment, the CDRH3 is about 17 amino acids long and a library comprising a variant CDRH3 is generated. The variant CDRH3 region comprises at least one structural amino acid position selected from the group consisting of the first N-terminal amino acid position, the second N-terminal amino acid position, the sixth position from the C-terminus, the fourth position from the C-terminus, and the third position from the C-terminus and mixtures thereof. The first N-terminal amino acid position has a variant amino acid that is selected from the group consisting of (in single letter code) R, L, or V. The second N-terminal amino acid position has a variant amino acid that is selected from the group consisting of I and L. The sixth amino acid position from the C-terminus has a variant amino acid that is selected from the group consisting of E, W and F. The fourth position from the C-terminus of the CDRH3 has a variant amino acid that is selected from the group consisting of W, R, G and M. The third amino acid position from the C-terminus has a variant amino acid that is selected from the group consisting of P, V, and L.

Another embodiment is a polypeptide comprising a variant CDRH3 comprising at least one structural amino acid position, wherein said at least one structural amino acid position is the third, fourth and/or sixth position from the C-terminus of the CDRH3, wherein the CDRH3 is at least 8 amino acids long and in one embodiment, is up to 24 amino acids long; wherein the fourth position from the C-terminus has a variant amino acid selected from the group consisting of M, R, G, and W, and the third amino acid position from the C-terminus has a variant amino acid selected from the group consisting of P, V, and L, and the sixth position from the C-terminus has a variant amino acid selected from the group consisting of E, W, and F. In an embodiment, at least one of the third, fourth, and/or sixth position from the C terminal has a tryptophan.

The variant CDRH3 region also comprises a non-structural amino acid position that has a variant amino acid. Non-structural amino acid positions can vary in sequence and in length. In some embodiments, one or more non-structural amino acid positions are located in between the N terminal and C terminal flanking regions. Said at least one non-structural position is or comprises a contiguous sequence of about 1 to 20 amino acids; more preferably 1 to 17 amino acids; more preferably 5 to 15 amino acids and most preferably about 9 to 12 amino acids. The non-structural amino acid positions can be substituted randomly with any of the naturally occurring amino acids or with selected amino acids. In some embodiments, said at least one non-structural position can have a variant amino acid encoded by a random codon set or a nonrandom codon. The nonrandom codon set preferably encodes amino acids that are commonly occurring at that position in naturally occurring known antibodies. Examples of nonrandom codon sets include DVK, XYZ, and NVT.

The invention also provides for 1) fusion polypeptides; 2) fusion polypeptides to viral coat proteins or portions thereof; 3) polynucleotides encoding any of the polypeptides; 4) replicable expression vectors comprising a polynucleotide encoding the polypeptides of the invention; 5) host cells comprising the vectors; 6) a library comprising a plurality of vectors of the invention and 7) a population of variant polypeptides or polynucleotides of the invention.

Monobody Variant CDRH3

As discussed previously, variant CDRH3 regions can generate peptide libraries that bind to a variety of target molecules, including antigens. These variant CDRH3 regions can be incorporated into other antibody molecules or used to form a single chain mini-antibody with an antigen binding domain comprising a heavy chain variable domain but lacking a light chain. Within the CDRH3, amino acid positions that are primarily structural have limited diversity and other amino acids not as important for structural stability can be varied both in length and sequence diversity. CDRH3 regions can be designed so that the diversity is limited at structural amino acid positions and varied at non-structural amino acid positions varying in size, from 1 to 20 amino acids, preferably 5 to 15 amino acids and more preferably, 9 to 12 amino acids. A CDRH3 scaffold is preferably selected to have structural amino acid positions at the N and/or C-terminal amino acids, providing for a central portion of the CDRH3 that can be randomized.

Polypeptides comprising a CDRH3 having such a structure include camelid monobody, VHH, camelized antibodies, antibody or monobody variable domain obtained from a naïve or synthetic library, naturally occurring antibody or monobody, recombinant antibody or monobody, humanized antibody or monobody, germline derived antibody or monobody, chimeric antibody or monobody, and affinity matured antibody or monobody.

A number of different combinations of structural amino acid positions and nonstructural amino acid positions can be designed in a CDRH3 template. One CDRH3 variant comprises an amino acid sequence having the formula;
A₁-A₂-A₃)ₙ-A₄-A₅, wherein
A₁ is an amino acid selected from the group consisting of R, L, V, F, W and K;
A₂ is an amino acid selected from the group consisting of I, L, V, R, W and S;
A₃ is any naturally occurring amino acid and n can be 1-17;
A₄ is an amino acid selected from the group consisting of W, G, R, M, S, A and H;
A₅ is an amino acid selected from the group consisting of V, L, P, G, S, E and W.

The amino acids to the left of the central portion of contiguous amino acids are referred to as the N terminal amino acids, and the amino acids to the right of the contiguous sequence are referred to as C terminal amino acids. Amino acids positions A₁ and A₂ are N terminal positions, A₃ represents the central portion that can be randomized, and A₄ and A₅ are C terminal positions.

In this particular embodiment, the first two N-terminal amino acid positions have limited diversity. To achieve limited diversity, the number of different amino acids substituted at each position is limited, for example, to seven amino acids or less, more preferably 4 amino acids or less and most preferably two amino acids or less. A₁ is an amino acid selected from the group consisting of R, L, V, F, W and K; and A₂ is selected from the group consisting of I, L, V, R, W and S. Other amino positions that have limited diversity include A₄ and A₅. A₄ is the fourth amino acid from the C-terminus of the CDRH3 and is selected from the group consisting of W, G, R, M, S, A and H. A₅ is the third amino acid position from the C-terminus and is selected from the group consisting of V, L, P, G, S, E, and W. Amino acid positions at A₃ can be any of the 20 naturally occurring amino acids, preferably L-amino acids.

(A₃)ₙ, is or comprises a contiguous amino acid sequence of about 1 to 17 amino acids. The amino acids can each be any of one of the 20 naturally occurring amino acids or can be selected amino acids. In some embodiments, the selected amino acids are each can be encoded by a nonrandom codon set. The nonrandom codon set preferably encodes amino acids found or commonly occurring at those positions in naturally occurring antibody or monobodies such as DVK, NVT, or XYZ.

A number of different combinations of structural amino acid positions and nonstructural amino acid positions can be designed in a CDRH3 template. One CDRH3 variant comprises an amino acid sequence having the formula;
A₁-A₂-A₃)ₙ-A₅-A₆-A₇, wherein
A₁ is an amino acid selected from the group consisting of R, L, V, F, W and K;
A₂ is an amino acid selected from the group consisting of I, L, V, R, W and S;
A₃ is any naturally occurring amino acid and n can be 1-17;
A₄ is an amino acid selected from the group consisting of W, G, R, M, S, A and H;
A₅ is an amino acid selected from the group consisting of V, L, P, G, S, E and W; and
A₆ and A₇ are any naturally occurring amino acid.

The amino acids to the left of the central portion of contiguous amino acids are referred to as the N terminal amino acids, and the amino acids to the right of the contiguous sequence are referred to as C terminal amino acids. Amino acids positions A₁ and A₂ are N terminal positions, A₃ represents the central portion that can be randomized, and A₄, A₅, A₆, and A₇ are C terminal positions. In some embodiments, amino acid positions A₆ and A₇ may be structural amino acid positions.

In this particular embodiment, the first two N-terminal amino acid positions have limited diversity. To achieve limited diversity, the number of different amino acids substituted at each position is limited, for example, to seven amino acids or less , more preferably 4 amino acids or less and most preferably two amino acids or less. A₁ is an amino acid selected from the group consisting of R, L, V, F, W and K; and A₂ is selected from the group consisting of I, L, V, R, W and S. Other amino positions that have limited diversity include A₄ and A₅. A₄ is the fourth amino acid from the C-terminus of the CDRH3 and is selected from the group consisting of W, G, R, M, S, A and H. A₅ is the third amino acid position from the C-terminus and is selected from the group consisting of V, L, P, G, S, E, and W. Amino acid positions at A₃, A₆ and A₇ can be any of the 20 naturally occurring amino acids, preferably L-amino acids.

(A₃)ₙ, is or comprises a contiguous amino acid sequence of about 1 to 17 amino acids. The amino acids can each be any of one of the 20 naturally occurring amino acids or can be selected amino acids. In some embodiments, the selected amino acids are each can be encoded by a nonrandom codon set. The nonrandom codon set preferably encodes amino acids found or commonly occurring at those positions in naturally occurring antibody or monobodies such as DVK, NVT, or XYZ.

Other CDRH3 variants comprise an amino acid sequence having the formula of:
A₁-A₂-A₃)ₙ-A₅-A₆-A₇-A₈-A₉; wherein
A₁ is an amino acid selected from the group consisting of R, L, and V;
A₂ is an amino acid selected from the group consisting of I, L, and V;
A₃ is any naturally occurring amino acid and n=1-17;
A₄ is an amino acid selected from the group consisting of E, W, and F;
A₅ is any naturally occurring amino acid;
A₆ is an amino acid selected from group consisting of W, G, R, and M;
A₇ is an amino acid selected from the group consisting of V, L, and P; and
A₈ and A₉ is any naturally occurring amino acid.

The amino acids to the left of the central portion of contiguous amino acids are referred to as the N terminal amino acids, and the amino acids to the right of the contiguous sequence are referred to as C terminal amino acids. Amino acids positions A₁ and A₂ are N terminal positions, A₃ represents the central portion that can be randomized, and A₄, A₅, A₇ and A₈ are C terminal positions. In some embodiments, amino acid positions A₈ and A₉ may be structural amino acid positions.

Another embodiment of a CDRH3 region comprises an amino acid sequence having the formula of R-A₂-A₃-R-(A₅)ₙ, wherein A₂ is L, I or M; A₃ and A₅ are any naturally occurring amino acid; and n is 1 to 20. A library of randomly generated 17 amino acid CDRH3 indicated that a consensus sequence R-L/I/M-A₃-R at the N-terminus may be preferred for some embodiments.

Another embodiment of a CDR3 region comprises an amino acid sequence having the formula of: R-L/I/M-(A₃)ₙW-A₅-A₆-A₇-A₈-A₉, wherein A₆ is W, G, R or M; A₇ is V, L or P; A₃, A₅, A₈ and A₉ can be any naturally occurring amino acid and n is 1 to 15. A library of randomly generated CDRH3 regions indicated that a consensus sequence may also include amino acids located near the C-terminal end of CDRH3, especially at position the third, fourth, and sixth positions from the C-terminal end of CDRH₃.

In particular embodiments, one of 4 CDRH3 scaffolds may be especially useful in designing libraries of diverse CDRH3 regions while minimizing the structural perturbations of the polypeptide or antibody variable domain. A “CDRH3 scaffold” comprises a N-terminal portion in which some or all of the positions are structural and a C terminal portion in which some or all of the amino acid positions are structural and wherein the scaffold can accommodate the insertion of a central portion or loop of contiguous amino acids that that can vary in sequence and in length. In some embodiments, the N terminal portion is about 1 to 4 amino acids. In some embodiments, the C terminal portion is about 1 to 6 amino acids. In some cases, the central portion is a contiguous sequence of about 1 to 20 amino acids or 9 to 12 amino acids.

In some embodiments, a CDRH3 scaffold comprises a N-terminal portion having a cysteine residue and a C terminal portion having a cysteine residue, wherein the cysteine residues in the N terminal and C-terminal portion of the CDRH3 form a disulfide bond that stabilizes the central portion insert, and wherein the central portion insert is a contiguous amino acid sequence of about 1 to 20 amino acids.

In one embodiment, the scaffold has a N terminal sequence of R-L/I/M-A₃-R, wherein A₃ is any naturally occurring amino acid and wherein the central portion insert is a contiguous amino acid sequence of about 1 to 20 amino acids. In another embodiment, the N terminal sequence is R—I-A₃—C, wherein A₃ is any naturally occurring amino acid and wherein the central portion insert is a contiguous amino acid sequence of about 1 to 20 amino acids. In other embodiments, the N terminal sequence comprises R—I, L-L, V-L, or R-L and wherein the central portion insert is a contiguous amino acid sequence of about 1 to 20 amino acids.

In some embodiments, the C terminus has a sequence of CWVTW and wherein the central portion insert is a contiguous amino acid sequence of about 1 to 20 amino acids. In other embodiments the C-terminal sequence comprises F—X—R—V, W—X—X-L, W—X-M-P, or W—V, wherein X can be any naturally occurring amino acid and wherein the central portion insert is a contiguous amino acid sequence of about 1 to 20 amino acids.

One CDRH3 scaffold having the central portion or loop of contiguous amino acids comprises an amino acid sequence A₁-A₂-(A₃)ₙ-A₄-A₅-A₆-A₇, wherein A₁ is R; A₂ is I; A₄ is W; A₅ is V; A₃, A₆, A₇ are any naturally occurring amino acid; and n=11. Another CDRH3 scaffold of interest comprises an amino acid sequence A₁-A₂-(A₃)ₙ-A₄-A₅-A₆-A₇-A₈-A₉, wherein A₁ is V; A₂ is L; A₄ is F; A₆ is R; A₇ is V; A₃, A₈, A₉ are any naturally occurring amino acid and n=11. Another CDRH3 scaffold of interest comprises an amino acid sequence A₁-A₂-(A₃)ₙ-A₄-A₅-A₆-A₇-A₈-A₉, wherein A₁ is R; A₂ is L; A₃,A₅, A₆, A₇,A₈, and A₉ are any naturally occurring amino acid; A₄ is W; and n=11. Another CDRH3 scaffold of interest comprises an amino acid sequence A₁-A₂-(A₃)ₙ-A₄-A₅-A₆-A₇-A₈-A₉, wherein A₁ is L; A₂ is L; A₄ is W; A₇ is L; A₃, A₅, A₆, A₈, and A₉ are any naturally occurring amino acid; and n=11.

In another embodiment, a particular CDRH3 variant can be utilized to generate a library of diverse CDRH3 regions that can be screened for binding to one or more antigens. One CDRH3 comprises an amino acid sequence having the formula of: A₁-A₂-A₃-A₄-(A₅)ₙ-A₆-A₇-A₈-A₉-A₁₀, wherein
A₁ is an amino acid selected from the group consisting of R, L and V;
A₂ is an amino acid selected from the group consisting of I, L and V;
A₃ is any naturally occurring amino acid;
A₄ is selected from the group consisting of C, R and N;
A₅ is any naturally occurring amino acid and n=1-16;
A₆ is an amino acid selected from the group consisting of C, S, F, T, E and D;
A₇ is an amino acid selected from the group consisting of W, G, R and M;
A₈ is an amino acid selected from the group consisting of V, L and P;
A₉ is an amino acid selected from the group consisting of T, V, L and Q; and
A₁₀ is an amino acid selected from the group consisting of W, G, S and A.

The amino acids to the left of the central portion of contiguous amino acids are referred to as the N terminal amino acids, and the amino acids to the right of the contiguous sequence are referred to as C terminal amino acids. Amino acids positions A₁, A₂, A₃, and A₄ are N terminal positions, A₅ represents the central portion that can be randomized, and A₆, A₇, A₈, A₉, and A₁₀ are C terminal positions. Another CDRH3 of interest has an amino acid sequence wherein A₁ is R; A₂ is I; A₄ is C; A₆ is C; A₇ is W; A₈ is V and n=1 to 6 or 1 to 7.

In some embodiments, cysteines may be incorporated into the CDRH3 design to improve the stability of the CDRH3 and/or to improve antigen binding capabilities. The cysteines are located in the N-terminal portion and the C-terminal portion of a CDRH3 that flank the central portion which varies in sequence and length. In some embodiments, the cysteines are immediately adjacent to the central portion of the CDRH3. The cysteines may form a disulfide bond that may stabilize the central portion that is varied randomly. Cysteines may be incorporated into the CDRH3 design to improve the affinity of antigen binding molecules that can be isolated from the library or to form a next generation library.

Methods of the Invention

A method for generating variant CDRH3 regions involves generating a library of antibody variable domains randomized at each amino acid position in the CDRH3. The library is sorted against a target molecule, such as Protein A. Multiple rounds of amplification and selection may take place. Preferably, at least three rounds of amplification and selection are conducted. At the fourth or fifth rounds, the sequence of each of the four most dominant clones is identified. The identity of the structural amino acid positions in any particular clone can be confirmed using, for example, combinatorial alanine scanning mutagenesis. A CDRH3 scaffold can then be prepared by limiting the diversity at the structural amino acid positions in a particular design and inserting a central portion of contiguous amino acids between those structural positions ranging from 1 to 20 amino acids, 1 to 17 amino acids, preferably 5-15 amino acids and more preferably 9 to 12 amino acids. The central portion can be randomized at one or more positions if desired.

Another aspect of the invention involves a method of designing a CDRH3 region that is well folded and stable for phage display. The method involves generating a library comprising polypeptides with variant CDRH3 regions, selecting the members of the library that bind to a target molecule that binds to folded polypeptide and does not bind to unfolded polypeptide, analyzing the members of the library to identify structural amino acid positions in the CDRH3 region, identifying at least one amino acid that can be substituted at the structural amino acid position, wherein the amino acid identified is one that occurs significantly more frequently than random (one standard deviation or greater than the frequency of any amino acid at that position) in polypeptides selected for stability, and designing a CDRH3 region that has at least one or the identified amino acids in the structural amino acid position. The method may further comprise selecting a CDRH3 that has structural amino acid positions at the N and/or C-terminus of the CDRH3. For example, a CDRH3 can be selected that has structural amino acid positions in one or more of the two N-terminal amino acids and/or at one or more of the six C-terminal amino acids. In one embodiment, all of the structural amino acid positions have been substituted with one of the identified amino acids. The identified amino acids are preferably selected from the group consisting of hydrophobic amino acids and/or arginine. Libraries with variant CDRH3 regions can be generated and sorted for members of the library that bind to a target antigen such as a cytokine.

It is contemplated that the sequence diversity of libraries created by introduction of variant amino acids in CDRH3 by any of the embodiments described herein can be increased by combining these CDRH3 variations with variations in other regions of the antibody, specifically in other CDRs of either the light or heavy chain variable sequences. It is contemplated that the nucleic acid sequences that encode members of this set can be further diversified by introduction of other variant amino acids in the CDRs of either the light or heavy chain sequences, via codon sets. Thus, for example, in one embodiment, CDRH3 sequences from fusion polypeptides that bind a target antigen can be combined with diversified CDRH1, or CDRH2 sequences, or any combination of diversified CDRs.

In another aspect of the invention, CDRH1 and CDRH2 residues are those of naturally occurring antibody variable domains or can be those from known antibody variable domains that bind to a particular antigen whether naturally occurring or synthetic. In some embodiments, the CDRH1 And CDRH2 regions may be randomized at each position. It will be understood by those of skill in the art that antigen binding molecules isolated using the methods of the invention may require further optimization of antigen binding affinity using standard methods. In one embodiment, the CDRH1 and CDRH2 sequences are those that are from the closest human germline sequence for CDRH1 and CDRH2 of the naturally occurring camelid monobody sequences.

Framework Region Changes

The polypeptides of the invention can comprise a variable domain from a source antibody. The source antibody variable domain comprises framework region sequences that may be modified to accommodate a variant CDRH3 and/or to improve structural stability of the variable domain. Alternatively, a variant CDRH3 region may be combined with a variable domain that is different from the source antibody and may include naturally occurring variable domains, modified variable domains, and consensus variable domains.

When the polypeptide is an antibody heavy chain variable domain, diversity at framework region residues may also be limited in order to preserve structural stability of the polypeptide. The diversity in framework regions is limited at those positions that form the light chain interface. Amino acids in positions at the light chain interface can be modified to provide for binding of the heavy chain to antigen in absence of the light chain. The amino acid positions that are found at the light chain interface in the VHH of camelid monobodies include amino acid position 37, amino acid position 45, amino acid position 47 and amino acid position 91. Heavy chain interface residues are those reidues that are found on the heavy chain but have at least one side chain atom that is within 6 angstroms of the light chain. The amino acid positions in the heavy chain that are found at the light chain interface in human heavy chain variable domains include positions 37, 39, 44, 45, 47 , 91, and 103.

In one embodiment, the polypeptide is a variable domain of a monobody and further comprises a framework 2 region of a heavy chain variable domain of a naturally occurring monobody, wherein amino acid position 37 of framework 2 has a phenylalanine, tyrosine, valine or tryptophan in that position. In another embodiment, the monobody variable domain further comprises a framework 2 region of a heavy chain, wherein the amino acid position 45 of the framework 2 region has an arginine, tryptophan, phenylalanine or leucine in that position. In another embodiment, the monobody variable domain further comprises a framework 2 region, wherein the amino acid position 47 has a phenylalanine, leucine, tryptophan or glycine residue in that position. In another embodiment, the monobody further comprises a framework 3 region of a heavy chain, wherein amino acid position 91 of the framework 3 region is a phenylalanine, threonine, or tyrosine.

It should be noted that in some instances framework residues may be varied relative to the sequence of a source antibody or antigen binding fragment or polypeptide, for example, to reflect a consensus sequence. As described above, framework residues 93 or 94 in the heavy chain of 4D5 may be varied. Another example of a framework residue that may be altered is heavy chain framework residue 71 of 4D5, which is R in about 1970 polypeptides, V in about 627 polypeptides and A in about 527 polypeptides, as found in the Kabat database.

Generating Diversity in CDRH3 Using Random and/or Nonrandom Codon Sets

To generate diversity in CDRH3, a database of known, generally natural, antibodies can be used as a guideline. In comparison to other CDRs, CDRH3 has the highest diversity in sequences and length, although the sequence diversity is not completely random (i.e., some amino acids occur more often than others). In one embodiment, a library is generated with a degenerate codon set such as NNK, which codes for all 20 amino acids and a stop codon. Clones that display functionally on the phage are analyzed for their sequences. Frequency of amino acids in the synthetically-generated library is then compared with the frequency of amino acids in known antibodies. Good agreement of amino acid frequency can be expected, although in some instances there may be increased frequency of certain classes of amino acids in the synthetic library compared to known antibodies. For example, a library generated with NNK can be expected to contain sequences that utilize more usage of aliphatic/hydrophobic amino acids. This procedure can be performed to obtain useful information on appropriate choice of amino acids, and thus codon sets, to include in generating CDRH3 diversity. In another embodiment, CDRH3 diversity is generated using the codon set NNS. NNS and NNK encode the same amino acid group. However, there can be individual preferences for one codon set or the other, depending on the various factors known in the art, such as efficiency of coupling in oligonucleotide synthesis chemistry.

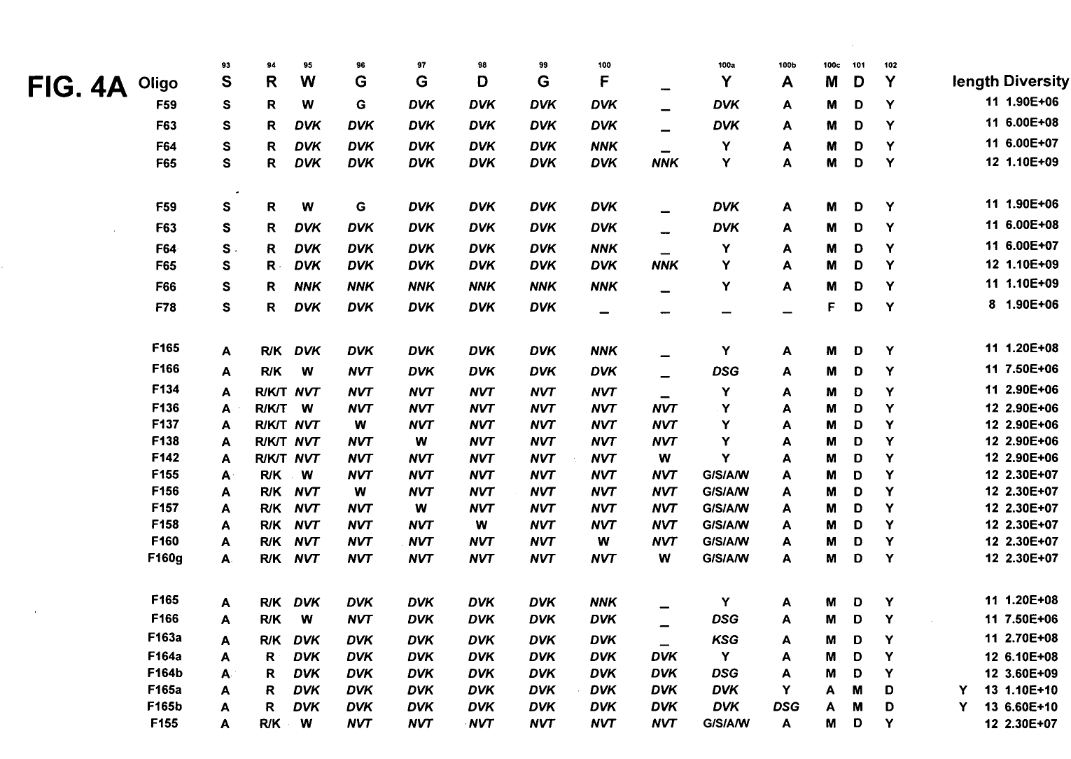

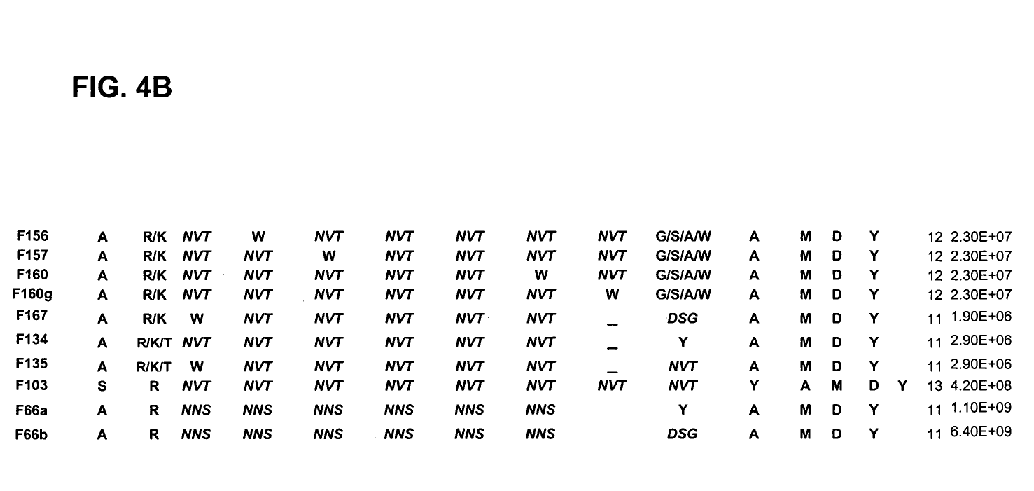

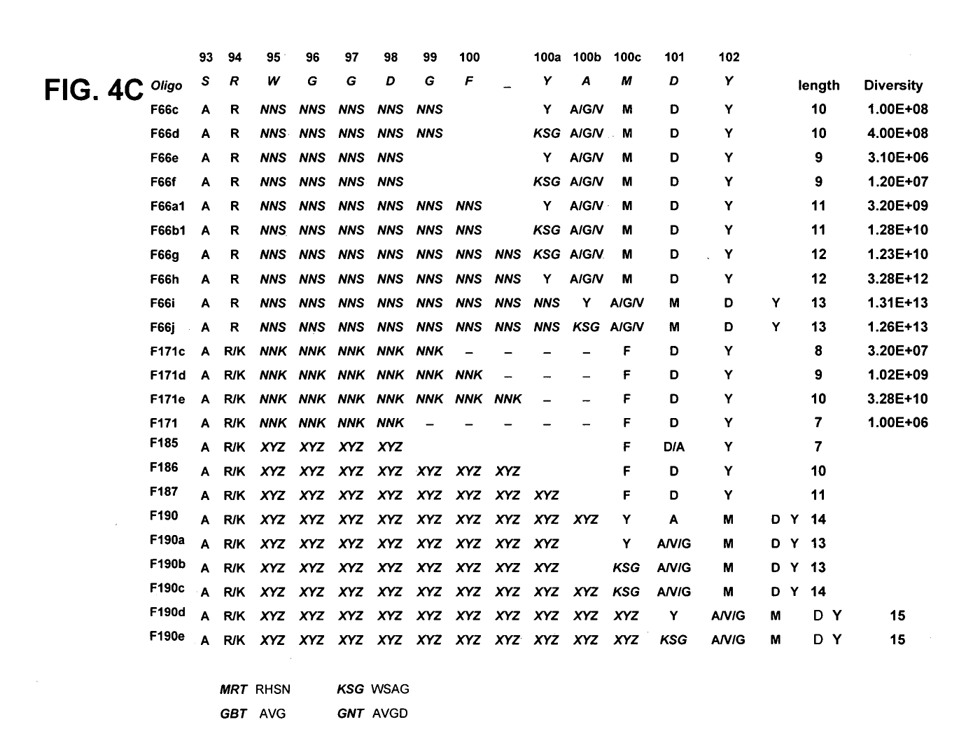

In some embodiments, the practitioner of methods of the invention may wish to modify the amount/proportions of individual nucleotides (G, A, T, C) for a codon set, such as the N nucleotide in a codon set such as in NNS. This is illustratively represented as XYZ codons indicated in FIG. 4. This can be achieved by, for example, doping different amounts of the nucleotides within a codon set instead of using a straight, equal proportion of the nucleotides for the N in the codon set. Such modifications can be useful for various purposes depending on the circumstances and desire of the practitioner. For example, such modifications can be made to more closely reflect the amino acid bias as seen in a natural diversity profile, such as the profile of CDRH3.

In some embodiments, a diversified CDRH3 library can be generated with a codon set such as DVK, which encodes 11 amino acids (ACDEGKNRSYW) and one stop codon. This excludes amino acids that occur infrequently in known antibodies. A CDRH3 library may also be generated using the codon set NVT, which encodes 11 amino acids (ACDGHNPRSTY), but does not encode stop codons or tryptophan (Trp, W). In some embodiments, the design of a codon set, such as NVT, may be doped with Trp.

The choice of CDRH3 residues to randomize can be determined according to the process described above. For instance, for CDRH3 of variable domains of monobodies, strucural amino acid postions are identified and diversity at these positions is limited, allowing for more extensive randomization of other nonstructural CDRH3 positions.

For antibodies like 4D5, the C-terminus is quite constant and has mainly two types of sequences in known antibodies (such as in the Kabat database): Y₁₀₀ₐAND₁₀₁(Y/V)₁₀₂ (sometimes Y₁₀₀ₐ can vary slightly) or F₁₀₀ₐD₁₀₁(Y/V)₁₀₂. For example, in the humanized 4D5 antibody, the C-terminus of H3 is YAMDY. This sequence can be kept mostly constant, although Y₁₀₀ₐ may vary sometimes. Various designs of H3 are illustrated in FIG. 4, in which generally DVK or NVT is used to randomize residues 95-100 or 96-100a. The most common lengths of human CDRH3s are 11-13 residues, and CDRH3 of humanized antibody 4D5 is within this range.

Preferably, but not necessarily, the length of the diversified portion is kept the same as that of the source antibody, since this length is expected to be structurally stable. However, the length of the diversified portion can be increased or decreased by increasing or decreasing the number of designed codons inserted during mutagenesis. These changes in CDRH3 length can introduce additional sequence and conformational diversity which may increase the efficiency of the library in generating high affinity antibodies, provided the changes in CDRH3 length do not compromise structural stability. For example, one embodiment of a VHH monobody scaffold can accommodate randomized loops of 10-15 residues without loss of structural stability.

In some embodiments, Y₁₀₀ₐ of template antibody 4D5, may be randomized more narrowly by using codons that encode fewer target amino acids, for example DSG (encoding GARWST) or GSA (encoding GSAW). For example, when humanized antibody 4D5 is a source antibody, residues encoded by DSG and KSG are the ones found most often in known antibodies and in antigen-specific binders isolated from a phage library generated using a DVK codon at this position. In some embodiments, framework residue 94 (right before the start of the CDRH3 Kabat consensus sequence) may be changed, for example, to reflect the framework consensus sequence. The same holds true for residue 93. Human framework residue 93 is mostly alanine. In humanized antibody 4D5, for example, residue 93 is serine, which may be substituted with alanine in H3 randomization (see FIG. 2).

Examples of oligonucleotides that can be used to randomize CDRH3 in a 4D5 template are illustrated in FIG. 4.

Fusion Polypeptides

Fusion polypeptide constructs can be prepared for generating fusion polypeptides that bind with significant affinity to potential ligands.

In particular, fusion polypeptides comprising diversified CDR(s) and a heterologous polypeptide sequence (preferably that of at least a portion of a viral polypeptide) are generated, individually and as a plurality of unique individual polypeptides that are candidate binders to targets of interest. Compositions (such as libraries) comprising such polypeptides find use in a variety of applications, in particular as large and diverse pools of candidate immunoglobulin polypeptides (in particular, antibodies and antibody fragments) that bind to targets of interest.

In some embodiments, a fusion protein comprises an antibody variable domain, or an antibody variable domain and a constant domain, fused to all or a portion of a viral coat protein. Examples of viral coat proteins include infectivity protein PIII, major coat protein PVIII, p3, Soc, Hoc, gpD (of bacteriophage lambda), minor bacteriophage coat protein 6 (pVI) (filamentous phage; J Immunol Methods. Dec. 10, 1999;231(1-2):39-51), variants of the M13 bacteriophage major coat protein (P8) (Protein Sci April 2000; 9(4):647-54). The fusion protein can be displayed on the surface of a phage and suitable phage systems include M13KO7 helper phage, M13R408, M13-VCS, and Phi X 174, pJuFo phage system (J Virol. August 2001; 75(15):7107-13.v), hyperphage (Nat Biotechnol. January 2001; 19(1):75-8). The preferred helper phage is M13KO7, and the preferred coat protein is the M13 Phage gene III coat protein.

Tags useful for detection of antigen binding can also be fused to either an antibody variable domain not fused to a viral coat protein or an antibody variable domain fused to a viral coat protein. Additional peptides that can be fused to antibody variable domains include gD tags, c-Myc epitopes, poly-histidine tags, fluorescence proteins (eg., GFP), or beta-galactosidase protein which can be useful for detection or purification of the fusion protein expressed on the surface of the phage or cell.

These constructs may also comprise a dimerizable sequence that when present as a dimerization domain in a fusion polypeptide provides for increased tendency for heavy chains to dimerize to form dimers of Fab or Fab′ antibody fragments/portions. These dimerization sequences may be in addition to any heavy chain hinge sequence that may be present in the fusion polypeptide. Dimerization domains in fusion phage polypeptides bring two sets of fusion polypeptides (LC/HC-phage protein/fragment (such as pIII)) together, thus allowing formation of suitable linkages (such as interheavy chain disulfide bridges) between the two sets of fusion polypeptide. Vector constructs containing such dimerization sequences can be used to achieve divalent display of antibody variable domains, for example the diversified fusion proteins described herein, on phage. Preferably, the intrinsic affinity of each monomeric antibody fragment (fusion polypeptide) is not significantly altered by fusion to the dimerization sequence. Preferably, dimerization results in divalent phage display which provides increased avidity of phage binding, with significant decrease in off-rate, which can be determined by methods known in the art and as described herein. Dimerization sequence-containing vectors of the invention may or may not also include an amber stop codon 5′ of the dimerization sequence. Dimerization sequences are known in the art, and include, for example, the GCN4 zipper sequence (GRMKQLEDKVEELLSKNYHLENEVARLKKLVGERG) (SEQ ID NO: 3).

Methods of Generating Libraries of Randomized Variable Domains Methods of substituting an amino acid of choice into a template nucleic acid are well established in the art, some of which are described herein. For example, libraries can be created by targeting solvent accessible and/or highly diverse positions in at least one CDR region for amino acid substitution with variant amino acids using the Kunkel method. See, for e.g., Kunkel et al., Methods Enzymol. (1987), 154:367-382. Generation of randomized sequences is also described below in the Examples.

The sequence of oligonucleotides includes one or more of the designed codon sets for the solvent accessible and highly diverse positions in a CDR. A codon set is a set of different nucleotide triplet sequences used to encode desired variant amino acids. Codon sets can be represented using symbols to designate particular nucleotides or equimolar mixtures of nucleotides as shown in below according to the IUB code.

IUB Codes

G Guanine

A Adenine

T Thymine

C Cytosine

R (A or G)

Y (C or T)

M (A or C)

K (G or T)

S (C or G)

W (A or T)

H (A or C or T)

B (C or G or T)

V (A or C or G)

D (A or G or T)

N (A or C or G or T)

For example, in the codon set DVK, D can be nucleotides A or G or T; V can be A or G or C; and K can be G or T. This codon set can present 18 different codons and can encode amino acids Ala, Trp, Tyr, Lys, Thr, Asn, Lys, Ser, Arg, Asp, Glu, Gly, and Cys.

Oligonucleotide or primer sets can be synthesized using standard methods. A set of oligonucleotides can be synthesized, for example, by solid phase synthesis, containing sequences that represent all possible combinations of nucleotide triplets provided by the codon set and that will encode the desired group of amino acids. Synthesis of oligonucleotides with selected nucleotide “degeneracy” at certain positions is well known in that art. Such sets of nucleotides having certain codon sets can be synthesized using commercial nucleic acid synthesizers (available from, for example, Applied Biosystems, Foster City, Calif.), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used according to the invention, have sequences that allow for hybridization to a variable domain nucleic acid template and also can include restriction enzyme sites for cloning purposes.

In one method, nucleic acid sequences encoding variant amino acids can be created by oligonucleotide-mediated mutagenesis. This technique is well known in the art as described by Zoller et al. Nucleic Acids Res. 10:6487-6504(1987). Briefly, nucleic acid sequences encoding variant amino acids are created by hybridizing an oligonucleotide set encoding the desired codon sets to a DNA template, where the template is the single-stranded form of the plasmid containing a variable region nucleic acid template sequence. After hybridization, DNA polymerase is used to synthesize an entire second complementary strand of the template that will thus incorporate the oligonucleotide primer, and will contain the codon sets as provided by the oligonucleotide set.

Generally, oligonucleotides of at least 25 nucleotides in length are used. An optimal oligonucleotide will have 12 to 15 nucleotides that are completely complementary to the template on either side of the nucleotide(s) coding for the mutation(s). This ensures that the oligonucleotide will hybridize properly to the single-stranded DNA template molecule. The oligonucleotides are readily synthesized using techniques known in the art such as that described by Crea et al., Proc. Nat'l. Acad. Sci. USA, 75:5765 (1978).

The DNA template is generated by those vectors that are either derived from bacteriophage M13 vectors (the commercially available M13mp18 and M13mp19 vectors are suitable), or those vectors that contain a single-stranded phage origin of replication as described by Viera et al., Meth. Enzymol., 153:3 (1987). Thus, the DNA that is to be mutated can be inserted into one of these vectors in order to generate single-stranded template. Production of the single-stranded template is described in sections 4.21-4.41 of Sambrook et al., above.

To alter the native DNA sequence, the oligonucleotide is hybridized to the single stranded template under suitable hybridization conditions. A DNA polymerizing enzyme, usually T7 DNA polymerase or the Klenow fragment of DNA polymerase I, is then added to synthesize the complementary strand of the template using the oligonucleotide as a primer for synthesis. A heteroduplex molecule is thus formed such that one strand of DNA encodes the mutated form of gene 1, and the other strand (the original template) encodes the native, unaltered sequence of gene 1. This heteroduplex molecule is then transformed into a suitable host cell, usually a prokaryote such as E. coli JM101. After growing the cells, they are plated onto agarose plates and screened using the oligonucleotide primer radiolabelled with a 32-Phosphate to identify the bacterial colonies that contain the mutated DNA.

The method described immediately above may be modified such that a homoduplex molecule is created wherein both strands of the plasmid contain the mutation(s). The modifications are as follows: The single stranded oligonucleotide is annealed to the single-stranded template as described above. A mixture of three deoxyribonucleotides, deoxyriboadenosine (dATP), deoxyriboguanosine (dGTP), and deoxyribothymidine (dTT), is combined with a modified thiodeoxyribocytosine called dCTP-(aS) (which can be obtained from Amersham). This mixture is added to the template-oligonucleotide complex. Upon addition of DNA polymerase to this mixture, a strand of DNA identical to the template except for the mutated bases is generated. In addition, this new strand of DNA will contain dCTP-(aS) instead of dCTP, which serves to protect it from restriction endonuclease digestion. After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell.

As indicated previously the sequence of the oligonucleotide set is of sufficient length to hybridize to the template nucleic acid and may also, but does not necessarily, contain restriction sites. The DNA template can be generated by those vectors that are either derived from bacteriophage M13 vectors or vectors that contain a single-stranded phage origin of replication as described by Viera et al. ((1987) Meth. Enzymol., 153:3). Thus, the DNA that is to be mutated must be inserted into one of these vectors in order to generate single-stranded template. Production of the single-stranded template is described in sections 4.21-4.41 of Sambrook et al., supra.

According to another method, a library can be generated by providing upstream and downstream oligonucleotide sets, each set having a plurality of oligonucleotides with different sequences, the different sequences established by the codon sets provided within the sequence of the oligonucleotides. The upstream and downstream oligonucleotide sets, along with a variable domain template nucleic acid sequence, can be used in a polymerase chain reaction to generate a “library” of PCR products. The PCR products can be referred to as “nucleic acid cassettes”, as they can be fused with other related or unrelated nucleic acid sequences, for example, viral coat proteins and dimerization domains, using established molecular biology techniques.

Oligonucleotide sets can be used in a polymerase chain reaction using a variable region nucleic acid template sequence as the template to create nucleic acid cassettes. The variable region nucleic acid template sequence can be any portion of the light or heavy immunoglobulin chains containing the target nucleic acid sequences (ie., nucleic acid sequences encoding amino acids targeted for substitution). The variable region nucleic acid template sequence is a portion of a double stranded DNA molecule having a first nucleic acid strand and complementary second nucleic acid strand. The variable region nucleic acid template sequence contains at least a portion of a variable domain and has at least one CDR. In some cases, the variable region nucleic acid template sequence contains more than one CDR. An upstream portion and a downstream portion of the variable region nucleic acid template sequence can be targeted for hybridization with members of an upstream oligonucleotide set and a downstream oligonucleotide set.

A first oligonucleotide of the upstream primer set can hybridize to the first nucleic acid strand and a second oligonucleotide of the downstream primer set can hybridize to the second nucleic acid strand. The oligonucleotide primers can include one or more codon sets and be designed to hybridize to a portion of the variable region nucleic acid template sequence. Use of these oligonucleotides can introduce two or more codon sets into the PCR product (ie., the nucleic acid cassette) following PCR. The oligonucleotide primer that hybridizes to regions of the nucleic acid sequence encoding the antibody variable domain includes portions that encode CDR residues that are targeted for amino acid substitution.

The upstream and downstream oligonucleotide sets can also be synthesized to include restriction sites within the oligonucleotide sequence. These restriction sites can facilitate the insertion of the nucleic acid cassettes [ie., PCR reaction products] into an expression vector having additional antibody sequence. Preferably, the restriction sites are designed to facilitate the cloning of the nucleic acid cassettes without introducing extraneous nucleic acid sequences or removing original CDR or framework nucleic acid sequences.

Nucleic acid cassettes can be cloned into any suitable vector for expression of a portion or the entire light or heavy chain sequence containing the targeted amino acid substitutions generated via the PCR reaction. According to methods detailed in the invention, the nucleic acid cassette is cloned into a vector allowing production of a portion or the entire light or heavy chain sequence fused to all or a portion of a viral coat protein (ie., creating a fusion protein) and displayed on the surface of a particle or cell. While several types of vectors are available and may be used to practice this invention, phagemid vectors are the preferred vectors for use herein, as they may be constructed with relative ease, and can be readily amplified. Phagemid vectors generally contain a variety of components including promoters, signal sequences, phenotypic selection genes, origin of replication sites, and other necessary components as are known to those of ordinary skill in the art.

In another embodiment, wherein a particular variant amino acid combination is to be expressed, the nucleic acid cassette contains a sequence that is able to encode all or a portion of the heavy or light chain variable domain, and is able to encode the variant amino acid combinations. For production of antibodies containing these variant amino acids or combinations of variant amino acids, as in a library, the nucleic acid cassettes can be inserted into an expression vector containing additional antibody sequence, for example all or portions of the variable or constant domains of the light and heavy chain variable regions. These additional antibody sequences can also be fused to other nucleic acids sequences, such as sequences which encode viral coat proteins and therefore allow production of a fusion protein.

Vectors

One aspect of the invention includes a replicable expression vector comprising a nucleic acid sequence encoding a gene fusion, wherein the gene fusion encodes a fusion protein comprising an antibody variable domain, or an antibody variable domain and a constant domain, fused to all or a portion of a viral coat protein. Also included is a library of diverse replicable expression vectors comprising a plurality of gene fusions encoding a plurality of different fusion proteins including a plurality of the antibody variable domains generated with diverse sequences as described above. The vectors can include a variety of components and are preferably constructed to allow for movement of antibody variable domain between different vectors and/or to provide for display of the fusion proteins in different formats.

Examples of vectors include phage vectors. The phage vector has a phage origin of replication allowing phage replication and phage particle formation. The phage is preferably a filamentous bacteriophage, such as an M13, fl, fd, Pf3 phage or a derivative thereof, or a lambdoid phage, such as lambda, 21, phi80, phi81, 82, 424, 434, etc., or a derivative thereof.

Examples of viral coat proteins include infectivity protein PIII, major coat protein PVIII, p3, Soc, Hoc, gpD (of bacteriophage lambda), minor bacteriophage coat protein 6 (pVI) (filamentous phage; J Immunol Methods. Dec. 10, 1999;231(1-2):39-51), variants of the M13 bacteriophage major coat protein (P8) (Protein Sci April 2000; 9(4):647-54). The fusion protein can be displayed on the surface of a phage and suitable phage systems include M13KO7 helper phage, M13R408, M13-VCS, and Phi X 174, pJuFo phage system (J Virol. August 2001; 75(15):7107-13.v), hyperphage (Nat Biotechnol. January 2001; 19(1):75-8). The preferred helper phage is M13KO7, and the preferred coat protein is the M13 Phage gene III coat protein. The preferred host is E. coli, and protease deficient strains of E. coli. Vectors, such as the fth1 vector (Nucleic Acids Res. May 15, 2001; 29(10):E50-0) can be useful for the expression of the fusion protein.

The expression vector also can have a secretory signal sequence fused to the DNA encoding each subunit of the antibody or fragment thereof. This sequence is typically located immediately 5′ to the gene encoding the fusion protein, and will thus be transcribed at the amino terminus of the fusion protein. However, in certain cases, the signal sequence has been demonstrated to be located at positions other than 5′ to the gene encoding the protein to be secreted. This sequence targets the protein to which it is attached across the inner membrane of the bacterial cell. The DNA encoding the signal sequence may be obtained as a restriction endonuclease fragment from any gene encoding a protein that has a signal sequence. Suitable prokaryotic signal sequences may be obtained from genes encoding, for example, LamB or OmpF (Wong et al., Gene, 68:1931 (1983), MalE, PhoA and other genes. A preferred prokaryotic signal sequence for practicing this invention is the E. coli heat-stable enterotoxin II (STII) signal sequence as described by Chang et al., Gene 55:189 (1987), and malE.

The vector also typically includes a promoter to drive expression of the fusion protein. Promoters most commonly used in prokaryotic vectors include the lac Z promoter system, the alkaline phosphatase pho A promoter, the bacteriophage γ_{-PL} promoter (a temperature sensitive promoter), the tac promoter (a hybrid trp-lac promoter that is regulated by the lac repressor), the tryptophan promoter, and the bacteriophage T7 promoter. For general descriptions of promoters, see section 17 of Sambrook et al. supra. While these are the most commonly used promoters, other suitable microbial promoters may be used as well.

The vector can also include other nucleic acid sequences, for example, sequences encoding gD tags, c-Myc epitopes, poly-histidine tags, fluorescence proteins (eg., GFP), or beta-galactosidase protein which can be useful for detection or purification of the fusion protein expressed on the surface of the phage or cell. Nucleic acid sequences encoding, for example, a gD tag, also provide for positive or negative selection of cells or virus expressing the fusion protein. In some embodiment, the gD tag is preferably fused to an antibody variable domain which is not fused to the viral coat protein. Nucleic acid sequences encoding, for example, a polyhistidine tag, are useful for identifying fusion proteins including antibody variable domains that bind to a specific antigen using immunohistochemistry. Tags useful for detection of antigen binding can be fused to either an antibody variable domain not fused to a viral coat protein or an antibody variable domain fused to a viral coat protein.

Another useful component of the vectors used to practice this invention is phenotypic selection genes. Typical phenotypic selection genes are those encoding proteins that confer antibiotic resistance upon the host cell. By way of illustration, the ampicillin resistance gene (ampr), and the tetracycline resistance gene (tetr) are readily employed for this purpose.

The vector can also include nucleic acid sequences containing unique restriction sites and suppressible stop codons. The unique restriction sites are useful for moving antibody variable domains between different vectors and expression systems. The suppressible stop codons are useful to control the level of expression of the fusion Protein And to facilitate purification of soluble antibody fragments. For example, an amber stop codon can be read as Gln in a supE host to enable phage display, while in a non-supE host it is read as a stop codon to produce soluble antibody fragments without fusion to phage coat proteins. These synthetic sequences can be fused to one or more antibody variable domains in the vector.

It is preferable to use vector systems that allow the nucleic acid encoding an antibody sequence of interest, for example a CDR having variant amino acids, to be easily removed from the vector system and placed into another vector system. For example, appropriate restriction sites can be engineered in a vector system to facilitate the removal of the nucleic acid sequence encoding an antibody or antibody variable domain having variant amino acids. The restriction sequences are usually chosen to be unique in the vectors to facilitate efficient excision and ligation into new vectors. Antibodies or antibody variable domains can then be expressed from vectors without extraneous fusion sequences, such as viral coat proteins or other sequence tags.

Between nucleic acid encoding antibody variable domain (gene 1) and the viral coat protein (gene 2), DNA encoding a termination codon may be inserted, such termination codons including UAG (amber), UAA (ocher) and UGA (opel). (Microbiology, Davis et al., Harper & Row, New York, 1980, pp. 237, 245-47 and 374). The termination codon expressed in a wild type host cell results in the synthesis of the gene 1 protein product without the gene 2 Protein Attached. However, growth in a suppressor host cell results in the synthesis of detectable quantities of fused protein. Such suppressor host cells are well known and described, such as E. coli suppressor strain (Bullock et al., BioTechniques 5:376-379 (1987)). Any acceptable method may be used to place such a termination codon into the mRNA encoding the fusion polypeptide.

The suppressible codon may be inserted between the first gene encoding a antibody variable domain, and a second gene encoding at least a portion of a phage coat protein. Alternatively, the suppressible termination codon may be inserted adjacent to the fusion site by replacing the last amino acid triplet in the antibody variable domain or the first amino acid in the phage coat protein. When the plasmid containing the suppressible codon is grown in a suppressor host cell, it results in the detectable production of a fusion polypeptide containing the polypeptide and the coat protein. When the plasmid is grown in a non-suppressor host cell, the antibody variable domain is synthesized substantially without fusion to the phage coat protein due to termination at the inserted suppressible triplet UAG, UAA, or UGA. In the non-suppressor cell the antibody variable domain is synthesized and secreted from the host cell due to the absence of the fused phage coat protein which otherwise anchored it to the host membrane.

In some embodiments, the CDR being diversified (randomized) may have a stop codon engineered in the template sequence (referred to herein as a “stop template”). This feature provides for detection and selection of successfully diversified sequences based on successful repair of the stop codon(s) in the template sequence due to incorporation of the oligonucleotide(s) comprising the sequence(s) for the variant amino acids of interest. This feature is further illustrated in the Examples below.

The light and/or heavy antibody variable domains can also be fused to an additional peptide sequence, the additional peptide sequence allowing the interaction of one or more fusion polypeptides on the surface of the viral particle or cell. These peptide sequences are herein referred to as “dimerization sequences”, “dimerization peptides” or “dimerization domains”. Suitable dimerization domains include those of proteins having amphipathic alpha helices in which hydrophobic residues are regularly spaced and allow the formation of a dimer by interaction of the hydrophobic residues of each protein; such proteins and portions of proteins include, for example, leucine zipper regions. The dimerization regions are preferably located between the antibody variable domain and the viral coat protein.

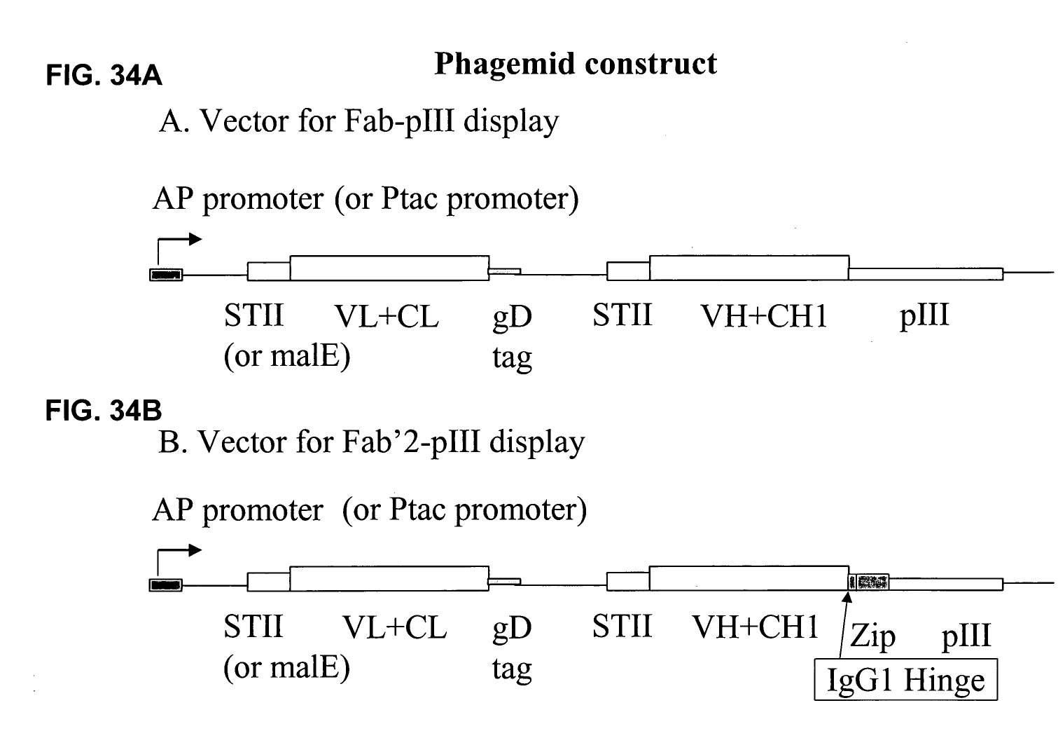

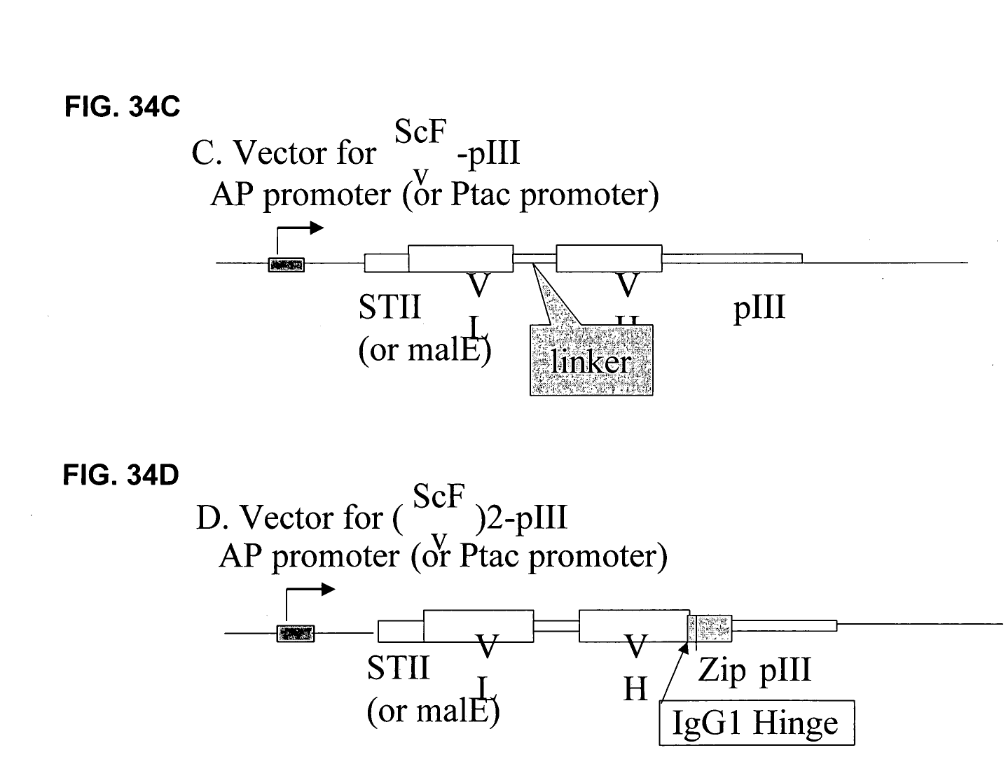

In some cases the vector encodes a single antibody-phage polypeptide in a single chain form containing, for example, both the heavy and light chain variable regions fused to a coat protein. In these cases the vector is considered to be “monocistronic”, expressing one transcript under the control of a certain promoter. Illustrative examples of such vectors are shown in FIGS. 34C and D. In FIG. 34C, a vector is shown as utilizing the alkaline phosphatase (AP) or Tac promoter to drive expression of a monocistronic sequence encoding VL and VH domains, with a linker peptide between the VL and VH domains. This cistronic sequence is connected at the 5′ end to an E. coli malE or heat-stable enterotoxin II (STII) signal sequence and at its 3′ end to all or a portion of a viral coat protein (shown in the FIG. 34 as the pIII protein). The fusion polypeptide encoded by this vector is referred to herein as “ScFv-pIII”. In some embodiments, illustrated in FIG. 34D, the vector may further comprise a sequence encoding a dimerization domain (such as a leucine zipper) at its 3′ end, between the second variable domain sequence (VH in FIG. 34D) and the viral coat protein sequence. Fusion polypeptides comprising the dimerization domain are capable of dimerizing to form a complex of two scFv polypeptides (referred to herein as “(ScFv)2-pIII)”).

In other cases, the variable regions of the heavy and light chains can be expressed as separate polypeptides, the vector thus being “bicistronic”, allowing the expression of separate transcripts. Examples of bicistronic vectors are schematically shown in FIGS. 34A and 4B. In these vectors, a suitable promoter, such as the Ptac or PhoA promoter, can be used to drive expression of a bicistronic message. A first cistron, encoding, for example, a light chain variable domain, is connected at the 5′ end to a E. coli malE or heat-stable enterotoxin II (STII) signal sequence and at the 3′ end to a nucleic acid sequence encoding a gD tag. A second cistron, encoding, for example, a heavy chain variable domain, is connected at its 5′ end to a E. coli malE or heat-stable enterotoxin II (STII) signal sequence and at the 3′ end to all or a portion of a viral coat protein.

Display of Fusion Polypeptides

Fusion polypeptides with an antibody variable domain can be displayed on the surface of a cell or virus in a variety of formats. These formats include single chain Fv fragment (scFv), F(ab) fragment, variable domain of a monobody and multivalent forms of these fragments. The multivalent forms preferably are a dimer of ScFv, Fab, or F(ab)′, herein referred to as (ScFv)₂, F(ab)₂ and F(ab)′₂, , respectively. The multivalent forms of display are preferred in part because they have more than one antigen binding site which generally results in the identification of lower affinity clones and also allows for more efficient sorting of rare clones during the selection process.

Methods for displaying fusion polypeptides comprising antibody fragments, on the surface of bacteriophage, are well known in the art, for example as described in patent publication number WO 92/01047 and herein. Other patent publications WO 92/20791; WO 93/06213; WO 93/11236 and WO 93/19172, describe related methods and are all herein incorporated by reference. Other publications have shown the identification of antibodies with artificially rearranged V gene repertoires against a variety of antigens displayed on the surface of phage (for example, H. R. Hoogenboom & G. Winter J. Mol. Biol. 227 381-388 1992; and as disclosed in WO 93/06213 and WO 93/11236).

When a vector is constructed for display in a scFv format, it includes nucleic acid sequences encoding an antibody variable light chain domain and an antibody variable heavy chain variable domain. Typically, the nucleic acid sequence encoding an antibody variable heavy chain domain is fused to a viral coat protein. One or both of the antibody variable domains can have variant amino acids in at least one CDR region. The nucleic acid sequence encoding the antibody variable light chain is connected to the antibody variable heavy chain domain by a nucleic acid sequence encoding a peptide linker. The peptide linker typically contains about 5 to 15 amino acids. Optionally, other sequences encoding, for example, tags useful for purification or detection can be fused at the 3′ end of either the nucleic acid sequence encoding the antibody variable light chain or antibody variable heavy chain domain or both.

When a vector is constructed for F(ab) display, it includes nucleic acid sequences encoding antibody variable domains and antibody constant domains. A nucleic acid encoding a variable light chain domain is fused to a nucleic acid sequence encoding a light chain constant domain. A nucleic acid sequence encoding an antibody heavy chain variable domain is fused to a nucleic acid sequence encoding a heavy chain constant CH1 domain. Typically, the nucleic acid sequence encoding the heavy chain variable and constant domains are fused to a nucleic acid sequence encoding all or part of a viral coat protein. One or both of the antibody variable light or heavy chain domains can have variant amino acids in at least one CDR. The heavy chain variable and constant domains are preferably expressed as a fusion with at least a portion of a viral coat and the light chain variable and constant domains are expressed separately from the heavy chain viral coat fusion protein. The heavy and light chains associate with one another, which may be by covalent or non-covalent bonds. Optionally, other sequences encoding, for example, polypeptide tags useful for purification or detection, can be fused at the 3′ end of either the nucleic acid sequence encoding the antibody light chain constant domain or antibody heavy chain constant domain or both.

Preferably a bivalent moiety, for example, a F(ab)₂ dimer or F(ab)′₂ dimer, is used for displaying antibody fragments with the variant amino acid substitutions on the surface of a particle. It has been found that F(ab)′₂ dimers have the same affinity as F(ab) dimers in a solution phase antigen binding assay but the off rate for F(ab)′₂ are reduced because of a higher avidity in an assay with immobilized antigen. Therefore the bivalent format (for example, F(ab)′₂) is a particularly useful format since it can allow the identification of lower affinity clones and also allows more efficient sorting of rare clones during the selection process.

Introduction of Vectors into Host Cells

Vectors constructed as described in accordance with the invention are introduced into a host cell for amplification and/or expression. Vectors can be introduced into host cells using standard transformation methods including electroporation, calcium phosphate precipitation and the like. If the vector is an infectious particle such as a virus, the vector itself provides for entry into the host cell. Transfection of host cells containing a replicable expression vector which encodes the gene fusion and production of phage particles according to standard procedures provides phage particles in which the fusion protein is displayed on the surface of the phage particle.

Replicable expression vectors are introduced into host cells using a variety of methods. In one embodiment, vectors can be introduced into cells using electroporation as described in WO/00106717. Cells are grown in culture in standard culture broth, optionally for about 6-48 hours (or to OD₆₀₀=0.6-0.8) at about 37° C., and then the broth is centrifuged and the supernatant removed (e.g. decanted). Initial purification is preferably by resuspending the cell pellet in a buffer solution (e.g. 1.0 mM HEPES pH 7.4) followed by recentriguation and removal of supernatant. The resulting cell pellet is resuspended in dilute glycerol (e.g. 5-20% v/v) and again recentrifuged to form a cell pellet and the supernatant removed. The final cell concentration is obtained by resuspending the cell pellet in water or dilute glycerol to the desired concentration.

A particularly preferred recipient cell is the electroporation competent E. coli strain of the present invention, which is E. coli strain SS320 (Sidhu et al., Methods Enzymol. (2000), 328:333-363). Strain SS320 was prepared by mating MC1061 cells with XL1-BLUE cells under conditions sufficient to transfer the fertility episome (F′ plasmid) or XL1-BLUE into the MC1061 cells. Strain SS320 has been deposited with the American Type Culture Collection (ATCC), 10801 University Boulevard, Manassas, Va. USA, on Jun. 18, 1998 and assigned Deposit Accession No. 98795. Any F′ episome which enables phage replication in the strain may be used in the invention. Suitable episomes are available from strains deposited with ATCC or are commercially available (CJ236, CSH18, DHF′, JM101, JM103, JM105, JM107, JM109, JM110), KS1000, XL1-BLUE, 71-18 and others).

The use of higher DNA concentrations during electroporation (about 10×) increases the transformation efficiency and increases the amount of DNA transformed into the host cells. The use of high cell concentrations also increases the efficiency (about 10×). The larger amount of transferred DNA produces larger libraries having greater diversity and representing a greater number of unique members of a combinatorial library. Transformed cells are generally selected by growth on antibiotic containing medium.

Screening for Binders

Phage display of proteins, peptides and mutated variants thereof, involves constructing a family of variant replicable vectors containing a transcription regulatory element operably linked to a gene fusion encoding a fusion polypeptide, transforming suitable host cells, culturing the transformed cells to form phage particles which display the fusion polypeptide on the surface of the phage particle, contacting the recombinant phage particles with a target molecule so that at least a portion of the particle bind to the target, and separating the particles which bind from particle that do not bind.

Variable domain fusion proteins expressing the variant amino acids can be expressed on the surface of a phage or a cell and then screened for the ability of members of the group of fusion proteins to specifically bind a target molecule, such as a target protein, which is typically an antigen of interest. Target proteins can also include protein L or Protein A which specifically binds to antibody or antibody fragments and can be used to enrich for library members that display correctly folded antibody fragments (fusion polypeptides). In another embodiment, a target molecule is a molecule that specifically binds to folded polypeptide and does not bind to unfolded polypeptide and does not bind at an antigen binding site. For example, for Protein A, the Protein A binding site of Vh3 antibody variable domains are found on the opposite B sheet from the antigen binding site. Another example of a target molecule includes an antibody or antigen binding fragment or polypeptide that does not bind to the antigen binding site and binds to folded polypeptide and does not bind to unfolded polypeptide, such as an antibody to the Protein A binding site. Target proteins, such as receptors, may be isolated from natural sources or prepared by recombinant methods by procedures known in the art.

Screening for the ability of a fusion polypeptide to bind a target molecule can also be performed in solution phase. For example, a target molecule can be attached with a detectable moiety, such as biotin. Phage that binds to the target molecule in solution can be separated from unbound phage by a molecule that binds to the detectable moiety, such as streptavidin-coated beads where biotin is the detectable moiety. Affinity of binders (fusion polypeptide that binds to target) can be determined based on concentration of the target molecule used, using formulas and based on criteria known in the art.

Target antigens can include a number of molecules of therapeutic interest. Included among cytokines and growth factors are growth hormone, bovine growth hormone, insulin like growth factors, human growth hormone including n-methionyl human growth hormone, parathyroid hormone, thyroxine, insulin, proinsulin, amylin, relaxin, prorelaxin, glycoprotein hormones such as follicle stimulating hormone(FSH), leutinizing hormone (LH), hemapoietic growth factor, fibroblast growth factor, prolactin, placental lactogen, tumor necrosis factors, mullerian inhibiting substance, mouse gonadotropin-associated polypeptide, inhibin, activin, vascular endothelial growth factors, integrin, nerve growth factors such as NGF-beta, insulin-like growth factor-I and II, erythropoietin, osteoinductive factors, interferons, colony stimulating factors, interleukins, bone morphogenetic proteins, LIF,SCF,FLT-3 ligand and kit-ligand.

The purified target protein may be attached to a suitable matrix such as agarose beads, acrylamide beads, glass beads, cellulose, various acrylic copolymers, hydroxyalkyl methacrylate gels, polyacrylic and polymethacrylic copolymers, nylon, neutral and ionic carriers, and the like. Attachment of the target protein to the matrix may be accomplished by methods described in Methods in Enzymology, 44 (1976), or by other means known in the art.

After attachment of the target protein to the matrix, the immobilized target is contacted with the library expressing the fusion polypeptides under conditions suitable for binding of at least a portion of the phage particles with the immobilized target. Normally, the conditions, including pH, ionic strength, temperature and the like will mimic physiological conditions. Bound particles (“binders”) to the immobilized target are separated from those particles that do not bind to the target by washing. Wash conditions can be adjusted to result in removal of all but the higher affinity binders. Binders may be dissociated from the immobilized target by a variety of methods. These methods include competitive dissociation using the wild-type ligand, altering pH and/or ionic strength, and methods known in the art. Selection of binders typically involves elution from an affinity matrix with a ligand. Elution with increasing concentrations of ligand should elute displayed binding molecules of increasing affinity.

The binders can be isolated and then reamplified or expressed in a host cell and subjected to another round of selection for binding of target molecules. Any number of rounds of selection or sorting can be utilized. One of the selection or sorting procedures can involve isolating binders that bind to protein L or an antibody to a polypeptide tag such as antibody to the gD protein or polyhistidine tag. Another selection or sorting procedure can involve multiple rounds of sorting for stability, such as binding to a target molecule that specifically binds to folded polypeptide and does not bind to unfolded polypeptide followed by selecting or sorting the stable binders for binding to an antigen (such as VEGF).

In some cases, suitable host cells are infected with the binders and helper phage, and the host cells are cultured under conditions suitable for amplification of the phagemid particles. The phagemid particles are then collected and the selection process is repeated one or more times until binders having the desired affinity for the target molecule are selected. Preferably at least 2 rounds of selection are conducted.

After binders are identified by binding to the target antigen, the nucleic acid can be extracted. Extracted DNA can then be used directly to transform E. coli host cells or alternatively, the encoding sequences can be amplified, for example using PCR with suitable primers, and then inserted into a vector for expression.

A preferred strategy to isolate high affinity binders is to bind a population of phage to an affinity matrix which contains a low amount of ligand. Phage displaying high affinity polypeptide is preferentially bound and low affinity polypeptide is washed away. The high affinity polypeptide is then recovered by elution with the ligand or by other procedures which elute the phage from the affinity matrix.

Preferably, the process of screening is carried out by automated systems to allow for high-throughput screening of library candidates.

In one embodiment, the invention provides for novel antibody variable domains or antibody fragments that bind to IGF1 (Insulin-like Growth Factor 1). Preferably, the antibody variable domains bind IGF1 with an IC₅₀ of less than 50□μM and more preferably less than 1 μM. In one embodiment, IGF1 -binding antibodies include members of the library created by substituting amino acid residues 95-100a of the CDR3 region of the variable region of the heavy chain of 4D5 with DVK codon sets or a combination of DVK and NNK codon sets. It has been discovered that some members of the library, as created above, have a particularly high affinity for IGF1. In particular, antibodies including the heavy chain CDR3 sequences SRWKYATRYAM (SEQ ID NO.: 68; amino acid position 93-100c ), SRSRGWWTAAM (SEQ ID NO.:69; amino acid position 93-100c), and SRASRDWYGAM (SEQ ID NO.: 70; amino acid position 93-100c) display high affinity binding to IGF1. Novel antibody variable domains that bind to IGF1, generated by substituting amino acids at positions in other CDRs, such as L1, L2, L3, H1 and H2 can also be generated according to the method described herein.

In another embodiment, the invention provides for novel antibody and antibody fragments that bind to mVEGF (murine Vascular Endothelial Growth Factor). Preferably, the antibody variable domains bind mVEGF with an IC₅₀ of less than 10□μM and more preferably less than 1 μM. In one embodiment, mVEGF-binding antibodies include members of the library created by substituting amino acid residues 95-100a of the CDR3 region of the variable region of the heavy chain of 4D5 with DVK codon sets or a combination of DVK and NNK codon sets. In has been discovered that some members of the library, as created above, have a particularly high affinity for mVEGF. In particular, antibodies including the heavy chain CDR3 sequences SRNAWAF (SEQ ID NO.:5 ; amino acid position 93-100c), SRNLSENSYAM (SEQ ID NO.: 6; amino acid position 93-100c), SRAGWAGWYAM (SEQ ID NO.: 7; amino acid position 93-100c), SRAAKAGWYAM (SEQ ID NO.:8; amino acid position 93-100c), and SRSDGRDSAYAM (SEQ ID NO.: 9 amino acid position 93-100c) display high affinity binding to mVEGF. Novel antibody variable domains that bind to mVEGF, generated by substituting amino acids at positions in other CDRs, such as L1, L2, L3, H1 and H2 can also be generated according to the method described herein.

In some cases these novel CDRH3 sequences can be combined with other sequences generated by introducing variant amino acids via codon sets into other CDRs in the heavy and light chains, for example through a 2-step process. An example of a 2-step process comprises first determining binders (generally lower affinity binders) within one or more libraries generated by randomizing one or more CDRs, wherein the CDRs randomized are each library are different or, where the same CDR is randomized, it is randomized to generate different sequences. CDR diversity from binders from a heavy chain library can then be combined with CDR diversity from binders from a light chain library (eg. by ligating different CDR sequences together). The pool can then be further sorted against target to identify binders possessing increased affinity. For example, binders (for example, low affinity binders) obtained from sorting an L3/H3, an H1/H2/H3 or an L3/H1/H2/H3 library may be combined with binders (for example, low affinity binders) obtained from sorting an L1/L2/H1/H2 or an L1/L2/L3 library, wherein the combined binders are then further sorted against a target of interest to obtain another set of binders (for example, high affinity binders). Novel antibody sequences can be identified that display higher binding affinity to either the IGF1 or mVEGF antigens.

In some embodiments, libraries comprising polypeptides of the invention are subjected to a plurality of sorting rounds, wherein each sorting round comprises contacting the binders obtained from the previous round with a target molecule distinct from the target molecule(s) of the previous round(s). Preferably, but not necessarily, the target molecules are homologous in sequence, for example members of a family of related but distinct polypeptides, such as, but not limited to, cytokines (for example, alpha interferon subtypes).

Generation of Antibody Variable Domain Libraries

In one aspect, libraries with diverse variable domains are generated using the heavy chain variable domain (VHH) of a monobody. The small size and simplicity make monobodies attractive scaffolds for peptidomimetic and small molecule design, as reagents for high throughput protein analysis, or as potential therapeutic agents. The diversified VHH domains are useful, inter alia, in the design of enzyme inhibitors, novel antigen binding molecules, modular binding units in bispecific or intracellular antibodies, as binding reagents in protein arrays, and as scaffolds for presenting constrained peptide libraries.

In one aspect of the invention, libraries with a plurailty of polypeptides comprising variant CDRH3 regions are formed by limiting diversity at structural amino acid positions and allowing for greater diversity at non-structural amino acid positions within the CDRH3. Preferably, the CDRH3 is from a monobody or variable domain of a monobody (VHH). An amino acid position is a structural position if it contributes to the stability of the polypeptide, such as a variable domain. Amino acid positions that can contribute to stability of the polypeptide can be identified using a method such as alanine scanning mutagenesis or shotgun scanning as described in WO 01/44463 and analyzing the effect of loss of the wild type amino acid on structural stability at positions in the CDRH3. If a wild type amino acid is replaced with a scanning amino acid in a position in a CDRH3 region, and the resulting variant exhibits poor binding to a target molecule that binds to folded polypeptide, then that position is important to maintaining the structure of the polypeptide. A structural amino acid position is a position in which, preferably, the ratio of polypeptides with wild type amino acid at a position to a variant substituted with a scanning amino acid at that position is at least about 3 to 1, 5 to1, 8 to1, or about 10 to 1 or greater. Once the structural amino acid positions are identified, diversity is limited at these positions in order to provide a library with a diverse CDRH3 region while minimizing the structural perturbations.

At least one structural amino acid position in a CDRH3 is substituted with an amino acid found at a frequency greater than the average frequency for any amino acid at that position in a population of polypeptides with randomized CDRH3 regions. Preferably, the frequency is at least 60% or greater than the average frequency for any amino acid at that position, more preferably, the frequency is at least one standard deviation (as determined using standard statistical methods) greater than or above the average frequency for any amino acid at that position. In one embodiment, at least one structural amino acid position in the CDRH3 is substituted with a hydrophobic amino acid or arginine or tyrosine, preferably selected from the group consisting of valine, isoleucine, leucine, tyrosine, tryptophan, phenylalanine, and arginine.

The variant CDRH3 region also comprises a non-structural amino acid position that has a variant amino acid. Non-structural amino acid positions can vary in sequence and in length. In some embodiments, one or more non-structural amino acid positions are located in between the N terminal and C terminal flanking regions. Said at least one non-structural position is or comprises a contiguous sequence of about 1 to 20 amino acids; more preferably 1 to 17 amino acids; more preferably 5 to 15 amino acids and most preferably about 9 to 12 amino acids. The non-structural amino acid positions can be substituted randomly with any of the naturally occurring amino acids or with selected amino acids. In some embodiments, said at least one non-structural position can have a variant amino acid encoded by a random codon set or a nonrandom codon. The nonrandom codon set preferably encodes amino acids that are commonly occurring at that position in naturally occurring known antibodies. Examples of nonrandom codon sets include DVK, XYZ, and NVT.

In another aspect, antibody libraries can be generated by mutating the solvent accessible and/or highly diverse positions in at least one CDR of an antibody variable domain. Some or all of the CDRs can be mutated using the methods of the invention. In some embodiments, it may be preferable to generate diverse antibody libraries by mutating positions in CDRH1, CDRH2 and CDRH3 to form a single library or by mutating positions in CDRL3 and CDRH3 to form a single library or by mutating positions in CDRL3 and CDRH1, CDRH2 and CDRH3 to form a single library.

A library of antibody variable domains can be generated, for example, having mutations in the solvent accessible and/or highly diverse positions of CDRH1, CDRH2 and CDRH3. Another library can be generated having mutations in CDRL1, CDRL2 and CDRL3. These libraries can also be used in conjunction with each other to generate binders of desired affinities. For example, after one or more rounds of selection of heavy chain libraries for binding to a target antigen, a light chain library can be replaced into the population of heavy chain binders for further rounds of selection to increase the affinity of the binders.

In one embodiment, a library is created by substitution of original amino acids with variant amino acids in the CDRH3 region of the variable region of the heavy chain sequence. According to the invention, this library can contain a plurality of antibody sequences, wherein the sequence diversity is primarily in the CDRH3 region of the heavy chain sequence, more specifically in amino acid residues 95-100a of CDRH3 of antibody 4D5.

In one aspect, the library is created in the context of the humanized antibody 4D5 sequence, or the sequence of the framework amino acids of the humanized antibody 4D5 sequence. Preferably, the library is created by substitution of at least residues 95-100a of the heavy chain with amino acids encoded by the DVK codon set, wherein the DVK codon set is used to encode a set of variant amino acids for every one of these positions. An example of an oligonucleotide set that is useful for creating these substitutions comprises the sequence (DVK)₇ ; an example of an oligonucleotide set having this sequence is oligonucleotide (F63) (SEQ ID NO: 10). In some embodiments, a library is created by substitution of residues 95-100a with amino acids encoded by both DVK and NNK codon sets. An example of an oligonucleotide set that is useful for creating these substitutions comprises the sequence (DVK)₆ (NNK) ; an example of an oligonucleotide set having this sequence is oligonucleotide (F65) (SEQ ID NO: 11). In another embodiment, a library is created by substitution of at least residues 95-100a with amino acids encoded by both DVK and NNK codon sets. An example of an oligonucleotide set that is useful for creating these substitutions comprises the sequence (DVK)₅ (NNK); an example of an oligonucleotide set having this sequence is oligonucleotide (F64) (SEQ ID NO: 12). Another example of an oligonucleotide set that is useful for creating these substitutions comprises the sequence (NNK)₆ ; an example of an oligonucleotide set having this sequence is oligonucleotide (F66) (SEQ ID NO: 13). Other examples of suitable oligonucleotide sequences are listed in FIG. 4 and can be determined by one skilled in the art according to the criteria described herein.

A library with mutations in CDRH3 can be combined with a library containing variant versions of other CDRs, for example CDRL1, CDRL2, CDRL3, CDRH1 and/or CDRH2. Thus, for example, in one embodiment, a CDRH3 library is combined with a CDRL3 library created in the context of the humanized 4D5 antibody sequence with variant amino acids at positions 28, 29, 30,31, and/or 32 using codon sets as described in FIG. 3. Examples of oligonucleotides useful in creating these substitutions include those that incorporate these codon sets. In another embodiment, a library with mutations to the CDRH3 can be combined with a library comprising variant CDRH1 and/or CDRH2 heavy chain variable domains. In one embodiment, the CDRH1 library is created with the humanized antibody 4D5 sequence with variant amino acids at positions 28, 30,31, 32 and 33 using codon sets as described in FIG. 3. Examples of oligonucleotide sets useful in creating these substitutions include those that incorporate these codon sets. A CDRH2 library may be created with the sequence of humanized antibody 4D5 with variant amino acids at positions 50, 52, 53, 54, 56 and 58 using the codon sets described in FIG. 3. Examples of oligonucleotide sets useful in creating these substitutions include those that incorporate these codon sets.

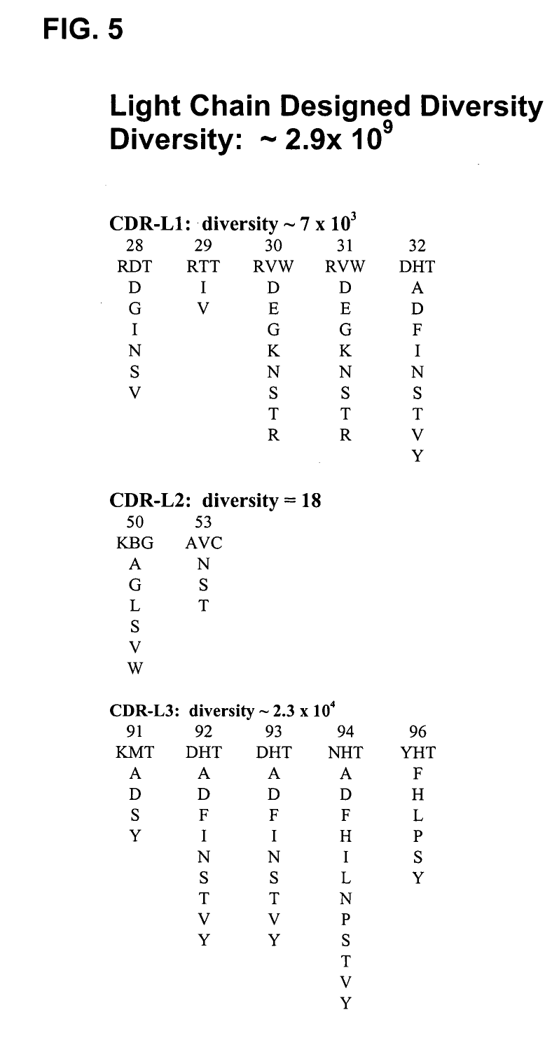

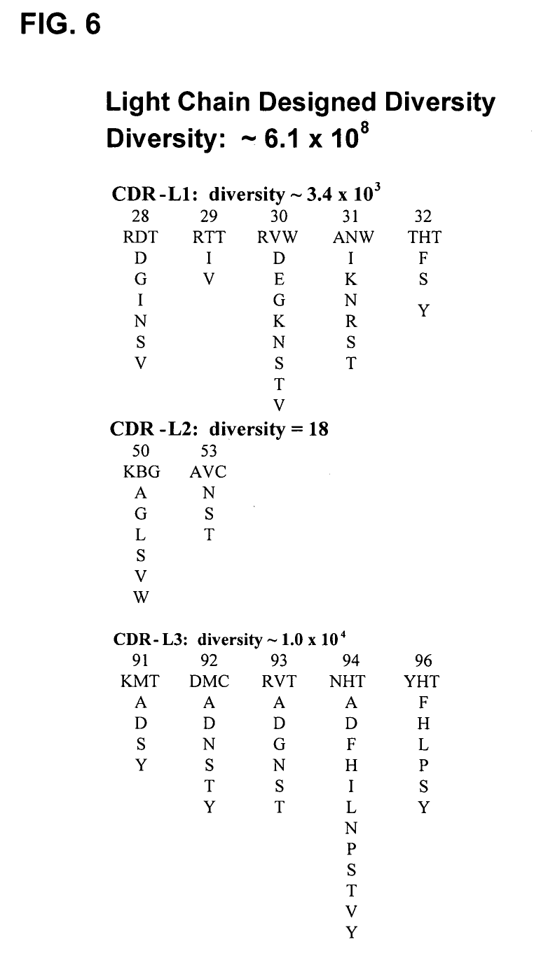

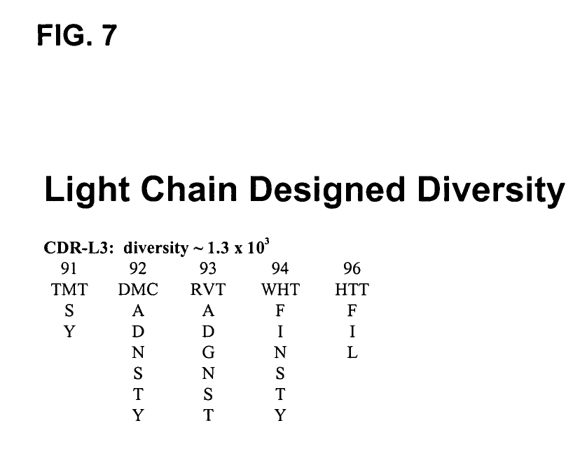

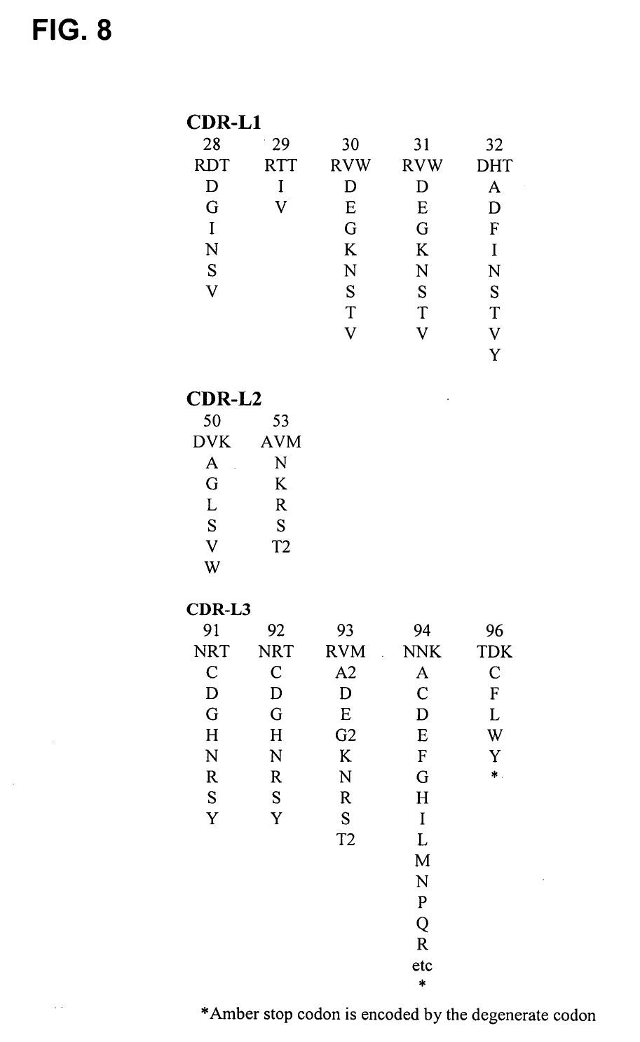

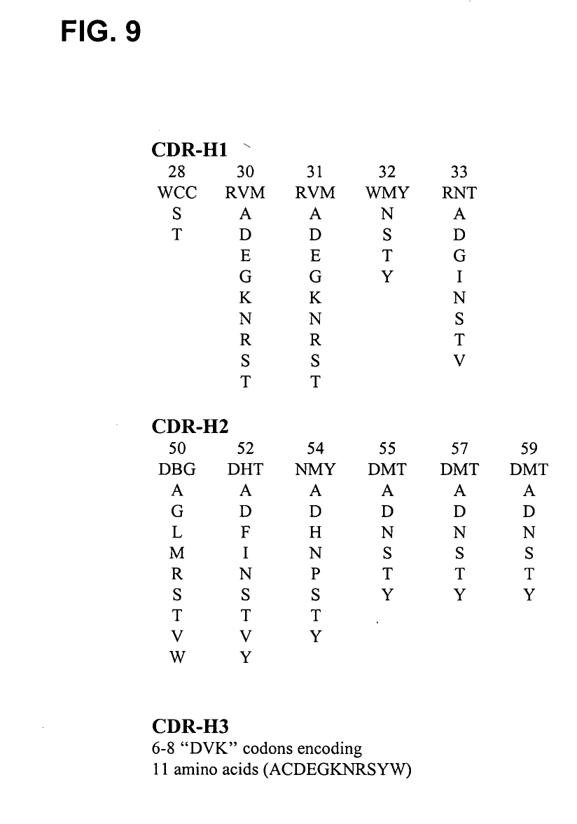

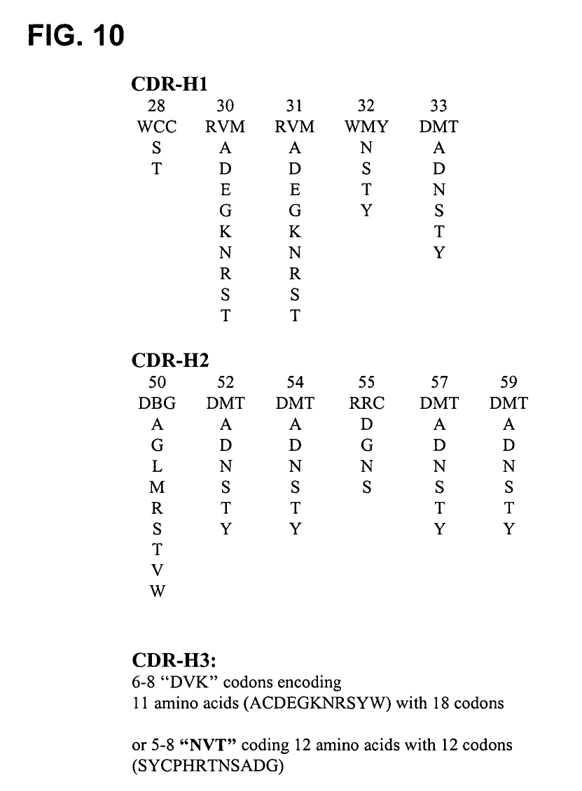

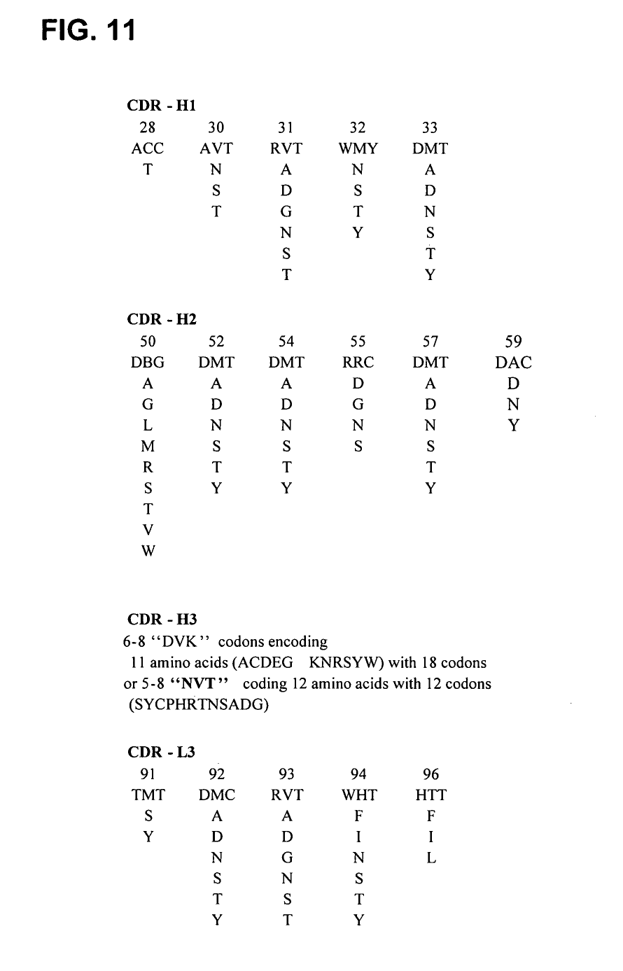

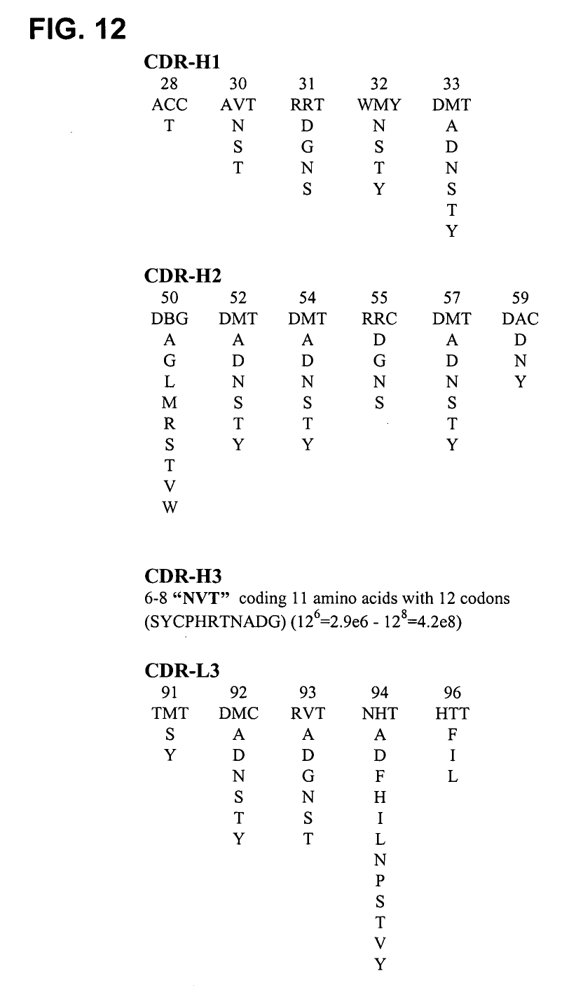

Any combination of codon sets and CDRs can be diversified according to the amino acid position selection criteria described herein. Examples of suitable codons in various combinations of CDRs are illustrated in FIGS. 5-13. FIGS. 5-7 also include illustrative calculations of designed diversity values of libraries generated according to the choice of codon sets used in the indicated CDRs and amino acid positions.

Having generally described the invention, the same will be more readily understood by reference to the following examples, which are provided by way of illustration and are not intended as limiting.

### EXAMPLE 1

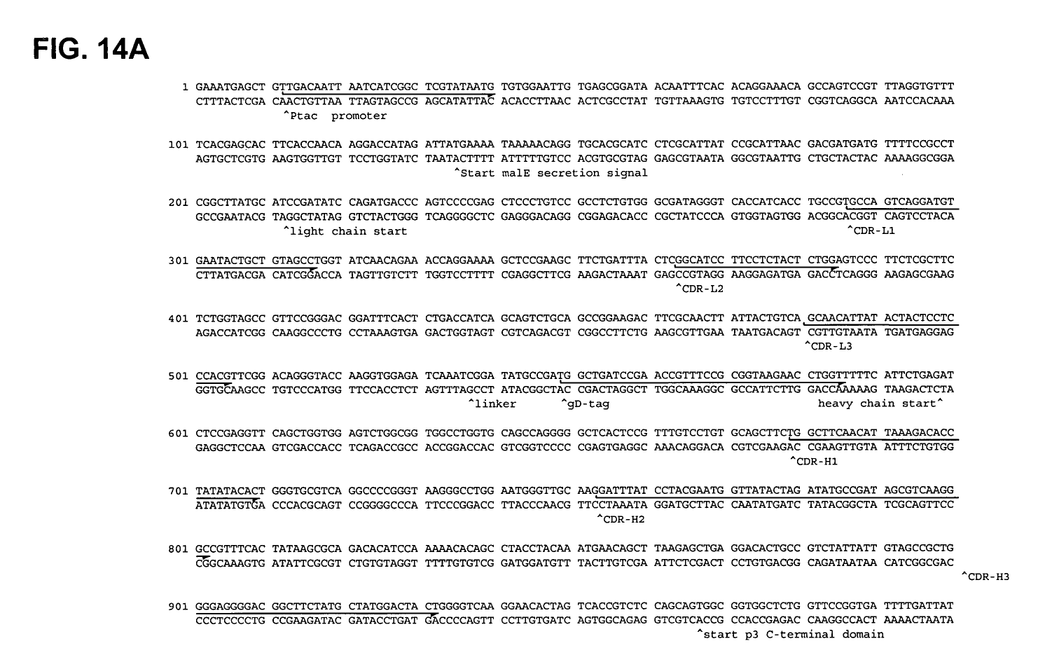

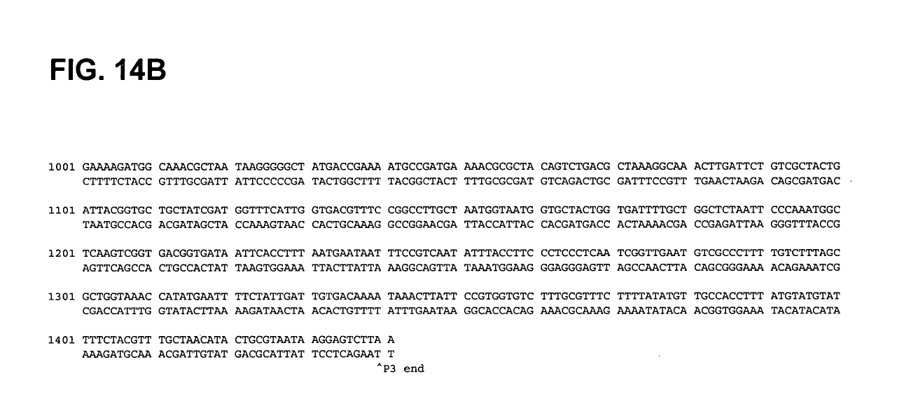

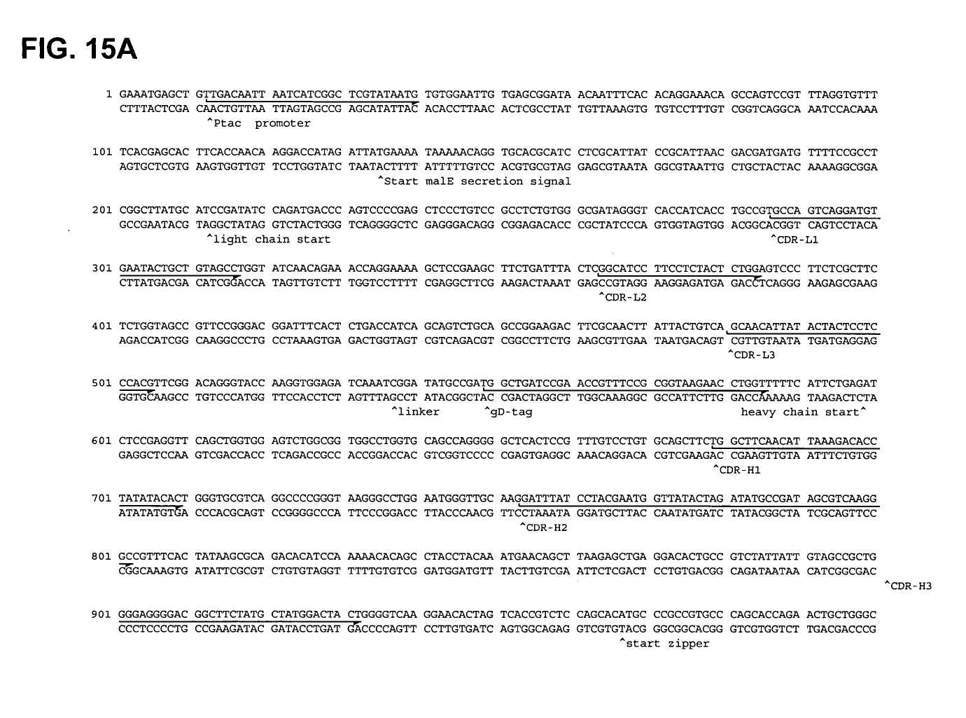

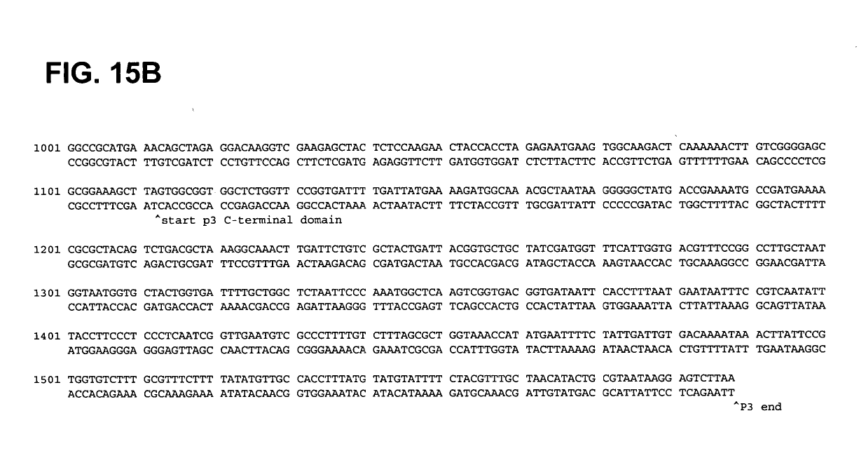

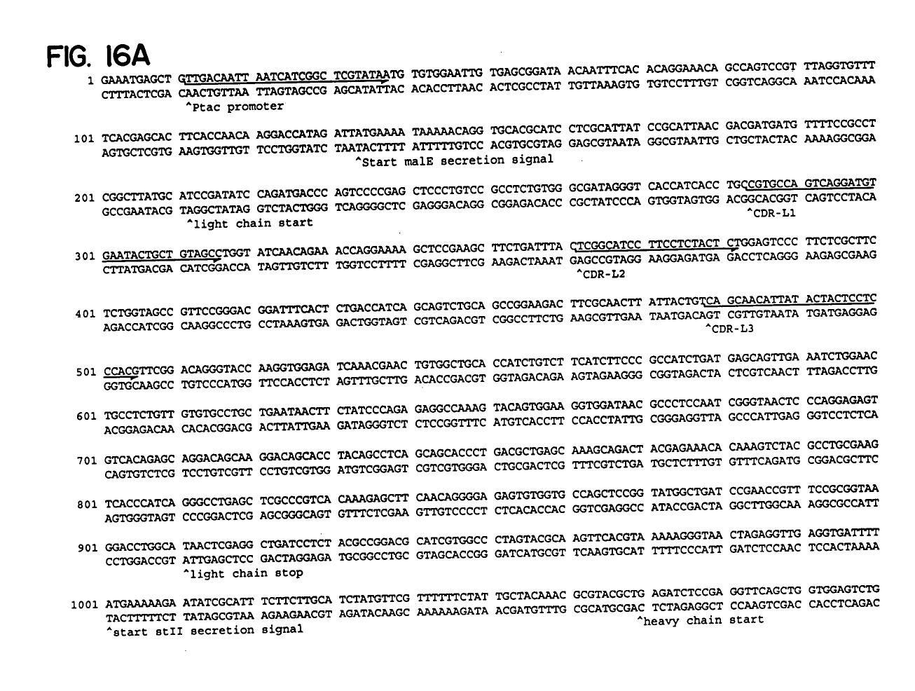

Vectors encoding fusion polypeptides comprising variant CDRs were constructed as follows. In general, vectors for antibody phage display were constructed by modifying vector pS1607 (Sidhu et al., J. Mol. Biol. (2000), 296:487-495). Vector pS1607, which has pTac promoter sequence and malE secretion signal sequence, contained a sequence of human growth hormone fused to the C-terminal domain of the gene-3 minor coat protein (p3). The sequence encoding hGH was removed, and the resulting vector sequence served as the vector backbone for construction of vectors of the present invention that contain DNA fragments encoding the anti-her2 humanized antibody 4D5 light chain and heavy chain variable domain sequences in the form of:
(i) single chain Fv (scFv) (SEQ ID NO.: 18; FIG. 14);
(ii) single chain Fv with zipper domain (scFvzip) (SEQ ID NO.: 19; FIG. 15);
(iii) Fab fragment (Fab) (SEQ ID NO.: 20; FIG. 16);
   or (iv) Fab fragment with zipper domain (Fabzip) (SEQ ID NO.: 21; FIG. 17). The humanized antibody 4D5 is an antibody which has mostly human consensus sequence framework regions in the heavy and light chains, and CDR regions from a mouse monoclonal antibody specific for Her-2. The method of making the anti-Her-2 antibody and the identity of the variable domain sequences are provided in U.S. Pat. Nos. 5,821,337 and 6,054,297. The resulting vectors (schematically illustrated in FIGS. 34A-D) comprise the humanized antibody 4D5 variable domains under the control of the IPTG-inducible Ptac promoter (sequences as shown in FIGS. 14-17) or the alkaline phosphatase phoA promoter (as described in U.S. Pat. No. 5,750,373).

A person skilled in the art can utilize the information provided above and in the sequences of FIGS. 14-17, in conjunction with standard molecular biology techniques to construct vectors of the invention. Construction of these vectors is described in greater illustrative detail below for Fab-zip.

Construction of Fab-Zip Construct and Characterization of its Function in Phage Display

Inclusion of the zipper region facilitates the formation and display of dimers of ScFv and F(ab) to form scFv₂ and F(ab)′₂, respectively.

Fab-zip vectors were constructed as described below and shown in FIG. 34B.

Methods and Materials

Construction of Anti-Her2 F(ab)′₂ vector: A phagemid construct comprising a sequence encoding an anti-Her2 polypeptide under the control of the Ptac promoter was generated using vector pS1607 as the backbone, as described above. malE secretion signal sequence was first fused to the N-terminal sequence of light chain (LC) to direct the LC synthesis to the periplasm of bacteria cell. A gD tag was added at the C-terminus of LC. Following the stop codon of LC, another ribosome binding site and STII signal sequence were fused to the N-terminus of heavy chain (HC) sequence and continued with the C-terminal domain of the pIII, a minor coat protein of M13 phage.

To generate F(ab)′₂ displayed on phage, the dimerizable leucine zipper GCN4 sequence was utilized. Cassette mutagenesis was performed to insert in between HC and pIII first the hinge sequence that came from full length IgG1 antibody (TCPPCPAPELLG) (SEQ ID NO:22)followed by GCN4 sequences (GRMKQLEDKVEELLSKNYHLENEVARLKKLVGERG)(SEQ ID NO:3). The GCN4 leucine zipper was expected to bring two sets of LC/HC-pIII fusion polypeptides together in the E. coli periplasm, which would allow the formation of disulfide bonds in the hinge region to secure the dimer formation before and after getting out of the E. coli periplasm.

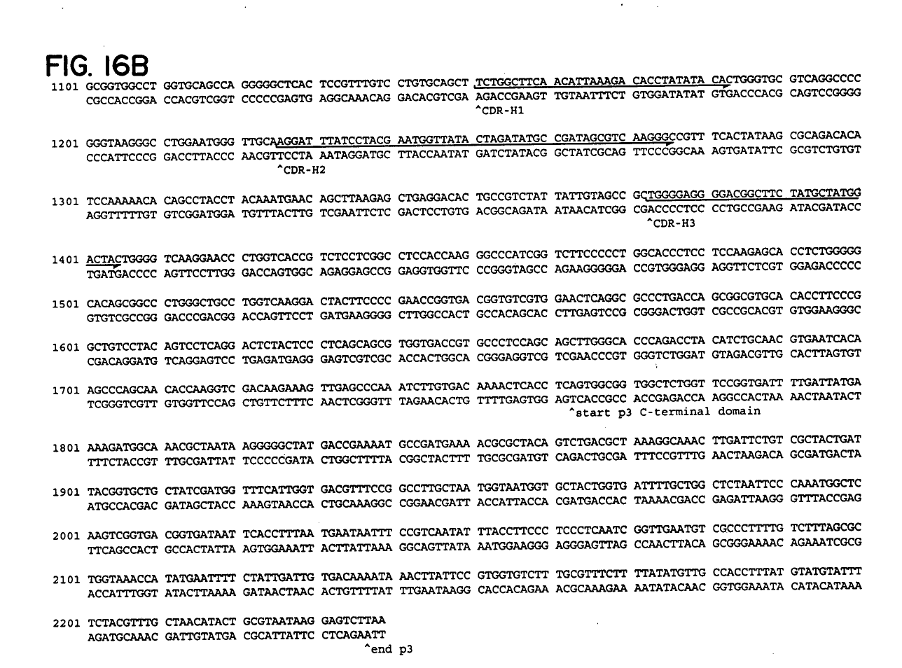

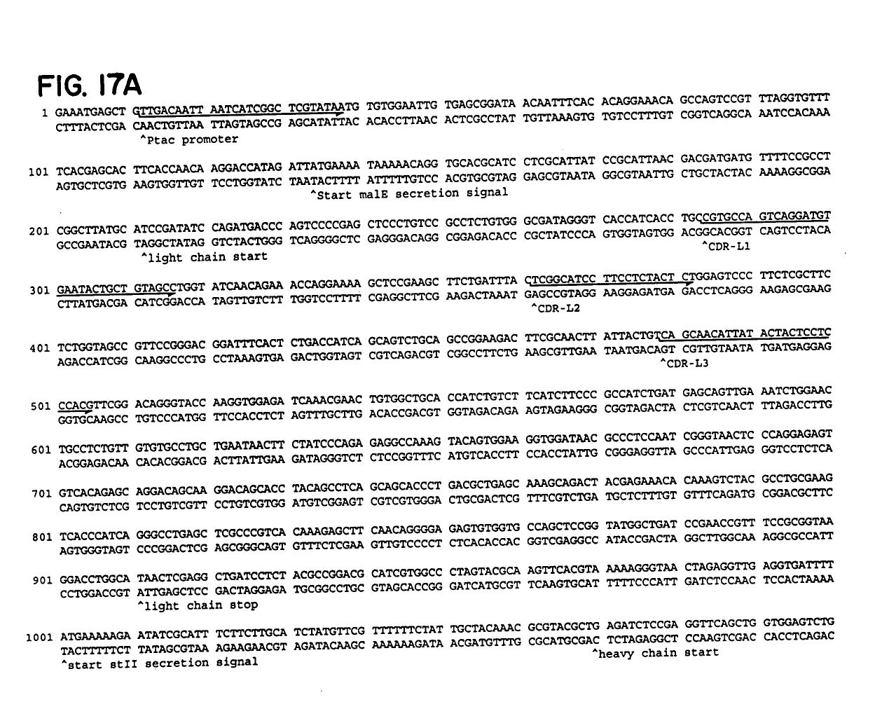

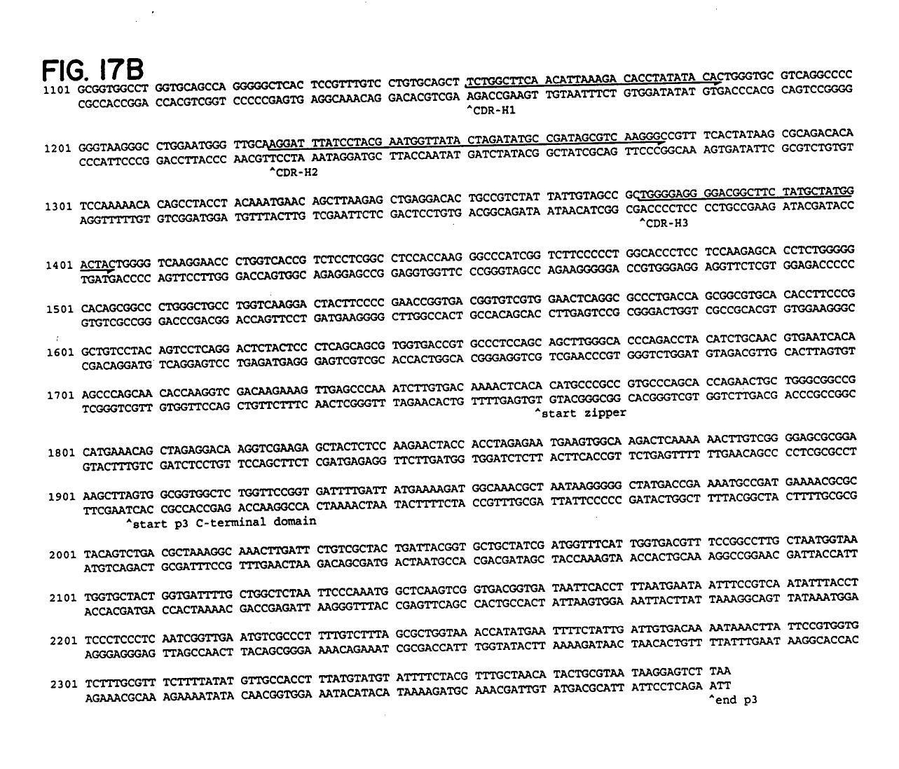

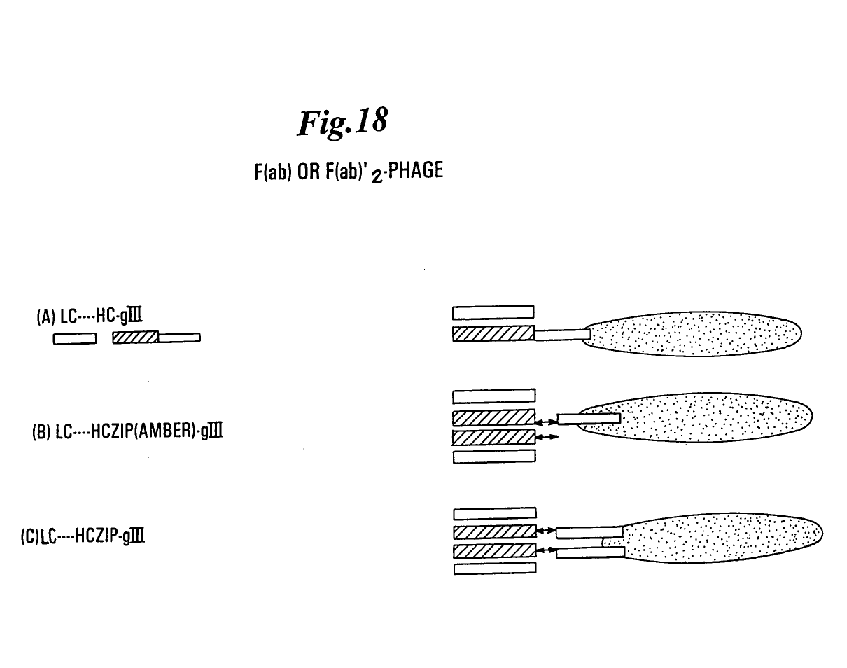

Two versions of the vector schematically illustrated in FIG. 34B were made. One had an Amber stop codon (TAG) after the GCN4 zipper sequence and one did not. These two constructs would theoretically produce one or both of the divalently displaying phage as depicted in FIG. 18. The Amber-less construct would make only the form (C) that would have two copies out of the five copies of pIII on phage as fusion polypeptide which would be stabilized with both the hinge disulfide and GCN4 zipper. The construct with Amber after the GCN4 should be able to produce either form (B) or (C) of the phage depending on the efficiency of suppression of the Amber stop codon in XL-1 bacterial strain.

The formation of F(ab)′₂ on phase: To demonstrate the formation of F(ab)′₂, or the divalent display of F(ab) on the phage, the expected function of divalent display was measured. With the avidity effect of divalent display, the phage binding to ligand-modified solid phase should demonstrate significantly decreased off-rate, the rate at which it detaches off the solid phase, if the density of the ligand on the solid support is high enough to allow divalent binding. For divalent interaction to detach off the plate, both interactions have to be broken simultaneously for the phage to come off, which predictably would occur with much less frequency.

To produce displaying phage, E. Coli strain XL-1 Blue (Stratagene, San Diego, Calif.) infected first with F(ab) or F(ab)′₂ phage and then VCS helper phage (Strategene, San Diego, Calif.) were grown in 2YT media at 37° C. for 20 h and phage was harvested as described (Sidhu et al., Methods Enzymol. (2000), 328:333-363). Briefly, phage was purified by first precipitating them from the overnight culture media with polyethylene glycol, and resuspended in PBS. Phage were quantitated by spectrophotometer with its reading at 268 nm (1 OD=1.13×10¹³/ml). Phage ELISA (Sidhu et al., supra) was first performed by titrating the phage in phage ELISA binding buffer (PBS with 0.5% BSA and 0.05% Tween 20) and its binding to ligand (Her-2 extracellular domain, Her-2ECD) coated on 96-well plate was quantified by HRP conjugated anti-M13 antibody followed by adding the peroxidase substrate, H202 and TMG (Kirkgaad) which can be read at wavelength 450 nm. The plate was coated with Her-2ECD at 2 μg/ml in PBS for 2 h at room temperature or 4° C. overnight, which is sufficient to allow divalent binding. We blocked the plate with 0.5% BSA and then 0.2% Tween20 for 1 hour before adding phage dilutions to the wells.

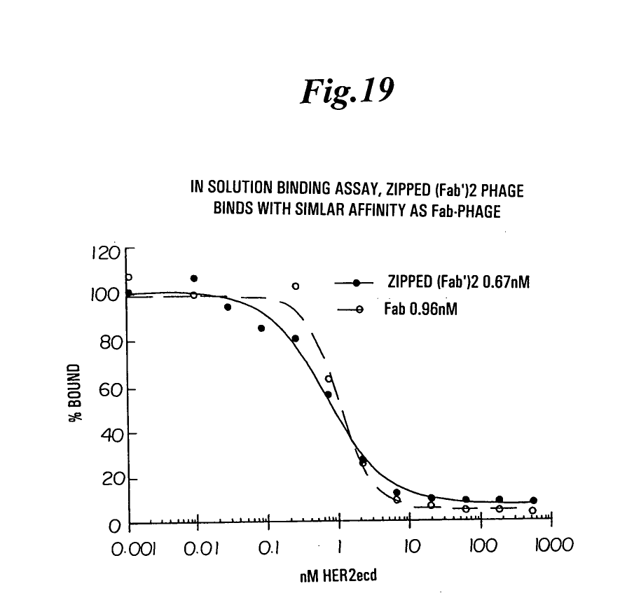

For the off-rate plate binding experiments or solution binding competition ELISA, a phage concentration was used of either F(ab)or F(ab)′₂ which gave about 90% of maximum binding to the coated plate. To show that the F(ab)′2 phage still maintain the same binding affinity where avidity plays less of a role, competition ELISA was performed by incubating the F(ab) or F(ab)′₂ phage with increasing concentrations of Her-2ECD ((0.1 to 500 nM) in solution for 5 hours at 37° C. The unbound phage was captured briefly (15 min.) with plates coated with HER-2ECD and measured with HRP-anti-M13 conjugate. The IC50, the concentration of Her-2ECD that inhibits 50% of the F(ab)-phage, represents the affinity (see FIG. 19).

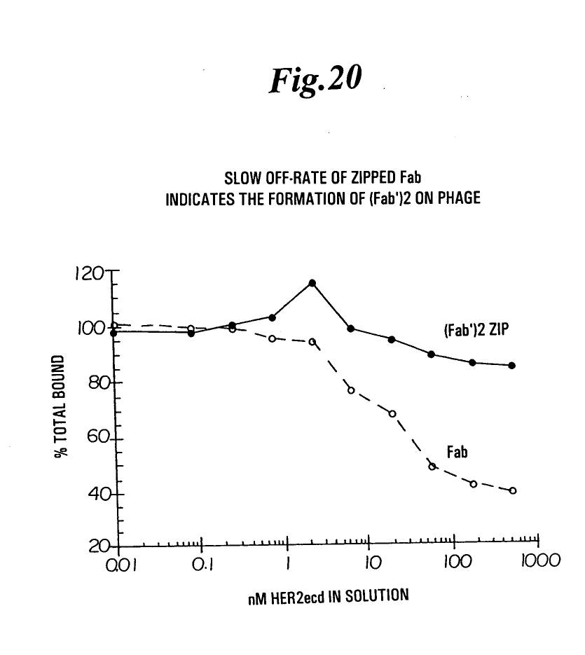

For the off-rate experiment, F(ab) or F(ab)′₂ phage was allowed to bind to Her-2ECD coated wells first, which were then washed to get rid of excess phage. Serial dilutions of Her-2ECD (0.1 nM to 500 nM) were added to the well and incubated for 5 hours at 37° C., during which time the phage was allowed to detach off the plate and the rebinding was inhibited by the Her-2ECD in the solution. Phage that still remained on the plate was then quantified with HRP-antiM13 conjugate. The relative proportion of remaining phage was calculated by dividing the OD at the particular Her-2ECD concentration with OD in the absence of Her-2ECD and shown as % in the FIG. 20.

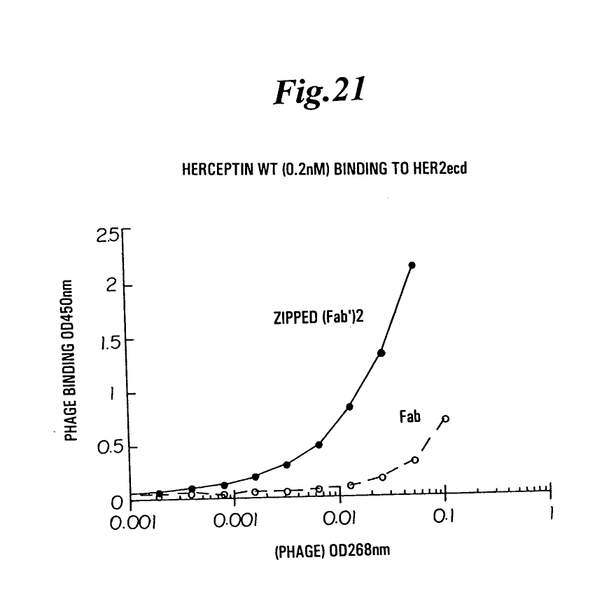

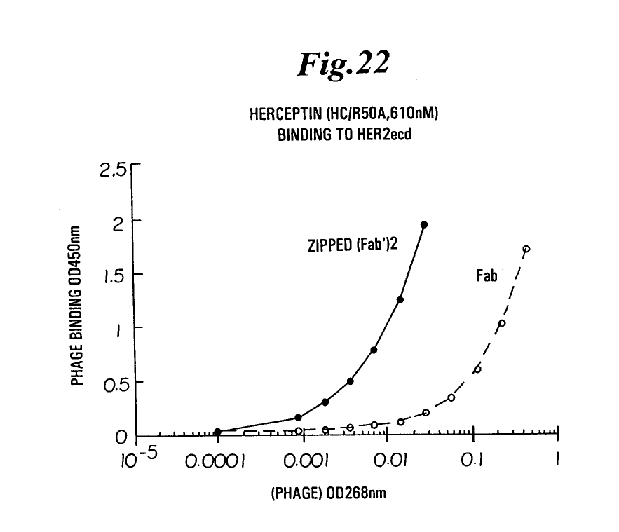

Another way to demonstrate the divalency of F(ab)′₂ phage was to show a difference in the amount of phage that is required to give detectable binding on the ligand coated solid support by standard phage ELISA method as compared to its non-divalent counterpart. We also want to examine the detectability of low affinity binder. We generated a humanized antibody 4D5 mutant by Kunkel site-directed mutagenesis (Kunkle et al., 1985) in its heavy chain, Arginine 50 changed to Alanine (R50A), in both F(ab) and F(ab)′₂ format. Standard phage dilution versus its binding signal on Her-2ECD coated plate phage ELISA was performed (FIG. 21 and FIG. 22). The display level of the mutant was equivalent for this two formats judged from its binding to antibody to gD tag.

Results

Binding properties: The F(ab)′₂ displaying phage has essentially indistinguishable affinity (1 nM) in solution as F(ab) phage (FIG. 19). This means that the insertion of hinge and GCN4 zipper to the C-terminus of HC did not perturb its binding capability. However, the avidity effect of the divalent F(ab)′2 is clearly demonstrated by the significant different behavior from the monovalent F(ab) phage in the plate binding experiments (FIG. 20 and 21). In FIG. 20, F(ab)′2 phage of either construct with or without Amber after the leucine zipper has a much slower rate to detach from the ligand coated plate than F(ab) phage. In FIG. 21, we saw a consistent 40-50 fold difference in the concentration of phage concentration to achieve the same binding signal. The binding of a low affinity R50A (650 nM) binder can be detected and captured at 40-50 fold lower concentration of phage with divalent F(ab)′2 display (FIG. 22). This difference is commonly seen comparing monovalent and divalent interaction, e.g. F(ab) vs. M(ab).

### EXAMPLE 2

### Library Design: L1, L2, L3, H1, H2

Libraries of antibody variable domains were designed to maximize diversity in the CDR regions while minimizing structural perturbations in the antibody variable domains. Structural perturbations in antibody variable domains are generally associated with improperly folded antibody domains resulting in low yield, for example when produced in bacterial cells. Low yields decrease the number of binders detected in screening. Diversity in the CDR regions was generated by identifying solvent accessible and highly diverse positions in each CDR for CDRs L1, L2, L3, H1 and H2, and designing an oligonucleotide comprising at least one tailored (i.e., non-random) codon set encoding variant amino acids for the amino acid position corresponding to the position of at least one solvent accessible residue at a highly diverse position in at least one CDR region. A tailored codon set is a degenerate nucleic acid sequence that preferably encodes the most commonly occurring amino acids at the corresponding positions of the solvent accessible residues in known, natural antibodies.

Solvent accessible residues in the CDRs were identified in the antibody variable domain template molecule by analyzing the crystal structure of the template molecule. Humanized antibody 4D5 is efficiently produced and properly folded when produced in a variety of host cells, including bacterial cell culture. The crystal structure for the humanized antibody 4D5 variable region is known and publicly available at http://www.rcsb.org (accession code IFVC).

The solvent accessible positions in the CDRs of the light chain and CDR1 and CDR2 of the heavy chain were identified using the Insight II program (Accelrys, San Diego, Calif.).

CDR residues were also analyzed to determine which positions in the CDRs were highly diverse. Highly diverse positions in the CDR regions for the heavy and light chains were identified by examining the sequences of known, naturally occurring antibodies in the Kabat database. The Kabat database is available at http://immuno.bme.nwu.edu. In the Kabat database, there were about 1540 sequences of the human light chain and 3600 sequences for the human heavy chain. The CDR sites were aligned and numbered as described by Kabat (see http://immuno.bme.nwu.edu). Highly diverse amino acid positions were identified by lining up and ranking the amino acid usage, from most frequently used to less frequently used for each CDR residue. For example, L3-91 (i.e., residue 91 of the light chain CDR3) was found to be Y (tyrosine) in 849 out of 1582 antibody sequences in the Kabat database, and it is the amino acid found most frequently at this position. Next on the list of frequency serine (occurring in 196 sequences), followed by arginine (169 sequences), alanine (118 sequences), glycine (61 sequences), histidine (41 sequences), with the remaining 35 sequences being one of the remaining amino acids. Illustrative diverse sites, with corresponding diversity list of amino acids, are shown in FIG. 1 (for the light chain) and FIG. 2 (for the heavy chain).

Amino acid residues found in a particular position that collectively constitute the most frequently occurring amino acids among the known, natural antibody sequences are selected as the basis for library design. The most frequently occurring amino acids were deemed to be those that most commonly found in the top 90% of the list of diverse amino acids (this group of amino acids is referred to herein as “target group of amino acids”). However, as described herein, the percent cutoff for a target group of amino acids can be varied, as described above, according to the circumstances and purpose of the diversity library that is to be achieved.

For humanized antibody 4D5, the positions identified as solvent accessible and highly diverse were:

Light Chain

| | |
|---|---|
| CDR1 | 28, 29, 30, 31, 32 |
| CDR2 | 50, 53 |
| CDR3 | 91, 92, 93, 94, 96 |

Heavy Chain

| | |
|---|---|
| CDR1 | 28, 30, 31, 32, 33 |
| CDR2 | 50, 52, 53, 54, 56, 58 |

Examples of amino acids that occur at high frequency in natural diversity (i.e., among known, natural antibody sequences) (referred to as “target group” or “natural diversity” in FIG. 3), and the designed diversity of amino acids by DNA codons (“Diversity<DNA codon>”) for each of these positions is shown in FIG. 3.

Codon sets encoding a specific group of amino acids (Diversity) were designed to include at least a certain percentage of the amino acids in the known, natural sequences (designated as “% covering” in FIG. 3). Of the amino acids encoded by a codon set, at least about 40% of the amino acid are target amino acids identified for a particular solvent accessible and highly diverse position (designated as “% good” in FIG. 3; amino acids encoded by a codon set that are target amino acids are shown in bold in column 3 of FIG. 3). However, as described herein, the % good value can be varied according to circumstance and objectives. The codon sets were selected such that they preferably encoded the amino acids with the highest occurrences at a particular position. The number of non-target amino acids coded by a codon set for a particular position was minimized. Effectiveness of codon set selection/design was evaluated in part based on the “% good” value. A high percentage meant very low non-target amino acids; a high value of “% good” was deemed more important than having more target amino acids among the amino acids coded by a particular codon set. Redundancy was included in calculating the “% good” value. For evaluation purposes, the “% covering” value was also calculated. This value represents the percentage of natural diversity covered by the “good” amino acids (of the amino acids encoded by a particular codon set). For example, for L3-91, when codon set KMT is used, the “good” amino acids are YSA, which is 75% of the YSAD amino acids encoded by the codon. YSA are amino acids that cover 1190 out of 1580 known, natural antibody sequences at this amino acid position. 1190/1580 equals 75%, which is the “% covering” value. Thus, in one design using KMT at L3-91, 75% of the library covers 75% of the natural diversity in CDRL3 at position 91.

The codon sets were also designed to exclude, when possible, cysteine and stop codons. The presence of cysteine residues tends to cause folding problems and stop codons can decrease the effective library size. In the design of the codon sets, it was also deemed desirable to minimize the number of nontarget amino acids.

The codon sets designed for each solvent accessible and highly diverse residue of humanized antibody 4D5 are shown in FIG. 3. At any particular residue, one or more codon sets could be used depending on the target amino acids that were identified. For example, combining two H1 oligonucleotides, one having residue H1-33 as KMT, the other having H1-33 as KGG, results in 100% of the codons used for H1-33 covering 86% (50%+30%) of the natural diversity at the H1-33 position. Other examples of instances where two codon sets can be beneficially used include using codons YKG and TWT at L3-96, and codons DGG and DHT at H2-50.

The various codon sets could be used to generate diverse libraries with diversity in one or more CDR regions, including CDRL1, CDRL2, CDRL3, CDRH1 and CDRH2. For example, FIGS. 5-13 and FIG. 24 show various illustrative versions of codon set designs that can be used to generate diversity. FIGS. 3A and 3B provide a summary of the amino acid coverage of these designs. In general, it is preferable, but not necessary, that the designs narrow the diversity to cover more of the natural diversity and exclude as much as possible the “non-target” amino acids. In some embodiments, a design that does not score the highest based on these criteria may be used to obtain the best binders for a particular target of interest.

### EXAMPLE 3

### Library Design: H3

In comparison to other CDRs, heavy chain CDR3 (H3) regions exhibit the greatest diversity in sequences and lengths, although the sequence diversity is not completely random (i.e., some amino acids were found to occur more often than other in particular amino acid positions).

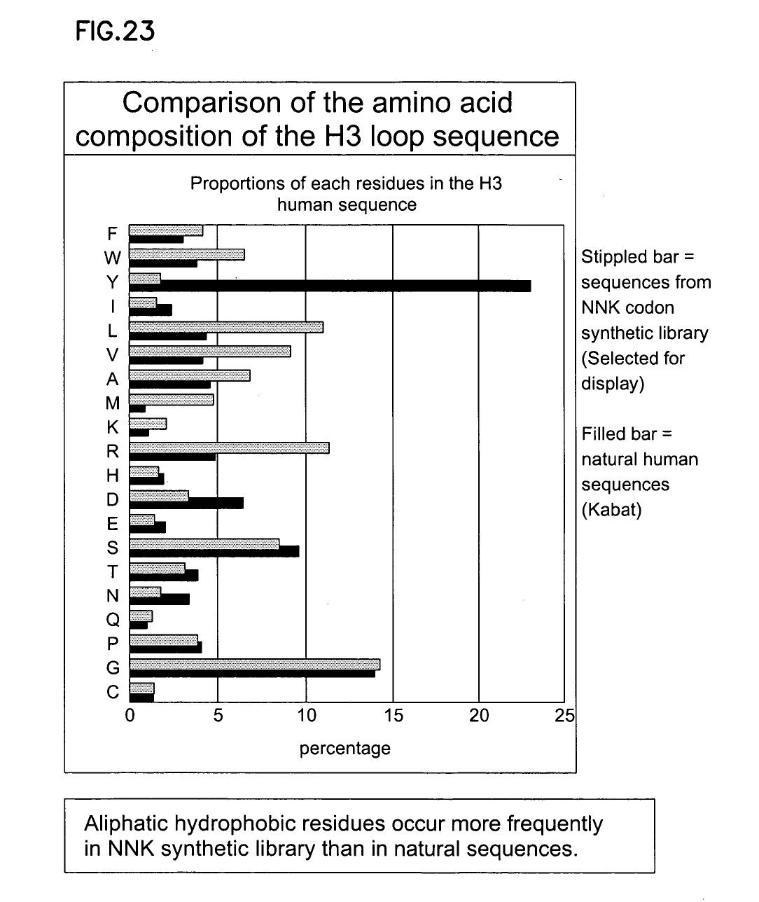

In a preliminary analysis to assess the amino acid preferences for each position in H3, a library with diverse H3 was generated using an NNK codon set for residues 95-100a of the humanized antibody 4D5 H3 region. The NNK codon set encodes all 20 amino acids and stop codons. This library was generated in a Fab phage display format and 400 clones that displayed functionally on phage were identified and sequenced. The amino acid sequence found in H3 regions in the NNK library were compared to those found in the Kabat database. A comparison of those amino acids is shown in FIG. 23. When the amino acid sequences in the NNK library and Kabat database were analyzed, it was determined there was good agreement in most amino acid usage between the library sequences and the sequences of Kabat. Interestingly, there appeared to be more aliphatic/hydrophobic amino acids in the NNK library than in the known, naturally occurring sequences. See FIG. 23.

Two codon sets were then used to design libraries containing diversified H3. One of the codon sets was DVK. DVK encodes ACDEGKNRSYW and a stop codon. Amino acids that do not occur frequently in known, natural antibody sequences were excluded, for example, LPHQFIMV. Another codon set used was NVT. NVT encodes ACDGHPRSTY and excludes W (tryptophan) and a stop codon. Tryptophan is favored in phage display and tends to dominate. Stop codons can decrease library diversity. In some instances, the NVT design was doped with W by walking W across the residues.

In terms of which residues to diversify in H3 (Kabat numbers 95 to 102) of 4D5, it was determined that the C-terminus of H3 was quite constant. The C-terminus of H3 had mainly two types of sequences in the Kabat database of human sequences, the sequences being either:
Y₁₀₀ₐAND₁₀₁(Y/V)₁₀₂ (sometimes Y₁₀₀ₐ can vary slightly)
or F₁₀₀ₐD₁₀₁(Y/V)₁₀₂.

In humanized antibody 4D5, the C-terminus of H3 is YAMDY. The libraries were designed to keep this part mostly constant, except Y₁₀₀ₐ was occasionally varied. Various designs of H3 (FIG. 4) which mostly used either DVK or NVT to diversify amino acid positions 95-100 or 96-100a were used to generate H3 libraries. The average human H3 length is 11-13 residues; humanized antibody 4D5 falls within this range. In most designs, humanized antibody 4D5 H3 CDR length was kept the same, or lengthened by one or two residues. In some designs, Y₁₀₀ₐ was diversified more narrowly by allowing it to be GARWST (using codon DSG) or GSAW (using codon KSG) since these variations were found in known, natural H3 sequences and in the phage libraries generated with DVK for that position.

In some designs, framework residue 93 was changed to alanine to reflect the natural consensus (humanized antibody 4D5 has the mouse residue serine). Framework residue 94 (right before the first H3 residue) was designed to be arginine, or arginine and lysine (using codon ARA) to reflect the natural sequence consensus.

### EXAMPLE 4

### Construction, Sorting and Analysis of scFv Libraries

Libraries with diversified CDRs were generated using vectors comprising 4D5 variable domains in the scFv or scFv-zip formats as described in Example 1. In total, five libraries were generated and the library characteristics were as follows:

| Library name | Format | CDR Diversity |
|---|---|---|
| ScFv-1 | zipper | H1, H2, H3 |
| ScFv-2 | Zipper | H1, H2, H3, L3 |
| ScFv-3 | Zipper | H3, L3 |
| ScFv-4 | No zipper | H1, H2, H3 |
| ScFv-5 | No zipper | H1, H2, H3, L3 |

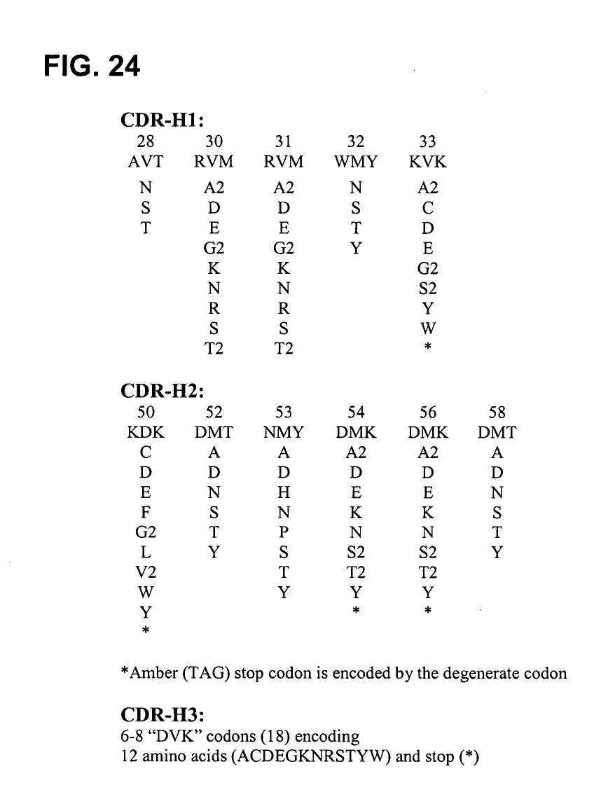

Libraries were constructed using the method of Kunkel (Kunkel et al., Methods Enzymol. (1987), 154, 367-382) with previously described methods (Sidhu et al., Methods Enzymol.(2000), 328, 333-363). For each library a “stop template” version of a scFv or scFv-zip display vector was used; in each case, a stop template with TAA stop codons within each of the CDRs to be randomized was used. Mutagenic oligonucleotides with degenerate codons at the positions to be diversified were used to simultaneously introduce CDR diversity and repair the stop codons. For example, for the construction of library scFv-1, three oligonucleotides were simultaneously used to introduce diversity into CDR-H1, CDR-H2, and CDR-H3. Successful incorporation of all the mutagenic oligonucleotides resulted in the introduction of the designed diversity at each position and simultaneously repaired the stop codons, thus generating an open reading frame that encoded either a scFv or scFv-zip fused to the C-terminal domain of the gene-3 minor coat protein. Residues in each CDR were chosen for diversification based on their accessibility and their level of diversity in the Kabat data base of natural antibodies (as described in Examples 2 and 3). For CDRs H1 and H2, the residues chosen for diversification and the diversity introduced at each position are shown in FIG. 24. For CDR-H3, diversity was introduced using an equimolar mixture of four mutagenic oligonucleotides (F59, F63, F64 and F65 in FIG. 4).

The mutagenesis reactions were electroporated into E. coli SS320 (Sidhu et al., Methods Enzymol.(2000), 328, 333-363), and the transformed cells were grown overnight in the presence of M13-VCS helper phage to produce phage particles that encapsulated the phagemid DNA and displayed scFv or scFv-zip fragments on their surfaces. Each library contained greater than 2×10¹⁰ unique members.

1. Selection of Specific Antibodies from the Naïve Libraries.

Phage from each library described above were cycled through rounds of binding selection to enrich for clones binding to targets of interest. Three target proteins were analyzed: Her2, IGF-1, and mVEGF. The binding selections were conducted using previously described methods (Sidhu et al., supra).

NUNC 96-well Maxisorp immunoplates were coated overnight at 4° C. with capture target (5 μg/mL) and blocked for 2 h with bovine serum albumin (BSA) (Sigma). After overnight growth at 37° C., phage were concentrated by precipitation with PEG/NaCl and resuspended in PBS, 0.5% BSA, 0.1% Tween 20 (Sigma), as described previously (Sidhu et al., supra). Phage solutions (10¹³ phage/mL) were added to the coated immunoplates. Following a 2 h incubation to allow for phage binding, the plates were washed 10 times with PBS, 0.05% Tween20. Bound phage were eluted with 0.1 M HCl for 10 min and the eluant was neutralized with 1.0 M Tris base. Eluted phage were amplified in E. coli XL1 -blue and used for further rounds of selection.

The libraries were subjected to two rounds of selection against each target protein (Her-2, IGF-1 or mVEGF), followed by a round of selection (round 2a) against an anti-gD epitope antibody to enrich for clones displaying scFv or scFv-zip (there is a gD epitope in the linker between the light chain and heavy chain regions of the scFv). Following the anti-gD enrichment, each library was selected for a third round against the target protein.

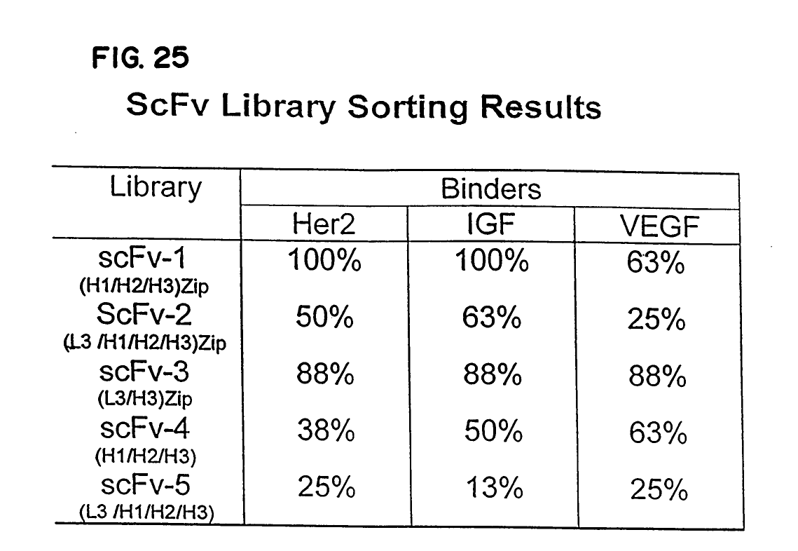

Individual clones from each round of selection were grown in a 96-well format in 500 UL of 2YT broth supplemented with carbenicillin and M13-VCS, and the culture supernatants were used directly in phage ELISAs (Sidhu et al., supra) to detect phage-displayed scFvs that bound to plates coated with target protein. The results for each library against each target Protein After three rounds of sorting are shown in FIG. 25, and it can be seen that each library produced binders against each target protein.

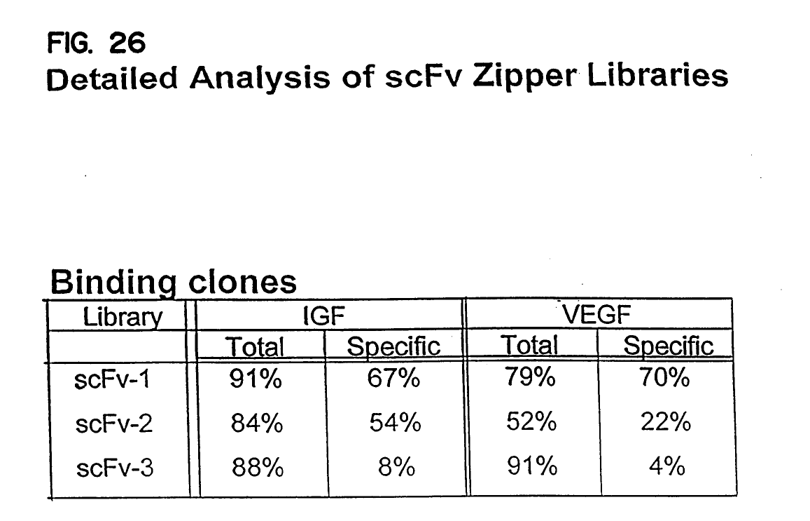

The three scFv-zip libraries (scFv-1, scFv-2 and scFv-3) were subjected to more detailed analysis. Phage clones from round 3 of selections against IGF-1 or mVEGF were analyzed for specific binding to target by doing phage ELISAs against both IGF-1 and mVEGF. Clones that bound only to the target against which they were selected were classified as specific while those that bound both targets were classified as non-specific. The results are shown in FIG. 26 which indicates the percentage of clones from each selection that bound targets (Total) and the percentage of clones that bound only the target against which they were selected (specific). All three libraries produced specific binding clones against target, although library scFv-3 produced considerably fewer specific binders than did libraries scFv-1 and scFv-2.

For library scFv-1, clones were also screened from round 2 of selection. From the IGF-1 selection, 140 out of 960 screened clones (15%) were positive. From the mVEGF selection, 24 out of 1152 screened clones (2%) were positive.

For one scFv library (library scFv-4), several hundred clones from round 3 of the IGF-1 or mVEGF binding selections were screened for specific binders. In this case, the IGF-1 selection yielded 35% specific binders while the mVEGF selection yielded 40% specific binders.

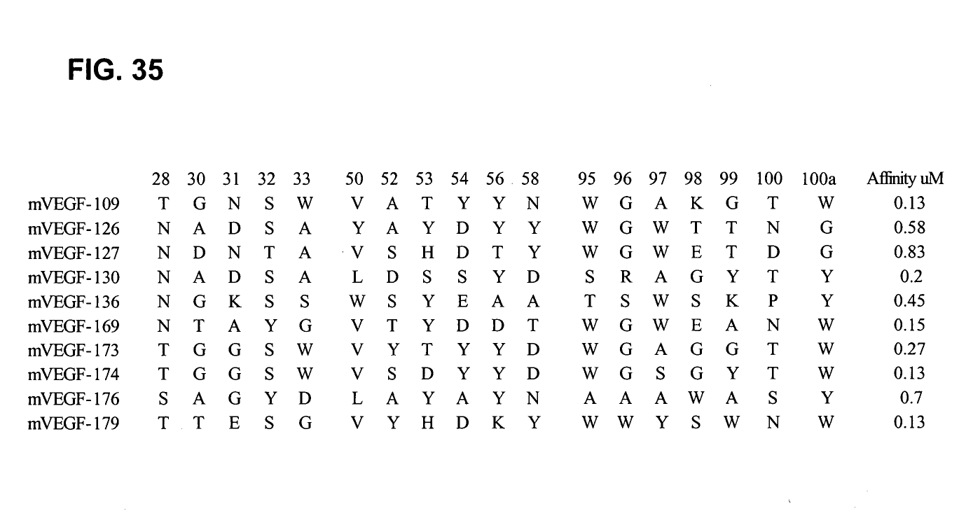

FIG. 35 shows the sequences and affinities of some positive binders from scFv and ScFv-zip libraries.

2. Sequencing of Antigen Specific Binders

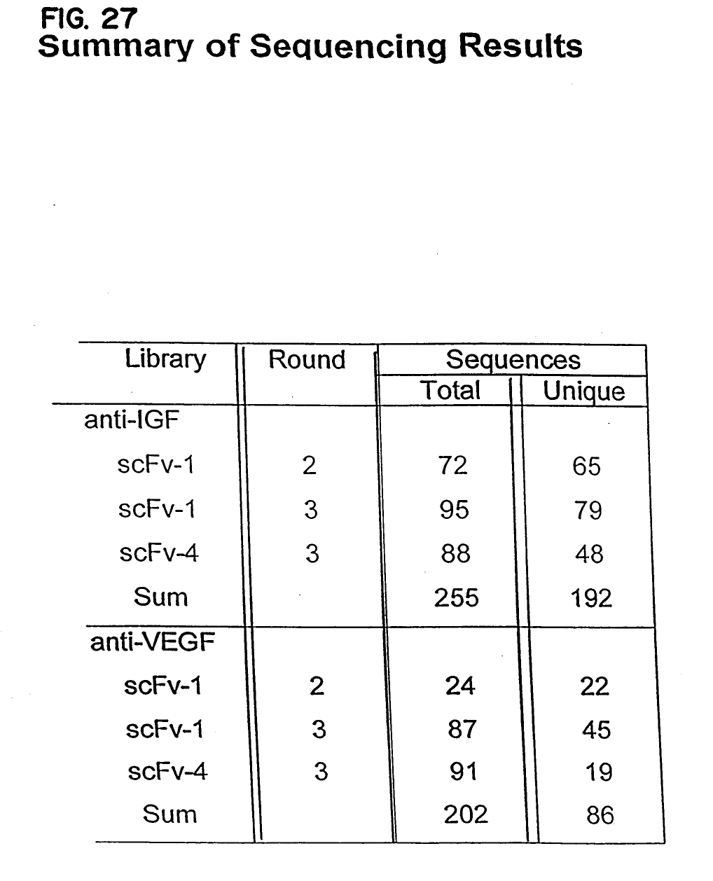

Representatives of the specific binding clones characterized above were sequenced using standard methods. The results are shown in FIG. 27.

From the IGF-1-specific binders, a total of 255 clones were sequenced and 192 of these were unique sequences. From the mVEGF-specific binders, 202 clones were sequenced and 86 of these were unique sequences. These results confirmed that the methods of the invention for generating CDR diversity and selecting for antigen specific binders resulted in a multiplicity of antigen specific antibody variable domains with different sequences.

The complete sequences from about 450 binding clones were analyzed for amino acid diversity at the heavy chain residues that were diversified in the library designs. The results indicated that all the designed substitutions occurred with a good distribution of amino acid type (data not shown).

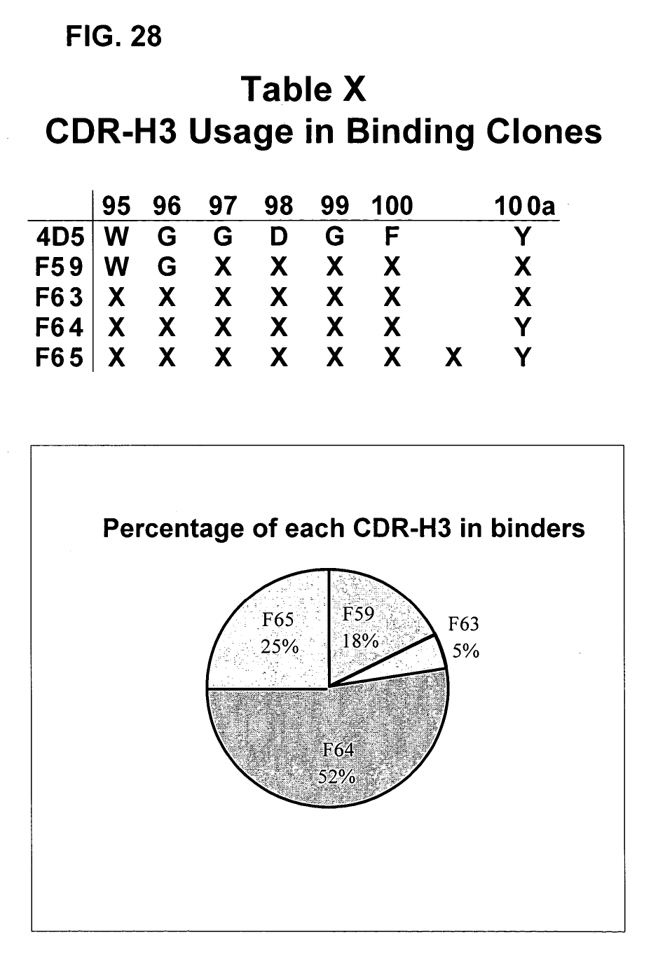

Analysis of the sequences of the CDR-H3 region indicated that all four different designs included in the naïve libraries (F59, F63, F64 and F65 in FIG. 4) were also present in the selected binding clones, as shown in FIG. 28. However, the four CDR designs were not equally common among the selected binders, indicating that some CDR designs may be better suited for generating positive binding clones against particular targets. In particular, the design F64 was the most prevalent (52%) while the design F63 was the rarest (5%) (FIG. 28).

### EXAMPLE 5

### F(ab)′₂ Libraries with L3/H3 Diversity

Libraries with diversity in CDRL3 and H3 were generated using the template Fab-zip 4D5 vector as described in Example 1. For CDRL3, oligonucleotide F61 (F61: GCA ACT TAT TAC TGT CAG CAA NRT NRT RVM NNK CCT TDK ACG TTC GGA CAG GGT ACC; the underlined nucleotides encoding amino acid residues/positions that were diversified; SEQ ID NO.:23). For CDRH3, oligonucleotides used were designated F59, F63, F6, F65, F66, and F78. (See FIG. 4). A pair of L3 and H3 oligonucleotides were used per Kunkle mutagenesis reaction, and libraries were made and amplified in E. coli as described in Example 4. In total, six libraries with different H3 diversity (generated with six different oligonucleotides as described above) were made and combined after amplification for sorting on targets. Sorting was done as in Example 4, except that this library underwent two tracks of sorting: with or without presorting on anti-gD before target sorts. The library was either directly sorted on targets (i.e., with no presort) or first on anti-gD as a presorted library before sorting on targets. After one presort, the library was sorted on targets two times and then on anti-gD once before sorting on targets another time. Library without presorting went through targets twice and then was enriched on anti-gD once and then another time on targets.

The hit rate was significantly better with presorted library sorted against mVEGF or IGF-1. For HER2ECD as targets, both presorting or no presorting worked well. The hit rate after two target sorts and one anti-gD sort was about 1-4%. A final sort on target after anti-gD sort resulted in a >90% hit rate. Hit rate was calculated as specific binders found per 100 clones screened. Specific binding clones were defined as in Example 4.

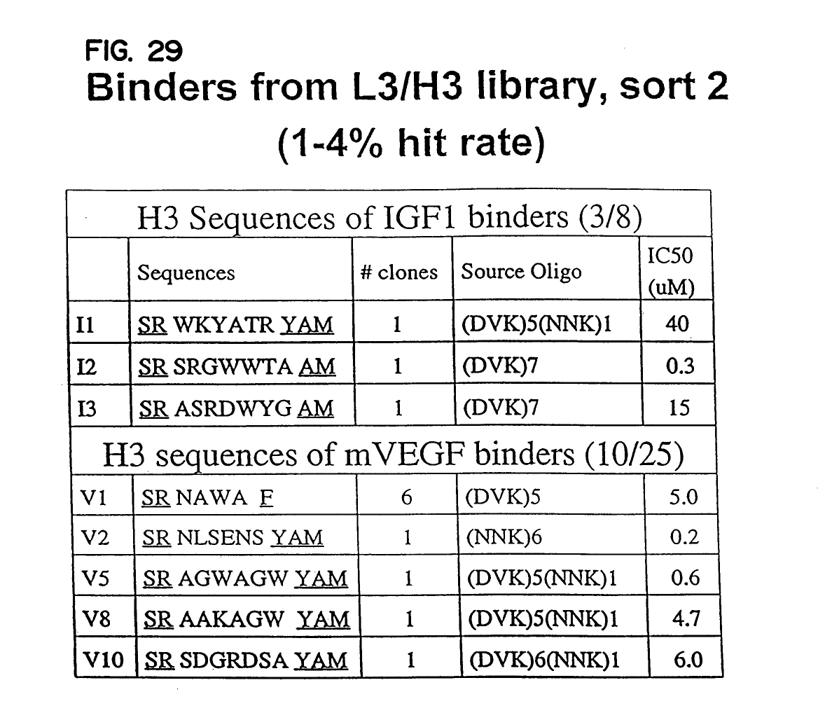

Positive clones were sequenced for their H3 sequences and IC50 (affinity) analyzed as previously described (Sidhu et. al, supra). FIG. 29 shows the results of analysis of each clone. Clones were obtained with variable sequences and affinity in the range of sub-micromolar and micro-molar. Most binders came from libraries with DVK design in H3. In column 2 of FIG. 29, underlined residues represent residues that were fixed in the source library of the clones. Most binding clones came from the library that fixed the Y₁₀₀ₐ position.

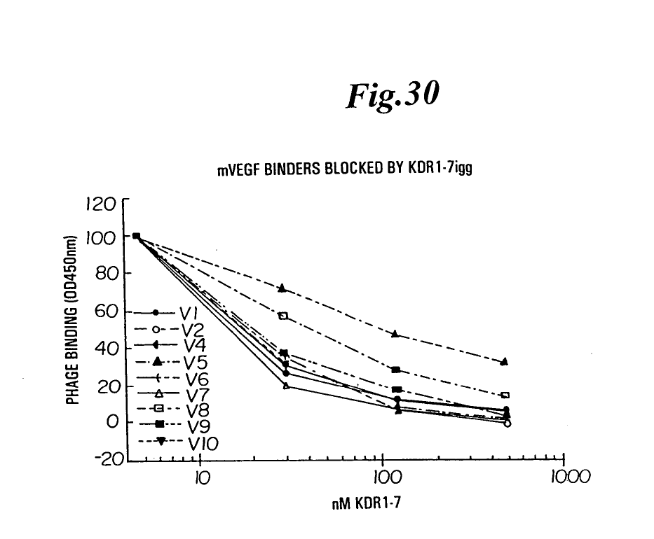

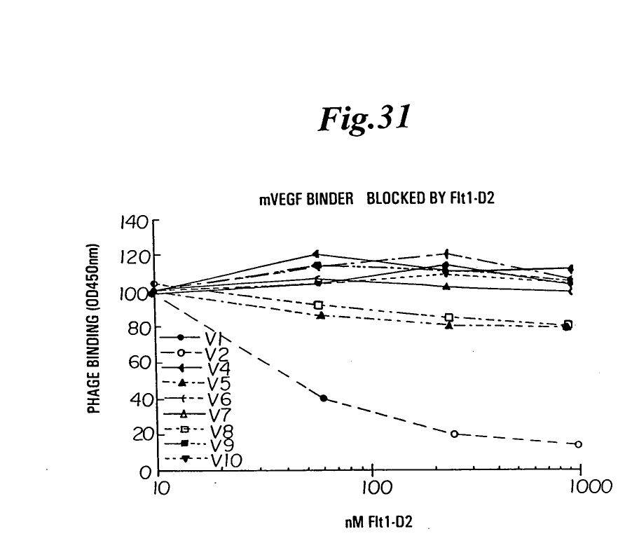

The binding epitope of some clones were also analyzed by competitive binding ELISA. Target (murine VEGF) was coated on 96-well NUNC-maxisorb plate. Phage clones binding to the target in the presence of blocking reagent which bound to a particular epitope on mVEGF was measured. Two mVEGF receptor fragments were used, Flt-D2 (Wiesmann et al., Cell (1997), 91:695-704) or KDR1-7-igg (Fuh et al., J. Biol. Chem. (1998), 273:11197-11204). The results showed that all analyzed binders bound to a similar epitope as KDR1-7-igg since they competed with each other (FIG. 30). Flt-D2 (Domain 2 of Flt-1), which has a smaller epitope on mVEGF, only blocked one clone but not the others (FIG. 31).

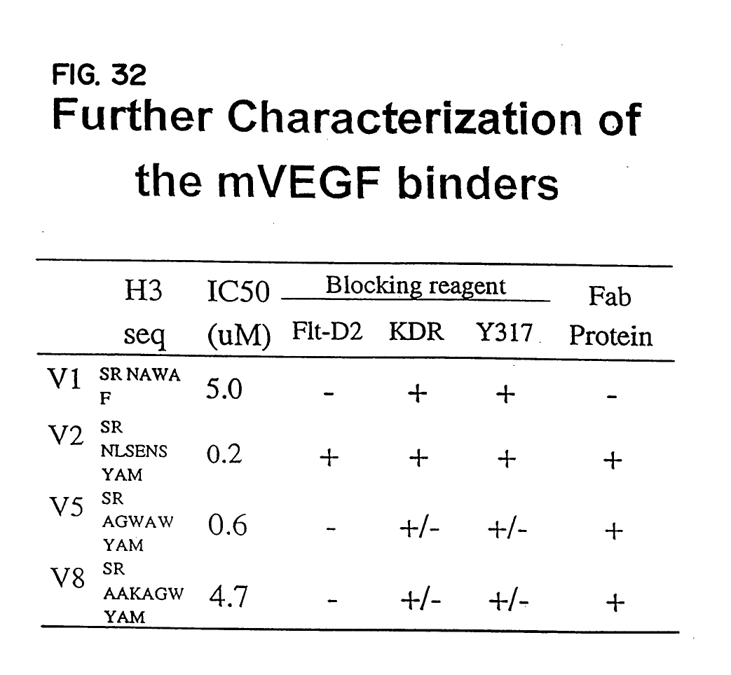

To demonstrate that the binding phage clones did present Fab polypeptide sequences that could become Fab antibody fragments, we generated Fab protein from some of the clones. Phage constructs from binding clones were transformed into E. Coli strain 27C7 that does not have amber stop suppressors, and the bacteria were grown to produce Fab protein. FIG. 32 summarizes the characterization of these clones. Fab protein was successfully generated from clones V2, V5 and V8.

### EXAMPLE 6

### Fab and F(ab)′₂ Libraries with H1/H2/H3 Diversity

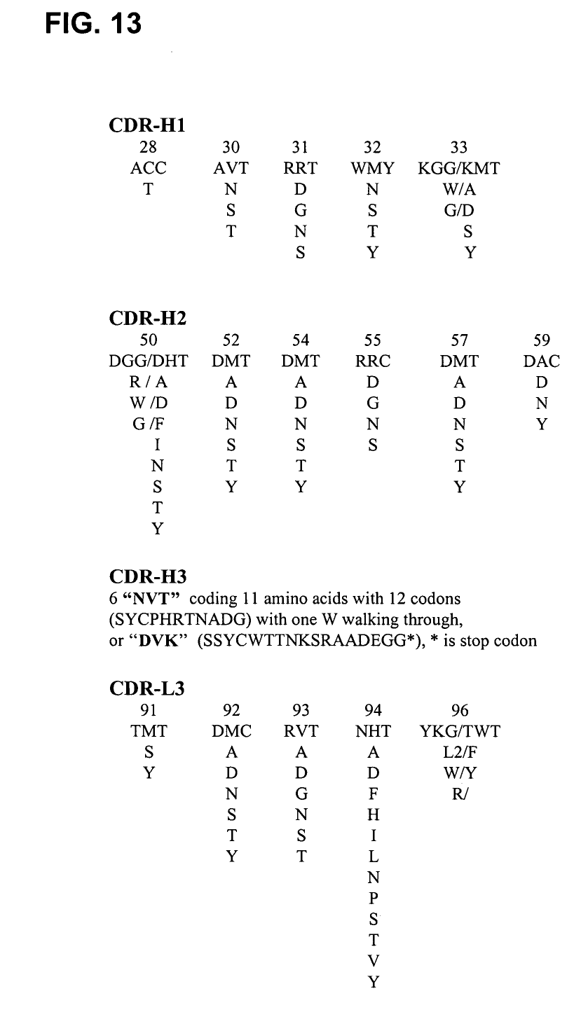

Libraries with diversified CDRs were generated using vectors comprising 4D5 variable domains as described above. Template construct was pV0116b for Fab or pV0116g for Fab′2, both of which had phoA promoter and stII signal sequences for both light chain and heavy chain. To make Fab H1/H2/H3 libraries, each mutagenesis reaction used oligonucleotides that coded for H1, H2 and H3 diversity. To ensure the incorporation of all three CDRs in the randomization scheme, a stop codon (TAA) was incorporated in each CDR that was intended to be diversified. Only clones that incorporated all three CDR oligonucleotides would have positive display since the stop codons would have been replaced. Oligonucleotides of different diversity were first combined to use as a source to diversify each CDR. For this experiment, two H1 oligonucleotides, F151 (GCA GCT TCT GGC TTC ACC ATT AVT RRT WMY KMT ATA CAC TGG GTG CGT CAG; SEQ ID NO: 14) and F152 (GCA GCT TCT GGC TTC ACC ATT AVT RRT WMY KGG ATA CAC TGG GTG CGT CAG; SEQ ID NO: 15) (See also FIG. 13) were pooled, and for H2, oligonucleotides F153 (AAG GGC CTG GAA TGG GTT GST DGG ATT WMT CCT DMT RRC GGT DMT ACT DAC TAT GCC GAT AGC GTC AAG GGC; SEQ ID NO: 16) and F154 (AAG GGC CTG GAA TGG GTT GST DHT ATT WMT CCT DMT RRC GGT DMT ACT DAC TAT GCC GAT AGC GTC AAG GGC; SEQ ID NO:17) (See also FIG. 13) were pooled. For H3, a DVK pool of oligonucleotides (F165, F166) and NVT pool (F134, F136, F137, F138, F142, F155, F156, F157, F158, F160, F160g) were used. FIG. 4 shows H3 positions that were subjected to diversification. Two Fab libraries were generated: one with DVK H3 pool and one with NVT H3 pool. The two libraries were amplified in E. Coli before being combined for sorting on the targets.

Mutagenized DNA was used to transform E. Coli strain SS320 by electroporation and size of the libraries were in the range of 10⁹. Transformed bacterial cells were grown up overnight in the presence of helper phage KO7 to produce displaying phage that could still infect other bacterial cells as described in Example 4.

Sorting on Mouse Vascular Endothelial Cell Growth Factor (mVEGF) and Human IG1-Fc (hFc)

DVK and NVT libraries were pooled for sorting on the targets. Sorting was performed as with other libraries as described above. The combined library was sorted first on target once, next sorted with anti-gD antibody which could get rid of the non-displaying clones, and next with two sorts on targets (S3, S4). About 96 clones from S3, S4 were screened. Positive clones were clones that had above background binding to the targets (binders) and not on other non-relevant protein (i.e., specific binders). For mVEGF as target, S4 provided the highest hit rate for positive specific binders. For human Fc, S3 and S4 provided high rate of specific binders.

| | mVEGF | | hFc | |
|---|---|---|---|---|
| | Total | Specific | Total | Specific |
| Library | binders | binders | binders | binders |
| Fab S3 | 36% | 1-2% | 88% | 83% |
| Fab S4 | 91% | 72% | 99% | 90% |
| F(ab)′₂ S3 | 42% | 3-5% | ND | ND |
| F(ab)′₂ S4 | 73% | 72% | ND | ND |

ND: Not determined

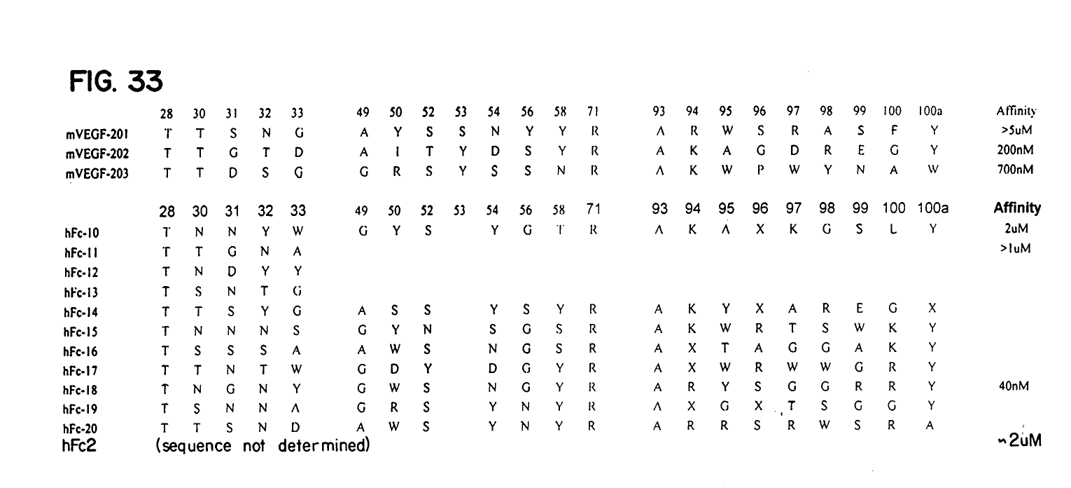

The DNA sequences of the binders and the binding affinity of the unique binders were analyzed. Examples of sequences and binding affinity of binders are shown in FIG. 33. For specific hFc binders, many distinct Fab clones, some of which binding at 40 nM, 2 uM and >5 uM individually were obtained. From the F(ab)′₂ library, clones with affinities at 41, 47 and 110 nM were obtained.

### EXAMPLE 7

### Identification of Amino Acid Preferences in CDRH3 and Framework Region Residues in Variable Domain of Llama Anti-HCG Camelid Monobody

Camelid antibodies have been previously shown to have 2 species including a classic IgG molecule with two heavy and two light chains and a heavy chain IgG molecule lacking a light chain (hereinafter designed “monobodies”). These monobodies are useful to generate synthetic libraries. Libraries generated using monobodies have several advantages over libraries generated using other antibodies or antigen binding fragment or polypeptides. Camelid monobodies have several advantages in antibody design. These molecules bind their targets with high affinity and specificity, and as such can be used as modules in the design of traditional antibodies. In certain cases, one may want to construct an antibody by first designing a high affinity heavy chain antibody or monobody which could then be converted to a Fab or IgG by pairing the monobody with an appropriately paired light chain. Secondly, these monobodies can be utilized to form novel antigen binding molecules (mini-antibodies) without the need for any light chain. These mini-antibodies are similar to other single chain type antibodies, but the antigen binding domain comprises a heavy chain variable domain but lacks a light chain variable domain. Thirdly, these molecules are ideal for the design of bi-specific antibodies. Fourthly, due to extensive use of CDRH3 and reduced binding surface due to absence of the light chain, camelid monobody libraries may more successfully target enzyme active sites. Finally, monobody libraries may be useful as scaffolds for the presentation of peptide libraries, facilitating the design of smaller mimics of the antibody-antigen interface and peptide libraries that include novel ligands for target antigens.

The absence of the light chain in camelid monobodies results in structural adaptation in the heavy chain to stabilize the structure due to loss of the light chain. Identifying structural amino acid positions in the CDRH3 that are important to stabilize the structure of monobodies is important in designing a library that provides for diversity in the CDRH3 while minimizing the effect on the structural stability of the monobody.

Some framework region sequences are also involved in maintaining the stability of the monobody. The framework sequence changes can also impact the design of a monobody for use in synthetic libraries. Identification of framework region residues may also be important in designing a library that provides for diversity while minimizing structural perturbations.

The llama anti-HCG monobody was used as the parent or wild type molecule for determining the effect of mutations in the wild type CDRH3 region and the framework regions on stability of the monobody.

Materials and Methods

The wild type anti-HCG scaffold was synthesized using nested oligonucleotide PCR. An optimal nucleotide sequence was generated for bacterial expression using a program which generates optimal nucleotide sequences based on an amino acid sequence for a given expression host, in this case E. coli. The nucleotide (SEQ ID NO:135) and amino acid sequences (SEQ ID NO: 136) of the llama anti-HCG monobody are shown in FIG. 37a and b. The crystal structure of llama anti-HCG VHH is known and has been published. Spinelli et al., (1996) Nature Structural Biology, 3:752-757.

Library Construction

Vectors encoding fusion polypeptides comprising variant CDRs were constructed as follows. In general, vectors for antibody phage display were constructed by modifying vector pS1602 (Sidhu et al., (2000)) J. Mol. Biol., 296:487-495). Vector pS1602, which has pTac promoter sequence and malE secretion signal sequence, contained a sequence of human growth hormone fused to the C-terminal domain of the gene-3 minor coat protein (p3). The sequence encoding hGH was removed, and the resulting vector sequence served as the vector backbone for construction of vectors of the present invention that contain DNA fragments encoding the Llama anti-HCG antibody (Spinelli, S., Frenken, L., Bourgeois, D., de Ron, L., Bos, W., Verris, T., Anguille, C., Cambilau, C., Tegoni, M., (1996) Nat. Struct. Biol. 3(9), 752-757). A FLAG epitope tag was inserted at the C-terminal end of the Llama construct. Optionally, the FLAG epitope tag can be followed by a Gly/Ser-rich linker followed by P3C. Stop codons for the Framework scan were inserted at positions 37, 45, and 47. The resulting phagemid was designated pCB36624.

The llama a-HCG construct was then used as a template for Kunkle mutagenesis (Kunkel, T. A., Roberts, J. D., & Zakour, R. R. (1987) Methods Enzymol. 154, 367-382). Single degenerate oligonucleotides were used in generating the CDR3 library. Two oligos were used to generate the Framework library; one covering positions 37-47 and a second covering residue 91. The IUB nucleotide code was used to specify mixtures of nucleotides at each position (K=G/T, N=A/C/G/T, R=A/G, S=G/C, W=A/T, Y=C/T). Mutagenesis and phage production were done as previously described (Sidhu, S. S., Lowman, H. B. Cunningham, B. C. & Wells, J. A. (2000) Methods Enzymol. 328: 333-363).

Alanine CDRH3 Scan

For the wild type CDR3 scan, stop codons were inserted at residues 93, 94, 100 and 101. Positions in the wild type CDRH3 of the llama monobody were substituted to alanine in a combinatorial manner using alanine shotgun scanning mutagenesis ((Weiss, G. A., Watanabe, C. K., Zhong, A. Goddard., Sidhu, S. (2000) Proc. Natl. Acad. Sci., 97(16), 8950-8954). Positions 96 to 101 were substituted and the resulting phage libraries were sorted against Protein A. Mutagenesis, phage production, and Protein A selection were done as described below.

Oligonucleotide for Alanine Scan of Wild Type Anti-HCG CDR3:

| | |
|---|---|
| 5′- | (SEQ ID NO:151) |
| GCCGTCTATACTTGTGGTGCTGGTGMAGSTGSTRCTKSGGMTKCCTGGGGTCA | |
| GGGTACC-3′ | |

Framework Region Libraries; Randomizing Framework Positions 37, 45, 47 and 91

A library of monobodies was generated with variants at four framework positions: residues 37, 45, 47 and 91. Stop codons for the framework scan were inserted at positions 37, 45, and 47. The library was generated using two oligonucleotide primers; one covering positions 37-47 and a second covering residue 91. NNS codons were used for each position, allowing for substitution of all 20 amino acids at each position. The resulting libraries were sorted against Protein A and individual clones sequenced after 2 rounds of sorting.

| | |
|---|---|
| Oligonucleotides for the Framework Scan | |
| Residues 37-47 | |
| 5′ | (SEQ ID NO:152) |
| GATATGGGCTGGNNSCGTCAGGCTCCGGGTAAAGAANNSGAANNSGTTGCCG | |
| CCA-3′ | |
| Framework Scan - Residue 91 | |
| 5′- | (SEQ ID NO:153) |
| GATACTGCCGTCTATNNSTGTGGTGCTGGTGAAGGCGGTACTTGGGATTCTTG | |
| GGGTCAG-3′ | |

Protein A Sorts

Like all monobodies, the llama anti HCG is a Vh3 family member and as such binds Protein A. More importantly Vh3 family members all bind Protein A on the B sheet directly opposite the light chain interface. Thus, Protein A binding is not directly perturbed by changes at the former light chain interface. Formation of the monobody—Protein A complex is mediated by interactions on the monobody which are on the side of the B-sheet opposite that of the former light chain interface. As such Protein A binding interaction is used as a surrogate for CDRH3 mediated stability. The variant monobodies that are selected by interaction with Protein A can be used to identify structural amino acid positions in the CDRH3 region.

Phage expressing the mutagenized HCG constructs were sorted against Protein A (Sigma). Protein A was coated onto Nunc 96 well Maxisorp™ plates at a concentration of 5 ug/ml. Plates coated with Protein A were initially blocked with %0.5 BSA for one hour. After overnight growth at 37° C., phage were concentrated by precipitation with PEG/NaCl and resuspended in phosphate buffered saline (PBS), 0.5% BSA, 0.1% Tween 20 (Sigma). Phage solutions (˜10¹² phage/ml) were added to coated immunoplates. Libraries were allowed to bind for 2 hours at room temperature, then washed 12 times with PBS containing %0.05 Tween 20. Bound phage particles were then eluted with 100 mM HCl for 10 min. The eluant was neutralized with 1.0 M Tris base. Eluted phage were amplified in E.Coli. XLI-blue and used for further rounds of selection.

DNA Sequencing and Analysis

Individual clones from each round of selection were grown overnight at 37° C., in a 96-well format, in 500 μl of 2YT broth supplemented with carbenicillin and M13-KO7 helper phage. Culture supernatants containing phage particles were used as templates for PCRs that amplified the DNA fragment encoding the V_{H}H domain. The PCR primers were designed to add M13(-21) and M13R universal sequencing primers at either end of the amplified fragment, thus facilitating the use of these primers in sequencing reactions. Amplified DNA fragments were sequenced using Big-Dye terminator sequencing reactions which were analyzed on an ABI Prism 3700 96-capillary DNA analyzer (PE Biosystems, Foster City, Calif.). All reactions were performed in a 96-well format.

The sequences were analyzed with the program SGCOUNT as described in WO 01/44463 published Jun. 21, 2001. SGCOUNT aligned each DNA sequence against the wild-type DNA sequence by using a Needleman-Wunch pairwise alignment algorithm, translated each aligned sequence of acceptable quality, and tabulated the occurrence of each natural amino acid at each position. Additionally, SGCOUNT reported the presence of any sequences containing identical amino acids at all mutated positions.

Results

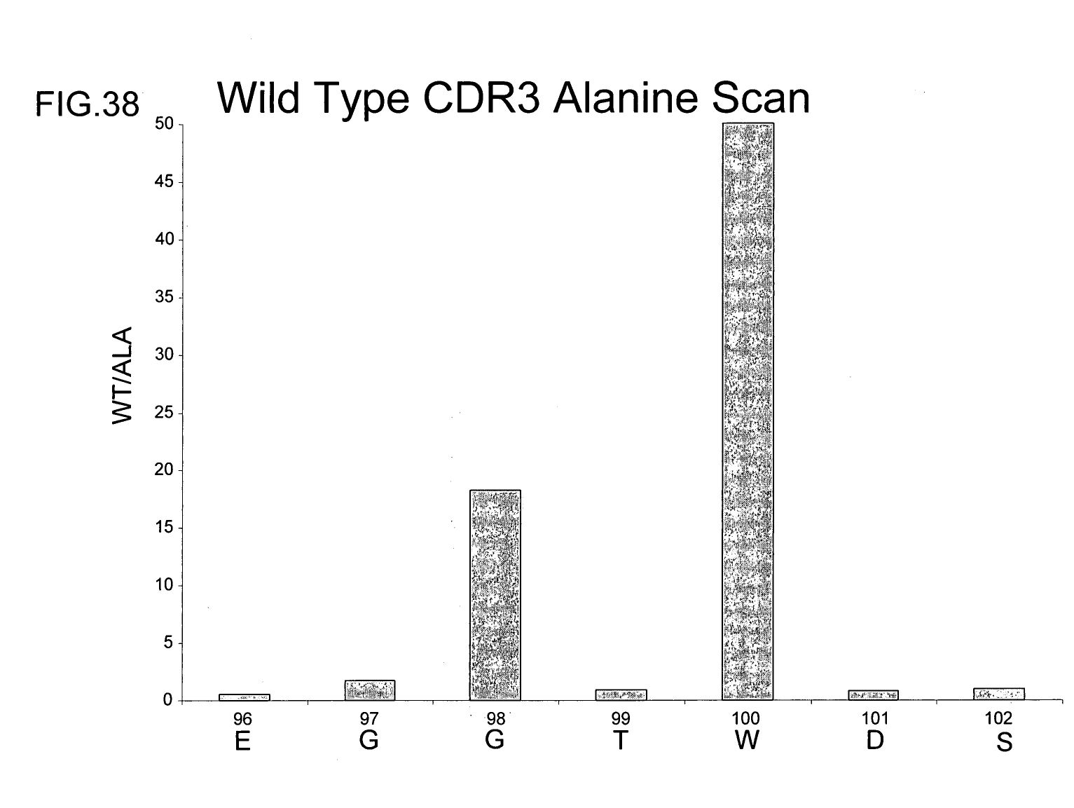

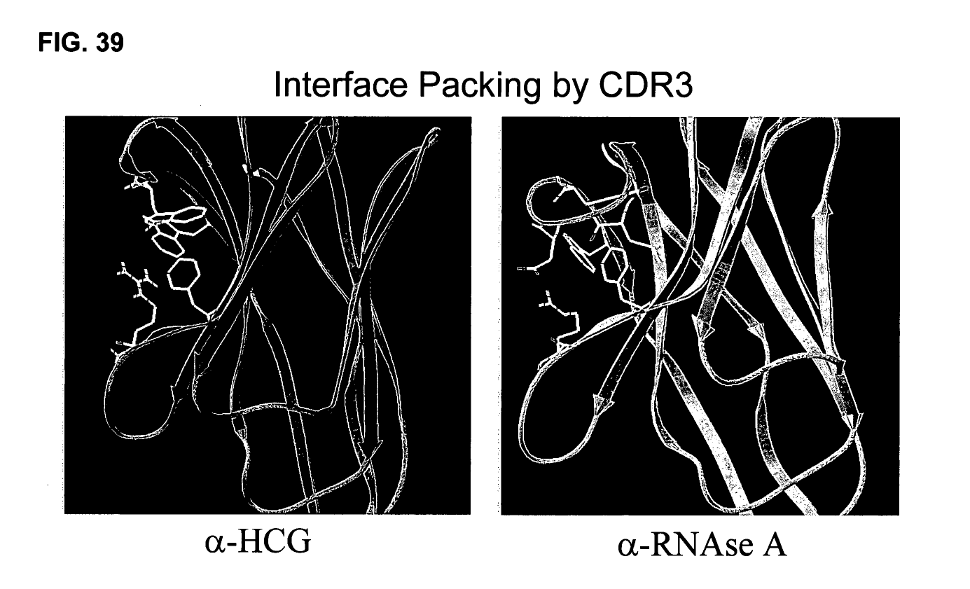

The CDRH3 region of camelid monobodies is involved in both antigen binding and stabilizing the structure of the monobody due to loss of light chain. Alanine scanning mutagenesis identified CDRH3 residues in the wild type CDRH3 sequence that were important for stabilizing the structure. The results are shown in FIG. 38. The results show that when amino acid positions 98 and 100 in CDRH3 are substituted with alanine there is a loss of stability of the monobody. At position 100, the tryptophan residue found in the parental sequence is preferred. This is consistent with crystal structure data for llama anti-HCG and for anti-RNAse monobody, which suggest that the trp at position 100 interacts with a phe at position 37 at the former light chain interface to form a minihydrophobic core. See FIG. 39.

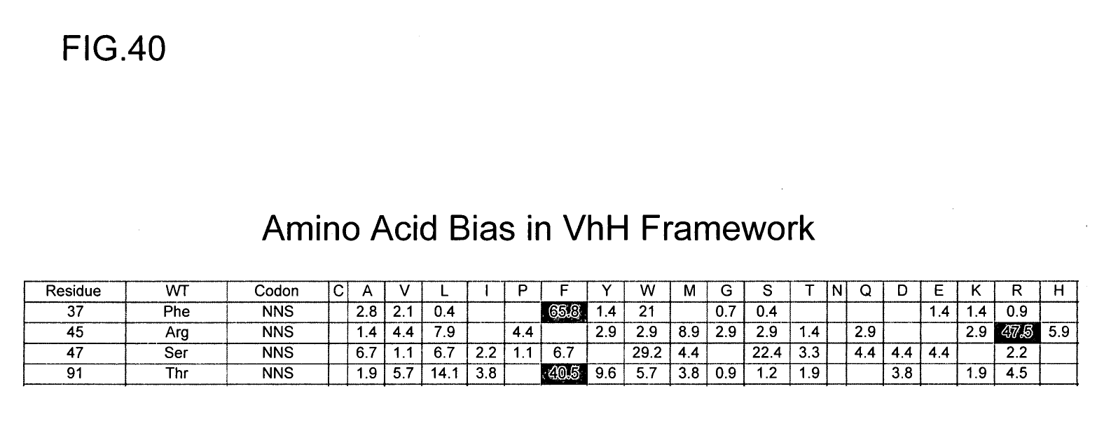

We also observed that some of the FR residues located at the site of the light chain interface in a three-dimensional structure were conserved. These residues are located at positions 37 (phe 37), 47 (Ser 47), 45 (Arg 45) and 91 (Thr 91). We examined whether these residues were involved in stability of the monobody by generating libraries of variant monobodies randomized at each of those positions. After the libraries were generated, the clones were sorted against Protein A. 105 clones were isolated and the monobodies were sequenced using standard methods. The results are shown in FIG. 40. The results show that at positions 37 and 45, the wild type amino acids of Phe and Arg , respectively, were preferred. At position 47, serine and tryptophan were preferred. At position 91, phenylalanine was preferred over wild type threonine at that position. These results indicate that positions 37 and 45 are more sensitive to substitution, while positions 47 and 91 can tolerate substitutions. Positions more sensitive to substitution are likely to be important for the stability of the molecule and therefore should be substituted with a more limited set of amino acids. This strategy will provide for maximizing the diversity of library while minimizing the structural perturbations.

As discussed previously, the results concerning the alanine scanning mutagenesis of the parental CDRH3 residues, indicated that trp 100 in CDRH3 is important structurally. The crystal structure suggests that this residue may interact with phe 37 to form part of the hydrophobic core at the former light chain interface. The results also show that substitutions at framework positions phe 37 and arg 45 can adversely effect the stability of the structure of the monobody. These results are also consistent with crystal structure data that suggest that arg 45 may also interact with phe 37 in forming the mini hydrophobic core.

These results indicate that in response to loss of the light chain binding partner residues from CDR3 pair with framework changes at the former interface forming a small hydrophobic core which replaces the light chain binding partner. Thus, in designing camelid monobodies as structural scaffolds for naïve or synthetic libraries, certain CDRH3 and/or framework region residues are more sensitive to substitution. For the design of stable monobodies, consideration must be given to the structural interactions between CDR3 and the former light chain interface.

### EXAMPLE 8

### Library Design: Identification of Amino Acid Preferences in CDRH3 of Camelid Monobodies

As discussed previously, the absence of the light chain in camelid monobodies results in structural adaptation in the heavy chain to stabilize the structure due to loss of the light chain. As shown in Example 7, this structural adaptation may include both framework and CDRH3 residues. The CDRH3 region adapts to the loss of the light chain by contributing residues to the former heavy chain/light chain interface. The CDRH3 region in camel monobodies is on average 4 residues longer than a human or murine counterpart. See FIG. 41. Identifying residues that contribute to structural stability in the CDRH3 is important in designing a library that provides for diversity while minimizing the effect on the structure of the monobody library. The appropriate design of heavy chain monobody libraries is improved by designing the CDRH3 insert so that the structural requirement of stabilizing the light chain interface can be met while allowing for variation of functional residues that participate in antigen binding.

We have discovered a method for identification of structural amino acid positions in CDRH3 of antibody variable domains, especially those domains belonging to the Vh3 family. Combinatorial libraries with amino acid positions in CDRH3 randomized were selected for interaction with Protein A as a readout of stability and expression. This approach allowed us to rapidly screen a large number of potential scaffolds in a short period of time and identify amino acid positions in CDRH3 that were important to the structural stability of the monobody.

Materials and Methods

Library Construction

As with the previous experiments, we chose the Llama anti HCG monobody as the parent molecule. Phagemid pS1602 (described in the Example 7) was used as a template for library construction. As before, the Llama anti-HCG was fused to the amino terminus of pIII. A FLAG epitope tag was inserted at the C-terminal end of the Llama construct. Stop codons were inserted at residues 93, 94, 100 and 101. The resulting llama anti-HCG construct was used as a template for the Kunkle mutagenesis. Mutagenesis and phage production were done as previously described.

Positions Gly95 and Trp103 in the wild type CDRH3 were chosen as the effective boundary for our library. Based upon the 152 available camelid VHH sequences (Harmsen, M. M., Ruuls, R. C., Nijman, I. J., Niewold, T. A., Frenken, L. G. J., de Geus, B., (2000), Molecular Immunol., 37, 579-590). The choice of Gly 95 and Trp 103 seemed the most conservative choice for N and C terminal boundaries for our CDRH3 libraries. A 17 residue peptide of all NNS codons was inserted in between Gly 95 and Trp 103. This 17 amino acid residue peptide is then numbered according to Kabat, starting at position 96, 97, 98, 99, 100, 100a, 100b, 100c, 100d, 100e, 100f, 100g, 100h, 100i, 100j, 101, and 102 (SEQ ID NO:137)as shown in FIG. 37c.

The Mutagenic Primer for Generating the CDRH3 Libraries Had the Following Sequence: NNS Library

| | |
|---|---|
| 5′- | (SEQ ID NO:154) |
| GCCGTCTATACTTGTGGTGCTGGTNNSNNSNNSNNSNNSNNSNNSNNSNNS | |
| NNSNNSNNSNNSNNSNNSNNSNNSTGGGGTCAGGGT-3′, | |

Protein A Sorts

Initial rounds of sorting against Protein A were performed as described in Example 7. Individual clones from the Protein A sort were isolated and a stop codon inserted at the 3′ end of the FLAG epitope tag. Proteins were then expressed in BL21 cells (available from Life Technologies, Inc. or Stratagene). The periplasmic supernatant was then run over a Protein A column and bound domains eluted with 0.1 M Glycine, pH 3.0. The monobody variants were further purified by size exclusion chromatography.

Thermal Stability

Thermal stabilities of the purified fragments were measured using a Aviv CD spectrometer model 202. (Protein Solutions, Lakewood, N.J.) The signal at 207 nM was used to monitor unfolding. A 0.5 degree celsius temperature step was used during thermal melts and the temperature range was 30-80 degrees celsius. Melting temperatures were determined for both the unfolding and folding transitions. All thermal melts were performed using a 1 mM protein solution in PBS.

Protein A Affinity

The affinity for Protein A for each of the individual clones was determined using a BiaCore 3000. Protein A was coated onto CM5 chip, as was BSA and VEGF. The latter two proteins were used as negative controls. Binding isotherms were calculated the steady state response differentials for a series of protein concentrations. The equilibrium dissociation constant (K_{d}) was determined by fitting the resulting curves (KaleidaGraph, Synergy Software) to the following equation:
Ra=Rmax+(Rmax−Rmin)/((1+C/K_{d})ⁿ)
Where “Ra” is the measured response differential, “Rmax” is the maximal response differential, “Rmin” the minimal response differential and “C” is the ligand concentration.
Analysis of NNS Library

To determine if there was any selection bias in the distribution of amino acids in the NNS library we calculated the Pearson residuals for the entire data set. The Pearson residual is defined as
eᵢⱼ=(nᵢⱼ−μij)/μᵢⱼ^{1/2}
where nᵢⱼ is the measured number of occurrences at residue j of amino acid i, and μᵢⱼ is the expected distribution of amino acid i at position j and is defined as:
μᵢⱼ=N(AAᵢ/N)(Resⱼ/N)
AAᵢ is the total number of amino acids of type i, Resⱼ is the total number of codons measured at position j, and N is the total number of codons measured in the entire data set. P-values were calculated for using the method of Bonferroni. Reci, J. A. (1998), Mathematical Statistics and Data Analysis, Wadsworth, Inc. (Pacific Grove, Calif.). The standard value of p<0.05 was adopted as a cutoff for statistical significance.

Results

In the structures of camelid monobodies reported to date, residues from wild type CDRH3 pack against phe37 and the former light chain interface (FIG. 39). However, the position and types of residues involved were dependent on the specific antibody. We examined if there were any biases, either by position or type, in a naive 17 residue CDRH3 library. As a scaffold we chose the Llama anti HCG structure. We exploited the Protein A-Vh3 interaction in elucidating the structural role of amino acid residues in the CDRH3 in a pool of monobody CDRH3 variants. To delineate the structural boundary within CDRH3 for potential antibody scaffolds, phage libraries were sorted against Protein A.

The Llama a-HCG antibody was chosen for two reasons: the crystallographic structure was known, which aided us in the analysis of our results and because it had already been expressed in bacteria and purified using a Protein A column. Positions Gly95 and Trp 103 were chosen as the effective boundary for our library. Based upon the 152 available camelid monobody sequences (Harmsen, M. M., Ruuls, R. C., Nijman, I. J., Niewold, T. A., Frenken, L. G. J., de Geus, B., (2000), Molecular Immunol., 37, 579-590) the choice of Gly 95 and Trp 103 seemed the most conservative choice for N and C terminal boundaries for our CDRH3 libraries. The 17 amino acid peptide length was selected as close to the average CDRH3 length in camel monobodies. See FIG. 41. A 17 residue peptide of all NNS codons was inserted in between Gly 95 and Trp 103. This 17 amino acid residue peptide is then numbered according to Kabat, starting at position 96, 97, 98, 99, 100, 100a, 100b, 100c, 100d, 100e, 100f, 100g, 100h, 100i, 100j, 101, and 102 FIG. 37c.

The resulting library had a complexity of 6.5×10²⁰. The initial titer after electroporation into SS320 cells was 4×10⁹. Thus, the actual library under sampled the theoretical library by 6.5×9-12. It is important to note that the goal of this library was not to completely sample the available sequence diversity in search of the tightest Protein A interaction, but instead to enumerate sequences in CDRH3 that did not perturb scaffold stability.

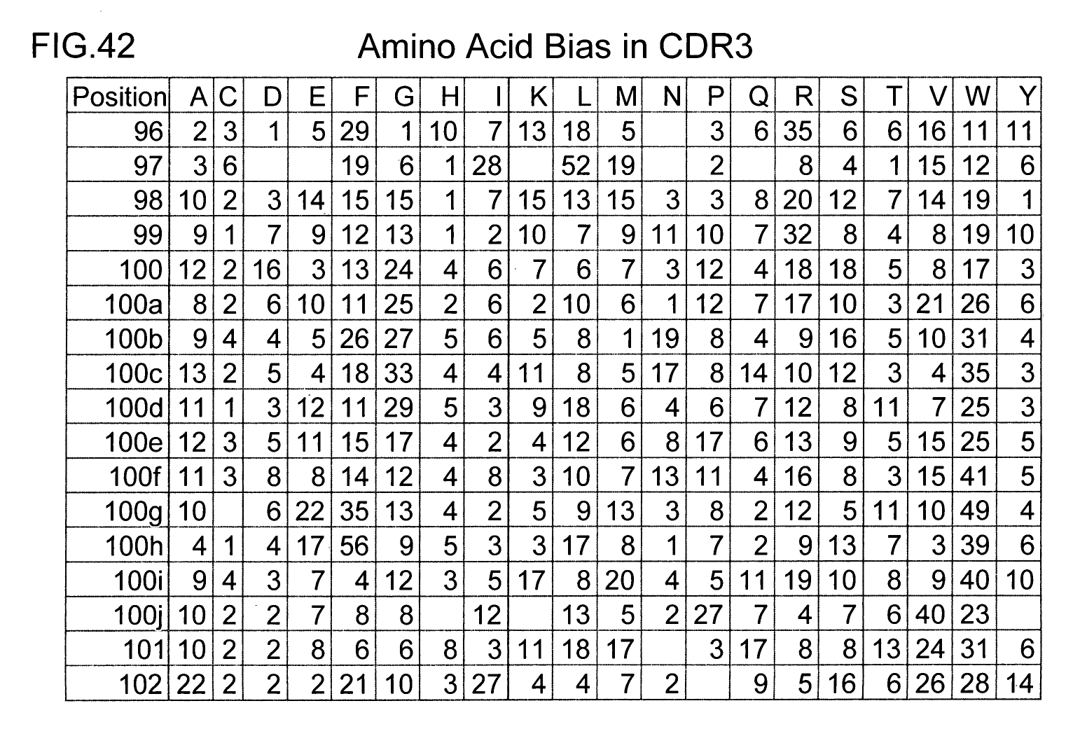

The resulting library was sorted for 5 rounds against Protein A. After rounds 3, 4 and 5, about 335, 324, and 50 clones were sequenced respectively. Of the clones sequenced at round 3, 222 out of 335 were unique. There is a clear bias in the types of sequences present after 3 rounds of sorting (FIG. 42). This bias is both in position and in residue type, clearly residues at the N and C-termini are non-randomly distributed as the general bias is toward hydrophobic residues.

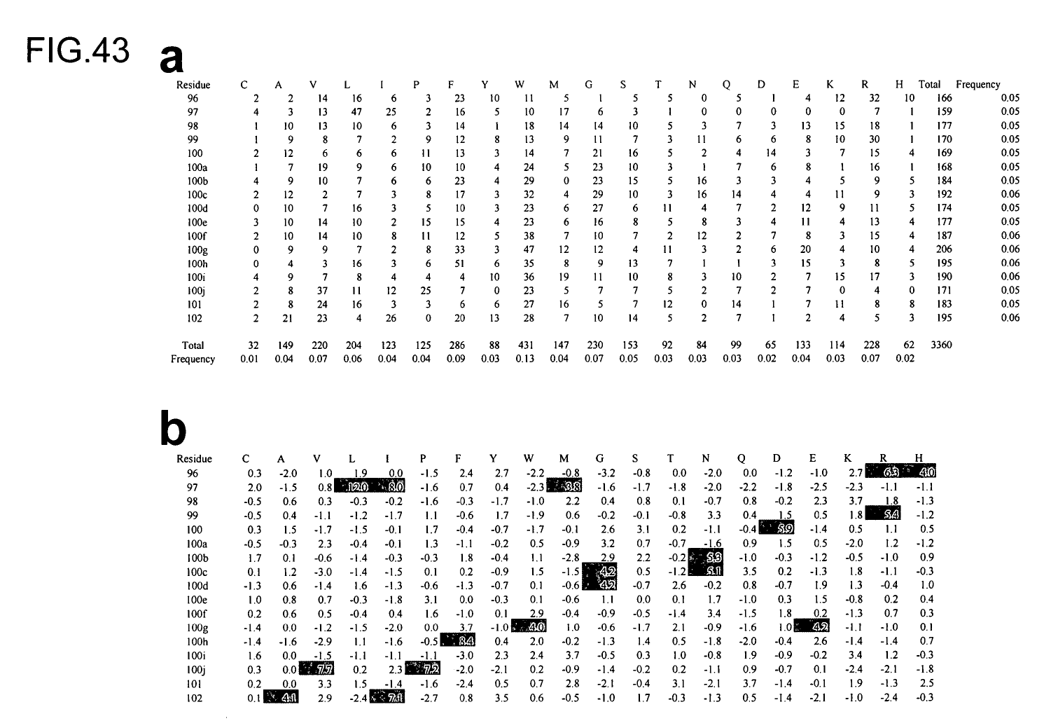

Aggregate analysis of this naive NNS library of monobodies provided the initial information regarding possible scaffolds for use as stable monobody structures and information about whether any of the amino acid positions showed a bias or preference for specific amino acid substitutions. The results are shown in FIG. 43a and b.

To determine if there was any selection bias in the NNS library, Pearson residuals were calculated based on normalized data. The normalized data was obtained by aligning the 222 sequences and tabulating the occurrence of each amino acid at each of the 17 positions within the peptide. The totals were then normalized by dividing by the number of times each amino acid was encoded by the redundant NNS codon; for example the NNS codon contains 3 unique codons for Arg, and thus, the Arg total at each position was divided by 3 to correct for the bias. The resulting normalized data set (FIG. 43a) was then analyzed for significant deviations from a random distribution.

An overall test for independence between amino acid frequency and residue was performed using a Chi-Squared test for independence (FIG. 43b). Pearson residuals were used to identify specific amino acid and residue combinations that were observed significantly more or less frequently than one would expect by chance under the hypothesis of independence. A Pearson residual was considered statistically significant if its magnitude exceeded 3.8. Based on the approximate normality of Pearson residuals, this cut-off corresponds to a p-value <0.05 even after a Bonferroni adjustment to account for the fact that 340 hypothesis were tested (20 amino acids at each of 17 residues). A Pearson residual, which is defined as the difference between observed and expected counts normalized by the square root of the expected count, above 3.8 is strong evidence for selection bias for the particular amino acid at the given position. A Pearson residual less than −3.8 is strong evidence for selection bias against the amino acid at the given position. The results are shown in FIG. 43(b).

Amino acids that deviated most significantly from random (p<0.05) showed a strong selection bias for particular amino acids at certain positions in the CDRH3 peptide. The N terminal end of the peptide was biased towards the sequence motif R(L/I/M)XR. Near the central portion of the peptide, the preference seemed to be for either glycine or hydrophilic residues. The C-terminal end of CDR3 (positions 100g-102) was characterized by an over representation of hydrophobes (Phe, Val, Ile and Trp) at particular positions. Both Trp and Gly occurred frequently throughout much of the peptide. However, only at a few positions did the occurrence of either of these residues rise significantly above background. In particular, the occurrence of glycine was most significant at positions 100c and 100d near the central portion of the peptide. It is possible that Gly at these positions enables some flexibility for turn formation. Trp occurred throughout the peptide, and may be involved in a number of nonspecific interactions. Yet, only at position 100g was the number of tryptophans significantly above background.

The aggregate analysis of a naïve NNS library was useful to provide some initial information about amino acid positions that may have a structural role in the CDRH3 region and to identify the most commonly occurring CDRH3 sequences that could be used as stable scaffolds. Amino acid positions are identified as structural positions in the CDRH3 using combinatorial alanine scanning mutagenesis as described in EXAMPLE 9.

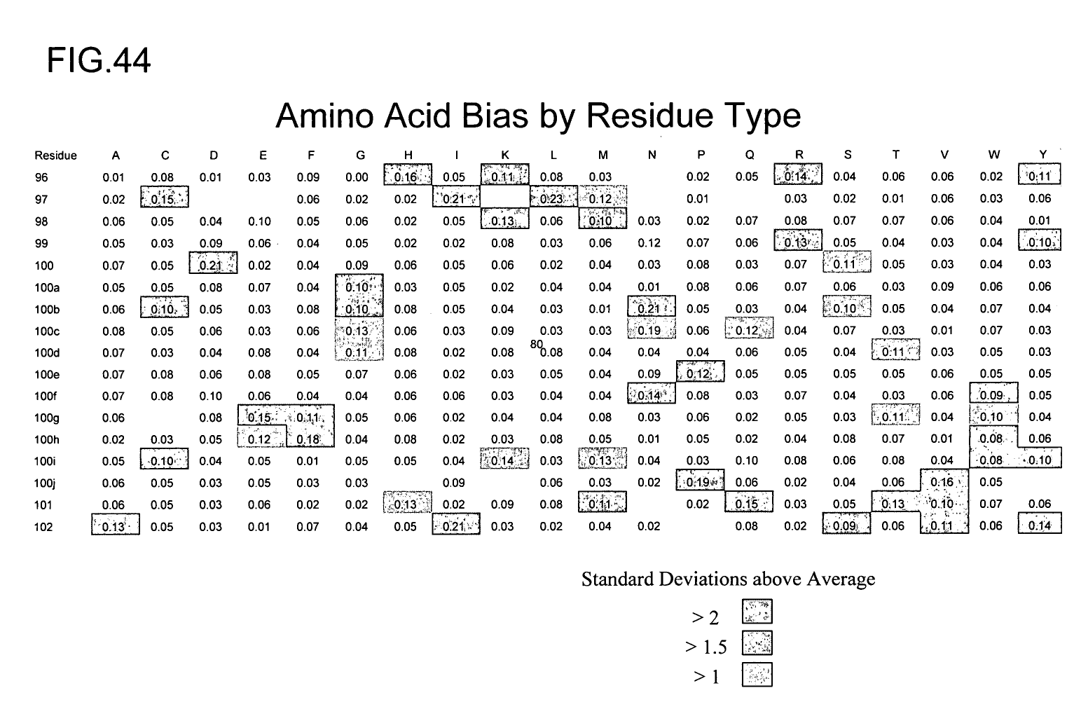

The sequence information from the NNS library was also analyzed for amino acid bias by residue position as described below to identify which amino acids were more frequently found in each position than would be expected in a random library. The total number of amino acids is calculated for each position and the average positional frequency for any amino acid is determined by dividing the total number of amino acids for each position by 20 (the expected number if the distribution was random). Those amino acids present at a position at a frequency of one standard deviation greater or above the average frequency for any amino acid at that position were selected as significant deviations from a random distribution. The results are shown in FIG. 44. This type of analysis can be utilized to select or identify amino acids that can be substituted at a structural amino acid position and maintain structural stability of the molecule.

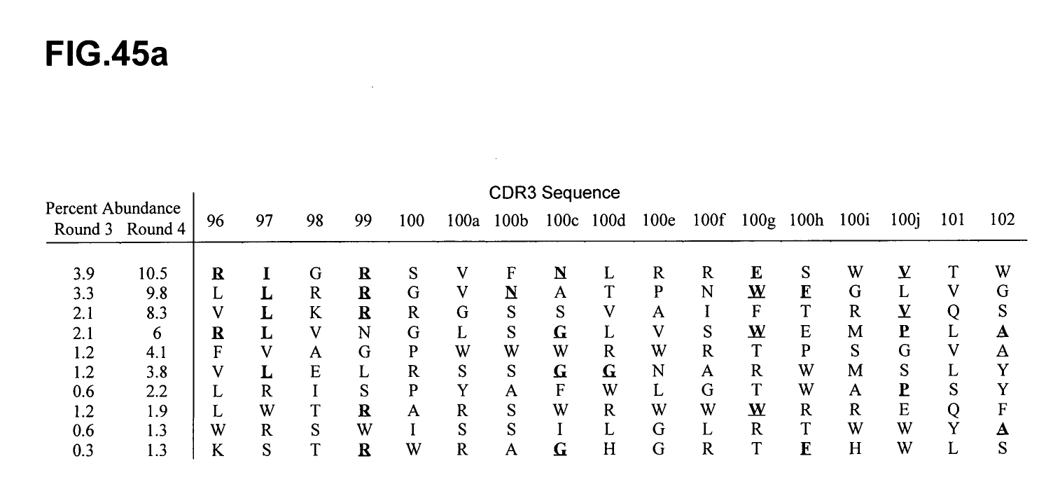

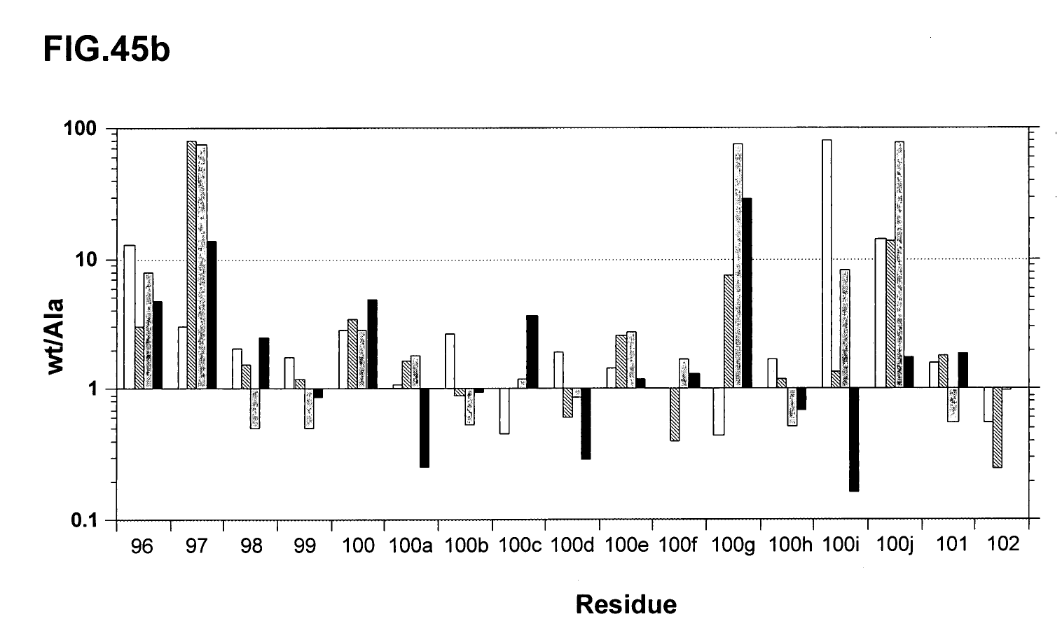

The sequence distribution of CDRH3 converges at rounds 4 and 5 to a smaller set of multiply represented sequences (FIG. 45a). FIG. 45 lists the top 10 sequence families after 4 rounds of panning. These top 4 sequences accounted for more than one-third of this population. While each of these sequences has some of the amino acid preferences as shown in FIG. 43b, none of these 10 sequences completely recapitulates the bias seen in the aggregate analysis of round 3. In the case of the most dominant clone “RIG”, which occurred 33 times in 324 sequences, Arg occurs at positions 96 and 99 in CDRH3 consistent with the earlier analysis. However, at position 97, a leucine in the aggregate analysis, is isoleucine in this clone, a conservative change. Position 100h normally a Phe is filled by a serine in this clone, while the amino acid at position 100j is a Val consistent with the aggregate analysis.

### EXAMPLE 9

### Alanine Shotgun Analysis

We also examined whether perturbations to four of the top 10 sequences (shown in FIG. 45a) affected scaffold stability by systematically substituting alanine for the parental residue at every position along CDR3 in each of these 4 scaffolds. To do this, we used alanine shotgun scanning in which a library is made where at every position one allows either the parental residue or alanine. The resulting library was sorted against Protein A. Protein A selection is used as a readout of stability and expression.

This technique assumes that residues integral to the stability of the scaffold will not be tolerant to ala substitution, thus there will be a low occurrence of alanine at that position after sorting against Protein A. Alternately, if the residue in question is not an important determinant in stability we would expect little or no effect on substitution to alanine, and as such we should see equivalent numbers of parent residues and alanine at that position.

Each of the 4 libraries included a 17 amino acid peptide with the same boundaries as for the NNS library. Equimolar DNA degeneracies are represented in the 1 UB code (M=A/C, N=A/C/G/T, R=A/G, S=G/C, W=A/T, Y=C/T)

| | |
|---|---|
| RIG ala scan library | |
| 5′- | (SEQ ID NO:155) |
| GCCGTCTATACTTGTGGTGCTGGTSSTRYTGSTSSTKCCGYTKYTRMCSYTS | |
| STSSTGMAKCCKSGGYTRCTKSGTGGGGTCAGGGT-3′, | |
| VLK ala scan library | |
| 5′- | (SEQ ID NO:156) |
| GCCGTCTATACTTGTGGTGCTGGTGYTSYTRMASSTSSTGSTKCCKCCGYTG | |
| STRYTKYTRCTSSTGYTSMAKCCTGGGGTCAGGGT, | |
| LLR ala scan library | |
| 5′- | (SEQ ID NO:157) |
| GCCGTCTATACTTGTGGTGCTGGTSYTSYTSSTSSTGSTGYTRMCGCGRCTS | |
| CARMCKSGKYTGSTSYTGYTGSTTGGGGTCAGGGT-3′, | |
| and the RLV ala scan library \ | |
| 5′- | (SEQ ID NO:158) |
| GCCGTCTATACTTGTGGTGCTGGTSSTSYTGYTRMCGSTSYTKCCGSTSYTG | |
| YTKCCKSGGMARYGSCASYTGCGTGGGGTCAGGG-3′ | |

Each of the 4 parent scaffolds showed a distinct pattern of residues tolerant to substitution (FIG. 45b). The amino acid positions sensitive to substitutions were distinct in each of the 4 scaffolds. In all four cases, the regions the most sensitive to substitution were at the N and C termini of the peptide while the central portion was in general more accepting of sequence perturbations.

In general, the patterns of wt sequence conservation were in good agreement with the consensus obtained from the aggregate analysis ( See FIGS. 45a and 45b). While each of the top 10 sequences had elements of the amino acid distribution observed in the aggregate analysis, there were significant differences, both amongst the top clones and in comparison with the aggregate consensus. This is not surprising since the aggregate consensus represents the average characteristics of several hundred clones, while each of the top clones represents a particular solution to the stabilization of the V_{H}H domain fold. It is notable that, while Trp was highly abundant throughout the loop in the aggregate analysis, the top four clones are not rich in Trp. Leucine and isoleucine were the aggregate consensus at position 97, and this side chain was highly conserved in comparison with Ala amongst all four scaffolds (FIG. 45b). (scaffolds are named after the sequence at positions 96, 97, and 98). Scaffolds VLK, LLR, and RLV all contain either a Trp/Phe at position 100 g, which in some cases appears to be intolerant to ala substitution. In contrast, the RIG scaffold contains a Glu at this position and for this scaffold this position does not appear to be as important structurally based on the shotgun scan. However, the RIG scaffold exhibited high conservation of a Trp100i residue in the alanine-scan, and thus, this residue may play a structural role that is similar to the role of Trp/Phe100g in the other scaffolds. The RIG and VLK scaffolds also showed conservation of Val100j in the alanine-scan, and this also agreed with the aggregate consensus. The only notable disagreement between the aggregate consensus and the individual alanine-scanning data occurs at position 99 where an Arg occurs in both the aggregate consensus and 3 of the top 4 scaffolds, and yet, Arg99 was not conserved in comparison with Ala in any of the shotgun scans.

These results indicate that amino acids located at the N and C-terminus of CDRH3 should be less diversified than other amino acids. Structural amino acid positions were identified as those positions that had a ratio of wild type amino acid to alanine of at least about 3 to 1 ,5 to1, 8 to 1,or greater or more preferably, about 10 to 1 or greater. The structural amino acid positions identified in the analysis include the first two N-terminal amino acid positions (positions 96 and 97 in this example) and one or more of the last 6 amino acid positions located at the C-terminus in the 17 amino acid peptide of CDRH3 (positions 100g, 100h, 100i, 100j, 101 and 102).

Physical Characteristics of Scaffolds

Each of the top 4 scaffolds expressed well in E. coli periplasmic expression systems. All 4 were monomeric as determined by size exclusion chromatography (data not shown).

TABLE 1
Physical characteristics of the scaffolds.

| Scaffold | Tm (C) | Protein A Affinity (uM) |
|---|---|---|
| Wild Type | 57 | 1.6 |
| RIG | 62 | 0.8 |
| VLK | 56 | 0.9 |
| LLR | 59 | 3.3 |
| RLV | 62 | 1.8 |

In addition, each of the 4 were as stable or more stable than the wild type anti-HCG scaffold used as a template. The melting curves from between 30 and 80 degrees celsius were fully reversible and indicated a two state folding transition (data not shown). Affinity of each of the scaffolds for Protein A was essentially as wild type as measured by BiaCore.

### EXAMPLE 10

### Stability of Ala Point Mutants

To ascertain how representative the shotgun data was in the context of soluble protein we made a series of ala point mutants of the RIG scaffold. 4 residues were chosen; positions 96 (Arg), 100(Ser), 100i(Trp) and 100j(Val). These four were chosen both to reflect a reasonable dynamic range as measured by the shotgun data, and to mirror those positions which were significant in the aggregate analysis of the NNS library. All four of the RIG ala mutants expressed well in E. coli and were monomeric as determined by size exclusion chromatography. The measured thermal stabilities were consistent with the shotgun analysis ( Table 2). Tryptophan 100i which was expected to be the most destabilizing mutant based on the shotgun data lowered the melting temperature by 10 degrees.

TABLE 2
Physical Characteristics of RIG alanine point mutants.

| | | | Reversible | Protein A |
|---|---|---|---|---|
| Mutant | WT/ala | Tm | Folding | affinity (μM) |
| RIG-Parent | N/A | 62 | yes | 0.8 |

Scaffold

| | | | | |
|---|---|---|---|---|
| R96A | 13 | ND | no | >10 |
| S100A | 2.8 | 60 | yes | 0.6 |
| W100iA | 79 | 51 | no | >10 |
| V100jA | 14 | 57 | yes | 0.8 |

ND value could not be determined.

W100iA was predicted to be the most destabilizing mutation, and indeed, this mutation abolished the reversible folding behavior seen in the wt RIG scaffold and reduced the apparent Tₘ by 10° C. The mutation R96A also abolished the reversible denaturation profile, and in this case, the apparent Tₘ could not be determined. The V100jA mutant retained a reversible denaturation profile, but the Tₘ was reduced by 5° C. In contrast, the S100A mutant exhibited a Tm almost indistinguishable from that of the wt RIG scaffold and also exhibited reversible denaturation behavior.

Protein A Binding Affinity of Point Mutants

In addition to thermal stability, we also measured the affinity of each of the alanine mutants for Protein A (Table 2). For two of the four mutants, S100A, and V100jA, the affinity was approximately wild type. However, both the R96A and W100iA mutants showed drastically attenuated binding affinities.

Results

Taken together, these data indicate that the mutations R96A and W100iA are extremely destabilizing for the structural integrity of the RIG V_{H}H domain, as they severely compromise both thermal stability and protein A affinity. The residue Val100j contributes more modestly to structural stability, as evidenced by a moderate decrease in thermal stability as a consequence of the V100iA mutation.

Finally, we directly perturbed the Protein A binding site in the RIG scaffold to ensure against the unlikely possibility that the selection process had generated CDR3 sequences with affinity for protein A. In classical VH_{H3} domains, the mutation T57E abolishes affinity for protein A, and Thr57 is conserved in the α-HCG sequence. The mutation T57E was introduced into the RIG scaffold, and we could not detect any binding interaction between the mutated protein and protein A by Biacore analysis. (data not shown) The CD spectra of the mutated protein was indistinguishable from that of the wt, indicating that the molecule was well-folded (data not shown).

The results presented here clearly indicate the added structural role of CDRH3 in camelid monobodies requires that one clearly delineate the structural residues for any given camelid scaffold. In addition, a scaffold is selected for which there is a contiguous stretch of CDRH3 residues tolerant to substitution.

We have provided a method for the identification of structural residues in Vh3 immunoglobulin domains. We have exploited the natural affinity of Protein A for a Vh3 domain and used it as a readout of scaffold stability and expression in combinatorial phage libraries. This approach has allowed us to rapidly screen over 10¹⁰ potential scaffolds and rapidly identify 4 potential scaffolds.

Each of the 4 scaffolds has the bimodal distribution of structural residues at the N and C termini. At the C terminal end, there is a strong dependence for either hydrophobic or aliphatic residues at the first three positions. The exact location of these residues is scaffold dependent, which is presumably because each of these scaffolds solves the interface ‘problem’ in a slightly different manner. The trend towards hydrophobic amino acids is consistent with the idea that CDRH3 residues might pack against the former light chain interface to form a small hydrophobic core that stabilized the VₕH domain fold. In the aggregate analysis (FIG. 43b), the N terminal end of CDRH3 has a consensus R(L/I/M)XR sequence. These residues may also play a role in stabilizing the structure.

We decided to test whether, in individual clones from the initial NNS library, there were specific residues in CDRH3, which had a significant impact on scaffold stability. We chose the 4 most represented sequences after 4 rounds of sorting and systematically changed every residue in CDRH3 to alanine and asked whether this perturbation affected expression. Using traditional methods, this approach would have required making 68 individual point mutants and measuring the resulting thermal stability, a tenable but tedious experiment which would have taken several months. Instead, we used a combinatorial technique—alanine shotgun mutagenesis (Weiss, G. A., Watanabe, C. K., Zhong, A. Goddard., Sidhu, S. (2000) Proc. Natl. Acad. Sci., 97(16), 8950-8954)—to assay all 17 residues in each scaffold in parallel. In this approach, degenerate oligos are used for mutagenesis where for every residue codons are chosen such that either the parent residue or alanine is allowed. By sorting the resulting libraries against Protein A, we were able to rapidly map out the structural residues of CDRH3 in 4 scaffolds. This approach allowed us to determine which residues were tolerant to substitution and could therefore be varied in a library and which were sensitive to substitution and needed to be substituted with a smaller set of amino acids.

In each scaffold, several residues near the boundaries of CDR3 were highly conserved in comparison with Ala, indicating that these side chains contributed significantly to stability. The accuracy of these predictions was directly confirmed for the RIG scaffold; three side chains (Arg96, Trp100i, and Val100j) were predicted to be important for stability by shotgun alanine-scanning, and an Ala substitution at each of these sites significantly reduced the thermal stability of point-mutated proteins (Table 2).

Comparing the pattern of structural residues in each of the 4 clones, a scaffold was selected to use in a library. From the perspective of a library, we wanted a scaffold in which the structural residues of CDRH3 were clustered near the ends of the peptide allowing for a long contiguous stretch of residues tolerant to variation in the central portion. The RIG clone was selected. Alanine substitution of parent residues in this clone attenuated expression by greater than tenfold when they occurred in the first two residues and at positions 100i and 100j. Thus, the long stretch of residues between 98 and 100i could be varied without any undue structural consequences.

While the shotgun approach allows for a rapid analysis of many potential scaffolds, stabilities of individual mutants were not measured directly and often times multiple alanine substitutions occur within one clone. As a check on the validity of the technique, we also made a series of 4 point mutants for the RIG clone and measured the resulting stability, and Protein A binding affinity. As shown in Table 2, the melting temperatures of the individual point mutants was consistent with the results of the shotgun data.

These results indicate that amino acid positions at the N and C termini of the 17 amino acid CDRH3 region are more sensitive to substitution and are likely to play a structural role in a monobody. The alanine scanning mutagenesis identifies structural amino acid positions that result in reduced structural stability when alanine is present at that position. The amino acids substituted at these positions should be limited in diversity to provide for structural stability of the variant monobodies.

Despite great differences in length and sequence, the CDR3s of both the natural anti-HCG and the in vitro-evolved RIG V_{H}H domains are utilized in similar mechanisms to stabilize the protein fold. In each case, a Trp residue near the C-terminus of the loop packs against the framework residue Phe37 to shield the former light chain interface from solvent, and these interactions appear to be influence protein stability. The stability of the RIG V_{H}H domain fold is also dependent upon an additional Arg residue near the N-terminus of CDR3, and it is possible that the hydrogen bonding interactions between Arg96 and residues in CDR1 provide additional stabilization energy that compensates for the entropy introduced by the extremely long CDR3 loop.

The top 4 V_{H}H domains all possess features which should make them ideal for the display of synthetic CDR3 libraries. The soluble proteins are monomeric and stable, and they exhibit reversible folding kinetics. Furthermore, protein stability is independent of the sequence within the central region of CDR3, and thus, it should be possible to present long, randomized loops without compromising the structural integrity of the scaffold. Based on the similar location and chemical nature of the CDR3 residues that are required for stability, it is likely that the four scaffolds employ similar structural strategies to shield the former light chain interface.

### EXAMPLE 11

### Peptide Length Dependence

In designing an antibody scaffold for naive or synthetic libraries one must ask to what extent peptide length is tolerated. This is especially the case in CDRH3 of camelid antibodies since they are on average significantly longer than traditional antibodies. As well, there is evidence from the germline that intramolecular disulfides between CDRH3 and the framework are exploited in stabilizing CDRH3 conformations. To determine the range of CDRH3 peptide lengths tolerated in our RIG clone we generated a phage library in which peptides from 10 to 15 residues were inserted. The functional CDRH3 boundary, between which the peptide was inserted, was based on the shotgun analysis and ala point mutants, which indicated the structural residues of CDRH3 in the RIG clone were at the N and C termini. More specifically, the N-terminal boundary was just after Ile 97 and the C-terminal boundary just before W100i.

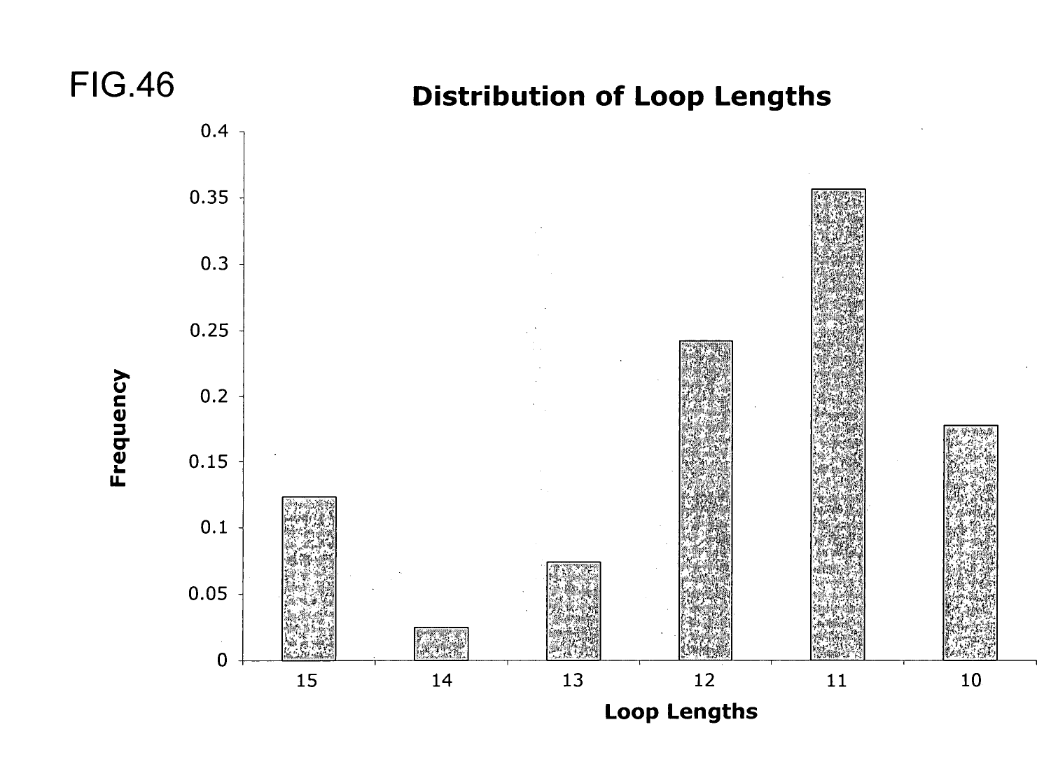

Each of the 7 libraries had nearly equivalent diversity after electroporation into SS320 cells (data not shown). Equivalent numbers of phage from each peptide length library were combined and sorted for two rounds against Protein A. At round 2, the display level was 96%. 202 clones were sequenced after round 2. In general the RIG scaffold was tolerant to insertion of peptides from between 10 to 15 residues in length. However there was a bias toward shorter peptide lengths (FIG. 46). Peptides of 11 residues were the most widely occurring. But there were a significant number of even 15 residue insertions which were tolerated. The broad distribution of peptide lengths and apparent lack of amino acid bias (data not shown) indicates that the structural role of CDR3 has been satisfied by the appropriate choice of boundary residues as discussed in the previous section.

Taken together the results of the NNS library, shotgun analysis, and peptide length library illustrate a new methodology in the design of antibody scaffolds for library design. Protein A selection allows for the elucidation of the structural and functional boundaries in CDR3. The accurate definition of functional boundaries should facilitate the rapid design of antibody scaffolds.

### EXAMPLE 12

### Generation of a Library of Variable Domains Using the RIG Scaffold

We next generated a library of variable domains using a RIG scaffold and varying the CDR3. The RIG scaffold was identified in EXAMPLE 8 and is shown in FIG. 45. In these studies, the CDR1 and CDR2 were not varied and were either from the native anti-HCG antibody or the human germline sequences from Dp47 because these human germline sequences were most similar to the native CDR1 and CDR2 sequences. The CDR3 region was fixed at the N-terminus with R—I— (amino acid positions 96, 97) and at the C-terminus with W—V (amino acid positions 100i and 100j). The loop in the middle was 11 amino acids long and varied randomly with all 20 known amino acids using NNS oligonucleotides as described in Example 9. Clones were selected by panning for binding to VEGF as described herein in the previous Examples. Clones were then sequenced and analyzed for bias for a particular amino acid at any position in the loop.

Design of a Heavy Chain VEGF Antibody

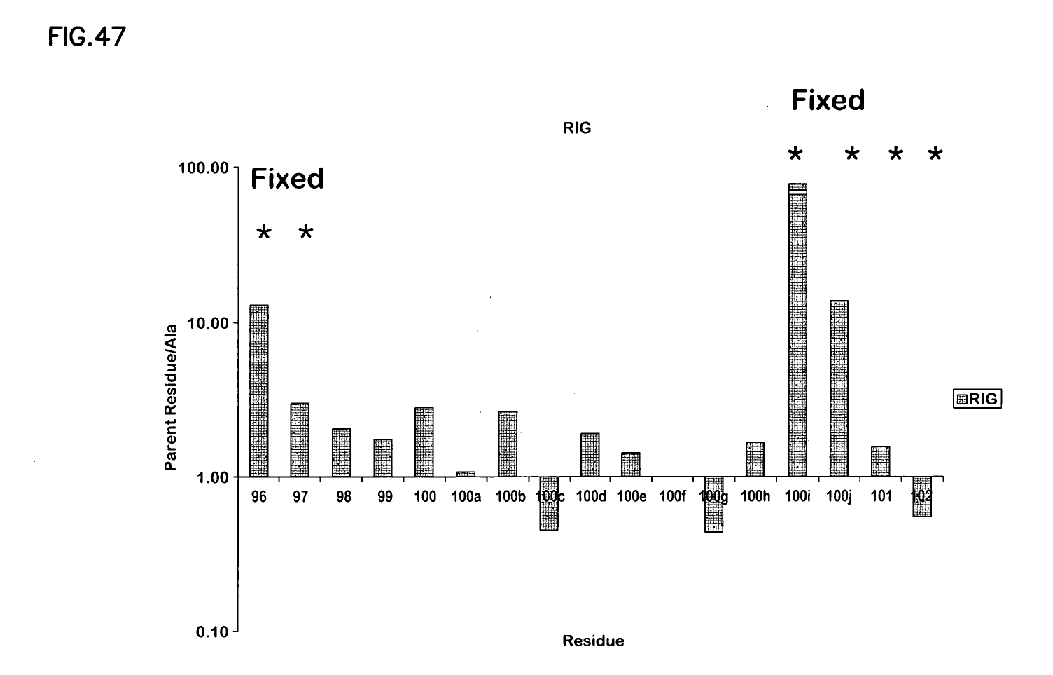

The RIG scaffold was used as a starting template in the design of a naïve antibody library. Residues 96, 97,100i, and 100j identified as structural in both the shotgun alanine scan and by x-ray crystallography (see EXAMPLE 9 and FIG. 45) were fixed as the boundaries of CDR3. A random 11-residue library was inserted between these fixed boundaries. See FIG. 47. The resulting library was sorted against human VEGF (see below).

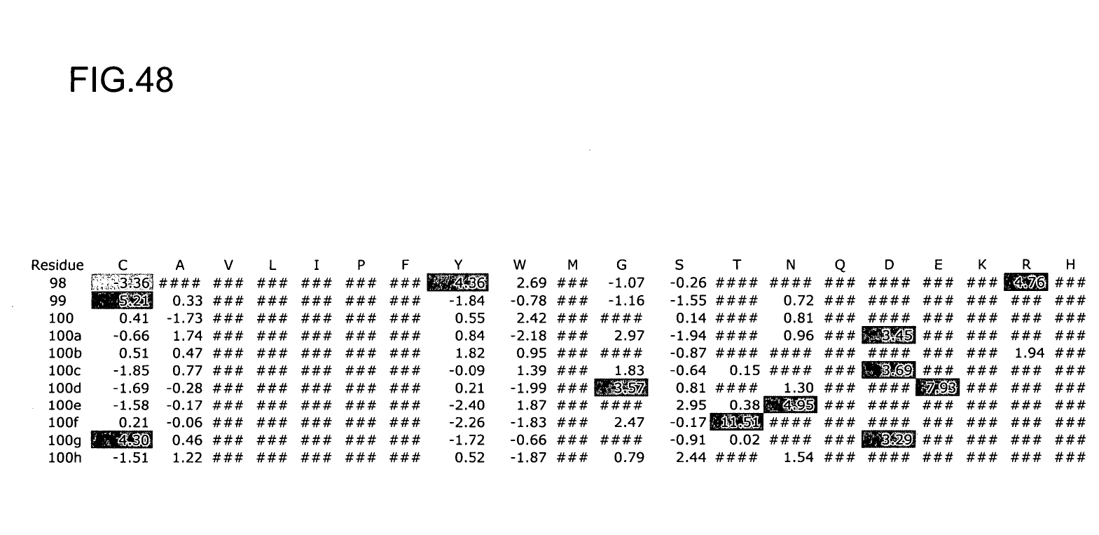

After three rounds of sorting, the distribution of amino acids in VEGF positive clones was assessed. As depicted in FIG. 48, individual clones were sequenced and residues that were statistically (p<0.05) over (dark gray) or under (light gray) represented were determined by Pearson analysis as described previously. This analysis indicated a strong preference for cysteine at two positions along the loop, residues 99 and 100h, the fourth from the N-terminus and fifth residue from C-terminus of the CDR3 region, respectively. Inspection of individual clones indicated that these cysteines appeared together, implying the formation of a disulfide constrained loop.

A subsequent library was made using the RIG scaffold and incorporating the cysteine residues at positions 99 and 100h of the CDR3 into the design. The N terminal amino acids were now R—I—X—C (residues corresponding to amino acid positions 96,97,98 and 99) and the C terminal amino acids were C—W—V-T-W (residues corresponding to amino acid positions 100h, 100i, 100j, 101 and 102). A loop of 6-7 amino acids between the N and C terminal ends were varied randomly.

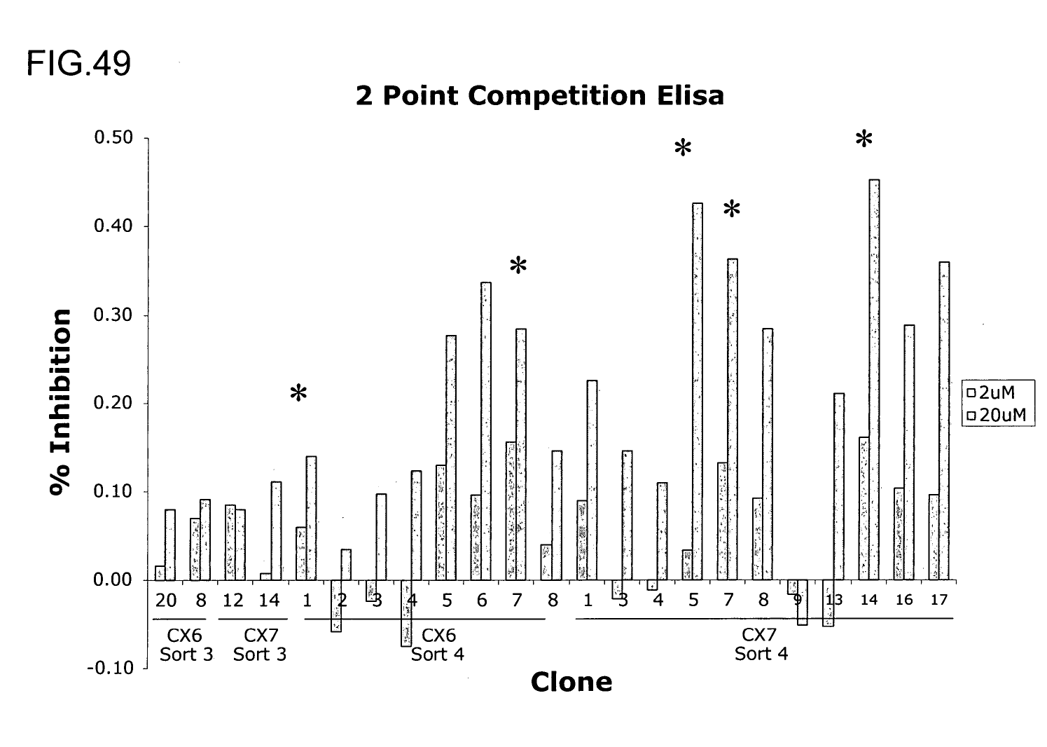

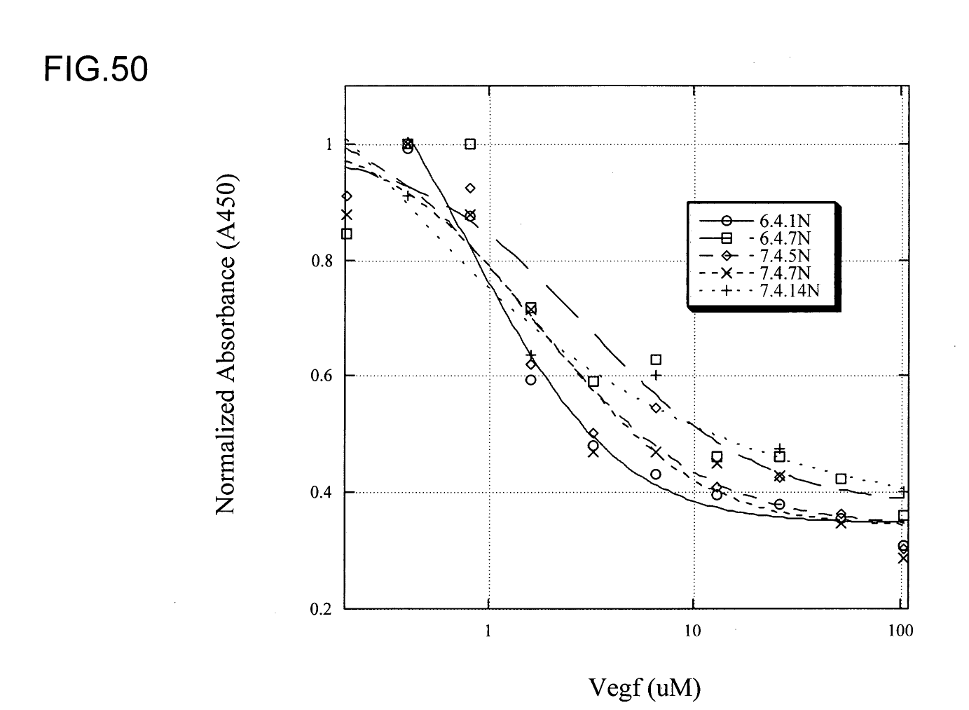

Individual clones of this library were analyzed for binding to VEGF using a competition ELISA with two concentrations, 2 μm and 20 μm. See FIG. 49. Those clones showing the ability to compete out VEGF at both concentrations are shown with asterisk. Based on the results of the 2 point competition elisa, five clones were selected that demonstrated appreciable binding to VEGF as determined by the elisa signal at 0 mM soluble VEGF and for which this activity was attenuated, in a dose dependent manner, by the two concentrations of soluble VEGF. The IC50 of these clones was determined from a full completion elisa with concentrations of soluble VEGF ranging from 0.1-100uM (FIG. 50). The results demonstrated that relatively high affinity binders in the low um range could be isolated from a library using RIG scaffold and varying a 6-7 amino acid loop in the CDR3.

These results show that the RIG scaffold as modified by incorporating cysteines near the N and C terminal ends of the CDR3 can be used to generate a library that provides high affinity binders for antigens such as VEGF. Variation in the CDR3 region is accomplished by randomizing a 6-7 amino acid loop between the cysteines and provides for minimizing the number of amino acid residues in the CDR3 that are targeted for diversity while still maintaining structural stability. Libraries having diversity in CDR1 and CDR2 may be also be designed and prepared and combined with diversity in the CDR3 region to further enhance the ability to isolate high affinity antigen binders from the library.

### EXAMPLE 13

### Crystal Structure of RIG Heavy Chain Scaffold

The crystal structure of the RIG scaffold with the native or parent CDR3 sequence was determined and analyzed in order to validate studies showing that CDR3 residues in VhH at positions W100i and V100j interact with framework residues F37 and R45 to stabilize the heavy chain in the absence of the light chain.

Protein expression and purification was done as previously described. Protein crystals were grown in 30% PEG 4K, 0.3 M Ammonium Sulfate, pH. 7.0 at 20° C. A molecular replacement solution was found using the published anti-HCG V_{H}H domain structure minus residues 96-102, as a search model (pdb accession code 1HCV). The initial molecular replacement solution underwent several rounds of model building and anisotropic TLS refinement. The molecular graphics program O was used for model building and Refmac5 was used for the refinement. Diffraction data was collected at the APS syncotron beamline 19. Structures were rendered in Pymol (DeLano Scientfic, San Carlos, Calif.).

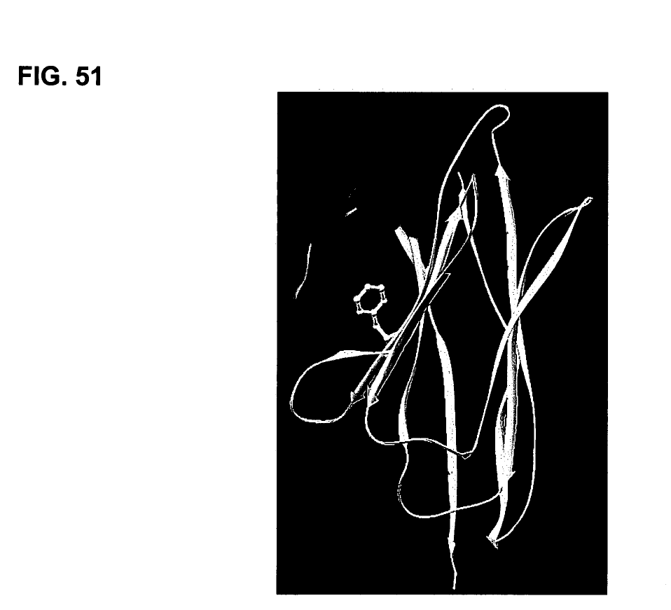

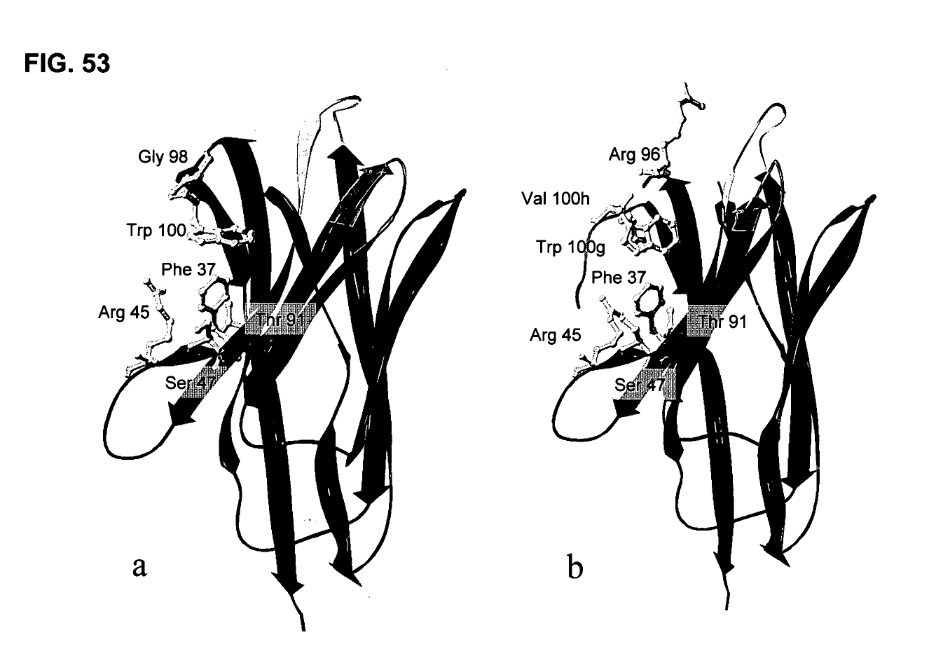

The overall tertiary structure (FIG. 51, FIG. 53) of the RIG scaffold does not differ from the parent anti-HCG scaffold. (Compare FIG. 39, FIG. 53). However, only residues 96, 97, and 100f-102 were well ordered. The central portion of CDR3 is essentially unstructured, which is consistent with the shotgun alanine scanning results described above.

The RIG crystals diffracted to 1.9 Angstrom resolution and the resulting structural model agreed well with the observed data set as confirmed by the finial refinement statistics 9 R=%20 and R-Free=%26.

TABLE 3
Data Collection and Refinement Statistics
A. Unit Cell

| Space Group | P2₁ |
|---|---|
| a(Å) | 34.21 |
| b(Å) | 120.71 |
| c(Å) | 52.25 |
| β(deg.) | 103.34 |
| Molecules per asymmetric unit | 8 |
| Solvent Content | %33 |

B. Diffraction Data

| | |
|---|---|
| Resolution (Å) | 20-1.9 |
| Total Number of Reflections | 30610 |
| Number of Reflections used for F-Free | 1635 (%5.1) |
| Completeness | 99.7 |
| <I/σ(I)> | 7.6 |

C. Refinement

| | |
|---|---|
| R_{work}^{a} | 20% |
| R_{free}^{a} | 26% |
| No. of protein atoms | 3592 |
| No. of water molecules | 328 |
| Rmsd bond length (Å) | 0.007 |
| Rmsd bond angles (deg.) | 1.016 |
| Rmsd torsion angles (deg.) | 4.911 |

^{a}R_{work} = Σ1Fo-Fc1/ΣFo where Fo and Fc are the observed and calculated structure factor amplitudes. R_{free} is the R factor for a randomly selected set (%5) of reflections not used in the refinement.

### EXAMPLE 14

### Framework Determinants of Scaffold Stability

In order to determine if any positional biases existed in the RIG framework, a framework scan was performed as described in EXAMPLE 7. Since some framework region sequences are also involved in maintaining the stability of the monobody, framework sequence changes can also impact the design of a monobody scaffold for use in synthetic libraries. Identification of framework region residues may also be important in designing a library that provides for diversity while minimizing structural perturbations. The RIG scaffold was used as the parent or wild type molecule for determining the effect of mutations at residue positions 37, 45, 47 and 91 and compared to the same type of study conducted with wild typ anti-HCG. (See EXAMPLE 7).

Results

A library of VhH monobodies with all 20 amino acids substituted at each of positions 37, 45, 47 and 91 in the framework region was generated using the methods of EXAMPLE 7. The library was sorted for the stability by binding to protein A. Several binders were isolated and sequenced. The sequences of the binders were analyzed for positional bias as described previously and compared to the wild type anti-HCG analysis as described in EXAMPLE 7.

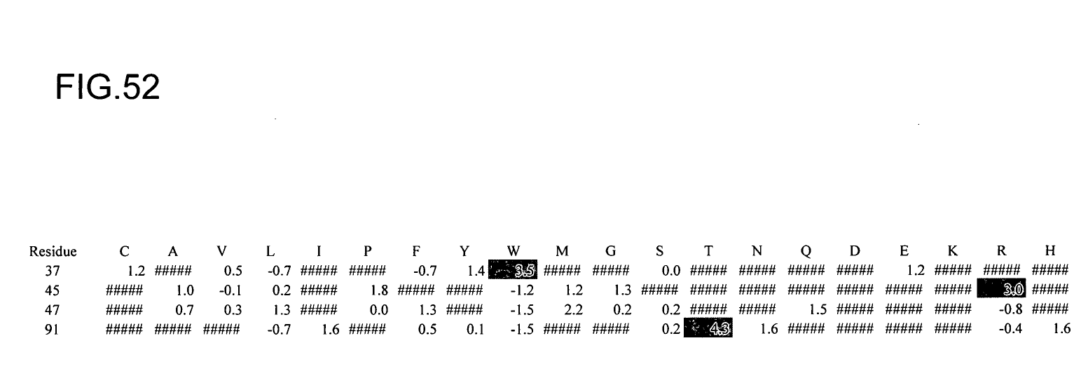

Position 37 was occupied almost exclusively by hydrophobic residues. Phe and Trp were the most prevalent amino acids in both V_{H}H domains, but the order of preference was inverted, as the anti-HCG domain was dominated by Phe (70%) while the RIG domain was dominated by Trp (55%). At position 45, the sequence distribution was more diverse but the wild-type Arg residue was clearly dominant in the anti-HCG domain (52%) and was the single most prevalent residue type in the RIG domain (20%). The preference for Arg45 most likely reflects the favorable interactions afforded by the amphipathic Arg side chain; the hydrophobic methylene carbons can pack against Phe37 at the hydrophobic core while the polar guanidino group can accommodate the aqueous solvent. Aside from Arg, both domains preferred hydrophobic residues at position 45 and the RIG domain in particular contained a substantial proportion of Trp, Phe and Leu residues. Overall, these results demonstrate that changes at positions 37 and 45 of V_{H}H domains relative to V_{H} domains contribute to protein stability, as they allow for favorable hydrophobic interactions amongst themselves and with CDR3. See FIG. 52

In contrast with positions 37 and 45, the residue types commonly found at positions 47 and 91 do not differ greatly between natural V_{H}H and V_{H} domains (FIG. 52). In V_{H}H domains, position 47 is commonly occupied by hydrophobic residues (Phe, Leu or Trp) or Gly residues, while this position is almost exclusively occupied by Trp in V_{H} domains.

In summary, selection for folded V_{H}H domains favors hydrophobic character at positions 37 and 45 which are occupied by hydrophobic residues in natural V_{H}H domains. In addition, it may be possible to further stabilize the anti-HCG and RIG V_{H}H domain folds by converting the small hydrophilic residues at the nearby framework positions 47 and 91 to the types of hydrophobic residues commonly found at these positions in natural V_{H}H and V_{H} domains. However, it should be noted that the selection experiments were conducted with phage-displayed V_{H}H domains at extremely low concentrations, and it is possible that the introduction of additional hydrophobicity at the former light chain interface may lead to aggregation at high protein concentrations.

All publications (including patents and patent applications) cited herein are hereby incorporated in their entirety by reference.

1. A polypeptide comprising a variant CDRH3 region, wherein the CDRH3 region comprises:
a) at least one structural amino acid position, wherein said structural amino acid position has a variant amino acid, wherein the variant amino acid is an amino acid found at that position in a randomly generated CDRH3 population at a frequency of at least one standard deviation above the average frequency for any amino acid at that position; and
b) at least one non-structural position, wherein the non-structural position has a variant amino acid.

2. A variable domain of a monobody comprising a variant CDRH3 region, wherein the variant CDRH3 region comprises:
a) at least one structural amino acid position, wherein said structural amino acid position has a variant amino acid, wherein the variant amino acid is an amino acid found at that position in a randomly generated population at a frequency of at least one standard deviation above the average frequency for any amino acid at that position; and
b) at least one non-structural position, wherein the non-structural position has a variant amino acid.

3. The polypeptide according to claim 1, wherein the polypeptide is an antibody variable domain of the Vh3 subgroup.

4. The polypeptide according to claim 1, wherein said at least one non-structural position is a contiguous amino acid sequence of about 1 to 20 amino acids.

5. The polypeptide according to claim 1, wherein said at least one structural amino acid position is one or both the first two amino acid positions at the N-terminus of a heavy chain CDRH3.

6. The polypeptide according to claim 1, wherein said at least one structural amino acid position is at least one of the last 6 amino acids at the C-terminus of a heavy chain CDRH3.

7. The polypeptide according to claim 5, wherein the first N-terminal amino acid position has a variant amino acid is selected from the group consisting of R, L, and V.

8. (canceled)

9. The polypeptide according to claim 5, wherein the first amino acid position at the N-terminus has a variant amino acid selected from the group consisting of R, L and V, and the second amino acid position at the N-terminus is selected from the group consisting of I and L.

10. The polypeptide according to claim 6, wherein said at least one structural amino acid position is a third and/or fourth amino acid position from the C-terminus.

11. The polypeptide according to claim 10, wherein the fourth amino acid position from the C-terminus has a variant amino acid selected from the group consisting of M, R, G and W and the third amino acid position from the C-terminus has a variant amino acid selected from the group consisting of P, L, or V.

12. The polypeptide according to claim 6, wherein the at least one structural amino acid position is selected from the amino acid position 100g, 100h, 100i, 100j, 101, 102 of SEQ ID NO:137 and mixtures thereof.

13-14. (canceled)

15. The polypeptide according to claim 1, wherein said at least one non-structural position has a variant amino acid encoded by a non-random codon set.

16. The polypeptide according to claim 1, wherein the said at least one structural amino acid position is the first two N-terminal amino acid positions, and the third and fourth positions from the C-terminus of the CDRH3 region.

17. (canceled)

18. The variable domain according to claim 2, wherein amino acid position 37 of the framework 2 region is a hydrophobic amino acid.

19. The variable domain according to claim 18, wherein amino acid position 37 is phenylalanine or tryptophan.

20. The variable domain according to claim 18, wherein the amino acid position 45 of framework 2 is selected from the group consisting of arginine, tryptophan, phenylalanine and leucine.

21. A variable domain of claim 2, further comprising a heavy chain framework 3 region, wherein the amino acid position 91 of the framework 3 region is a phenylalanine tyrosine, or threonine.

22. The polypeptide of claim 1 which is a fusion polypeptide.

23. The polypeptide of claim 22 which is a fusion polypeptide fused to at least a portion of a viral coat protein.

24. The polypeptide of claim 23, wherein the viral coat protein is selected from the group consisting of p111, pv111, Soc, Hoc, 9pD, pV1 and variants thereof.

25. A polynucleotide molecule encoding a polypeptide of claim 1.

26. A replicable expression vector comprising a polynucleotide molecule of claim 25.

27. A host cell comprising the vector of claim 26.

28. A library comprising a plurality of vectors of claim 26, wherein the plurality of vectors encode a plurality of polypeptides.

29. A polypeptide comprising a variant CDRH3 region, wherein the CDRH3 region comprises:
a)______ a N terminal portion that comprises at least one structural amino acid position, wherein said structural amino acid position has a variant amino acid, wherein the variant amino acid is an amino acid found at that position in a randomly generated CDRH3 population at a frequency of at least one standard deviation above the average frequency for any amino acid at that position;
b)______ a central portion that comprises at least one non-structural position, wherein the non-structural position has a variant amino acid; and
c)______ a C-terminal portion that comprises at least one structural amino acid position, wherein said structural amino acid position has a variant amino acid, wherein the variant amino acid is an amino acid found at that position in a randomly generated CDRH3 population at a frequency of at least one standard deviation above the average frequency for any amino acid at that position.

30. The polypeptide according to claim 29, wherein the polypeptide is a heavy chain variable domain of a monobody.

31. The polypeptide according to claims 29, wherein said at least one non-structural position is a contiguous amino acid sequence of about 1 to 17 amino acids.

32. The polypeptide according to claims 29, wherein said at least one structural amino acid position is one or both the first two amino acid positions at the N-terminus of a heavy chain CDRH3.

33. The polypeptide according to claims 29, wherein said at least one structural amino acid position is at least one of the last 6 amino acids at the C-terminus of a heavy chain CDRH3.

34. The polypeptide according to claim 32, wherein the first N-terminal amino acid position has a variant amino acid is selected from the group consisting of R, L, and V.

35. (canceled)

36. The polypeptide according to claim 32, wherein the first amino acid position at the N-terminus has a variant amino acid selected from the group consisting of R, L and V, and the second amino acid position at the N-terminus is selected from the group consisting of I and L.

37. The polypeptide according to claim 32, wherein the N terminal portion is no more than 4 amino acids.

38. The polypeptide according to claim 33, wherein said at least one structural amino acid position is a third and/or fourth amino acid position from the C-terminus.

39. The polypeptide according to claim 38, wherein the fourth amino acid position from the C-terminus has a variant amino acid selected from the group consisting of M, R, G and W and the third amino acid position from the C-terminus has a variant amino acid selected from the group consisting of P, L, or V.

40. The polypeptide according to claim 33, wherein the at least one structural amino acid position is selected from the amino acid position 100g, 100h, 100i, 100j, 101, 102 of SEQ ID NO:137 and mixtures thereof.

41. (canceled)

42. The polypeptide according to claim 33, wherein the C-terminal portion is not more than 6 amino acids.

43. (canceled)

44. The polypeptide according to claim 29, wherein said at least one non-structural position has a variant amino acid encoded by a non-random codon set.

45. The polypeptide according to claim 29, wherein the central portion is no more than 20 amino acids.

46. The polypeptide according to claims 29, wherein the said at least one structural amino acid position is the first two N-terminal amino acid positions, and the third and fourth positions from the C-terminus of the CDRH3 region.

47. (canceled)

48. The variable domain according to claim 30, wherein amino acid position 37 of the framework 2 region is a hydrophobic amino acid.

49. The variable domain according to claim 48, wherein amino acid position 37 is phenylalanine or tryptophan.

50. The variable domain according to claim 30, wherein the amino acid position 45 of framework 2 is selected from the group consisting of arginine, tryptophan, phenylalanine and leucine.

51. A variable domain of claim 30, wherein the amino acid position 91 of the framework 3 region is a phenylalanine, tyrosine or threonine.

52. The polypeptide of any of claim 29 which is a fusion polypeptide.

53. The polypeptide of claim 52 which is a fusion polypeptide fused to at least a portion of a viral coat protein.

54. The polypeptide of claim 53, wherein the viral coat protein is selected from the group consisting of p111, pv111, Soc, Hoc, 9pD, pV1 and variants thereof.

55. A polynucleotide molecule encoding a polypeptide of claim 29.

56. A replicable expression vector comprising a polynucleotide molecule of claim 55.

57. A host cell comprising the vector of claim 56.

58. A library comprising a plurality of vectors of claim 56, wherein the plurality of vectors encode a plurality of polypeptides.

59. A polypeptide comprising a CDRH3, wherein the CDRH3 comprises an amino acid sequence having the formula of A₁-A₂-(A₃)ₙ-A₄-A₅; wherein
A₁ is an amino acid selected from the group consisting of R, L, V, F, W and K;
A₂ is an amino acid selected from the group consisting of I, L, V, R, W and S;
A₃ is any naturally occurring amino acid and n can be 1-17;
A₄ is an amino acid selected from the group consisting of W, G, R, M, S, A and H;
A₅ is an amino acid selected from the group consisting of V, L, P, G, S, E and W.

60. A polypeptide comprising a CDRH3, wherein the CDRH3 comprises an amino acid sequence having the formula of A₁-A₂-(A₃)ₙ-A₄-A₅-A₆-A₇; wherein
A₁ is an amino acid selected from the group consisting of R, L, V, F, W and K;
A₂ is an amino acid selected from the group consisting of I, L, V, R, W and S;
A₃ is any naturally occurring amino acid and n can be 1-17;
A₄ is an amino acid selected from the group consisting of W, G, R, M, S, A and H;
A₅ is an amino acid selected from the group consisting of V, L, P, G, S, E and W; and
A₆ and A₇ are any naturally occurring amino acid.

61. A variable domain of a monobody comprising a CDRH3, wherein the CDRH3 comprises an amino acid sequence having the formula of A₁-A₂-(A₃)ₙ-A₄-A₅-A₆-A₇; wherein
A₁ is an amino acid selected from the group consisting of R, L, V, F, W and K;
A₂ is an amino acid selected from the group consisting of I, L, V, R, W and S;
A₃ is any naturally occurring amino acid and n can be 1-17;
A₄ is an amino acid selected from the group consisting of W, G, R, M, S, A and H;
A₅ is an amino acid selected from the group consisting of V, L, P, G, S, E and W; and
A₆ and A₇ are any naturally occurring amino acid.

62. The polypeptide according to claim 59, wherein
A₁ is R;
A₂ is I;
A₄ is W;
A₅ is V; and
n=11.

63. The polypeptide according to claim 59, wherein
A₁ is L;
A₂ is L;
A₅ is L; and
n=11.

64. The polypeptide according to claim 59, wherein
A₁ is V;
A₂ is L;
A₄ is R;
A₅ is V; and
n=11.

65. The polypeptide according to claim 59, wherein
A₁ is R;
A₂ is L; and
n=11.

66. The polypeptide according to claim 59, wherein n is 9 to 12.

67. (canceled)

68. The polypeptide according to claim 59, wherein the amino acid or amino acids of A₃ are encoded by a nonrandom codon set.

69. A polypeptide comprising a CDRH3, wherein the CDRH3 comprises an amino acid sequence having the formula of A₁-A₂-(A₃)ₙ-A₄-A₅-A₆-A₇-A₈-A₉; wherein
A₁ is an amino acid selected from the group consisting of R, L, and V;
A₂ is an amino acid selected from the group consisting of I, L, and V;
A₃ is any naturally occurring amino acid and n=1-17;
A₄ is an amino acid selected from the group consisting of E, W, and F;
A₅ is any naturally occurring amino acid;
A₆ is an amino acid selected from group consisting of W, G, R, and M;
A₇ is an amino acid selected from the group consisting of V, L, and P; and
A₈ and A₉ are any naturally occurring amino acid.

70. The polypeptide according to claim 69, wherein the polypeptide is a variable domain of a monobody.

71. The polypeptide according to claim 70, wherein
A₁ is R;
A₂ is I;
A₆ is W;
A₇ is V; and
n=9.

72. The polypeptide according to claim 70, wherein
A₁ is L;
A₂ is L;
A₄ is W;
A₅ is L; and
n=9.

73. The polypeptide according to claim 70, wherein
A₁ is V;
A₂ is L;
A₄ is F;
A₆ is R;
A₇ is V; and
n=9.

74. The polypeptide according to claim 70, wherein
A₁ is R;
A₂ is L;
A₄ is W; and
n=9.

75. (canceled)

76. The polypeptide according to claim 70, wherein A₃ is encoded by a nonrandom codon set.

77. A polynucleotide molecule encoding a polypeptide of any of claim 59.

78. A replicable expression vector comprising a polynucleotide molecule of claim 77.

79. A host cell comprising the vector of claim 78.

80. A library comprising a plurality of vectors of claim 78, wherein the plurality of vectors encode a plurality of variant polypeptides.

81. A method of generating a polypeptide comprising a variant CDRH3, wherein said polypeptide is capable of binding a target molecule of interest, said method comprising:
a) identifying at least one structural amino acid position in CDRH3; and
b) replacing the amino acid at said at least one structural amino acid position with a variant amino acid found at that position in a population of polypeptides with randomized CDRH3 at a frequency at least one standard deviation above the average frequency for any amino acid at that position; and
c) replacing at least one non-structural amino acid position with a variant amino acid, wherein the variant amino acid is any of the naturally occurring amino acid or is encoded by a nonrandom codon set.

82. The method according to claim 81, wherein identifying at least one structural amino acid position comprises:
a) generating a population of variant CDRH3 regions from a parent CDRH3 by replacing each amino acid position in the CDRH3 with a scanning amino acid; and
b) identifying a structural amino acid position in the CDRH3 as an amino acid position that when substituted with a scanning amino acid, the substituted polypeptide has a decrease in binding with a target molecule as compared to the parent CDRH3 region, wherein the target molecule specifically binds to a folded polypeptide and does not bind to unfolded polypeptide.

83. The method according to claim 81, wherein identifying at least one structural amino acid position comprises:
a) generating a population of polypeptides with randomly generated variant CDRH3 regions, wherein each amino acid position in the variant CDRH3 regions is randomized;
b) selecting members of the population that interact with a target molecule, wherein the target molecule specifically binds to a folded polypeptide and does not bind to an unfolded polypeptide;
c) determining the sequence of the selected members; and
d) identifying a structural amino acid position as a position that when substituted with a scanning amino acid the substituted polypeptide has a decrease in binding with the target molecule as compared to polypeptide with parent CDRH3 region.

84. The method according to claim 81, wherein the polypeptide is a variable domain of a camelid monobody.

85. A polypeptide prepared according to the method of claim 81.

86-89. (canceled)

90. A variable domain of a monobody comprising a CDRH3, wherein the CDRH3 comprises an amino acid sequence having the formula of R-A₂-A₃-R-(A₅)ₙ; wherein A₂ is L, I or M, A₃ and A₅ are any naturally occurring amino acid, and n=1 to 20.

91. A variable domain of a monobody comprising a CDRH3, wherein the CDRH3 comprises an amino acid sequence having the formula of R-A₂-(A₃)ₙ-W-A₅-A₆-A₇-A₈-A₉; wherein A₃, A₅, A₆, A₇, A₈ and A₉ are any naturally occurring amino acid and A₂ is L, I or M, and n=1 to 20.

92. A method for designing a CDRH3 scaffold comprising:
a) generating a library of polypeptides with variant CDRH3 regions;
b) selecting members of the library that bind to a target molecule that binds to folded polypeptide and does not bind to unfolded polypeptide;
c) analyzing the binders to identify structural amino acid positions in the CDRH3 region; and
d) selecting as a scaffold, a binder that has a structural amino acid position at the N and/or C-termini of the CDRH3 and not in a central position of the CDRH3.

93. The method according to claim 92, further comprising:
e) identifying an amino acid that can be substituted at the structural amino acid position, wherein the amino acid is selected from the group of amino acids that occur at that position more frequently than randomly expected;
f) forming a scaffold with at least one identified amino acid in at least one structural amino acid position.

94. The method according to claim 92, wherein the structural amino acid positions are selected from the group consisting of the first N-terminal amino acid, the second N-terminal amino acid and the last six C-terminal amino acid, and mixtures thereof.

95. The method according to claim 94, wherein the identified amino acids are selected from the group consisting of arginine, tyrosine, phenylalanine , tryptophan, and valine.

96. A polypeptide comprising a CDRH3, wherein the CDRH3 comprises an amino acid sequence having the formula of:
A₁-A₂-A₃-A₄-(A₅)ₙ-A₆-A₇-A₈-A₉-A₁₀
wherein A₁ is an amino acid selected from the group consisting of R, L and V;
A₂ is an amino acid selected from the group consisting of I, L and V;
A₃ is any naturally occurring amino acid;
A₄ is selected from the group consisting of C, R and N;
A₅ is any naturally occurring amino acid and n=1-16;
A₆ is an amino acid selected from the group consisting of C, S, F, T, E and D;
A₇ is an amino acid selected from the group consisting of W, G, R and M;
A₈ is an amino acid selected from the group consisting of V, L and P;
A₉ is an amino acid selected from the group consisting of T, V, L and Q; and
A₁₀ is an amino acid selected from the group consisting of W, G, S and A.

97. (canceled)

98. The polypeptide according to claim 97, wherein the polypeptide is a camelid monobody.

99. The polypeptide of claim 96, wherein
A₁ is R;
A₂ is I;
A₄ is C;
A₆ is C;
A₇ is W;
A₈ is V;
A₉ is T;
A₁₀ is W; and
n=7.

100-101. (canceled)

102. A polynucleotide molecule encoding a polypeptide of claim 96.

103. A replicable expression vector comprising a polynucleotide molecule of claim 102.

104. A library comprising a plurality of vectors of claim 103, wherein the plurality of vectors encode a plurality of variant polypeptides.

105. A CDRH3 scaffold comprising a N-terminal portion in which some or all of the positions are structural; and a C terminal portion in which some or all of the amino acid positions are structural, and wherein the scaffold can accommodate the insertion of a central portion or loop of contiguous amino acids that can vary in sequence and in length.

106. The CDRH3 scaffold of claim 105, wherein the N-terminal portion has a cysteine residue and the C terminal portion has a cysteine residue, wherein the cysteine residues in the N terminal and C-terminal portion of the CDRH3 scaffold form a disulfide bond that stabilizes the central portion insert, and wherein the central portion insert is a contiguous amino acid sequence of about 1 to 20 amino acids.

107. The CDRH3 scaffold of claim 105, wherein the N-terminal portion has a N terminal sequence of R-L/I/M-A₃-R, wherein A₃ is any naturally occurring amino acid, and wherein the central portion insert is a contiguous amino acid sequence of about 1 to 20 amino acids.

108. The CDRH3 scaffold of claim 105, wherein the N terminal sequence is R—I-A₃-C, wherein A₃ is any naturally occurring amino acid, and wherein the central portion insert is a contiguous amino acid sequence of about 1 to 20 amino acids.

109. The CDRH3 scaffold of claim 105, wherein the N terminal sequence comprises R—I, L-L, V-L, or R-L and wherein the central portion insert is a contiguous amino acid sequence of about 1 to 20 amino acids.

110. The CDRH3 scaffold of claim 105, wherein the C terminus has a sequence of CWVTW, and wherein the central portion insert is a contiguous amino acid sequence of about 1 to 20 amino acids.

111. The CDRH3 scaffold of claim 105, wherein C-terminal sequence comprises F—X—R—V, W—X—X-L, W—X-M-P, or W—V, wherein X can be any naturally occurring amino acid and wherein the central portion insert is a contiguous amino acid sequence of about 1 to 20 amino acids.

112. The CDRH3 scaffold of claim 105, wherein the N terminal portion is about 1 to 4 amino acids.

113. The CDRH3 scaffold of claim 105, wherein the C terminal portion is about 1 to 6 amino acids.

114. The CDRH3 scaffold of claim 105, wherein the central portion is a contiguous sequence of 9 to 12 amino acids.
